SEQUENCE LISTING (71 sequences)

SEQ ID NO: 1 - DNA, 696 bp, Homo sapiens
atgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcattttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtac

SEQ ID NO: 2 - DNA, 363 bp, Homo sapiens
atgcaggccctgcagcagcaaccagtttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctggctgactaaaggccacagctga

SEQ ID NO: 3 - DNA, 738 bp, Homo sapiens
atgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcattttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcagttgccctttggctgccagctaccaggcaccaggctcaaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactga

SEQ ID NO: 4 - DNA, 558 bp, Homo sapiens
atgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcatgctattttcttgaccccttcatgggactcccaacaggggtaccccatttactcagcctgccctgctcaacctcttgcaggagggagcacacgagtgaacgagtgcaggaaccagctggctgctttagtgctgtgaggagtaaactccatgcaggccctgcagcagcaaccagtttttccagatttgctcaaagcaatcccagtgagcatccacgtcaatgtcattctcttctctgccatccttattgtgttaaccatggtggggacagccttcttcatgtacaatgcttttggaaaaccttttga

SEQ ID NO: 5 - DNA, 46837 bp, Homo sapiens
aggagatacttgaaggcagtttgaaagacttgttttacagattcttagtccaaagatttccaattagggagaagaagcagcagaaaaggagaaaagccaagtatgagtgatgatgaggccttcatctactgacatttaacctggcgagaaccgtcgatggtgaagttgccttttcagctgggagctgtccgttcagcttccgtaataaatgcagtcaaagaggcagtcccttcccattgctcacaaaggtcttgtttttgaacctcgccctcacagaagccgtttctcatcatgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcatgtaagtagcaattgcatttgagttatttaatgctttaggcagactcttcccagtgttgcgaggaattatatttgagaattttgccgtgtttactgcaggactttttaaatcggtgtgaaccatatgaaaaacctatgactctgagcaatttcttcttcctagtttttattattttatacttgctttttattataatatagagttaattcattgttacataattaaggtttttggaaatattggcaattaagatgcttaagtattaatatttatgtaaaaaattatggagtctttttaaaaaagtaaacttggggaaataggaaagctgtaaagaatgatctttatgctttttgttctttataaaaagaaccaaggtcatgggctccgtatttaaccaggttgccacctttctcatgattttgtttcctgctccccactccctcccattattcctgctaagacctttcctgctgctaaatattcagttttcatttttaactaatttggaatcatttggctatagaaatttaaaatgatctgctgtgctaactgggaaagaaatggatgcctatttagtatagaacattttaaactgattgacctgcaaatcatgtagagaatatgagagagattttcttgttgtgatttttgtgaaatggaagtgtaatccacagtatttataacctgtttatcttaagaagagaatttttaaaaattaccatgtgaataggcaactcattaaatgaaaattaataggaagtcatttgttatatctcttacaacacacattcagaagttattattatttcagaagggctggtttggaacaaccttatgaagacacagtcagtaaattactgcataaatcactcttcaggaaaggaggttaccaactgaagcatttaaaatgaattattattttgcccaggttttttttttctttctagtataggtagaaggctaaattaattgaatttattattaacatatgcagtgcctaattaaatttcagtgctggtctatttatatttctgcaacattccttatatcttcttagcagtcattggacaccaaccttcagctcacataggttactaagtgatatgaattttcatagggctccagaaaatttccaagaattggttgttagctttttaattgatgaagtggataccagttcttttcactgaatggcttttattcattaaggtaatggggctgttagagttgcttagttttcctggggaaggggaaggaagaaaacaaagcagaatgtcatgtgatatgcaactgtattaaaaaaccgaaaaggaaaaaagttgagagagatgatttaaccgtgagtcaccggcagccaaagcgtgagtaaagcttctcacagatgaatttagacaaaagcggagaaggtactggtgaattttctggagcctttacattttctacagtgaaatggagataaactttactcatgccataggacatgtttcaaaacaataataagatgttttctgaacacttactacatactaagcactttatatgctttgtctcatttaatccttacacagccacattcttctggggtttagcgaatgatttttgtggttgtgtcctatgcttgtcctgtctaaggatgaagttgttctaattgggtgcccctccttttgctttctgtgaggacttgcagaactggtggggtttaaacagtaccctcacttatctcacagaatttcattagctcccagatacccctgacattctccccctagcctagtgaagaaaatcttccatttacttgttcattctgcagtgacagctccatcaatatacaatagactatacatattaagtgtactgtatatactatacatgttaaaaatctcattcattttggtgaggcccagctaagaatacttacagtagagcttttttttttttcctaagcataaaagtatctttttcaatgcagcatgagacagagttgggaaaaccaaaataaatagatccaatggactccccaaagaggataatattcatttaaataaacacccctctcagtgttaaaactttctaatcaacatgcctttgggacacattgcaccctcaaagtttacactcccattgcaacgcagctttgtggttcacgttttttccattcagaatgtcattaccctgtcaatgatgtttcatcaacgtttgcttggatgagaatcctctgatattcttcctgatagaaatgtataagccctgttcatataaatgaataaaagatctaaccttactttctcagtagtggcttccttggagcaaaaagcagggacctccagagagctcaggtggatgactcttttctgtttcttccagagctcaacttacaattagtgcacaattcatttcccagaatgtcttctttcttattgtgcctttagaaagttattaagcaaacatttgaattcacagaatcttaccagtgtaagaggaatggaaaaggtaacttatcaaggtaacaatcacttcgtggccagttttttcggctcactgcaactacccctcctgggttcaagcgattctcctgcctcagcctcccaagtagctgggactacaggcgtgcaccaccatgcccagctaattttttttttgtatttttagtaaagacagggtttcaccatgtgggccaggctggtctcatggcaagttttctttgtgttgtcatgttattatcaattaataggaatttatatttcagttctgttaggtggataaacactattttgcatacctaaatgtttcatttatatcagcactggccaataaaaatatactataagcaggccgggtgcagtggctcacgcctgtaatcccaacttttgggaggccaacactttgggaggacacagggtcaggagatcgagaccatcctggctaacctggtgaaatcccgtctctactaaaaatacaaaaaattagccgggcgtggcgggggcgcctgtagtcccagctacttgggaggctgaggcaggagaatggcacgaacccgggaggtggagcttgcagtgagccgagatctcgccactgcactccagccttggtgacagagcaagactctgtctcaaaaaaaaaaaaaaaaaaaaaaaaaaaaaaatatatatatatatatatatattacaagccacaagccacatatgtacttttaaatgtcctagtagccatattagaaaacaaaagtaaaaaggaatagatgaaattaattttaatgatttttttaaacccaagatatccaaaatattatcattttaacatataattaaaaatttattgagatgttttacattgtttttttttttcttgacgctgtcttggaaatctagagtgtattttacatttacattacatctaattcagtctagccacatttcacatgctcatttttttgtgtgtggttttatggagcagagagtttaataggcaagaaagaaaagagaaggcagaagaaaatggctcccctgtacagagacgcgggggtggggcgctccaaagccaaaagaggaggtccctaagtatggtagacaccagccaggaatatatgcagtgtctggaggaggggatgtctgatttgcatagggtcacatgctcatttttatggctactgtattggtcagtacagatttagatggggatttgctctgaacaagttggtgattgatggtgttcatattttaattgaatttttcctgggttctgctgtacacttgtatgtgtgttagtttcatgtgcaatgcttgtgtcacttttaaaacccagatatatggcataacatgagaatgaaaaatggaccagaaaaatagtttggcaatgtagtcatgtttgttcctattaaatgttccctattgaccactctatctcttttaattataacaagaatctgccctgccagcatgcccagttacgctgggaaaacttctgcctcatttactctggctgattctcatccacttatgggtcagtggttcattttctagaggtcaccagcattcatacctagcatacaatttcattcattataatgaaggaatgttttcccttcaaagagacacaactagtgggcttaatttttcttgatatgtcacctgtaaaattttaatgatgatgtttaaactctaaatgtagccatcaagacaaaaactgcaaattttgagcctcagtgtgtgtgggtgggtttctgtttcggtaatttgaaacattgcagaatatcatcaaaatatgatacccaagaatcatatggtatcaatcattcctagatatactgatctattcattgccaagatagttcaatgagctggcaaaaacatatggaattattttcttaaaatgtgaaaaataaaatttaaccaatcatgtatcacagcttgcaactttagtcatactttgaaaagcattttaatttggacctcatgattgaaaatttataaaaagctgaacagaaattagttcacttcatattttagaaaagcagagtttctttactaaatgaggcatttgacccaaattggagagaaaatgttgaaactacttctgtgagcaagcaggtggcttctcaaacacatgttgggatgaaatggttgggcctcagggtctcagtgcctgtcactgagagttggcactctctatctccatggtctcctccaagtgtgactcttgtctcttgctgacctgacctgccccaagtgactcactggtcatgaccctgcacaccttgcgtctctcctatcaccctgccgatggcagagctacaaaggtctttgatgtagctctgtctgatatcctgtgtttccccctatggtctgtgtggaagcaggtatgggggtgtgtaaaggggaagcctatgaagttcatctgcaaagactacctggttaggggaggagaggaagaagctatatgcaccatttcaccagcaagcatgggctcttctgccttttagcttaggggtcctgttgtctagtctcactcacctattaaaacagtccagcaaatagaggttttgtttacctcccattaaaaaaaaagcaattaatggaatagaagataataatgtatgagaagcactattgtgaaagaaaaaccttcaacttctctcagccaaataattgcttcctcctctgtccttcccagaccttgatgtttgctctattatttcaaaacaactatattataaatatttgagaatgtgtatttccctgcaaggagatcttaatccccaagaaggcaggagctgtgtattattcatcccagtgctccttcaaaccagggcctcagacagtgcatggcccaaagaggtacttaataaacgtttggtaaactgacactattgaaattaagcaacctggatttgaggtgggtctctgccactcacaagagattacgctttgagaaaattccatcacttcattgattttcagttcttgcatctgtatatgggagacgatactaggtgatttctgacatctctcaccgtttaaatgctctgtgatctatacaacgaggggctcgctgttctagacaagttccttccagctttacagttgcataacccttctaatcttagtcacatgatgactccactgacagatttttggccaccatcattagacatgctgagttacgtgtgcctttgctctgatcctcaaaactcatgatttttaaagttttctgaaatatctaccatttatcaggatccagatggatttcatgaccaaagtggatgtttcttttctctcccattacaatcttttactttttgtgtgggaaattgcatgttaaagaaagggaaattgaagaatgggatgctttggaattctggcaagatggattagtgggttccagaaagtaggggcagccacaaataccgaaataaatgagatcgtattattgagaaagcacaaatggaagaaggtcaaaagcaagaagaagctgacatcctgcttcctccaatttttgctttctctgtttttccaagaaactcctcttccaagccttgctgaaaaactccactttcctaaatctaacttcttaaactgataatggcaagaagttaggaatgaccaatatgtttaacactccaagagtatttgttttgttttgttttgagactgggtctcactctgtcgtccagcctggagtgcagtggtgtgatcacagctcactgcagccttgacctctcctgcccaaggaatcctcccacctcagcctcctgagtagaagggaccacaggcatgtgccactacacctggctaacttttaaaaaatttttttgtagagatggggttgcccaggcaggtctcaaactgctgggctcaagcaatcctcctgcattggcctcccaaagtgctggaattatgggcataagccactacgcctgacctctccaagggcattctttacccagaagaggaacttggcagaacttatcctccaattggtgaggaatatggagaaaatgactttaagcaaaggaacttctggttctgcctacctaatccagaaaaagaagttttatttctcccttcccctagtaactatcttcccatattcacataaaaaagtacagaatcaacattgttcaagaattataattttacttgtaagcacatgtgcacacgcacacccatataccttccttccctttaaatcatcccacaccctaatagtagtaaaatcattgacccgagcatacctgggagaggaagaggagtctgacaggggcaggttctaagtggcactcctggaacttaaccctggtgtatatgaactttacctattgaaggatgactcctcaactgttctcacaatttgctgctctgctttcttttctaatttctgaaggtgactcatcttccccaaggactttcagacttctcagaagaaaaaaatattgggtgggtctctgccactggcaaaagattagactttgagaatcataaaagtatatcagtatatactcattaatattgaattactataattaatattatgatattgatataatgatagaatgatattgataaaagcaatattcaataatgaatattatttcagctgcccacttattgggtgcctcataggtgccaggcattttgtatgtattatctacaacccttacatgggacatattatgatgctgtttctcttgaagaaatatggaaactggaaacagagaggtcaccacaattttccaaagtcacatagctaataggtagcagacttgggattcaaattcatatgcatatggtaaatcatgctcttcctctgctacattttgcccccttagaatatgaaaaagggatacaaagagatgaagaaaatatgtaagattatccttcaatttcactatcttttttaaagttttttttattatactttaaattctgaaatacatgtgcagaacatgcaggtttgttacataggtatacacgtgccatgatggtttgttgcacccatcaacctgtcatctacattaggtatttttcctaatgctatccctcccctagtcccccacccaccgacaagccccggtgtgtgatattcccctccctgtgtccatgtgttctcattgttcaactcccacttatgagtgagaacatgcggtgtttggttttctgttcctgcgtcagtttgctgagaatgatggtttccagcttcattcatgttcctgcaaaaggcatgaactcattttatggctgcataccattctatggtatacatgtgccacattttcttaatctagtctatcattgatgggcatttggcttagttccaagtccttgctattgtgaacagtgctgcaataaacatatgtgggcatatgtctttatagtagaatgatttataatcctttgggtatagacccagtaatgggattgctgggtcaaatggcatttctggttctaggtccttcaggaattgccacactgtcttccacaatagctgaactagtttacactcccaccaacagtgtaaaagcgttcctatttctccacatcctctccaacatctgttgcttcctgactttttaatgattgccattctaattggagtgagatggtatctcagtgtggttttgattagcatttctttaatgacaagtgatgatgagctttttttcatgtttgttggccgtataaatgtcttcttttgagaagtgtccgttcatatcctttgcccactttttaacggggttttttcttgtaaatttgtttaacttccttgtagattctggatattagtcctttgtcagatgggtagattgcaaaaattttcttccattctgcaggttgcccgttcactctgataatagtttcttttgctgcgcagaagtttttttagtttaattagatcccatttgtcaattttggcttttgttgccattgcttttggtgtttagtcatgaagtctttgcccacgcctatgtcctgaatggtaatgcctaggttttcttctaggatttttatggttttaggtcttatgtttaaatctttaatccatcttgagttaatttttgtataaggtataaggaaggggtccagtttagttttctgcatatggctagccagttttcccaacaccatttattaaatagggaatcctttccccgttgcttgtttttgtcaggtttgtcaaagagcagatggttgtagatgtgtggcattatttctgaggcctctgttctgtttcattggtctctgtatctgttttgatacaagtaccatgctgttttggttactgtagacttgtagtataatttgaagtcaggtagcgtaatacctccagttttgttctttttgcttaggattgtcttgactattcaggctcttttttggttccatatgaaatttaaagtagttttttctaattctgtgaagaaagtcaatggtagcttgatgggaaaagcattgaatctataagttactttgggcagtatggccattttcatgatattaattcttcctatccgtgagcatggaatgtttttccatttgtttgtgtcctttcttatttccttgagcagtagtttgtagttctccttgaagaggtccatcacatcccttgtaagttgtattcctaggtattttgttctctttgtagcaattgtgaatggaagttcactcataagtttgctctctgtttgtctgttattggtgtataggaatgcttgtgatttttgcacattgattttgtatcctgagactttgccgaagttgcttatcagcttaaggagattttgggctgagacgacagggttttctaaatatacaatcatctcatctgcaaacagagacaatttgacttcctctcttcctatttgaatacgctttatttctttctcttgcctgtttgccctggccagaacttccaatactgtgttgaataggagtgttcaccacctattttaagaatagtattgaagcctcacaaaagctggttctcatgtaaccatctgagaatatttggccttatgacttgaattcattcattgcctttttatttcacattttagtgatcctgatgtctaaatcttaatctttgatccttgcaaggtaaaatagccaagtcaagcctgtttaataatattggttgaggaagtcacatgcttatgatcaatctttgggttatgtaattatattaccttaatgttggcagtttaggtgtaaggcagagatatctgatcacatgtgtggttagctaatttaagatcactgccaactaaaatcttcatggtatgatcttcaaagttagctactttgaccacagcaatgatttcaccacagcaattaacaaaatggcagactctttcctgaggtggcatgaacagttttaaaacaaagtcaaggaccaaaagaaaagcaggcacatggcatttgattcatttcaaaaactagtattgtattaagagccaaggggatagaattgtagcatcaattaaaatcttgtttgaaaaaaaataaaaacaaacgtccatttttatctctcaaatatattagggttttcataaagttataggtgtatttttaaaaaaacaaaactcatatacattagactgaaaaatttgcctgtcatctcatcatgcagctaaatgcaattgtctatggcgagatacactcttattagaggtatgatagcactaactaatagcaaactttgtacctggtagtctaatttatgcagggttcatatttcgctccctctcagcatgctgtaactgtggcaaagccattctcaggagttattaccccaacataatcatacccctgtggattaggagcagttaaatgggtcctgttatcagagacacatatgtgccacagccgctgtcatccctaagccaccgtgggtgattaagactcactgatgggactacctctgaataggcttgagtgaggtggatacacttcagctgagagaaattcaggtaaggggctgagaaaatcagattttgaatggttttatcataccatcaggtctccttttaagtgctggggtcatggatttcattcaacctgaccacatagcctttggaagcttggctcaatacctgagtgtgagattatgggtgatattaaaggagatgaatgcattgagctgatgtcagagaatgtcttttactggattttcataatgactggctgcagatgggctgaggggaaagtcagatcaagagcatttctgtaagaagaaagaaatcttccctcttattctctttcaagaaaatgaatagctgagcaccaagaggccaaatacttttttaaaaaacacatccttttatgtagagaaggacaggttgagacgaaaaacaggacctctgaaactgtctttatagctttaatctaggaagaaattgcggcaccattgctgacatcattatcagaggctgccttagttctgagagcttcacagatggccttttctgcattttacatctggccagatgaaggcaaaaaggttgatgaaaccaaaactattagatcagtggtccataactctggctgcactttagaatcatctggggagtccttaaaactactgatcctgaggctccatcccagaccaattgaatcaggatctctgggggtgggacctgagcattggaatgctttaaaagttcctcagggactctaatgtgcagccaaggctgagaatgactgaggtggatggtggccaagacaggtgaggccaagagttagaagcccttactgttagggaaggcagaagccacaaggaggaggggagggggaaggagcagtatttagcatttcttccacacagttggggggttttctgatcaaaattaggctgggatctttcgcttccattttcatgaaggtcttatgctttgtccacagccacctggcctcagggcaatgagcaatctgattgatgaattttcagtaagaaactgagcacacttggctctcagccccagtggcttcccctgtttccaaatctgcccaccagttacaggagcctgctcaccaactggttgggttaaagaagtcggctctgtccttggcagggaggccttcagctgtctggccctgtctgtgactgcgtgggtgaagctgcctaatttggggaacttgatggaagatctaagctatgttctctaacagttttatcagaaataaagttaacttttgacctccatctgcctgtctcctgtggaagggctctgctccttccaaagagactctcaggggttctccttagaggtgtgttatcagtccaaagatcattttagaccagccattacagaggatgtccaagaaattgcacaagggaaatagaagtaagaatgagagcaatatatcagatagtaaggaaagtatgtgactggtttcaaataagtaattaaccataaatccaaagttcctgcattagttatagcaatagtcatccacctggtcaggaaaaaaaaagtaaaagactgagctgcaagatagaaagtttgcctgaattccacgtacttcaaggactactatggaggattccttggtcccaatgcagagacatgtactctgaccacccatgggccaaatccctcttacccaccctcagtgattcctcctgggacctcactacattgagttcttacattccccactcttttcgggggaaatataacccttctgctcttctcatgatgttaaaaatatttagacaattacttaaaatttacaacaatcttccacataaatgatctcacttatgtttcttgtgagctctgtgatttatttctattatcatctatgctgatagttaaggaaactgagatatcgagaccaacatgcatagtaaatggcaaaaccagatgaattttaaactttcctaactccaaaagccacatcctgcccaacccgccatgctgccgctcagttaatgcctggctgtttgtctccccattggcccctctacccattgtgctttgatggcacactgtattccaactgcctgagactcctggttaatgccattacgtacagacttaggttgaatttactaggatttttaagatttgtaggataagagatatgactgttagactggaatcagcaatagataaaaggttaacaaagtttcagaataaaatataatagaaaaccccagcagatagaagtaaaatgaatggtaagacaactgaaatgaaagccatacatttagaaatatcaaataaaacacagattaatggcagacaataaaggaacatacttagcagttagtaaaaacactttttaccttattcttattaccctccagtaaccttttttttttttttttttgagacaaagtctcgctgtgtcgtccaggctggagggtagtgatgtgatctcggctcactgcaacttctgcctcttgggctgaagagattctcctgccccagcctcctgagtagctgggactataggggcccatcactgcacctggctaatttttgtatttttagtagagacggggtttcaccatgttggccaggctggtcttgaactcctggcctcaggtgatccacccgcctcagcctcccaaagtgctgagattacaagcgtgagccaccaagcccggctgtaacctattgaaaataacattacttgacatgtgagacaaattatttgtaagttaaagagtttatgtgccttcaatgctcccacccttccctcccctaaaaagattattgagtgcccatgatgtgcctaagccttctggtagactctgagaatgtgaagagagttagaggatacttgttcaacaacccagaatctagcagaagtgttttgaaatgaccagccacttgggagagctatgctagcatatcattcaggatgggctgggtcatgttttaataacaagtaagtttgaactttataggtttgaaacaaaaaagatgtgtttcttgctcacagttcatattttttggagattggctggagcttcattccaggatactgccaccatctggaatgttgacagttaccgtaacagagaaagagttgtggaggctgtcacactggcaattaaattcgctgacctggaactgacacatgccacctccacttatattttattggccaaggcttgttacacagccacatctaacttcagagaggtcaagatgagcaaatcctaccacgagctatagatggatgagtagcactcattattatcacaattattttatttgttataaaaactccaagaaggagaggtactctatgtgaggaataggcataggaaaatcagagggaagttcttaaagatgagctagatttcactagatggcattgaagaaacattccaagtaaatgaacagcataagcaaatgcatgaagaacttattgtggtcctagtacagacgatgcgtgagagtgtggaggagggaaaaggtaagactggagaggaaggcaggaaccagaacaggacagactgtgttcactgtcaggcagttaagtctatcttgtaggcaacatgaagccttttaaggaaggcaagcagctatgtgacaggacagaagatggattggaagaaaatcaaaagagaagtggggaccagtcataaggctcctccaatatttggggatccaaaccaaagcactggcagagaaatagaaaggaagggaaatatttccaaaatattcaaaacagaaaccaagagaacttgatgacagaagatgaacccaggtttctactgaatggacagttgttacattctctgagataaggaatacagaaggaaaaagttgagagggaatatgaaattatttttaatcatgtttaatttgatcacttgtggaacatcaaccagagatgtccatcagttacaccaatttgtaggttttgagagaggcctgaggtagaggcagagaagtgggaatcagcagattagcggcagtagttggaaccataactgaagatgagatttcccagggaggggagttgaacaggaaggtagacaaaggctgagtgcagtggctcatacctgtaatcccagcactttgggaggctgcagtgggtagatcacaaacaagatcaggagttcaagaccagcctggccaacatggtgaaaccccgtctctactaaaaaaacacaaaaattagccgggcgtggtggcacacacttgtaatcccagcctcaggaggctgaggcaggagaattgcttgaacccgggaggcagaggttgcagtgagccaagatcgtgccattgcactccagcctgggcgacagagcgagactccgtccccaccgaccccctaaaaaagaaagtagacaaagatgtgtcctagaaaacactaatatgaatgggtagagaggggagacttgtcaaggagatcgaggggagagtcagtgaggtgaaaccttagaggatagaccttcaccaaggaagtaacagtcaacaggcctaaatgccacagagatgtcaaatgagagacactggaaatggttctttgaatattacagccagaacatcacaggagaccatttccaaagcagtcttgatgaagtggtggggaagggaggtccctgaaggagctaaggagggactgggagatcatgacccagagataagtgttgcaggtataaaagggaaagactgagatcaggaaatagccacaggatccctaaggccaggcttcgggtggggtggtgtgtgtgtgtgtgtgtgtgtgtgtgtgtgtgtataaaatgggaagaccttgagtataattctgtactaagaagatagagcagtagagaggaaaaggctgaagacagatgggagggtaaatagtgaattagaaaggtctgtgcaaagatgagaaaggatgagtctgaaagcacagggagaagggccagccttggacaggagaaaacatttctttcactaagaataaagggaaggttgggtatacaccacaaagaaggtgtagatgggtggcaatggagttctgtcaagttgagggcattccatgatagcctcacctttctctgtgaaatgagagagtttagtttacaagatatttctgagcacttcataagccaggttcatagactgaaagaagtggaggtggagtgtgataggtccttaagaataggggaagtttggaatatctgacaagggacagagaggaaaaaactagaaaaggctttgcagaatgtgggcccacagatcagaggctaaggggtccccatttgtgcaggagagtaagggcagggaggcaaggctcacagcccaaaatatagagcccctcaccaaatgactgcaggagggcagctttcctatgagagcatccctatcactgttttcactccgagtcattaacttacgacttactcagctctgtttcgtaatagcagactcgagtaatgagggtatgacagcctctctctgcatgccaaggtatgcagcgtggatttcctttttcgctttctctctcctgtggcttaggtgccttctgttctgctaccaggatagagaacccagtgactagtttcttctagctctctttttctgactaggtatcttgtcagaaatttctgcttaccagacttcatggagagggaatcaagctttgaatcagggttgaaaaagtagagcttaatatatatattacaaaatgccactcacgttcttgaggttaccttgtatctataccacaactagcattcttttagaaagcaccattaccgaagtaatccctttcctgggaattcacccaaaaaggttattcccacttatcccccatctccaaaataaaaaagaaaaatgtgtgtgcttagagatgttcctggaagcatgagctgtaatactgaaccaatagaagacgaactaacagattgcagggcatccgtttggcaaaaaacttatgcagttatctaaatgatagttatgaagacaatatgtataacacattatattctgagtgaaaagaacagaaggtgatttcaaaactgcattgggataatagtaatataggaaatagtgagatgaacaaagatttcaaaggaacaaaaataaagaaaaattttgctttttatattggtaggcgtatgggtgaaattccaattttaattttaatttcatagttataaaattgttcataaaaaaaggtcccccatagacagttgggctttgggacaaactaacagaaacagagtaggaagaaaattcatcttccttcaatcccctttctctgcttaaaacaaaacaaaaaagagctttgtcatgttcaggtgtgcaacgaattctttttccaaatctggaactttacatctgctattaaacaggagtcagtttccatgtaacatgttgacaatcccccaagtgtgttggaataattttttttaatgaggagatttgaaattccatttcaattgccaacctgcctctttcaacttctaaaaacaaagtaaaacaaaacaaaaacacactgggtcctatcaccccctcttgctactactattttatctccattgccctgaattctttccaaacttctttccacccagctttgatttgttttcagtcgggtttattgaggcataatttacgtacagtaaaattcatccttcttagatttagaggtctatgcattttgactaatgcatattggcttataatgactaccacaacaaagatatagaacacacccatcactcccgggttctccctgtcccttttttggtccatctcctctcctacccccaaccccttggcaaccactgatctgttttctgtccttatcattttgctttttccaggatgttgtatataaggaatcatgcagcatgcagcctctcgagtctgacttcttccagttagcacagttatttaagatctatccgagttattgtgagtagcagcatcttttttattgctgactagtatttcatcacatggatgggccacaacttgtttatctgttcacctgtcaatggatactaagttgtttccagtttttggcaaatatgaataaagtaaacatttgcatacagatttttgtgtggacacatgttttcaattctcttaggtaaatactgagcaatgggattgctgggttatatgttaagtctatgttcagttttctaagttctgaaacaattggatatctatatgcaaaaatacaaaattaaccttgactcaaacttgtaccatacacaaaaaataacctgaaagagatcacaggtgtaaatgtaaacctagaactaaaaacttcaaggagagaaacatagcagaaaatatttgtgaccttgcattaggcaaagacttcttagatttgacatttgaaacatgatacataaaaaatcttgataaattggagttcataaaaataagaactactcttcaaaagacactgataagagaatgaaaatacaagccacagacagagaaaatatttgtgaatttcttatctgataaagggtttgtatccagcatgcataaagacttttcaaaactcaataataaacaatccaataagaaatgcacaaaagatacaaacacatcatcgaaaatgacctatgaaaggcaaataagcccacaaaaaaattctcaacatcattagacacttgggaaatgcaaattaaaacaacactgagataaactacatatctattaaatggctaccactttaaaaacctgtcaagtgccagccagaatgtggaacaagtaggactctcttacattgctagtgggaaggtgaatggtacagccactttggaaaacactccgcagcttcttatagttacacataggacctgggggtagaggatggattgactgtaaaggggcaaaacttcctggtaggggtggggaaagtttttagaagggtgaaattgttctatatcttgattattgtggtggttacacaactgtctgcatttgtcaaaactcacagaactgtacactaaaaaggtatatttttatgctctgctaattttactttaatcttaaaaataggaaggaaaaaataaaatcaatgccactgtgcgactttgggcaagttacttcacttctctgtgccttggtcttttgaaatctatacattaaggataaaataataccttcctcatactgttagaattaaatgtgctaatttatgttttatatatataaagtacttggccgggtgtggtgactcacacctgtaatcccagcactgtgggaggccgaggtgggcagatcacttgaggacaggagttcaagactagcctggtcaacatggtgaaaccctgtctctactaaaaatacaaaaattagctgggcttggtggtgcgtgcttgtaatcccagctacttgagtggctgaggtgggaggatcacttgaactccagaggtggaggctgcagtgggctccacccactgcctgggtgacagagctagactccatctcgaagaaaaaataagtacttgaaacatagcaaatgttttataattattggcttttttttcttattgttattacttgcattattgctgtttgaagaagtttggtgataatggagagaaaagggcaattaggggtctgggatggtttaagtatgaggagaccgagacacattgactcagagtgaagaaatcaaagataagagaatgaaagaaagggagggtaattctgaacgcacagacaaagttatgttactaacgtggcatcggctgtgtgttgtaataaataactccctttcacttgtcaatagctaatcaaatactttctggagaccagaagtgacttgctggatcaatgacaactcctccacagatcaaaatgttcaaatcctttttctgtgttgtaatcctaaatctaaaagaacagagagaccaagcaaatctacctcccaacatcattaaagtgacaactctcagtatttatttgaatggtctgctctcagcttcaaccaaggaaaagtcaaattagtgttggtcagaaaacagaagggtgttacgagagttctggctggtcattacagacttggggatttttgattaaaagaagaagaagaagaagaaacctgataaagtgtaaatatagcaagcagggattagtgtcctgctgggtcatgttctcacaacagtgagaatttcagagatttcataagaattaaactgctccacatgacaatttattttaccttctggcttttccaggaggcaaatcagtgcaacttctttctgcctttgtttcaatttggtaacaaccctcaattttaggacaggctaaacctagccaccctatcagagatgatgaagtagccatctttttaacaggtggggagatgaatggaatcagggtttgtttgtttgtttgtttaataactgctagtaaaaaccaagtcaatagctgactgagtgtaagggaggctccagaaggcaggttattgtagtatagatgtgactcgacttatgatgatgttacttcccgataaacccgtcataagttgaaatatcgttaaattgaaaatgcttttaatacaccgaatctaccgaacatcatagcttagctcagcctacgttaaatgtgctcaggacgcacattgcctacagctgagccaaatcacctggcaacacaggacactgtagagtatcggttgctggcccttgtgatgctgtgactgactgggagctgcgcttagtgcctctacccagcattgagagtttcttatcgcttattactagcctgggaaaagaccaaaattcaaaactcaaagtgcggtttctaccgaatgcttataactttcagaccatcatgatgttgaaaaatcgaaccatcgtaggttgggatccatcctataagacgagctacactgccggaagtgtaagactgctatgctgccggaagatggggcatagtggacaactgcaagtcctgacaacaggaggtcagcatctgcgacctttaacatccacattgacactaccacagtcttccaaacagagctgatgatatagtttggatgtcgtccctgcccaaatctcatgtcgaatcgtaatccccagtgttggaggtggggcctggtgggaggtgattgggtcatgggggcagagttcttatgaatggtttagcacggtccccccttggtactgtatagtgagtgagttctcatgcgatctggttgtttaaaagtgtgtggcacctcccctctctctctttctcctactctggccatgtgaagtgttggctcccgctttgccttccaccatgattgttaaattcccagaggtctccctagaagctaatgctgccacgtacagcctggagaactgtgagccaattaaacctcatctctttttaaattacccagtctcaggccgggcgcggtgactgacacctgtaatctcagcactttgggaggctcaggcaggaagatgatttgaggtcaggagttcgagaccagcctggccaacatggtgaaaccccatctctactgaaaatataaaaattagccaggcatggtggcgggtgcctgtaatcccagctacttgggaggctgaggcaggagaatcgcttgaacctgggagtcagaggttgcagagagccaaaatggagccactgtactccagcctgggcaatggagtgagaccctgtctcaaaaaatatatatatattacccagactcaggtatttctttacctgagactatgagagaatggactaatatagctgaagaattttattttatttttaaaaaacttttacgtttgggggtacctgtaaaagtctgttacataggtaaactcctgtcatgaggatttgttgtacagattctttcctgctcctccccctcctcccaccctccatcctcaagaagatcccagtgtctgttgtttccttctttgtgttcgtaagttctcatcatgtagctcccacgtataagtgagaacatgcagtatttggttttctgtccctgtgttagtttgctaaggatgatagcctccaactccatctatcttcctgcaaaagacatgatctcattcatttttattgctgcatagtattccatggtgtatatgtaccacattttctttatccagtctgtcattgatgggcatttaggttgattctgtgtcttcagaattgtgaatagtgctgcaacgaacattcgtgtgcttgtgtctttatagtagaatgatttctattcttctggtagtaatgggattgctgggtcaaatggtcgttctgcttttagctctttgcagaatcaccatactgctttccacagtggttgaactaatttacactcccactaacagtgtataagtgttcccttttctctgcaaccttgccagcctctgttatcttttgactttttaataaaaaccattctaattagtgtgatggtatttcattgttgttttgatttgcatttctctaatgatcagtgatgttgagctttttttcatgttcgttggctgcaggtacatcttcttttgaaaagtgtctgctcatgtcctttgcccactttttaatggggttgtttttctcttgtaaatttaagttcctcatagatgctgggtattagacctttgtcagatgtatagcttgcaaatattttctcccattctgtaggttgtctgcttactcttttgattgtttcttttaccatgcagaagctcctaagtttaattagatcccatttgtcaatttttgcttttgttgcaattgcttttggtgtctttgtcatgaaatctttgccaggtcctatgtccagaatgatattgcctaggttgtcttctagggtttttatagttttgggttttacatttaaatctttaatccatcttgagttgatttttgtgtttggtgtaaggaaggggtccagtttcaatattctgcatatggctagccagttatcccagcattatttattgagtaaggagtatctcctccgttgcttgtttttcccaggtttgttgaagatcagatggttgtaggtgtgtggccttattttggggctctctatcctgttcatttggtctatgtgcctgtttttgtaccagtaccattctgttttggttactgtagccctatagcatatttcaaagttgggtaacatgatgcctccagctttattctttttgcttagaattaccttggccatttgggctctttttggtaccatatgaagtttaaaatagttttttttctagttatgtgaagaatgtcgttggtaatttgataggaataacatgtaatgatattgattcttcctatccatgagcatgggatgtttttccatttgtttgtgtcttctctgatttcttcaagcagtgttttgtaactcatattgtagagattattcacctccttgcttagctgtattcctaggtattgtattctttctgtagtaattgtgaatgggattgcttttctgatttggccctcagcttggtattgttggtgtataggaatgctagtgattttttgtatcctgagactttgctgaagttatttatcagctgaaggagcttttgggctgagactagggggttttttagatatagaatcatgttctctgcaaacagatttagtttgacttcctctcttcctacttggatgccctttatttctttctcttgcctgatttccctggccaggacttccagtaccatgttgaataggcgtggtgagagagggcattcttgtcttgtgccagttttcagggagaatgcttccaccttttgcccattcagtacgatgtttgtggtggtttgtcatatatggctattattattttgaggtgtgttcctttaatacctagtttattgacagtttttaacatgaagcagtgtttaattttattaaaagtcttttctgcctctgttgagatagtcatgtggcttttgtctttagttctgtttatgtgatgaatcacatttgttgatttccttatgttggaccaaccttgcatcccagggatgaagcctacttgattgtggtggtttagctttttgatatactactggattcagtttgcaagtattttgttgaggatttttgcattgatgttcatcacggatatcggcctgaagtttctttttttgttgtgtctctgtcaggttttggtatcagaatgatgctggcctcctagaatgagttggggaggagttcctcctcctcaatttttttggaataggttctgtaggaatggtaccagctcttctttatacatctggtagagtttggctgtgaagccatcaggtcctgggattttttagttggtagggtatttattactgattccctaaatagaccgataatgattttagaagtggagtcggttttttcctggtccagtcttgggaaggtgtatgtatccaggaatttatttagctcttctaggttttctagtttgtgtgcatatgggtgttcatagtagtttctgatggttgtttttatttccgtgggatcagtggtaacattctcttcatcatttcttttttttttttttttttttttttttgagacggagtctcgctctgtcgcccaggctggagtgcagtggcgcgatctcggctcactgcaagctccgcctcccgggttcacgccattctcctgcctcagcctcccgagtagctgggactacaggcgcccgctaccacgtccggctaattttttgtatttttagtagagacggggtttcaccgtgttagccaggatggtctcgatctcctgacctcgtgatccgcccgcctcggcctcccaaagtgctgggattacaggcgtgagccaccgcgcccggcccatttctaattgtgtttatttgaatcctctctcttctcttctttattaggctagctagtggcctatctatcttattaattttttcaaaaaaccagctcctggatttcttgatcttttgaatggtttttcatgtatcaatccttcagttcagctctgattttggttatttcttgtcttgtgctagctttggggttgacttgttcttgcttctctaattctttcagttctgatgttagtttgttagtttgagatctaactttttgatgtggacatttagtgctataaatttaactcttaacactgccttagctgtgtcccagagagtctggtatgttgtatctttgttctcattagtttgaaaaaacttcttgatttctgtcttaatttaattatttatccaaagtcattcaggaacatgttgtttaatttccatgtaattgcatggttttgagcgattttcttagtcttgacttctatttttattgtaccgtggtctgagggtgtttgatatgactttggttcttttgcatttgctgaggattgttttatgtccaattatgtggttgattttagagtatgtgccatgtggtgatgagaagaatgtatattctgttggttttgggtacagagttctgtagaggtctattagatccatttggtccaatgttgagttcagatcctgaatatctttgctaatttcctgcctccatgatctaatactgtcagtaaagcactgaagtctcctactactattgtgtgggagtctatgtctctttataggtctctaagaacttgctttatgaatctgggtgcttctgtgttggatgcatatatatttaggatagttagatcttcttgttgaattgaaccctttaccattatgtaacgcccttctttgtctttttttttctttgttggtttgaagtcttctttgtctgaaattaggattgcaacccctgcttttttctgttttctgtttgcttggtagattttcctccatccctttattttggacctatgggtgtccttacatattttatctttatctatccatccagccatccagccatccattcatccgtatcatttttaaccaataaggacttttaaaagcgcaaccacaacaccattaacataaccaataaaatctataacaatgataaaatatcatctaatactcagtccatgtccaattttccctcgctatctcaaaatcgtcttcttagaaatggtctgttcaaatgagatcacatggacagaggaaggcgaacctcacactgtggggactgttgtggggtggggggaggggggagggatagcattgggagatatacctaatgctagatgacaagttagtgggtgcagcgcaccagcatggcacatgtatatgtatgtaactaacctgcacaatgtgcacatgtaccctaaaacttaaagtataataataaaaaataaaaataaaaaaataaaaagtgaaaaaagaaaaaaaaaaaaaaaagaaatggtctgttcaaatcacaaaccagattcagaaacaatagccatacattacattttattaatatgtctcttaaatttcttttaatctattacagtctttggaatttttatgtcttcgtttatccttccaattattaaaaaaaaagtatttttgtattcattgaatagacaatgcttgcagaaaagtaaaaaaaaaaaaatttagtacaaaaaggtacatagtgagctgttccttagtctcccttcccagaagcaatgttaccacttttgtacaaatagtctctgcctagacacacatgccagtccctaaggtggctgtaacaaggtggttaagagtgagaacatgaattcaaattcctattatgccactcactaagtataaatcttggtcatggtacatgcctctgtgcctcagtttttaataatggtacctacctcatagggctgttgagagaattaaatcagataagtgcttaaataactattaatatttattattattcacattcccttttggcttttttcccaaatagcagagtggtgcacatatgtcttcatttatttggcttgttttttcacctcacatcacattttgatgaataattccatacatgttgttatagatttgcttcattctttgtaatcattgactaatattccattgtatgaatatgctactgctaaacatgtacgttatttccaacctcttattatcaagaaatgctgcaatgaatatccttgtaatactttagtggattcatgtgcaaaaatattcataggataaaatcctgaaagttaaattgctgagctaaagggtatgtgcattttaatgctttatagattgcccagctgcctcaaaggaggttataacaatttacactcccaagaaaaatgcacaagggtccccatttccccataccctagctaacacaggatattgctaaatgctttcatctttgtaaacatgatgtattgaaaatggtatctcaaagttttaatgtgcatttttctgattgtgagaagataaaggaaatgtagtacaactaaacatcagcagtcaaatgacctggccatgactcctgagtgaggacactggtaaacaccatcaggatccaaacacctctgtatttacgaagaggatgctccctattggatagcactaagcttatttcatgtatgtacatatgtagttagttaattacatccagcggtggcaaagggcttgttctgacccaatgaaactttctctcctggcccccttccagcatgtggtcaggagtagagtgttgtggccatgaggcatgcatttgtacagatgactacttactcctccttgaaacatttttttccatttgcttccctgctgtctcactcatgggtctgctcctaattcacaaatcactcttttcccagtcttcttggttgggttttctcctcttctgtgcttgtagacatgggggagccccagggcttctctcttgaactacagcttctccctgggttcatctccttgggatgtctgttccaatgggtttaaatactagggctaggactagggagaggtcattgaggcacttagcacataaagtttaaggaaacattctttatcaggcccatgcaagtgcaggactggccctggagggtgactaccaccttaccttttccaccctaggcaccttgtttgccttaccctagccccagtcctctttaacaccccagtcttctccctggacctccagaaacataaatcctatttgacatttctacttggaggttttaaggtaactcaaacgtaaaatatctaagacagaactcttgcatcacttcccatcctggggcccaagcctgtctcttctactagtctatctcagttaacagcatcaccatttattcagttgctcaggacaaaaaatttgaagtaatccttgactcttctttttttttttttgagacggattctcactctgttgcccaggctggagtgcagtggtgggaccttggctcactgcaacctctgcctcctgggttcaagcaattctcctgcctcagtcttctgacctcgtgatccactcgcctcggcctcccaaagtgctgggattacaggcgtgagccaccgcacccggcctggattcttttttttttttttaaacaagtcctatcttccatctccaaaatgtatcccaaatctgacaacctctcccaccgtaggccagcccccatctctcccctctgaaaatagcctcccttagatctctggacatttgttcttccccacccccttgtgatcactattcagcattcagaatgatcttttaatattatgaaggagactgtgttcctctcctacttaaaattctctagtggcttcctaataaatttagaataaaaagccaactcctcgccatggtcaccaggccaggatccagtgggtacaacaatctgttcccagcacaataatcccacttctgcctccccaccattcaccaggctccactgcactggctcattcctgcctcagttgtgcttcttttccctggaaagttctttctgtagatctttaaagggtgatttccttctcaacattcaggtgtcagctcgtttcactttcctgaccatgcccatccactcagagatcactcaaaatcccattaccctattttatttctccatcatatgtatcactatctgaaactatcttgttgttgatgcaggctattttcttgaccccttcatgggactcccaacaggggtaccccatttactcagcctgccctgctcaacctcttgcaggagggagcacacgagtgaacgagtgcaggaaccagctggctgctttagtgctgtgaggagtaaactccatgcaggccctgcagcagcaaccaggtaggggtgcctgcaaccccagggccccagagggtgtgttacaatgctctcgtagctctgccatctgtggacagcagtgtgttgtcagctcagtgggccctttgcttcatcatgtagggtggctgccctctgcctgtgagggcaaagggccagggtgacagtctttttgggtacccacaatttgtgcatcctgaattcttgtttggtgcccaagaagaatggggtcacacagatgaactgaaggatggtgaatgcagagaattagcaatgaaagtggctctcagcagagagagaagctgaaaagggaatgggaagggcaggtcactctcccctgaagtcaagtcacatctctccaatgtccagccaccatctctgaagtcaagtttcctctctctgatgtccagccacttctcctctctactggctgagtctggggtatttataggcagaggataggtggtggggcaggccatacataattttggaaaaggcaacattctattggtaaaaagacattattcataaagaaccaattgggaaagagcgggcacacagggatggaagttctcactttgggctgcaggtttcaggcttttcagctcaaaagtgaggtttttccagggacctgccccgtctgcctaaaatttctacacttctgtcattgataccgctggataacagctctcctataaagttcaggggcttaaaacaaaaatttactaattcacccggacagttcttgatagggtctctcctaactgttgcagttacatagcaactgggttggagtcatcttttctgggcttgacatccaggacagcttcttcccttgtgtgtctggtgcctcagtgctcctccaggcagcctttctctccagaagagtagcctggacttcttggcaactcaagcttccaaaagaaaaaaaagcagagactgctggttctcttatcaaacaggcctggaactggcacaatgtacttctgctgccatattcaggaggtcaaagcaatcgaaggccaatccaagttcaaaggcattggagaaaaatgagaagtgtcacttaaatgagaagtgacacatgcgtaaaagggggaaaagcattgattgtggccatttttggagataagctatcacgtttatttgtttgtttgctttctaatggtctgtcttctcccattaggttataagctctgtgagacaacaggaatcttgtccatcttgttttatggctctacttccaacacctagaataatgcctggcacatagtaggtgctcagtgaataacttaagcacttgatacatgtttggggaactaaaatgaacagaattaaacttccccagattggtcctgccagatttgctgatgccaagcatgctgatgcctcaccagatgaaagaagccctaaaatgtagggtttcgctttctctgcaaacaagaaaaacttgccctgaacacaaaatctagaaatagatttggcgtgttttctacattgaaatatttcccgtagtaccagaaattattttcccacagctttgtgctacattaaaatattgaagttgactgaaaatatctccattctttaatcttggtgtagactagaaacaatttttttgtaacaaagtaaatatgaaaacttcctaatatttgaactccccagatatccccagatatctccaaacttaaaatatcattgcaagttaagataaatttttttaaatgactaccgagaaaggtcattaaaggcttggttattaaaatgtacagatttgggttataaagccagaacttatttgtttaaatcattacatatgaccaagcacagaaaataaattacctcaaatctcctctttgctaatttttactggtaaactctataaaatgatcctatctttaaacctttttgtaaaccccttataagttagtaagtgagaatgtattcatcagaaggattttagtgatgtttgaaattaaaaaagagagatttgatttttaaaattatacttgcagactactgctaatgaaacttcttctaaccctagttttgtcttatcttcagtttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggtaagtacaaaattctacctctgaagacaaatgtgcttttcaatatgtcaaaaagaccgtctacctaaatataaagttataatcttaacatatatacatggatgcacactgtagtattatacataataacaaaaagtgtggaaataccacacttgctcaacagtagggaattcaataaatactttatggacatctatatgaataactgtgatgctgacattaaattatatttttgaagatgtaatcaagaggataaacgcttgtctcaaaaagttacatgggaaaagcagtatgtaaacttatataaacattgtgaacctaatttgattatatatataaaatatagggaatatacatataaaatacatatatgtatatatgggggctatatatatatatatatgaaagagatagatagatagatagatagatagatagatagacagacagacagacaatacagactccaatctgttggtcgtggttgtctctgacccatgacactatgggggacttttatttttgctcatacttttcaatatttcttagtgttcaataatgtgctattatttatacataataataaaaataaataaatggcatcaaaaaagagtaaagggccagtgttccgcccacatatgagcagccatattcaagcctgtagacactttgtgtagcctaatgctaggtgtatctgggcaaggataaactctaaagccagaaattagttcatcaataaacatgtgctactcaatagctagggctgatggaaaagaatataaaacccagtctgtgccaaatggtgcttactatctgcaagtgggagaaggagaaagacgagaaaatgaaaaatgtgtgtataatttatatgtagctgttctgtaggagatctctgacttcaccccattctaactttgcaaaaagatccaacactttgtcagattcctgggaggcaagtaattttattgatggtttcatggagggatacagaacgataacaactcacacaaagcaaacaatgtaatgaaaatctctattcgactgtttctttttctcctgaagttgccctttggctgccagctaccaggcaccaggctcaaggtactttcttgctcttgacactactcccttctctcatacaattcaaccccaaccacaaacgtgtatagatctctctctctataaaacaaaggcctgtagttaacaggaggtcacttgcagtgtagcctctgttcattgttacttgtgcacactgcttagggtctcaccccatccacattctgctaatcacattattcacccatccaatgtagatctctccagtggagattctgctaatattttctttagatttgtcacaagtatataatacagttttaaattgtacagatgattatcctataacagaagagtctaagccttttacatctttgtatctctaacgaaagcattcagcatgaagctctgtacacagcagacaattcaatatgaatttgctgacttgaaacagcaagcctagaaaggagatgttaacttggtcacttagacagaacaggtttcagcaatcagaattcagatgacatggaactggtagaacaggcgctttgaagcaataggacatgagccagtgaggagagggatggaatatcataaacaaaggccaagggctttgcaatcagagctgaagagccaagagcacaggctcagggtgtgggcagactgaatgagaaagtgattcaatcacatgtgaaagtccagatgagaagagagagttgggattacttctgctcaccaaacatccaaaaccaaacaggtggatccgggtggtgtgctgttttactgatgaccattacacagaattttaacagaaggaatgtaaagcagtggttctcaaactggagttcccagatgagcagcgtctgcatcatctggaaacctgataaagcagcaaattctcaggccctaccccagacccactgaatcagaaacttgggggtctgggggaagatggccatctgtattttaacaatctcccttcaggagattctgaggctggctcaagtttgaactacaggtagttggttcaacaggtgttggtggactgacaaacaaaaagagactccgaggtaactccaagatggtaatgtcagaaagcagctaccacccctagggcttgggggaaccaacaaaagagtttggcattgccagaacctagaatcttgaggagaggccccagagcattgtgtctcagaccttgaggacttggcactgggacaccatgaggggtttctgggtgtgggaaagggctggaaactccccagttgctgccaccagggagaactacaagtgaagtggaaggtgtgggcctttctcccttttcttttctcgtcttctctctccccctggtgctcatgtttgacagaaaacagctgaaaaggcagaactagtttggggagtcttgacctggcatcataaagcagagaaaagcaaagctggagtgaaggtgagacacaacagctcattagcagcaacagccgtctagcgctccagcttctgaatgaaattctgaagaacagcgcacttggaagacaaattatttgacagttctgacagacgaccaaactaacagcatttgaaaagcaagatgactcagagaatacagaatttaatccaaatcccagatcctatctctgcctttggccaggcctaatgcaaggagaacctgagcacacaaatatatgcaggaatgatcaggacctgtgcctgcattctattctgtctcacccaccttcaaatttgttgtaaaaacatgggctcaataaaggtttgtgaatcagggaaggaagagaaggggagaaaggaagggaaggagccagctccagatctgtgtcttgcagaggataaaggccagtgtttttagatcacccagtgtttttctaagcccccaatacttattttgaaatatcaaaatgttcaataactaaaaaaaaaaccgttacaacaataaaacatgtttgagggtcagatggactggcagtttgtgacctctggggataaacaggtcactttggaatcacagacttcctcattccccttaaatctcatatggtacccagaagcccttggaactttggaaggtgtttattcacagttgtaatgtccatgcagaccctggctctaagacccaattgtgtaagggtaggtttgtagcccttatcccaaacattctaagtgtgagccaatgcgtcacacactcagaggccagagactgtattggggtcctttatttcacgtacgagtcacattccattaagagaccccagaagtcagctctcttccactgactggttctcttcccttgtttctcttgccaatgtgtgctgcccaggtggcagtgctcactgtcagcagagaagaaaaatgctttcctccttggacctcttttctctttttctcctccctactcacattcaggttccctaagcttccccgctccttgtgctgaagtcattctatggtcatttcttcaactgtctacttccctgctggatgggcacccaagacttggcatcctggggcatgtagaaaggggaaagggaagggaagggaaaagaagtcctcccaattgtctatctggacctttccacactgcccagagtactgctatgggcatctccttatgtctcccgatgtggtgcatgccagaccctgcaggtagaaaaggaaagaaagcaacccattggaccaggccagcaaaggcttcagtcacacagctggctcatacttatggcttcatattctgttgcctcttgaaccagacatttcttccactctcataacctccagtttagctcgtattcctcagcattctccatgtaatattgttgcatgaaacccatgcaagtcagccaatttgctctttctcatcttgtcatttataaattgatgctgagaagtctttttcccaaggtttttagtaataccttcatcatcccccatagttcattttgggcagtgattcgcttctttgactgtacattagaatcatctgaagaactttctaaaactactgatctcaggtctcacacaacactaattaaattatagtctctggtggggtagggcttgggcactgctattttaccttaagctctctggagtcattctaatttgtagccagtgctaagggttgcaatataagtgaatatatttcacgtatttgtcaaacattgactgagtgcccattatgtgccagccattataataggcactggtgatcccacagtgaatcaggcacacaatgctgtcttcacggagcttgttgtctagtgggagagtcaaacaaaagtgtatatcaataattaagtgattacagattgcaataattacaataagggtgataaacaggttgctataatatacaatagtattgcctttcaccagacatttcttaaagaggtaaatcatctatcagacaccttttaaaaatctcatctaatttcaaaagtgtacataaataattaagtgattacagtttgcaataattacaatgagggtgataaacaagttgctatgaaagagaaagatagcagagatctggctttgagatggtggtcagggaagactgctccaatagctgagttctaaagctaagaaggaactgagaaccttcacagaacgtcccaagtagaagagaaagcacactgaagactctagggaaagaggtctgcttgttgtaggaaccgaaagaaggccaatgtggctaggtgctgggtagtgaggggaaatggcacaaggaaaaaatgaggttagagagatcagttgataccagttaatgttggaccctaaacattaaccatggtaagtcttttagaattgattctaattgcaatgaaaaccttttgaaagattttaaaaagatatctgatagctgatttacctctctaagaaatgtctggtgaaagacaataccatcacattggagatggaaaaagatgaatggattctaaaaacattctgaaagtacattcaaaatgtttttcaggtagcttatgcaactaataaatagtggtggcattctgggtaagacaagggaggagcaggcttgcagtttagggcaagaaaggggtggggagaagagctcagcactaaaatcatgtgttccatttggggcacatcgagtctgagttgctatgagaccaccaagtggagatgccaagtaaatagtcagttacatgaatctggagttcagtgaagaggtctagaagaaagatgtatatttgggcattattcggatatagatattatgtaaagcaataaaattggatgagatcacctagggagagaatgcacatagataaaaactgacctaggaccacttcatgtctaaaacaccaaaagcaatgtcaacaaaagccaaaattgacaaatgggatctaattaaactaaagagcttctgcacagcaaaagaatcagagtgaacaggcaacccacaaaatggaagaaaattttcacaacctactcatctgacaaagggctaatatccagaatctacagtgaactcaaacaaatttacaagaaaaaaacaaacaaccccatcaaaaagtgggtgaaggacatgaacagacacttctcaaaagaagacatttatgcagccaaaaaacacatgaaaaaatgctcaccatcactggccatcagagaaatgcaaatcaaaaccacaatgagataccatctcacaccagttcaaatggcaatcattaaaaagtcaggaaacaacaggtgctggagaggatctggagaaataggaacacttttacactgttggtgggactataaactagttcaaccattggggaagtcattgtggcgattcctcagggatctagaactagaaataccatttgacccagccatcccattactgggtatatacccaaaggactataaatcatgctgctataaagacacatgcacatgtatgtttattgcggcactattcacaatagcaaagacttggaaccaacctaaatgtccaacaatgatagactggattaagaaaatgtggcacatatacaccatggaatactatgcagccataaaaaatgatgagttcatgtcctttgtagggacatggatgaagctggaaaccatcatcctcagcaaactatcgcaaggacaaaaaaccaaacaccgcatgttctcacccataggtgggaattgaacgatgagaacacatggacacgggaaggggaacatcacacactggggactgttgtggggtgggaagaggggggagggatagcattaggagatatacctaatgctaaatgacgagttattgggtgcagcacaccagcatggcacatgtatacatatgtacctaacctgcacattgagcacacgtaccctaaaacttaaagtataataataataaaataaaataaaataaaaaaacaaaaattgatgtaggaccaattcctgaagaacactgacagttaattttttggtttaggaggaggagaagccagcaaacgacactgagtagcaatatccaaagaaaaagaggaaaaaggaaaactgggagattatgagtgtcccagagggaatgtttcaagattaccatcagcagtgagctttgtgtaaaggtggcctcctgtaatagaggtgcgggcaggagaaggcagaatagggaaaagggggtgaaaaagcttccctcaagatttataatacagtggaagagagagacagagagagagaaagagaaagagagagagagaacttaaggaggtagaggaagagagagaaccaaaaaagagggagctgagtatagaagcaattagattcatagtttttagttgcggcagtgatatttgagtgggggccttttatatattccattctaggtgtttcccagttgatgggagagggtcttacctagatctgcatgtaaaagggagtaggccagctggcagacttgacatggatcagtggtagaacatcctagcagttctgtgaatactctctgagaatgacatgaaaggtattggttcaggccttttggaggtgataaaaaccaccaaaatgtggacttattgcaaattgtatttgttaccatttgcaatgattataattattctcttatatataggctttcttacatatagcttctcttacacatagcgtcatgagttatgccttctcttacatatagtgtctgctatgagttatgccaagcagggcaaacaaaattgctgcctttctttaaaaagaggacgctcctagtatgggcctaattaattatgataattacagctatggcatggaacataagcacattcatatacacaaagacaataaaataaagaacagttcaaaaacagaacagttacattatatatcagtttcagtatgaataataccctggactctgaaatatgtctggggcacattattctgtaattggtggtgaaaaaaaatctgcatcttatctctacgccaatccttatgaaggagctgtttttcaggagttcgagaaagagacacagggatgtccagtcatcaaagccgcagagcctgaggaagaataaaggatttgtggcaggaaaacccagtaatgaatgtattctgctagtttctcagcatagaacttagaaaagaggccacagaaggaaaagagaagtaatgttagacagctgatgttggcaatgggcaaagaatttcattctcatatgcagggcagtggctaaaggggcagtgttgtgaggaatcacattccaggtcattcatgtccagggtgtggtaggaggactgtgtttcatttattttgtacatggcccagctatgaccttgtgcaaggaaatgcttaatcattttctatcacagttgcaaagagattcttatcctactcagaatgtacgtcttctcttctgtttcatttacagtcacaacccaagtccttgctttgacctccaaagcaccacttgatgtatccacttcagaaacacacacagacagctcacctctttctatcccttaccttcctcccctcttcactcaaaccacctacttcctttgcattcactcttccttcagcctgaaataatcttcctccaaatatctaccttctcactccctcacttctctcaagatacacttaaatgttatattccctatgaggcctcccctggccatccctccccagccttcctatcccccctctctgctttattttattctccttaatatatatcacactctgataaaccattgaatgtacttatcaagttattgcctctctctccctttccattgtctccatcactgagagctctgtgaagaaaaggatttgtacctgtttcatttagtgctgtacccccagttcccacaacagcgcaacaggcactcaataaatagttgttgaataagtgaataaaacagaagtagctgcatattttctggtaacaaatgatattcttctgaaaatgtcatattttcagacatatttccgaaaataaattcaaattagataaacattgtatttttagaccatttcttctttgcattaatcatccttctcaataatataacatttgtaaaacttaggttagatatgggctcttcaactttccattacagaagataaagtgaaaaggctagacccaatggtgttattccttcatctacatctatcctttggaaacacatgaccaaattgcttgccatcacaatctcaaaatctaccctttggtattaactcacttcacttgtccctctgtccctttttagatggtagccatcggtctctggagcagtgtagagtcagaacaacttctatttggggaagaaatcattgctggtgaccttactttcaattactaactttctagtgacatttacataattttagagaaaattaacacctacacttgtaaagttgtggctttcccacacctatttatcatctctcaatattccttgaaaaggaaattatcaatttatcattctatattggcaatgaaatgcccctaatatctgtcacctataagacaattgaagatgatgtgttgaaagctttctgaaaatgctgatcattactttaaatggaattgaaattccagtttattatttccaaaaatatgatcttactgatcataggataacatttcataacatttcagagatttcttccccttcgaggagccaaacccataggacctctggactcccacagatcctggcagggagttcccactcataaaagcacaggtgccctcagagtcattcagggatgaagaagcaaccctcattggccatgtcctacgttccccatatagtaaggactggaggagaccagtgctcaattctgcagtctcagacagctgtcagaggagagtcatgaatgtgcagtgtctagcacattacaaacgtttgttaattgactgatcattcatggtgtgacagccctataactcagctatcctattcagtcagaaattaactcagtaatcaaagtcattaaaaggaagaaaaaaaaaacctacagtaccagacagatggtggggaaatcagacagatgaaaggaaaaatggctgtaggtcattgagtaagacactgggcagcaaaacctgggccttgtgcctggttatactccacattatagctccagcaaggtttggcaggatttccacagtcctggcttattctaacctttcttgggagcaggagcagtgttggtcagatagacagacacataaggaatctgtccaactggcaccgtgtgaatttgggctcttggtgtacatggataactgggaaaaagaggagagagacatgtaggactgatcctaccgtttgtgaagtcttgggcaagagtatgaatgaaaacccacttctcttcccctgcctggctccactgcacacagtaaagagcctcaagcataggtgtgtggacattgcaccatgtatccaagctctgaccatgcctcttgaaacagctattcctcagccaccctctgaccatgggaggaatgacccaggagaaatgaccacataggtcttgaaaatgggctcagggctatttacgaagtcaattccggggtcccaggagtatggactaaaatgtgagtcaggcatgccagatgggtatgttctattgacttcaaggattcctcatgctgtgggaaggaacctctccagaagagagacagagcagaaccctctaaatgtggggcacaaagcaggagcccctcttgctggattcaaagggtcatactggaagagtgtaggttgagtcttattctcacatcactcatatcacttacacacttcttttatagccttagcagccatgccacaaagagaaactcttcatgcatatcttttggtccataagtcataaatagttatccttgatcccatgtcttttttagagccatggacagagagaagcaaaaatataccaagttcacactgagttgtctcccttcatatctcttagcagtcactgaaaggttatgagactcaggctgggtttctatcctctgtccctgaaacgacaacgttgacctcgtgatcaaccctagaatccagagcaagatctcagactgtcctctctactaccagacagcacactttgttttgggggctgtgtgcttgaaatattagctatggcaaaaggctttgagtcttatgacaccccaagtaacttttactttaggaatttgaaatacagccttgctgtaatgctgtctccttaacaaagcagtacctttgaaatatttaacaacttgaaaaggaaaccgagcttgaattttcctttcaggtgctcaggaaataatgtttcacttctgtctgaaattcaccatctcctcagacaaagaaggctcttatggtaaaaggaatggcattttctccacaattttcgaataaaagataaagagaaaacagcactgcagcctttttgttaggatctaacaataaagaaataatacggttttgccaggggagagctctggttttaagctcagaatacaaaaataggctgacaaaattttacaaaggaatattctcagctaccactctgaggatggtagaaagtgaaatttcaagaaaattatattatttgattatttgatgatgattaagctgattggccagtcctatgtgaaattctaaagtagaagaaatgtgatgttgtcttttctgctcacctctcccctcattcctacccccaaatctctgcctctaccccaaacccagcctgacctttgggaaaggaatgggggctgtcacttgcaccgtagctcctcctgcctgcagtactctccccccacagtccagcctccctctgctcagctaacttttcctatagctccttctggctacttctaaggaaccttccataatgctgcccaccctctcagtaacagcccctctactatcccttgattacatgcactgtaattggttagtaattgattttatgtcctctgccaaattatactccatgagagcaaaaatcatgagtattatctttaatgtttaaatctccacaactatcccccatataagtctagagaataaatgaatgagtgaattaatgaatagaaactcaagccattatgttgccactcctaggatatttggattaactttaactgaagagtaaaaagcatttacctgtcctaaaggagacataaaattagtgggagagtatttggagaaaaaaaagacactgtaatacattcttttgtgttgcctaccctgatgtagtcaggtgtccctgatatggggtgggctgaggatttgaaataaaatacctaatttgacattgtaagtggaggaatcaggatttgaaaccaaattggtttgacctaaatcaagctattatgataaggacttgcaagaaaaaaggaatccataaaaaccatatgaatagctaccaattagtaagcatttatatgtgtcaattacaaagccaaatgcaaatcatgctttatttatattagccaccttttatagatgaagaaattgagacctgaatattaaaattgcctacttttacatagtaagtaaaggaatcagggtttgaacccaaattggtttcacctaaggtagaaaaccatcccagcaagtctcctattaactggaaccctattgtggtggcctgagatataacagtagctgtggaagcgctgtagagtcctggccatcctatgtgctcctgatctggtccctcctgccacctgcttctgctccctgtgccatccacccatctggaagtctcccagtgtccatcttcgggggagacactcaccagagtttccagcttccagccagtatggagtgcccctgtcccacagcaatctcaccgaaatcacagctacatctgttaaaattaggctaccaatgagtgatagatgagggggaaaaataataatagtgtactaaacaaaacaaatgtttatttttctcacacataaaaatctagaggttgaagtccagggctggtccagaggctccaaggatctgggatttagactccctctttcttgtttttccacagcatatggcttccatttctggggccacattggtccaaaatgtatgctggggctccagccattgcatccatatttcagccacaggaaggaggaagtggggaagaaaggacaggcccctaatacctgtatagttcaagaagactatcccgcccatacttcccaaccacccttagttgaacaatgctgtcttaattcaagacactcacatgtctagccaaaaatctgaattctgttacaaacaaggagaatagagatgtgcgccacctcaatacctcatccatagctaccttttcctttgtgcagctgtggccaagtgaaagctgaaggagctgtggtaacccttctgaagggaggctggggcctttcacaagaggctgcatgattgacatttatcctgcatggcctgtgaagtacagagaaatattttctcttgaagccacatcatagcagtggctgctttgtagcctgattccaccattatgcctttaaagtgcctagcaattcagccttcacatcatgcaaagaggaatatctcccagtctttgtaagatcagcttaattctaaccacctccttacctcccactgcactcctacacgcacacacaaatcttcttcactcagagcagaaccataacccaagccctaccacctagagactgaagaatcaggctcatgattacaaatatgcaataattttttgtgtggataatgtcaatggggatgatggtaagagaattccttggtttacacattgaccctcttccctgtcccttacaatcaggaaatatttgtcccaacaccttgtttcttctgttgcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactgaaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaagcttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagactacaattcagtctagccacaaaggggattttttttttttggtaacaggctagagcccggttctgtaagtctttagctgaaatggtccagtacaaaagcactggaaatgagtgggctaggaggacaaggaccgtctcctgcgtgaggagttggttggaggtccccaaggccaggtaccccctgcactcttattggattcctctctgtcttcttggagttttgaaaaactccttcgaacaccaggcttttttctttagaaaacaagtctccaatcgttctctgttccgtagaaagagaaagaaaacctggagcagctgctgaaaaatctaatgaggaactaagaggcaaacccacca

SEQ ID NO: 6 - DNA, 2087 bp, Homo sapiens
acagaagccgtttctcatcatgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcattttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactgaaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaagcttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgct

SEQ ID NO: 7 - DNA, 1444 bp, Homo sapiens
tgcctccccaccattcaccaggctccactgcactggctcattcctgcctcagttgtgcttcttttccctggaaagttctttctgtagatctttaaagggtgatttccttctcaacattcaggtgtcagctcgtttcactttcctgaccatgcccatccactcagagatcactcaaaatcccattaccctattttatttctccatcatatgtatcactatctgaaactatcttgttgttgatgcaggctattttcttgaccccttcatgggactcccaacaggggtaccccatttactcagcctgccctgctcaacctcttgcaggagggagcacacgagtgaacgagtgcaggaaccagctggctgctttagtgctgtgaggagtaaactccatgcaggccctgcagcagcaaccagtttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctggctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagactacaattcagtctagccacaaaggggattttttttttttggtaacaggctagagcccggttctgtaagtctttagctgaaatggtccagtacaaaagcactggaaatgagtgggctaggaggacaaggaccgtctcctgcgtgaggagttggttggaggtccccaaggccaggtaccccctgcactcttattggattcctctctgtcttcttggagttttgaaaaactccttcgaacaccaggcttttttctttagaaaacaagtctccaatcgttctctgttccgtagaaagagaaagaaaacctggagcagctgctgaaaaatctaatgaggaactaagaggcaaacccacca

SEQ ID NO: 8 - DNA, 2398 bp, Homo sapiens
aggagatacttgaaggcagtttgaaagacttgttttacagattcttagtccaaagatttccaattagggagaagaagcagcagaaaaggagaaaagccaagtatgagtgatgatgaggccttcatctactgacatttaacctggcgagaaccgtcgatggtgaagttgccttttcagctgggagctgtccgttcagcttccgtaataaatgcagtcaaagaggcagtcccttcccattgctcacaaaggtcttgtttttgaacctcgccctcacagaagccgtttctcatcatgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcattttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcagttgccctttggctgccagctaccaggcaccaggctcaaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactgaaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaagcttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgct

SEQ ID NO: 9 - DNA, 2259 bp, Homo sapiens
acagaagccgtttctcatcatgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcatgctattttcttgaccccttcatgggactcccaacaggggtaccccatttactcagcctgccctgctcaacctcttgcaggagggagcacacgagtgaacgagtgcaggaaccagctggctgctttagtgctgtgaggagtaaactccatgcaggccctgcagcagcaaccagtttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactgaaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaagcttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgct

SEQ ID NO: 10 - protein, 232 aa, Homo sapiens
MPSQQKKIIFCMAGVFSFACALGVVTALGTPLWIKATVLCKTGALLVNASGQELDKFMGEMQYGLFHGEGVRQCGLGARPFRFSFFPDLLKAIPVSIHVNVILFSAILIVLTMVGTAFFMYNAFGKPFETLHGPLGLYLLSFISGSCGCLVMILFASEVKIHHLSEKIANYKEGTYVYKTQSEKYTTSFWVIFFCFFVHFLNGLLIRLAGFQFPFAKSKDAETTNVAADLMY

SEQ ID NO: 11 - protein, 120 aa, Homo sapiens
MQALQQQPVFPDLLKAIPVSIHVNVILFSAILIVLTMVGTAFFMYNAFGKPFETLHGPLGLYLLSFISGSCGCLVMILFASEVKIHHLSEKIANYKEGTYVYKTQSEKYTTSFWLTKGHS

SEQ ID NO: 12 - protein, 245 aa, Homo sapiens
MPSQQKKIIFCMAGVFSFACALGVVTALGTPLWIKATVLCKTGALLVNASGQELDKFMGEMQYGLFHGEGVRQCGLGARPFRFSFFPDLLKAIPVSIHVNVILFSAILIVLTMVGTAFFMYNAFGKPFETLHGPLGLYLLSFISVALWLPATRHQAQGSCGCLVMILFASEVKIHHLSEKIANYKEGTYVYKTQSEKYTTSFWVIFFCFFVHFLNGLLIRLAGFQFPFAKSKDAETTNVAADLMY

SEQ ID NO: 13 - protein, 185 aa, Homo sapiens
MPSQQKKIIFCMAGVFSFACALGVVTALGTPLWIKATVLCKTGALLVNASGQELDKFMGEMQYGLFHGEGVRQCGLGARPFRFSCYFLDPFMGLPTGVPHLLSLPCSTSCRREHTSERVQEPAGCFSAVRSKLHAGPAAATSFSRFAQSNPSEHPRQCHSLLCHPYCVNHGGDSLLHVQCFWKTF

SEQ ID NO: 14 - protein, 258 aa, Artificial Sequence
  source: /note=“Description of Artificial Sequence Synthetic polypeptide”
MPSQQKKIIFCMAGVFSFACALGVVTALGTPLWIKATVLCKTGALLVNASGQELDKFMGEMQYGLFHGEGVRQCGLGARPFRFSFFPDLLKAIPVSIHVNVILFSAILIVLTMVGTAFFMYNAFGKPFETLHGPLGLYLLSFISGSCGCLVMILFASEVKIHHLSEKIANYKEGTYVYKTQSEKYTTSFWVIFFCFFVHFLNGLLIRLAGFQFPFAKSKDAETTNVAADLMYGSRADYKDHDGDYKDHDIDYKDDDDK

SEQ ID NO: 15 - DNA, 145 bp, Artificial Sequence
  source: /note=“Description of Artificial Sequence Synthetic polynucleotide”
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 16 - DNA, 145 bp, Artificial Sequence
  source: /note=“Description of Artificial Sequence Synthetic polynucleotide”
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 17 - DNA, 119 bp, Artificial Sequence
  source: /note=“Description of Artificial Sequence Synthetic polynucleotide”
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 18 - DNA, 130 bp, Artificial Sequence
  source: /note=“Description of Artificial Sequence Synthetic polynucleotide”
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 19 - DNA, 696 bp, Artificial Sequence
  source: /note=“Description of Artificial Sequence Synthetic polynucleotide”
atgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtac

SEQ ID NO: 20 - DNA, 130 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 21 - DNA, 145 bp, Artificial Sequence
  source: /note=“Description of Artificial Sequence Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 22 - DNA, 145 bp, Artificial Sequence
  source: /note=“Description of Artificial Sequence Synthetic polynucleotide”
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 23 - DNA, 279 bp, Artificial Sequence
  source: /note=“Description of Artificial Sequence Synthetic polynucleotide”
gtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 24 - DNA, 277 bp, Artificial Sequence
  source: /note=“Description of Artificial Sequence Synthetic polynucleotide”
gtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 25 - DNA, 660 bp, Artificial Sequence
  source: /note=“Description of Artificial Sequence Synthetic polynucleotide”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 26 - DNA, 658 bp, Artificial Sequence
  source: /note=“Description of Artificial Sequence Synthetic polynucleotide”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 27 - DNA, 1673 bp, Artificial Sequence
  source: /note=“Description of Artificial Sequence Synthetic polynucleotide”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 28 - DNA, 1671 bp, Artificial Sequence
  source: /note=“Description of Artificial Sequence Synthetic polynucleotide”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 29 - DNA, 1725 bp, Homo sapiens
ctatggagtttgcataacaaacgtttggcagctcgctctcttacactccattaacaagctgtaacatatagctgcaggttgctataatctcattaatattttggaaacttgaatattgagtatttctgagtgctcattccccatatgccagccacttctgccatgctgactggttcctttctctccattattagcaattagcttcttaccttccaaagtcagatccaaggtatccaagatactagcaaaggaatcaactatgtgtgcaagttaagcatgcttaatatcacccaaacaaacaaagaggcagcatttcttaaagtaatgaagatagataaatcgggttagtcctttgcgacactgctggtgctttctagagttttatatattttaagcagcttgctttatattctgtctttgcctcccaccccaccagcacttttatttgtggagggttttggctcgccacactttgggaaacttatttgatttcacggagagctgaaggaagatcatttttggcaacagacaagtttaaacacgatttctatgggacattgctaactggggcccctaaggagaaaggggaaactgagcggagaatgggttaaatccttggaagcaggggagaggcaggggaggagagaagtcggaggagtataaagaaaaggacaggaaccaagaagcgtgggggtggtttgccgtaatgtgagtgtttcttaattagagaacggttgacaatagagggtctggcagaggctcctggccgcggtgcggagcgtctggagcggagcacgcgctgtcagctggtgagcgcactctcctttcaggcagctccccggggagctgtgcggccacatttaacaccatcatcacccctccccggcctcctcaacctcggcctcctcctcgtcgacagccttccttggcccccaccagcagagctcacagtagcgagcgtctctcgccgtctcccgcactcggccggggcctctctcctcccccagctgcgcagcgggagccgccactgcccactgcacctcccagcaaccagcccagcacgcaaagaagctgcgcaaagttaaagccaagcaatgccaaggggaggggaagctggaggcgggctttgagtggcttctgggcgcctggcgggtccagaatcgcccagagccgcccgcggtcgtgcacatctgacccgagtcagcttgggcaccagccgagagccggctccgcaccgctcccgcaccccagccgccggggtggtgacacacaccggagtcgaattacagccctgcaattaacatatgaatctgacgaatttaaaagaaggaaaaaaaaaaaaaaacctgagcaggcttgggagtcctctgcacacaagaacttttctcggggtgtaaaaactctttgattggctgctcgcacgcgcctgcccgcgccctccattggctgagaagacacgcgaccggcgcgaggagggggttgggagaggagcggggggagactgagtggcgcgtgccgctttttaaaggggcgcagcgccttcagcaaccggagaagcatagttgcacgcgacctggtgtgtgatctccgagtgggtgggggagggtcgaggagggaaaaaaaaataagacgttgcagaagagacccggaaagggccttttttttggttgagctggtgtcccagtgctgcctccgatcctgagcctccgagcctttgcagtgcaa

SEQ ID NO: 30 - DNA, 60 bp, Homo sapiens
ctgccttctgagagcgctataaaggcagcggaagggtagtccgcggggcattccgggcgg

SEQ ID NO: 31 - DNA, 60 bp, Homo sapiens
ctctaggcgggctctgctcttctttaaggagtcccacagggcctggcccgcccctgacct

SEQ ID NO: 32 - DNA, 2000 bp, Homo sapiens
taaagagttgtgagttgtgtaggtgagttgccatggagctacaaatatgagttgatattctgaaatcctagacagccatctccaaggttaagaaaaatccttatgcactcacttgcaaagatatccacagcatgctcttaatggagaaaaacaaagccttagatcaaatatgtaaagtaatttttagttttttgaaaaggtatgtttgggctatagataaatctgttcaaaaaacatgagagaagataataatggttgaaaggagacacagtgcttgccctcaagaagtttttgtctagtgagggagagagaacttgtatgtaaataaaattgtgttactaaggtagatagtgagaagtaacttaagagaggatcagataaggtattaagagaatacagaaaagggtctggattaattctgaacagcatcaaagaatgttcttgcaagagatagtgttttcaccagatcttgaaggtatggatgagggtatacagagtgagtatattcagattctactttaaaacaaatactttcctctgttgtagtggagttgagctatacatccaacaataatgaaaaaatacacgcatatatacatatatggagagagatacatattttagtacatgtagcaattgattaataaatgtacagtttaagtcgcatgcaaaaccttggagtgatagcaaacttcattgtaggatgtttagcagcatctctggtctctactcactagatcccaatagcatctccctaggtgtgacaaccaaaaatgtctccaggcattgacctctggaggcaaaaaaagccctttattaagaaccagtggtatacataagtaaaacatacacaagagattcctcccctcttctctgtatgtgaataaaaattgcaaagttcatgacctggattttccttttaggtttcttctttagtggttcttaacttcattgggtgaagtaagcctttgaagatctgttgaaagctgttgactcattcacttctcaggaaaacgcacatgctgactaccatttcagagaatttgcatcagggttctctggggaggagttctgagttctgtttccaggagctcgtagaattgtcatggtctgcatatgcaaggcaggtggattacggaaggttgatgtacagaggtctgtattttggagcctcttctgtatttacttcagaacactaacaatcaggcgagaatgttctggtttatcaaacccttccttctgcctttcatcttaaccatgcattagttttaacaaagttcatcccaacagaagacaaaacactgatgaggtaggatagctccagctcctcctccctctcttctagtcttgatttccatgtagtccagtttattccttccctgattgtccaggagaatgagaaaaagaaaaaacagagtctagtgggtaagaaagggccacctggacggcttgatttggattgtgaaataaaacacacacacatgcacacgtagaataagtggctaaaatctgagtaaatcgtgaactctctgtatcctccacccattgaatactcctaaaagactttctagaaattcaaggacttattaatatagaaacctggccattgttcctcttctcctccccatgtggtatgagagcacctgtggcaggctcccagagaccacggacctcttcctctaggcgggctctgctcttctttaaggagtcccacagggcctggcccgcccctgacctcgcaacccttgagattagtaacgggatgagtgaggatccgggtggcccctgcgtggcagccagtaagagtctcagccttcccggttcgggaaaggggaagaatgcaggaggggtaggatttctttcctgataggatcggttgggaaagaccgcagcctgtgtgtgtctttcccttcgaccaaggtgtctgttgctccgtaaataaaacgtcccactgccttctgagagcgctataaaggcagcggaagggtagtccgcggggc

SEQ ID NO: 33 - DNA, 1400 bp, Homo sapiens
ggctgctcggaaaacaggacgaggggagagacttgctcaataagctgaaagttctgcccccgagagggctgcgacagctgctggaatgtgcctgcagcgtccgcctcttggggacccgcggagcgcgccctgacggttccacgcctggcccgggggtctgcacctctcctccagtgcgcacctggagctgcgtcccgggtcaggtgcggggagggagggaatctcagtgtccccttccagccttgcaagcgcctttggcccctgccccagcccctcggtttgggggagatttcagaacgcggacagcgccctggctgcgggccataggggactgggtggaactcgggaagcccccagagcaggggcttactcgcttcaagtttggggaaccccgggcagcgggtgcaggccacgagacccgaaggttctcaggtgcccccctgcaggctggccgtgcgcgccgtggggcgcttgtcgcgagcgccgagggctgcaggacgcggaccagactcgcggtgcaggggggcctggctgcagctaacaggtgatcccgttctttctgttcctcgctcttcccctccgatcgtcctcgcttaccgcgtgtcctccctcctcgctgtcctctggctcgcaggtcatggcagcgccaggcggcaggtcggagccgccgcagctccccgagtacagctgcagctacatggtgtcgcggccggtctacagcgagctcgctttccagcaacagcacgagcggcgcctgcaggagcgcaagacgctgcgggagagcctggccaagtgctgcaggtagcggccgcgcgggcctgcgtagagagaagcggagcggggcgtccacgccttggggagggaagggcgtccccagcgggcgagagtggggtgcgggcggcggagcccctgggcgccagctgcttctcccagaggcccgactttcggtctccggtcctccacgccgcccttctggtgggagggtggctccatcagtctcgggcccgaaatgaacttacctgggaaactcgcctttggggagagtgggttctaggagccccgtctctctttttcctctctgaaggaaacttggagtgcctcttggggtacagtgggtccctgttgccttcttgggagcttgtttaaatgaaatgaatagggaaacccagctcttgaccaggaggagtccttgaaacactcaagctaagtaggcgggctaccattcagttagagaccaggatgcaagctagaacccaggggagcgcggggtgtgccaagtacttcatcagcaggctgtgggacccctggggaaagccaccctcagtctctaaacccaaacatgccgtaactagatgtcacaaacataaagaaattagagtttctaaaacctttcattatag

SEQ ID NO: 34 - DNA, 900 bp, Homo sapiens
cggaaggttgatgtacagaggtctgtattttggagcctcttctgtatttacttcagaacactaacaatcaggcgagaatgttctggtttatcaaacccttccttctgcctttcatcttaaccatgcattagttttaacaaagttcatcccaacagaagacaaaacactgatgaggtaggatagctccagctcctcctccctctcttctagtcttgatttccatgtagtccagtttattccttccctgattgtccaggagaatgagaaaaagaaaaaacagagtctagtgggtaagaaagggccacctggacggcttgatttggattgtgaaataaaacacacacacatgcacacgtagaataagtggctaaaatctgagtaaatcgtgaactctctgtatcctccacccattgaatactcctaaaagactttctagaaattcaaggacttattaatatagaaacctggccattgttcctcttctcctccccatgtggtatgagagcacctgtggcaggctcccagagaccacggacctcttcctctaggcgggctctgctcttctttaaggagtcccacagggcctggcccgcccctgacctcgcaacccttgagattagtaacgggatgagtgaggatccgggtggcccctgcgtggcagccagtaagagtctcagccttcccggttcgggaaaggggaagaatgcaggaggggtaggatttctttcctgataggatcggttgggaaagaccgcagcctgtgtgtgtctttcccttcgaccaaggtgtctgttgctccgtaaataaaacgtcccactgccttctgagagcgctataaaggcagcggaagggtagtccgcggggcattccgggcggggcgcgagcagagacaggtgagtt

SEQ ID NO: 35 - DNA, 1269 bp, Homo sapiens
agggctatttgtacctcaacgagggcttctctccaagaaagccctgaatccttttcctcctttttcctgcagattcactataggacactttttgaagcaagagcatgcattttccccctggcgctctgcagcggttctcagagcccagtgtcactcacataggtgggactgctctcagttcagagagcgctgggacacttaagatgaaaagtccctggaagttagcaaacagccatctgtcactctggcatcgatttactaaaagtgacttctagggtattctaaaccacttttaaaaaacaaatgagtcacttcgacttcctcaccccgcaagagataggaaggcagcagtggagtgctcgctcaggagctgtatttgtttagcgattagcctagagctttgattttagggcaaaagcgagccagacagtgcggcagacgtaaggatcaaaaaggccacctatcattcgccggggacgcctgcctccttaccctgataacgtaactatttctctgcataggattttagtttttgtgtttttgttttgttttattctgtttaatcacttcaagtatctcatccattatttgaagcgggctcggaggaaacgtgccgcatcctccagtccttgtgcgtctgtttaggtctctccgaagcaggtccctctcgactcttagatctgggtctccagcacgcatgaaggggtaagggtgggggggtcccctattccggcgcgcggcgttgagcactgaatcttccaggcggaggctcagtgggagcgccgagaactcgccagtaccgcgcgctgcctgctgcctgctgcctcccagcccaggacttgggaaaggagggaggggacaagtggagggaaagtggggccgggcggggggtgcctgggaagccaggctgcgctgacgtcactgggcgcgcaattcgggctggagcgctttaaaaaacgagcgtgcaagcagagatgctgctccacaccgctcaggccgcgagcagcagcaaggcgcaccgccactgtcgccgctgcagccagggctgctccgaaggccggcgtggcggcaaccggcacctctgtccccgccgcgcttctcctcgccgcccacgccgtggggtcaggaacgcggcgtctggcgctgcagacgcccgctgagttgcagaagcccacggagcggcgcccggcgcgccacggcccgtagcagtccggtgctgctctccgcccgcgtccggctcgtggccccctacttcgggcaccgaccggt

SEQ ID NO: 36 - DNA, 463 bp, Homo sapiens
tgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaa

SEQ ID NO: 37 - DNA, 2204 bp, Homo sapiens
cccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttcccttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 38 - DNA, 381 bp, Artificial Sequence
  source: /note=“Description of Artificial Sequence Synthetic polynucleotide”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgg

SEQ ID NO: 39 - DNA, 1013 bp, Artificial Sequence
  source: /note=“Description of Artificial Sequence Synthetic polynucleotide”
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 40 - DNA, 291 bp, Artificial Sequence
  source: /note=“Description of Artificial Sequence Synthetic polynucleotide”
aggagatacttgaaggcagtttgaaagacttgttttacagattcttagtccaaagatttccaattagggagaagaagcagcagaaaaggagaaaagccaagtatgagtgatgatgaggccttcatctactgacatttaacctggcgagaaccgtcgatggtgaagttgccttttcagctgggagctgtccgttcagcttccgtaataaatgcagtcaaagaggcagtcccttcccattgctcacaaaggtcttgtttttgaacctcgccctcacagaagccgtttctcatc

SEQ ID NO: 41 - DNA, 1369 bp, Artificial Sequence
  source: /note=“Description of Artificial Sequence Synthetic polynucleotide”
aaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgct

SEQ ID NO: 42 - DNA, 82 bp, Artificial Sequence
  source: /note=“Description of Artificial Sequence Synthetic oligonucleotide”
gtaagtatcaaggttacaagacaggtttaaggagaccaatagaaactgggcttgtcgagacagagaagactcttgcgtttct

SEQ ID NO: 43 - DNA, 51 bp, Artificial Sequence
  source: /note=“Description of Artificial Sequence Synthetic oligonucleotide”
gataggcacctattggtcttactgacatccactttgcctttctctccacag

SEQ ID NO: 44 - DNA, 225 bp, Artificial Sequence
  source: /note=“Description of Artificial Sequence Synthetic polynucleotide”
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 45 - DNA, 122 bp, Artificial Sequence
  source: /note=“Description of Artificial Sequence Synthetic polynucleotide”
aacttgtttattgcagcttataatggttacaaataaagcaatagcatcacaaatttcacaaataaagcatttttttcactgcattctagttgtggtttgtccaaactcatcaatgtatctta

SEQ ID NO: 46 - DNA, 22 bp, Artificial Sequence
  source: /note=“Description of Artificial Sequence Synthetic oligonucleotide”
ttgtcgacgcggccgcacgcgt

SEQ ID NO: 47 - DNA, 44 bp, Artificial Sequence
  source: /note=“Description of Artificial Sequence Synthetic oligonucleotide”
ctcctgggcaacgtgctggttattgtgaccggtcgctagccacc

SEQ ID NO: 48 - DNA, 24 bp, Artificial Sequence
  source: /note=“Description of Artificial Sequence Synthetic oligonucleotide”
taagagctcgctgatcagcctcga

SEQ ID NO: 49 - DNA, 39 bp, Artificial Sequence
  source: /note=“Description of Artificial Sequence Synthetic oligonucleotide”
aagcttgaattcagctgacgtgcctcggaccgtcctagg

SEQ ID NO: 50 - DNA, 14 bp, Artificial Sequence
  source: /note=“Description of Artificial Sequence Synthetic oligonucleotide”
gcggccgcacgcgt

SEQ ID NO: 51 - DNA, 39 bp, Artificial Sequence
  source: /note=“Description of Artificial Sequence Synthetic oligonucleotide”
ctcctgggcaacgtgctggttattgtgaccggtgccacc

SEQ ID NO: 52 - DNA, 24 bp, Artificial Sequence
  source: /note=“Description of Artificial Sequence Synthetic oligonucleotide”
taagagctcgctgatcagcctcga

SEQ ID NO: 53 - DNA, 34 bp, Artificial Sequence
  source: /note=“Description of Artificial Sequence Synthetic oligonucleotide”
aagcttgaattcagctgacgtgcctcggaccgct

SEQ ID NO: 54 - DNA, 33 bp, Artificial Sequence
  source: /note=“Description of Artificial Sequence Synthetic oligonucleotide”
ctcctgggcaacgtgctggttattgtgaccggt

SEQ ID NO: 55 - DNA, 6 bp, Artificial Sequence
  source: /note=“Description of Artificial Sequence Synthetic oligonucleotide”
gccacc

SEQ ID NO: 56 - DNA, 9 bp, Artificial Sequence
  source: /note=“Description of Artificial Sequence Synthetic oligonucleotide”
taagagctc

SEQ ID NO: 57 - protein, 159 aa, Artificial Sequence
  source: /note=“Description of Artificial Sequence Synthetic polypeptide”
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 58 - DNA, 483 bp, Artificial Sequence
  source: /note=“Description of Artificial Sequence Synthetic polynucleotide”
ggtaccatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgataa

SEQ ID NO: 59 - DNA, 474 bp, Artificial Sequence
  source: /note=“Description of Artificial Sequence Synthetic polynucleotide”
atcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcga

SEQ ID NO: 60 - protein, 112 aa, Artificial Sequence
  source: /note=“Description of Artificial Sequence Synthetic polypeptide”
MGVEKQVIRPGNGPKPAPGQTVTVHCTGFGKDGDLSQKFWSTKDEGQKPFSFQIGKGAVIKGWDEGVIGMQIGEVARLRCSSDYAYGAGGFPAWGIQPNSVLDFEIEVLSVQ

SEQ ID NO: 61 - DNA, 78 bp, Artificial Sequence
  source: /note=“Description of Artificial Sequence Synthetic oligonucleotide”
ggatcccgggctgactacaaagaccatgacggtgattataaagatcatgacatcgactacaaggatgacgatgacaag

SEQ ID NO: 62 - DNA, 2208 bp, Artificial Sequence
  source: /note=“Description of Artificial Sequence Synthetic polynucleotide”
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcaatcaccccaggaaccagactcctcttcgggcatcggcaagaaaggccagcagcccgcgaagaagagactcaactttgggcagacaggcgactcagagtcagtgcccgaccctcaaccactcggagaaccccccgcagccccctctggtgtgggatctaatacaatggcagcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggagcaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctacacgtttgaggacgtgccttttcacagcagctacgcgcacagccaaagcctggaccggctgatgaaccccctcatcgaccagtacctgtactacctgtctcggactcagaccacgagtggtaccgcaggaaatcggacgttgcaattttctcaggccgggcctagtagcatggcgaatcaggccaaaaactggctacccgggccctgctaccggcagcaacgcgtctccaagacagcgaatcaaaataacaacagcaactttgcctggaccggtgccaccaagtatcatctgaatggcagagactctctggtaaatcccggtcccgctatggcaacccacaaggacgacgaagacaaattttttccgatgagcggagtcttaatatttgggaaacagggagctggaaatagcaacgtggaccttgacaacgttatgataaccagtgaggaagaaattaaaaccaccaacccagtggccacagaacagtacggcacggtggccactaacctgcaatcgtcaaacaccgctcctgctacagggaccgtcaacagtcaaggagccttacctggcatggtctggcagaaccgggacgtgtacctgcagggtcctatctgggccaagattcctcacacggacggacactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacaaatggcgtttattctgagcctcgccccatcggcacccgttacctcacccgtaatctg

SEQ ID NO: 63 - protein, 736 aa, Artificial Sequence
  source: /note=“Description of Artificial Sequence Synthetic polypeptide”
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGASTNDNTYFGYSTPWGYFDFNRFHCHFSPRDWQRLINNNWGFRPKRLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTSGTAGNRTLQFSQAGPSSMANQAKNWLPGPCYRQQRVSKTANQNNNSNFAWTGATKYHLNGRDSLVNPGPAMATHKDDEDKFFPMSGVLIFGKQGAGNSNVDLDNVMITSEEEIKTTNPVATEQYGTVATNLQSSNTAPATGTVNSQGALPGMVWQNRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 64 - DNA, 4653 bp, Artificial Sequence
  source: /note=“Description of Artificial Sequence Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtaggagatacttgaaggcagtttgaaagacttgttttacagattcttagtccaaagatttccaattagggagaagaagcagcagaaaaggagaaaagccaagtatgagtgatgatgaggccttcatctactgacatttaacctggcgagaaccgtcgatggtgaagttgccttttcagctgggagctgtccgttcagcttccgtaataaatgcagtcaaagaggcagtcccttcccattgctcacaaaggtcttgtttttgaacctcgccctcacagaagccgtttctcatcgccaccatgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcattttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgtcctaggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 65 - DNA, 16 bp, Artificial Sequence
  source: /note=“Description of Artificial Sequence Synthetic probe”
ccagcagagtcagggc

SEQ ID NO: 66 - DNA, 23 bp, Artificial Sequence
  source: /note=“Description of Artificial Sequence Synthetic primer”
gatacagctagagtcctgattgc

SEQ ID NO: 67 - DNA, 23 bp, Artificial Sequence
  source: /note=“Description of Artificial Sequence Synthetic primer”
gatctgccaagtacctcactatg

SEQ ID NO: 68 - DNA, 4653 bp, Artificial Sequence
  source: /note=“Description of Artificial Sequence Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtaggagatacttgaaggcagtttgaaagacttgttttacagattcttagtccaaagatttccaattagggagaagaagcagcagaaaaggagaaaagccaagtatgagtgatgatgaggccttcatctactgacatttaacctggcgagaaccgtcgatggtgaagttgccttttcagctgggagctgtccgttcagcttccgtaataaatgcagtcaaagaggcagtcccttcccattgctcacaaaggtcttgtttttgaacctcgccctcacagaagccgtttctcatcgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtactaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgtcctaggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 69 - DNA, 17 bp, Unknown
  source: /note=“Description of Unknown Poly(A) signal sequence”
aaataaaatacgaaatg

SEQ ID NO: 70 - protein, 736 aa, Artificial Sequence; identical to SEQ ID NO: 63
  source: /note=“Description of Artificial Sequence Synthetic polypeptide”
  VARIANT (168)..(168): /replace=“R”
  VARIANT (204)..(204): /replace=“S”
  VARIANT (266)..(266): /replace=“G”
  VARIANT (311)..(311): /replace=“K”
  VARIANT (411)..(411): /replace=“Q”
  VARIANT (460)..(460): /replace=“E”
  VARIANT (493)..(493): /replace=“T”
  VARIANT (562)..(562): /replace=“N”
  VARIANT (576)..(576): /replace=“E”
  VARIANT (587)..(587): /replace=“A”
  VARIANT (609)..(609): /replace=“D”
  SITE (1)..(736): /note=“Variant residues given in the sequence have no preference with respect to those in the annotations for variant positions”

SEQ ID NO: 71 - RNA, 5000 nt, Artificial Sequence
  source: /note=“Description of Artificial Sequence Synthetic polynucleotide”
  misc_feature (1)..(5000): /note=“This sequence may encompass 50, 60, 70, 100, 200, 500, 1000, 2000, 3000, 4000, or 5000 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2024/0139344 A1
(43) Pub. Date: May 2, 2024

(54) COMPOSITIONS AND METHODS FOR TREATING CLRN1-ASSOCIATED HEARING LOSS AND/OR VISION LOSS

(71) Applicant: Akouos, Inc., Boston, MA (US)

(72) Inventors: Emmanuel John Simons, Brookline, MA (US); Robert Ng, Newton, MA (US)

(21) Appl. No.: 18/269,307

(22) PCT Filed: Dec. 22, 2021
(86) PCT No.: PCT/US21/64924

§ 371 Date: Jun. 23, 2023

Related U.S. Application Data
(60) Provisional application No. 63/131,413, filed on Dec. 29, 2020.

(51) Int. Cl. A61K 48/00; C07K 14/705; C12N 15/86

(52) U.S. Cl. CPC A61K 48/0058; A61K 48/0075; A61K 48/0091; C07K 14/705; C12N 15/86; C12N 2750/14122; C12N 2750/14143; C12N 2750/14152; C12N 2830/008; C12N 2830/42; C12N 2830/50; C12N 2840/445

(57) ABSTRACT

The present disclosure provides constructs comprising a coding sequence operably linked to a promoter, wherein the coding sequence encodes a clarin 1 protein. Exemplary constructs include AAV constructs. Also provided are methods of using disclosed constructs for the treatment of hearing loss and/or deafness. Also provided are methods of using disclosed constructs for the treatment of vision loss.

### CROSS-REFERENCE TO RELATED APPLICATIONS

This application is the National Stage of International Application No. PCT/US2021/064924, filed Dec. 22, 2021, which claims priority to U.S. Provisional Patent Applications 63/131,413 filed on Dec. 29, 2020, the entire contents of which is hereby incorporated by reference.

### BACKGROUND

Hearing loss can be conductive (arising from the ear canal or middle ear), sensorineural (arising from the inner ear or auditory nerve), or mixed. Most forms of nonsyndromic deafness are associated with permanent hearing loss caused by damage to structures in the inner ear (sensorineural deafness), although some forms may involve changes in the middle ear (conductive hearing loss). The great majority of human sensorineural hearing loss is caused by abnormalities in the hair cells of the organ of Corti in the cochlea (poor hair cell function). The hair cells may be abnormal at birth, or may be damaged during the lifetime of an individual (e.g., as a result of noise trauma or infection).

Vision loss, also known as visual impairment or vision impairment, is a decreased ability to see, e.g., to a degree that is not correctable by means such as eyeglasses. The causes of vision loss are extremely varied and range from conditions affecting the eyes to conditions affecting the visual processing centers in the brain. For example, vision loss in patients suffering from Usher III syndrome or retinitis pigmentosa occurs as the light-sensing cells of the retina gradually deteriorate and eventually atrophy. Other examples of losses of vision loss include diabetic retinopathy, glaucoma, age-related macular degeneration, and cataracts.

### SUMMARY

The present disclosure provides the recognition that diseases or conditions associated with hearing loss can be treated via, e.g., the replacement or addition of certain gene products. The present disclosure further provides that gene products involved in the development, function, and/or maintenance of inner ear cells can be useful for treatment of diseases or conditions associated with hair cell and/or supporting cell (e.g., supporting hair cell) loss. The present disclosure thus provides for the administration of compositions that result in expression of gene products involved in the development, function, and/or maintenance of inner ear cells including supporting cells and hair cells, and/or the use of such compositions in the treatment of hearing loss, or diseases or conditions associated with hearing loss. In some embodiments, a gene product can be encoded by a CLRN1 gene or a characteristic portion thereof. In some embodiments, a gene product can be clarin 1 protein or a characteristic portion thereof.

The present disclosure further provides the recognition that diseases or conditions associated with vision loss can be treated via, e.g., the replacement or addition of certain gene products. The present disclosure further provides that gene products involved in the development, function, and/or maintenance of eye cells can be useful for treatment of diseases or conditions associated with eye cell and/or supporting cell (e.g., supporting eye cell) loss. The present disclosure thus provides for the administration of compositions that result in expression of gene products involved in the development, function, and/or maintenance of eye cells including supporting cells, and/or the use of such compositions in the treatment of vision loss, or diseases or conditions associated with vision loss. In some embodiments, a gene product can be encoded by a CLRN1 gene or a characteristic portion thereof. In some embodiments, a gene product can be clarin 1 protein or a characteristic portion thereof.

The present disclosure further provides that AAV particles can be useful for administration of compositions that result in expression of gene products involved in the development, function, and/or maintenance of inner ear cells, and/or the treatment of hearing loss, or diseases or conditions associated with hearing loss. The present disclosure further provides that AAV particles can be useful for administration of compositions that result in expression of gene products involved in the development, function, and/or maintenance of eye cells, and/or the treatment of vision loss, or diseases or conditions associated with vision loss. As described herein, AAV particles comprise (i) a AAV polynucleotide construct (e.g., a recombinant AAV polynucleotide construct), and (ii) a capsid comprising capsid proteins. In some embodiments, an AAV polynucleotide construct comprises a CLRN1 gene or a characteristic portion thereof. In some embodiments, AAV particles described herein are referred to as rAAV-CLRN1 or rAAV-CLRN1 particles. In some embodiments, AAV particles described herein are referred to as rAAV Anc80-CLRN1 or rAAV Anc80-CLRN1 particles.

The present disclosure further provides compositions comprising polynucleotide constructs comprising a CLRN1 gene or a characteristic portion thereof. In some embodiments, a construct may further include regulatory elements operably attached to a coding sequence. In certain embodiments, included regulatory elements facilitate tissue specific expression at physiologically suitable levels.

Also provided herein are methods of administering constructs and compositions described herein. In certain embodiments, administration involves surgical intervention and the delivery of rAAV particles comprising therapeutic constructs. In certain embodiments AAV particles may be delivered to the inner ear of a subject in need thereof by surgical introduction through the round window membrane. In some embodiments, efficacy of an intervention is determined through established tests, and measurements are compared to control or reference measurements.

### Definitions

The scope of the present disclosure is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include the plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of a group is present in, employed in, or otherwise relevant to a given product or process. In some embodiments, more than one, or all group members are present in, employed in, or otherwise relevant to a given product or process. It is to be understood that the present disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where embodiments or aspects are referred to as “comprising” particular elements, features, etc., certain embodiments or aspects “consist,” or “consist essentially of,” such elements, features, etc. For purposes of simplicity, those embodiments have not in every case been specifically set forth in so many words herein. It should also be understood that any embodiment or aspect can be explicitly excluded from the claims, regardless of whether the specific exclusion is recited in the specification.

Throughout the specification, whenever a polynucleotide or polypeptide is represented by a sequence of letters (e.g., A, C, G, and T, which denote adenosine, cytidine, guanosine, and thymidine, respectively in the case of a polynucleotide), such polynucleotides or polypeptides are presented in 5′ to 3′ or N-terminus to C-terminus order, from left to right.

Administration: As used herein, the term “administration” typically refers to administration of a composition to a subject or system to achieve delivery of an agent to a subject or system. In some embodiments, an agent is, or is included in, a composition; in some embodiments, an agent is generated through metabolism of a composition or one or more components thereof. Those of ordinary skill in the art will be aware of a variety of routes that may, in appropriate circumstances, be utilized for administration to a subject, for example a human. For example, in some embodiments, administration may be systematic or local. In some embodiments, a systematic administration can be intravenous. In some embodiments, administration can be local. Local administration can involve delivery to cochlear perilymph via, e.g., injection through a round-window membrane or into scala-tympani, a scala-media injection through endolymph, perilymph and/or endolymph following canalostomy. Local administration can also involve delivery to the eye via, e.g., an intra-ocular injection (e.g., a vitreous injection, an intravitreal injection, a subretinal injection, a suprachoroidal injection (e.g., using the Orbit™ Subretinal Delivery System (Orbit SDS) (Gyroscope Therapeutics)), or a retinal injection). See, e.g., Ochakovski et al., “Retinal Gene Therapy: Surgical Vector Delivery in the Translation to Clinical Trials”, Front. Neurosci. Apr. 3, 2017 or “OCT—Assisted Delivery of Luxturna” by Ninel Z Gregori and Janet Louise David, https://www.aao.org/clinical-video/oct-assisted-delivery-of-luxturna (Jul. 19, 2018), the disclosure of each of which is hereby incorporated by reference in its entirety. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Allele: As used herein, the term “allele” refers to one of two or more existing genetic variants of a specific polymorphic genomic locus.

Amelioration: As used herein, the term “amelioration” refers to prevention, reduction or palliation of a state, or improvement of a state of a subject. Amelioration may include, but does not require, complete recovery or complete prevention of a disease, disorder or condition.

Amino acid: In its broadest sense, as used herein, the term “amino acid” refers to any compound and/or substance that can be incorporated into a polypeptide chain, e.g., through formation of one or more peptide bonds. In some embodiments, an amino acid has a general structure, e.g., H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than standard amino acids, regardless of whether it is prepared synthetically or obtained from a natural source. In some embodiments, an amino acid, including a carboxy- and/or amino-terminal amino acid in a polypeptide, can contain a structural modification as compared with general structure as shown above. For example, in some embodiments, an amino acid may be modified by methylation, amidation, acetylation, pegylation, glycosylation, phosphorylation, and/or substitution (e.g., of an amino group, a carboxylic acid group, one or more protons, and/or a hydroxyl group) as compared with a general structure. In some embodiments, such modification may, for example, alter circulating half-life of a polypeptide containing a modified amino acid as compared with one containing an otherwise identical unmodified amino acid. In some embodiments, such modification does not significantly alter a relevant activity of a polypeptide containing a modified amino acid, as compared with one containing an otherwise identical unmodified amino acid.

Approximately or About: As used herein, the terms “approximately” or “about” may be applied to one or more values of interest, including a value that is similar to a stated reference value. In some embodiments, the term “approximately” or “about” refers to a range of values that fall within ±10% (greater than or less than) of a stated reference value unless otherwise stated or otherwise evident from context (except where such number would exceed 100% of a possible value). For example, in some embodiments, the term “approximately” or “about” may encompass a range of values that within 10%, 9%, 8%, 7%, 6%, 5%, 4%, 3%, 2%, 1%, or less of a reference value.

Associated: As used herein, the term “associated” describes two events or entities as “associated” with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biologically active: As used herein, the term “biologically active” refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Characteristic portion: As used herein, the term “characteristic portion,” in the broadest sense, refers to a portion of a substance whose presence (or absence) correlates with presence (or absence) of a particular feature, attribute, or activity of the substance. In some embodiments, a characteristic portion of a substance is a portion that is found in a given substance and in related substances that share a particular feature, attribute or activity, but not in those that do not share the particular feature, attribute or activity. In some embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to a sequence and/or structural identity specified above, shares at least one functional characteristic with the relevant intact substance. In some embodiments, a characteristic portion may be biologically active.

Characteristic sequence: As used herein, the term “characteristic sequence” is a sequence that is found in all members of a family of polypeptides or nucleic acids, and therefore can be used by those of ordinary skill in the art to define members of the family.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of a polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share a sequence element.

Combination therapy: As used herein, the term “combination therapy” refers to those situations in which a subject is simultaneously exposed to two or more therapeutic regimens (e.g., two or more therapeutic agents). In some embodiments, two or more agents may be administered simultaneously. In some embodiments, two or more agents may be administered sequentially. In some embodiments, two or more agents may be administered in overlapping dosing regimens.

Comparable: As used herein, the term “comparable” refers to two or more agents, entities, situations, sets of conditions, subjects, populations, etc., that may not be identical to one another but that are sufficiently similar to permit comparison therebetween so that one skilled in the art will appreciate that conclusions may reasonably be drawn based on differences or similarities observed. In some embodiments, comparable sets of agents, entities, situations, sets of conditions, subjects, populations, etc. are characterized by a plurality of substantially identical features and one or a small number of varied features. Those of ordinary skill in the art will understand, in context, what degree of identity is required in any given circumstance for two or more such agents, entities, situations, sets of conditions, subjects, populations, etc. to be considered comparable. For example, those of ordinary skill in the art will appreciate that sets of agents, entities, situations, sets of conditions, subjects, populations, etc. are comparable to one another when characterized by a sufficient number and type of substantially identical features to warrant a reasonable conclusion that differences in results obtained or phenomena observed under or with different sets of circumstances, stimuli, agents, entities, situations, sets of conditions, subjects, populations, etc. are caused by or indicative of the variation in those features that are varied.

Construct: As used herein, the term “construct” refers to a composition including a polynucleotide capable of carrying at least one heterologous polynucleotide. In some embodiments, a construct can be a plasmid, a transposon, a cosmid, an artificial chromosome (e.g., a human artificial chromosome (HAC), a yeast artificial chromosome (YAC), a bacterial artificial chromosome (BAC), or a P1-derived artificial chromosome (PAC)) or a viral construct, and any Gateway® plasmids. A construct can, e.g., include sufficient cis-acting elements for expression; other elements for expression can be supplied by the host primate cell or in an in vitro expression system. A construct may include any genetic element (e.g., a plasmid, a transposon, a cosmid, an artificial chromosome, or a viral construct, etc.) that is capable of replicating when associated with proper control elements. Thus, in some embodiments, “construct” may include a cloning and/or expression construct and/or a viral construct (e.g., an adeno-associated virus (AAV) construct, an adenovirus construct, a lentivirus construct, or a retrovirus construct).

Conservative: As used herein, the term “conservative” refers to instances describing a conservative amino acid substitution, including a substitution of an amino acid residue by another amino acid residue having a side chain R group with similar chemical properties (e.g., charge or hydrophobicity). In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM25D log-likelihood matrix disclosed in Gonnet, G. H. et al., 1992, Science 256:1443-1445, which is incorporated herein by reference in its entirety. In some embodiments, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein.

CONSERVATIVE AMINO ACID SUBSTITUTIONS

| For Amino Acid | Code | Replace With |
|---|---|---|
| Alanine | A | D-ala, Gly, Aib, β-Ala, Acp, L-Cys, D-Cys |
| Arginine | R | D-Arg, Lys, D-Lys, homo-Arg, D-homo-Arg, Met, Ile, |
| | | D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, β-Ala, Acp |
| Isoleucine | I | D-Ile, Val, D-Val, AdaA, AdaG, Leu, D-Leu, Met, D- |
| | | Met |
| Leucine | L | D-Leu, Val, D-Val, AdaA, AdaG, Leu, D-Leu, Met, D- |
| | | Met |
| Lysine | K | D-Lys, Arg, D-Arg, homo-Arg, D-homo-Arg, Met, D- |
| | | Met, Ile, D-Ile, Orn, D-Orn |
| Methionine | M | D-Met, S-Me-Cys, Ile, D-Ile, Leu, D-Leu, Val, D-Val |
| Phenylalanine | F | D-Phe, Tyr, D-Thr, L-Dopa, His, D-His, Trp, D-Trp, |
| | | Trans-3,4 or 5-phenylproline, AdaA, AdaG, cis-3,4 or |
| | | 5-phenylproline, Bpa, D-Bpa |
| Proline | P | D-Pro, L-I-thioazolidine-4-carboxylic acid, D-or-L-1- |
| | | oxazolidine-4-carboxylic acid (Kauer, U.S. Pat. No. |
| | | 4,511,390) |
| Serine | S | D-Ser, Thr, D-Thr, allo-Thr, Met, D-Met, Met (O), D- |
| | | Met (O), L-Cys, D-Cys |
| Threonine | T | D-Thr, Ser, D-Ser, allo-Thr, Met, D-Met, Met (O), D- |
| | | Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, AdaA, |
| | | AdaG |

Control: As used herein, the term “control” refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. For example, in one experiment, a “test” (i.e., a variable being tested) is applied. In a second experiment, a “control,” the variable being tested is not applied. In some embodiments, a control is a historical control (e.g., of a test or assay performed previously, or an amount or result that is previously known). In some embodiments, a control is or comprises a printed or otherwise saved record. In some embodiments, a control is a positive control. In some embodiments, a control is a negative control.

Determining, measuring, evaluating, assessing, assaying and analyzing: As used herein, the terms “determining,” “measuring,” “evaluating,” “assessing,” “assaying,” and “analyzing” may be used interchangeably to refer to any form of measurement, and include determining if an element is present or not. These terms include both quantitative and/or qualitative determinations. Assaying may be relative or absolute. For example, in some embodiments, “Assaying for the presence of” can be determining an amount of something present and/or determining whether or not it is present or absent.

Engineered: In general, as used herein, the term “engineered” refers to an aspect of having been manipulated by the hand of man. For example, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Excipient: As used herein, the term “excipient” refers to an inactive (e.g., non-therapeutic) agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to generation of any gene product (e.g., transcript, e.g., mRNA, e.g., polypeptide, etc.) from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional: As used herein, the term “functional” describes something that exists in a form in which it exhibits a property and/or activity by which it is characterized. For example, in some embodiments, a “functional” biological molecule is a biological molecule in a form in which it exhibits a property and/or activity by which it is characterized. In some such embodiments, a functional biological molecule is characterized relative to another biological molecule which is non-functional in that the “non-functional” version does not exhibit the same or equivalent property and/or activity as the “functional” molecule. A biological molecule may have one function, two functions (i.e., bifunctional) or many functions (i.e., multifunctional).

Gene: As used herein, the term “gene” refers to a DNA sequence in a chromosome that codes for a gene product (e.g., an RNA product, e.g., a polypeptide product). In some embodiments, a gene includes coding sequence (i.e., sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory sequences (e.g., promoters, enhancers, etc.) and/or intron sequences that, for example, may control or impact one or more aspects of gene expression (e.g., cell-type-specific expression, inducible expression, etc.). As used herein, the term “gene” generally refers to a portion of a nucleic acid that encodes a polypeptide or fragment thereof; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid. In some embodiments, a gene may encode a polypeptide, but that polypeptide may not be functional, e.g., a gene variant may encode a polypeptide that does not function in the same way, or at all, relative to the wild-type gene. In some embodiments, a gene may encode a transcript which, in some embodiments, may be toxic beyond a threshold level. In some embodiments, a gene may encode a polypeptide, but that polypeptide may not be functional and/or may be toxic beyond a threshold level.

Hearing loss: As used herein, the term “hearing loss” may be used to a partial or total inability of a living organism to hear. In some embodiments, hearing loss may be acquired. In some embodiments, hearing loss may be hereditary. In some embodiments, hearing loss may be genetic. In some embodiments, hearing loss may be as a result of disease or trauma (e.g., physical trauma, treatment with one or more agents resulting in hearing loss, etc.). In some embodiments, hearing loss may be due to one or more known genetic causes and/or syndromes. In some embodiments, hearing loss may be of unknown etiology. In some embodiments, hearing loss may or may not be mitigated by use of hearing aids or other treatments.

Heterologous: As used herein, the term “heterologous” may be used in reference to one or more regions of a particular molecule as compared to another region and/or another molecule. For example, in some embodiments, heterologous polypeptide domains, refers to the fact that polypeptide domains do not naturally occur together (e.g., in the same polypeptide). For example, in fusion proteins generated by the hand of man, a polypeptide domain from one polypeptide may be fused to a polypeptide domain from a different polypeptide. In such a fusion protein, two polypeptide domains would be considered “heterologous” with respect to each other, as they do not naturally occur together.

Identity: As used herein, the term “identity” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; nucleotides at corresponding positions are then compared. When a position in the first sequence is occupied by the same residue (e.g., nucleotide or amino acid) as a corresponding position in the second sequence, then the two molecules (i.e., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4: 11-17, which is herein incorporated by reference in its entirety), which has been incorporated into the ALIGN program (version 2.0). In some embodiments, nucleic acid sequence comparisons made with the ALIGN program use a PAM120 weight residue table, a gap length penalty of 12 and a gap penalty of 4.

Improve, increase, enhance, inhibit or reduce: As used herein, the terms “improve,” “increase,” “enhance,” “inhibit,” “reduce,” or grammatical equivalents thereof, indicate values that are relative to a baseline or other reference measurement. In some embodiments, a value is statistically significantly difference that a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment. In some embodiments, an appropriate reference is a negative reference; in some embodiments, an appropriate reference is a positive reference.

Nucleic acid: As used herein, the term “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments, a nucleic acid has a nucleotide sequence comprising at least one element that encodes, or is complementary to a sequence that encodes, a polypeptide. In some embodiments, a nucleic acid has enzymatic activity.

Operably linked: As used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with coding elements of interest; in some embodiments, control elements act in trans to or otherwise at a from the functional element of interest. In some embodiments, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In some embodiments, for example, a functional linkage may include transcriptional control. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Pharmaceutical composition: As used herein, the term “pharmaceutical composition” refers to a composition in which an active agent is formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for, e.g., administration, for example, an injectable formulation that is, e.g., an aqueous or non-aqueous solution or suspension or a liquid drop designed to be administered into an ear canal. In some embodiments, a pharmaceutical composition may be formulated for administration via injection either in a particular organ or compartment, e.g., directly into an ear, or systemic, e.g., intravenously. In some embodiments, a formulation may be or comprise drenches (aqueous or non-aqueous solutions or suspensions), tablets, boluses, powders, granules, pastes, capsules, powders, etc. In some embodiments, an active agent may be or comprise an isolated, purified, or pure compound.

Pharmaceutically acceptable: As used herein, the term “pharmaceutically acceptable” which, for example, may be used in reference to a carrier, diluent, or excipient used to formulate a pharmaceutical composition as disclosed herein, means that a carrier, diluent, or excipient is compatible with other ingredients of a composition and not deleterious to a recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting a subject compound from one organ, or portion of a body, to another organ, or portion of a body. Each carrier must be is “acceptable” in the sense of being compatible with other ingredients of a formulation and not injurious to a patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polyadenylation: As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. In some embodiments, a 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, a poly(A) tail can be added onto transcripts that contain a specific sequence, the polyadenylation signal or “poly(A) sequence.” A poly(A) tail and proteins bound to it aid in protecting mRNA from degradation by exonucleases. Polyadenylation can be affect transcription termination, export of the mRNA from the nucleus, and translation. Typically, polyadenylation occurs in the nucleus immediately after transcription of DNA into RNA, but additionally can also occur later in the cytoplasm. After transcription has been terminated, the mRNA chain can be cleaved through the action of an endonuclease complex associated with RNA polymerase. The cleavage site can be characterized by the presence of the base sequence AAUAAA near the cleavage site. After mRNA has been cleaved, adenosine residues can be added to the free 3′ end at the cleavage site. As used herein, a “poly(A) sequence” is a sequence that triggers the endonuclease cleavage of an mRNA and the additional of a series of adenosines to the 3′ end of the cleaved mRNA.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may include one or more pendant groups or other modifications, e.g., modifying or attached to one or more amino acid side chains, at a polypeptide's N-terminus, at a polypeptide's C-terminus, or any combination thereof. In some embodiments, such pendant groups or modifications may be acetylation, amidation, lipidation, methylation, pegylation, etc., including combinations thereof. In some embodiments, polypeptides may contain L-amino acids, D-amino acids, or both and may contain any of a variety of amino acid modifications or analogs known in the art. In some embodiments, useful modifications may be or include, e.g., terminal acetylation, amidation, methylation, etc. In some embodiments, a protein may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, a protein is antibodies, antibody fragments, biologically active portions thereof, and/or characteristic portions thereof.

Polynucleotide: As used herein, the term “polynucleotide” refers to any polymeric chain of nucleic acids. In some embodiments, a polynucleotide is or comprises RNA; in some embodiments, a polynucleotide is or comprises DNA. In some embodiments, a polynucleotide is, comprises, or consists of one or more natural nucleic acid residues. In some embodiments, a polynucleotide is, comprises, or consists of one or more nucleic acid analogs. In some embodiments, a polynucleotide analog differs from a nucleic acid in that it does not utilize a phosphodiester backbone. Alternatively or additionally, in some embodiments, a polynucleotide has one or more phosphorothioate and/or 5′-N-phosphoramidite linkages rather than phosphodiester bonds. In some embodiments, a polynucleotide is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a polynucleotide comprises one or more modified sugars (e.g., 2′-fluororibose, ribose, 2′-deoxyribose, arabinose, and hexose) as compared with those in natural nucleic acids. In some embodiments, a polynucleotide has a nucleotide sequence that encodes a functional gene product such as an RNA or protein. In some embodiments, a polynucleotide includes one or more introns. In some embodiments, a polynucleotide is prepared by one or more of isolation from a natural source, enzymatic synthesis by polymerization based on a complementary template (in vivo or in vitro), reproduction in a recombinant cell or system, and chemical synthesis. In some embodiments, a polynucleotide is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a polynucleotide is partly or wholly single stranded; in some embodiments, a polynucleotide is partly or wholly double stranded. In some embodiments, a polynucleotide has a nucleotide sequence comprising at least one element that encodes, or is the complement of a sequence that encodes, a polypeptide. In some embodiments, a polynucleotide has enzymatic activity.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means.

Recombinant: As used herein, the term “recombinant” is intended to refer to polypeptides that are designed, engineered, prepared, expressed, created, manufactured, and/or or isolated by recombinant means, such as polypeptides expressed using a recombinant expression construct transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of a polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some embodiments, a reference is a negative control reference; in some embodiments, a reference is a positive control reference.

Regulatory Element: As used herein, the term “regulatory element” or “regulatory sequence” refers to non-coding regions of DNA that regulate, in some way, expression of one or more particular genes. In some embodiments, such genes are apposed or “in the neighborhood” of a given regulatory element. In some embodiments, such genes are located quite far from a given regulatory element. In some embodiments, a regulatory element impairs or enhances transcription of one or more genes. In some embodiments, a regulatory element may be located in cis to a gene being regulated. In some embodiments, a regulatory element may be located in trans to a gene being regulated. For example, in some embodiments, a regulatory sequence refers to a nucleic acid sequence which is regulates expression of a gene product operably linked to a regulatory sequence. In some such embodiments, this sequence may be an enhancer sequence and other regulatory elements which regulate expression of a gene product.

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe (e.g., virus), a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to a qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Treatment: As used herein, the term “treatment” (also “treat” or “treating”) refers to any administration of a therapy that partially or completely alleviates, ameliorates, eliminates, reverses, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of a given disease, disorder, and/or condition.

Variant: As used herein, the term “variant” refers to a version of something, e.g., a gene sequence, that is different, in some way, from another version. To determine if something is a variant, a reference version is typically chosen and a variant is different relative to that reference version. In some embodiments, a variant can have the same or a different (e.g., increased or decreased) level of activity or functionality than a wild type sequence. For example, in some embodiments, a variant can have improved functionality as compared to a wild-type sequence if it is, e.g., codon-optimized to resist degradation, e.g., by an inhibitory nucleic acid, e.g., miRNA. Such a variant is referred to herein as a gain-of-function variant. In some embodiments, a variant has a reduction or elimination in activity or functionality or a change in activity that results in a negative outcome (e.g., increased electrical activity resulting in chronic depolarization that leads to cell death). Such a variant is referred to herein as a loss-of-function variant. For example, in some embodiments, a CLRN1 gene sequence is a wild-type sequence, which encodes a functional protein and exists in a majority of members of species with genomes containing the CLRN1 gene. In some such embodiments, a gain-of-function variant can be a gene sequence of CLRN1 that contains one or more nucleotide differences relative to a wild-type CLRN1 gene sequence. In some embodiments, a gain-of-function variant is a codon-optimized sequence which encodes a transcript or polypeptide that may have improved properties (e.g., less susceptibility to degradation, e.g., less susceptibility to miRNA mediated degradation) than its corresponding wild type (e.g., non-codon optimized) version. In some embodiments, a loss-of-function variant has one or more changes that result in a transcript or polypeptide that is defective in some way (e.g., decreased function, non-functioning) relative to the wild type transcript and/or polypeptide. For example, in some embodiments, a mutation in a CLRN1 sequence results in a non-functional or otherwise defective clarin 1 protein.

### BRIEF DESCRIPTION OF THE DRAWING

FIG. 1A depicts a simplified endogenous AAV genome; FIG. 1B depicts a simplified recombinant AAV (rAAV) construct capable of expressing a CLRN1 gene.

FIG. 2 depicts an exemplary rAAV construct comprising a CLRN1 gene.

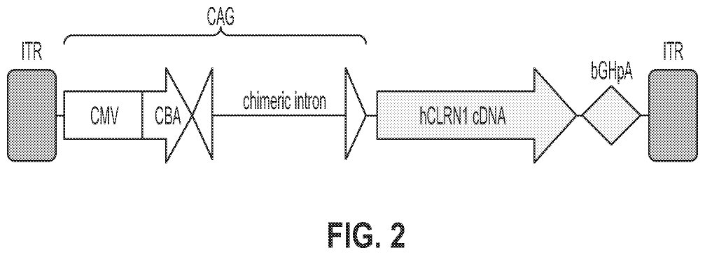

FIG. 3 depicts an exemplary rAAV construct comprising a CLRN1 gene.

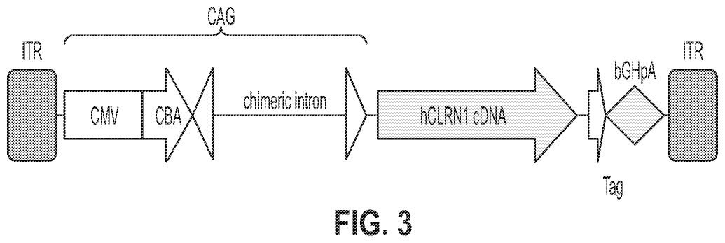

FIG. 4 depicts an exemplary rAAV construct comprising a CLRN1 gene.

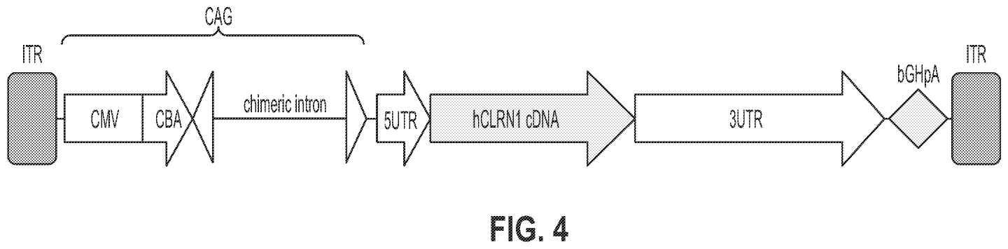

FIG. 5 depicts an exemplary rAAV construct comprising a CLRN1 gene.

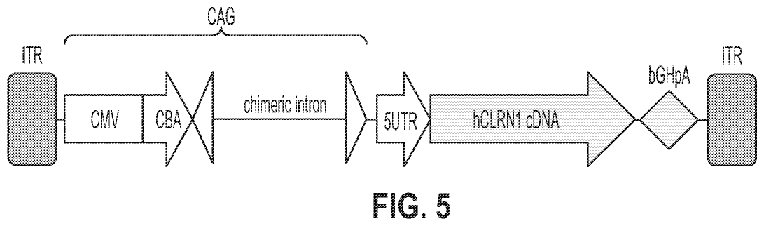

FIG. 6 depicts an exemplary rAAV construct comprising a CLRN1 gene.

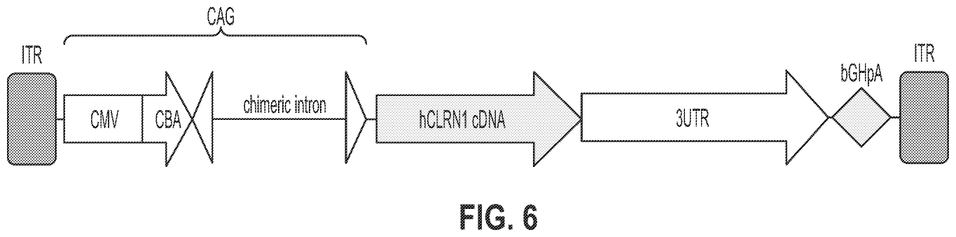

FIG. 7 depicts an exemplary rAAV construct comprising a CLRN1 gene.

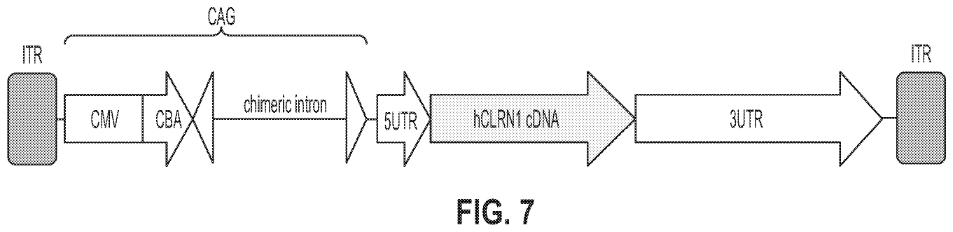

FIG. 8 depicts an exemplary schematic of retinal architecture of the eye.

FIG. 9 is a schematic of a representative anatomy of the human ear.

FIG. 10 is a schematic representation of an inner ear, indicating fluid continuity of perilymph between the vestibular system, on the left, and the cochlea (scala tympani, scala vestibuli), on the right. FIG. 10A is a schematic of a coiled cochlea. The number of cochlear turns shown is representative of a mouse inner ear. FIG. 10B is a schematic showing a cross-section of the cochlea. In the schematic, scala tympani and scala vestibuli are filled with perilymph, while scala media is filled with endolymph (Talaei 2019, incorporated herein in its entirety by reference).

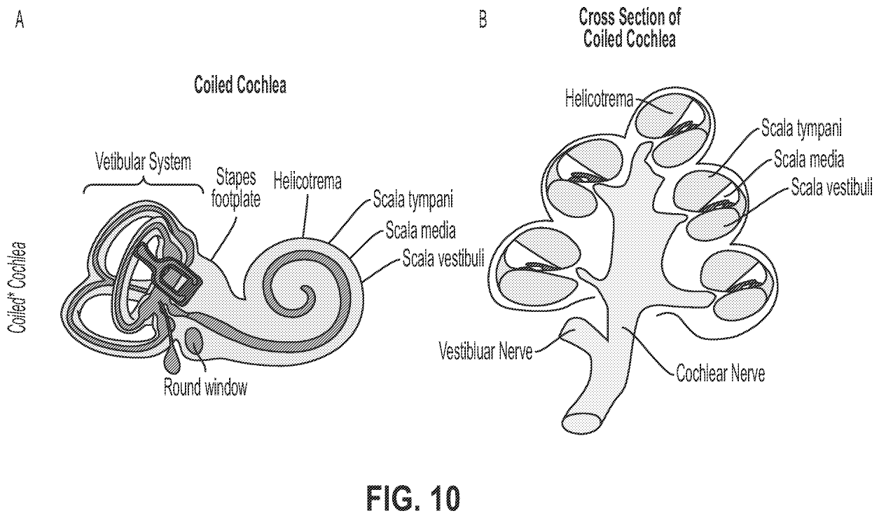

FIG. 11 is a schematic representation of an administration method as described herein. FIG. 11A includes an image of a delivery device as described herein (see, e.g., WO 2021/242926, which is incorporated herein in its entirety by reference). A delivery device as shown is intended for intracochlear administration of injected fluid through the round window membrane, with a stopper to guide insertion depth. FIG. 11B includes an images showing an expected flow of injected fluid through scala tympani to scala vestibuli (via communication at the helicotrema at the cochlear apex) and then out of the cochlea through a vent placed in the stapes footplate of a delivery device within the oval window (Talei 2019, which is incorporated herein in its entirety by reference).

FIG. 12 includes representative fluorescent images depicting in-vivo cochlear transduction of naturally occurring AAV serotypes and an AAV Anc80 variant in neonatal mice via round window membrane delivery. Mice (P1) were injected with different AAV capsids (AAV1, AAV2, AAV8, AAV6 [not shown], and AAV Anc80) comprising a construct encoding enhanced GFP (eGFP). Phalloidin was used to label actin. Quantification of eGFP-positive inner hair cells (IHCs) and outer hair cells (OHCs) showed transduction efficiency between approximately 90 to 100% from the base to the apex after delivery of rAAV Anc80 comprising a construct encoding enhanced GFP (rAAV Anc80-eGFP) (Landegger 2017, which is incorporated herein in its entirety by reference).

FIG. 13 includes representative fluorescent images depicting in-vivo vestibular transduction of rAAV Anc80 particles in neonatal mice via round window membrane delivery. Mice (P1) were injected with AAV Anc80-eGFP. Phalloidin staining was used to label actin. Transduction was observed in both type I and type II hair cells of the utricle FIG. 13A, as well as cells of the semicircular canal cristate FIG. 13B (Landegger 2017, which is incorporated herein in its entirety by reference).

FIG. 14 includes representative fluorescent images depicting in-vivo cochlear transduction of rAAV Anc80 particles in adult mice via posterior semicircular canal delivery. Mice (7 weeks old) were injected with rAAV Anc80-eGFP particles. FIG. 14A includes a low-magnification view of a mid-modiolar section of an injected cochlea, showing eGFP signal in IHCs, referred to as (I), OHCs, referred to as (O), spiral limbus, referred to as (SL), Reissner's membrane, referred to as (RM), and spiral ganglion, referred to as (SG). FIG. 14B1 and FIG. 14B2 include high-magnification views of the organ of Corti from apical (FIG. 14B1) and mid (FIG. 14B2) regions of the cochlea. Quantification of eGFP-positive cells showed that approximately 100% of the IHCs were transduced, whereas the OHC transduction decreased from apex to base. FIG. 14C is a low magnification view showing that eGFP signal was detected in a subset of cells (neurons and satellite glial cells) in the spiral ganglion (Suzuki 2017, incorporated herein in its entirety by reference).

FIG. 15 includes representative fluorescent images depicting in-vivo vestibular transduction of rAAV Anc80-eGFP in adult mice via posterior semicircular canal delivery. Mice (7 weeks old) were injected with rAAV Anc80-eGFP. FIG. 15A1 and FIG. 15A2) include low-magnification view of a section through the vestibule, showing eGFP signal in both utricle and saccule. FIG. 15B and FIG. 15C include high-magnification views of sections through vestibular end-organs (FIG. 15B: utricle; FIG. 15C: crista ampularis), showing eGFP expression in supporting cells and hair cells. Filled arrowheads indicate example transduced supporting cells (hair cells not indicated) (Suzuki 2017, incorporated herein in its entirety by reference).

FIG. 16 includes representative fluorescent images depicting in-vivo cochlear and vestibular transduction of naturally occurring AAV2 serotype compared to rAAV Anc80 variant in adult mice via round window membrane delivery with canal fenestration. Mice (4 weeks old) were injected with different AAV particles (AAV2 and rAAV Anc80 shown here; AAV1, AAV8, and AAV9 not shown) encoding eGFP. Compared to AAV2, rAAV Anc80 mediated transduction showed comparable rates of IHC and OHC transduction (FIG. 16A1 vs. FIG. 16A2) but broader transduction of spiral ganglion cells (FIG. 16B1 vs. FIG. 16B2) and hair cells of the saccule (FIG. 16C1 vs. FIG. 16C2: whole mounts; FIG. 16D1 vs. FIG. 16D2: sections) (Omichi 2020, incorporated herein in its entirety by reference).

FIG. 17 includes a Western blot showing HEK cell expression of CLRN1 protein, using transduction of exemplary rAAV-CLRN1 constructs described herein. Lanes are noted along the top of the figure, with predicted protein sizes noted on the left of the figure. Lanes labeled with a “+” indicate PNGase F treatment. Lane 1: pre-stained PageRuler™ protein ladder. Lane 2: untransfected/negative control. Lane 3: untransfected/negative control (+). Lane 4: transduction with an rAAV-CLRN1wild-type (wt) particle with a multiplicity of infection (MOI) of 6.7×10⁴. Lane 5: transduction with rAAV-CLRN1wt particle (+) with a multiplicity of infection (MOI) of 6.7×10⁴. Lane 6: transduction with an rAAV Anc80-CLRN1wt particle with a multiplicity of infection (MOI) of 1.3×10⁵. Lane 7: transduction with an rAAV Anc80-CLRN1wt particle (+) with a multiplicity of infection (MOI) of 1.3×10⁵. Lane 8: transduction with an rAAV Anc80-CLRN1wt particle with a multiplicity of infection (MOI) of 2.3×10⁵. Lane 9: transduction with an rAAV Anc80-CLRN1wt particle (+) with a multiplicity of infection (MOI) of 2.3×10⁵. Lane 10: transduction with an rAAV Anc80-CLRN1-codon modified particle with a multiplicity of infection (MOI) of 2.3×10⁵. Lane 11: t transduction with an rAAV Anc80-CLRN1-codon modified particle (+) with a multiplicity of infection (MOI) of 2.3×10⁵. Bands also indicate whether CLRN1 “iso1” or Isoform A is glycosylated or de-glycosylated.

FIG. 18A is a graphical representation of RNA expression in cochlear explants following transduction of wild-type (WT) newborn (P2) mice cochlear explants with rAAV Anc80 particles comprising Clarin 1 proteins as disclosed herein (rAAV Anc80-CLRN1). FIG. 18A depicts RNA expression analysis, and demonstrates expression of the mRNA encoding CLRN1 in cells of explants receiving rAAV Anc80-CLRN1 particles (construct according to SEQ ID NO: 64). No expression was detected in explants receiving vehicle (“mock”). Results indicate average expression. FIG. 18B depicts a fluorescence image showing CLRN1 protein expression WT newborn mice.

FIG. 19 illustrates a perspective of a device for delivering fluid to an inner ear, according to aspects of the present disclosure.

FIG. 20 illustrates a sideview of a bent needle sub-assembly, according to aspects of the present disclosure.

FIG. 21 illustrates a perspective view of a device for delivering fluid to an inner ear, according to aspects of the present disclosure.

FIG. 22 illustrates a perspective view of a bent needle sub-assembly coupled to the distal end of a device, according to aspects of the present disclosure.

### DETAILED DESCRIPTION OF CERTAIN EMBODIMENTS

### Hearing Loss

Generally, an ear can be described as including: an outer ear, middle ear, inner ear, hearing (acoustic) nerve, and auditory system (which processes sound as it travels from the ear to the brain). In addition to detecting sound, ears also help to maintain balance. Thus, in some embodiments, disorders of the inner ear can cause hearing loss, tinnitus, vertigo, imbalance, or combinations thereof.

Hearing loss can be the result of genetic factors, environmental factors, or a combination of genetic and environmental factors. About half of all people who have tinnitus—phantom noises in their auditory system (ringing, buzzing, chirping, humming, or beating)—also have an over-sensitivity to/reduced tolerance for certain sound frequency and volume ranges, known as hyperacusis (also spelled hyperacousis). A variety of nonsyndromic and syndromic-related hearing losses will be known to those of skill in the art (e.g., Usher syndrome, DFNB4, and Pendred syndrome, respectively). Environmental causes of hearing impairment or loss may include, e.g., certain medications, specific infections before or after birth, and/or exposure to loud noise over an extended period. In some embodiments, hearing loss can result from noise, ototoxic agents, presbycusis, disease, infection or cancers that affect specific parts of the ear. In some embodiments, ischemic damage can cause hearing loss via pathophysiological mechanisms. In some embodiments, intrinsic abnormalities, like congenital mutations to genes that play an important role in cochlear anatomy or physiology, or genetic or anatomical changes in supporting and/or hair cells can be responsible for or contribute to hearing loss.

Hearing loss and/or deafness is one of the most common human sensory deficits, and can occur for many reasons. In some embodiments, a subject may be born with hearing loss or without hearing, while others may lose hearing slowly over time. Approximately 36 million American adults report some degree of hearing loss, and one in three people older than 60 and half of those older than 85 experience hearing loss. Approximately 1.5 in 1,000 children are born with profound hearing loss, and another two to three per 1,000 children are born with partial hearing loss (Smith et al., 2005, Lancet 365:879-890, which is incorporated in its entirety herein by reference). More than half of these cases are attributed to a genetic basis (Di Domenico, et al., 2011, J. Cell. Physiol. 226:2494-2499, which is incorporated in its entirety herein by reference).

Treatments for hearing loss currently consist of hearing amplification for mild to severe losses and cochlear implantation for severe to profound losses (Kral and O'Donoghue, 2010, N. Engl. J. Med. 363:1438-1450, which is incorporated in its entirety herein by reference). Recent research in this arena has focused on cochlear hair cell regeneration, applicable to the most common forms of hearing loss, including presbycusis, noise damage, infection, and ototoxicity. There remains a need for effective treatments, such as gene therapy, which can repair and/or mitigate a source of a hearing problem (see e.g., WO 2018/039375, WO 2019/165292, and WO 2020/097372, each of which is incorporated in its entirety herein by reference).

In some embodiments, nonsyndromic hearing loss and/or deafness is not associated with other signs and symptoms. In some embodiments, syndromic hearing loss and/or deafness occurs in conjunction with abnormalities in other parts of the body. Approximately 70 percent to 80 percent of genetic hearing loss and/or deafness cases are nonsyndromic; remaining cases are often caused by specific genetic syndromes. Nonsyndromic deafness and/or hearing loss can have different patterns of inheritance, and can occur at any age. Types of nonsyndromic deafness and/or hearing loss are generally named according to their inheritance patterns. For example, autosomal dominant forms are designated DFNA, autosomal recessive forms are DFNB, and X-linked forms are DFN. Each type is also numbered in the order in which it was first described. For example, DFNA1 was the first described autosomal dominant type of nonsyndromic deafness. Between 75 percent and 80 percent of genetically causative hearing loss and/or deafness cases are inherited in an autosomal recessive pattern, which means both copies of the gene in each cell have mutations (for example, in Usher syndrome). Usually, each parent of an individual with autosomal recessive hearing loss and/or deafness is a carrier of one copy of the mutated gene, but is not affected by this form of hearing loss. Another 20 percent to 25 percent of nonsyndromic hearing loss and/or deafness cases are autosomal dominant, which means one copy of the altered gene in each cell is sufficient to result in deafness and/or hearing loss. People with autosomal dominant deafness and/or hearing loss most often inherit an altered copy of the gene from a parent who is deaf and/or has hearing loss. Between 1 to 2 percent of cases of deafness and/or hearing loss show an X-linked pattern of inheritance, which means the mutated gene responsible for the condition is located on the X chromosome (one of the two sex chromosomes). Males with X-linked nonsyndromic hearing loss and/or deafness tend to develop more severe hearing loss earlier in life than females who inherit a copy of the same gene mutation. A characteristic of X-linked inheritance is that fathers cannot pass X-linked traits to their sons. Mitochondrial nonsyndromic deafness, which results from changes to mitochondrial DNA, occurs in less than one percent of cases in the United States. The altered mitochondrial DNA is passed from a mother to all of her sons and daughters. This type of deafness is not inherited from fathers. The causes of syndromic and nonsyndromic deafness and/or hearing loss are complex. Researchers have identified more than 30 genes that, when altered, are associated with syndromic and/or nonsyndromic deafness and/or hearing loss; however, some of these genes have not been fully characterized. Different mutations in the same gene can be associated with different types of deafness and/or hearing loss, and some genes are associated with both syndromic and nonsyndromic deafness and/or hearing loss.

In some embodiments, deafness and/or hearing loss can be conductive (arising from the ear canal or middle ear), sensorineural (arising from the inner ear or auditory nerve), or mixed. In some embodiments, nonsyndromic deafness and/or hearing loss is associated with permanent hearing loss caused by damage to structures in the inner ear (sensorineural deafness). In some embodiments, sensorineural hearing loss can be due to poor hair cell function. In some embodiments, sensorineural hearing impairments involve the eighth cranial nerve (the vestibulocochlear nerve) or the auditory portions of the brain. In some such embodiments, only the auditory centers of the brain are affected. In such a situation, cortical deafness may occur, where sounds may be heard at normal thresholds, but quality of sound perceived is so poor that speech cannot be understood. Hearing loss that results from changes in the middle ear is called conductive hearing loss. Some forms of nonsyndromic deafness and/or hearing loss involve changes in both the inner ear and the middle ear, called mixed hearing loss. Hearing loss and/or deafness that is present before a child learns to speak can be classified as prelingual or congenital. Hearing loss and/or deafness that occurs after the development of speech can be classified as postlingual. Most autosomal recessive loci related to syndromic or nonsyndromic hearing loss cause prelingual severe-to-profound hearing loss.

As is known to those of skill in the art, hair cells are sensory receptors for both auditory and vestibular systems of vertebrate ears. Hair cells detect movement in the environment and, in mammals, hair cells are located within the cochlea of the ear, in the organ of Corti. Mammalian ears are known to have two types of hair cells—inner hair cells and outer hair cells. Outer hair cells can amplify low level sound frequencies, either through mechanical movement of hair cell bundles or electrically-driven movement of hair cell soma. Inner hair cells transform vibrations in cochlear fluid into electrical signals that the auditory nerve transmits to the brain. In some embodiments, hair cells may be abnormal at birth, or damaged during the lifetime of an individual. In some embodiments, outer hair cells may be able to regenerate. In some embodiments, inner hair cells are not capable of regeneration after illness or injury. In some embodiments, sensorineural hearing loss is due to abnormalities in hair cells.

As is known to those of skill in the art, hair cells do not occur in isolation, and their function is supported by a wide variety of cells which can collectively be referred to as supporting cells. Supporting cells may fulfil numerous functions, and include a number of cell types, including but not limited to Hensen's cells, Deiters cells, pillar cells, Claudius cells, inner phalangeal cells, and border cells. In some embodiments, sensorineural hearing loss is due to abnormalities in supporting cells. In some embodiments, supporting cells may be abnormal at birth, or damaged during the lifetime of an individual. In some embodiments, supporting cells may be able to regenerate. In some embodiments, certain supporting cells may not be capable of regeneration.

### Vision Loss

Eyes are organs of the visual system that provide animals with vision, receive and process visual detail, and enable several photo response functions that are independent of vision. Generally, eyes detect and convert light into electro-chemical impulses in neurons. In higher organisms, such as mammals, the eye is a complex optical system which collects light from the surrounding environment, regulates its intensity through a diaphragm, forces it through an adjustable assembly of lenses to form an image, converts this image into a set of electrical signals, and transmits these signals to the brain through complex neural pathways that connect the eye via the optic nerve to the visual cortex and other areas of the brain. Eye anatomy generally includes a cornea, pupil, iris, lens, anterior chamber, posterior chamber, lacrimal fluid, limbus, ciliary muscle, suspensory ligament, vitreous chamber, sclera, choroid, retina, macula/fovea centralis, optic nerve, and blind spot.

Vision loss can be the result of genetic factors, environmental factors, or a combination of genetic and environmental factors. Vision loss is any reduction in the ability to see, including blurred vision, cloudy vision, double vision, blind spots, poor night vision, and loss of peripheral vision (tunnel vision). Vision loss may affect one or both eyes, it may occur gradually or suddenly, and it may be partial or complete. Environmental causes of vision impairment or loss may include, e.g., certain medications, specific infections before or after birth, and/or exposure to physical contact of objects with the eye. In some embodiments, intrinsic abnormalities, like congenital mutations to genes that play an important role in eye anatomy or physiology, or genetic or anatomical changes in supporting and/or eye cells can be responsible for or contribute to hearing loss.

Vision loss is one type of human sensory deficits, and can occur for many reasons. In some embodiments, a subject may be born with vision loss or without vision, while others may lose vision slowly over time. Approximately 26.9 million American adults report some degree of vision loss, and 7.8 million American adults 65 years and older report experience significant vision loss.

Retinitis pigmentosa is a genetic disorder that causes loss of vision due to the deterioration of retinal cells (e.g., photoreceptor cells). Usually, the rod cells of the retina are affected first, leading to early night blindness (nyctalopia) and the gradual loss of peripheral vision. In other cases, early degeneration of the cone cells in the macula occurs, leading to a loss of central acuity. In some cases, the foveal vision is spared, leading to “donut vision”; central and peripheral vision are intact, but an annulus exists around the central region in which vision is impaired.

Symptoms of retinitis pigmentosa include trouble seeing at night and decreased peripheral vision (side vision). As peripheral vision worsens, people may experience “tunnel vision”. Complete blindness is possible, but not common. Onset of symptoms is generally gradual and often in childhood. Retinitis is generally inherited from a person's parents. Mutations in more than 50 genes are involved. The underlying mechanism involves the progressive loss of rod photoreceptor cells in the back of the eye. This is generally followed by loss of cone photoreceptor cells. Diagnosis is by an examination of the retina finding dark pigment despots. Other supporting testing may include an electroretinogram, visual field testing, or genetic testing (see e.g., “Facts About Retinitis Pigmentosa”. National Eye Institute. May 2014, retrieved 18 Apr. 2020, the contents of which are hereby incorporated in its entirety herein.)

There is currently no cure for retinitis pigmentosa. Efforts to manage the problem may include the use of low vision aids, portable lighting, or orientation and mobility training. Vitamin A palmitate supplements may be useful to slow worsening. A visual prosthesis may be an option in certain people with severe diseases. Retinitis pigmentosa is estimated to affect 1 in 4,000 people (see e.g., “Facts About Retinitis Pigmentosa”. National Eye Institute. May 2014, retrieved 18 Apr. 2020, the contents of which are hereby incorporated in its entirety herein and Openshaw, Amanda (February 2008), “Understanding Retinitis Pigmentosa,” University of Michigan Kellogg Eye Center. Archived from the original on 2017-08-29, the contents of which is hereby incorporated by reference in its entirety herein)

As is known to those of skill in the art, the eye is composed of a variety of cell types. The cells located in the optic nerves that connect the eyes to the brain are the cells that transmit electrical signals to the brain so that the brain can comprehend the signals as images. Retinal cells (e.g., photoreceptor cells) are located in the back of the eye and are what convert light into electrical signals. Other cells include rods and cones that allow people to perceive colors and shapes. CLRN1 expression in the eye has been identified in the following exemplary layers and/or cells of the eye as follows:

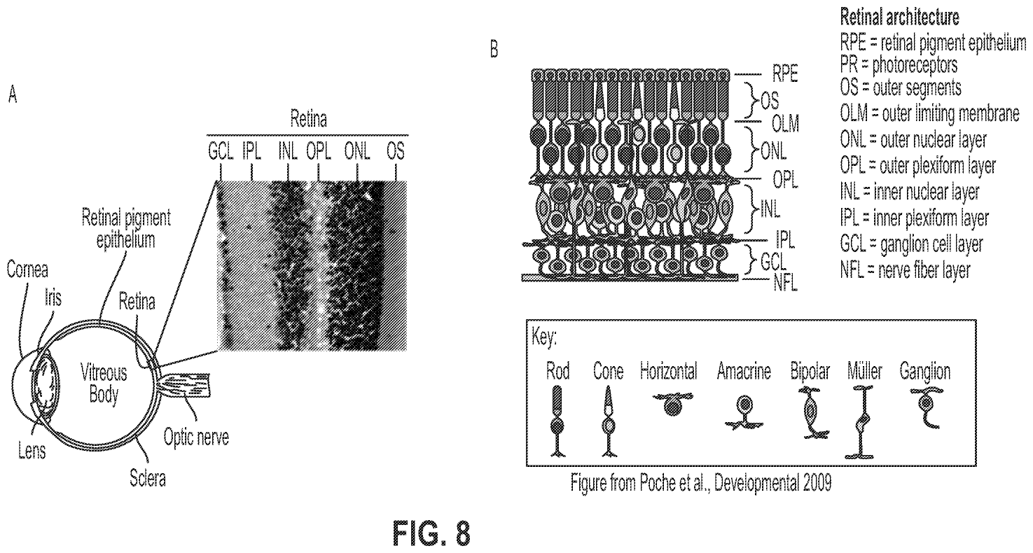

CLRN1 Expression in Eye (FIG. 8)

| | | | |
|---|---|---|---|
| Inner Nuclear Layer (INL), | mouse | mRNA, IHC | Geller et al., PLoS Genet |
| Muller glia cells | | | 2009, which is incorporated |
| | | | herein by reference in its |
| | | | entirety |
| Photoreceptors (PR), synaptic | mouse | Western, RT- | Zallocchi et al., Hear Res |
| and connecting cilium | | PCR, IHC | 2009, which is incorporated |
| | | | herein by reference in its |
| | | | entirety |
| PR and INL | zebra fish | Western Blot | Phillips et al., Gene Expr |
| | | | Patterns 2013, which is |
| | | | incorporated herein by |
| | | | reference in its entirety |
| Muller cells | mouse | RNAscope, | Xu et al., J Pathol 2019, |
| | | scRNA | which is incorporated |
| | | | herein by reference in its |
| | | | entirety |

### Usher Syndrome

Usher syndrome is a condition that affects both hearing and vision. Usher syndrome can also affect balance. Some major symptoms of Usher syndrome are deafness or hearing loss and eye diseases, such as retinitis pigmentosa.

Deafness or hearing loss in Usher syndrome can be caused by abnormal development of hair cells (sound receptor cells) in the inner ear. Children with Usher syndrome commonly are born with moderate to profound hearing loss, depending on the type of Usher syndrome. Less commonly, hearing loss from Usher syndrome appears during adolescence or later. Usher syndrome can also cause severe balance problems due to abnormal development of the vestibular hair cells, sensory cells that detect gravity and head movement.

Retinitis pigmentosa initially causes night-blindness and a loss of peripheral vision through the progressive degeneration of cells in the retina, which is the light-sensitive tissue at the back of the eye and is crucial for vision. As retinitis pigmentosa progresses, the field of vision narrows until only central vision remains, a condition called tunnel vision. Cysts in the macula and cataracts (clouding of the lens) can sometimes cause an early decline in central vision in people with Usher syndrome.

Usher syndrome affects approximately 4 to 17 per 100,000 people and accounts for about 50 percent of all hereditary deaf-blindness cases. The condition is thought to account for 3 to 6 percent of all children who are deaf, and another 3 to 6 percent of children who are hard-of-hearing. (see, e.g., Boughman, J. A., et al. (1983), “Usher syndrome: definition and estimate of prevalence from two high-risk populations,” Journal of Chronic Diseases, 36(8), 595-603; Kimberling, W., et al. (2010), “Frequency of Usher syndrome in two pediatric populations: implications for genetic screening of deaf and hard of hearing children,” Genetics in Medicine, 12(8), 512-516; Berson, E. L. (1998), “Treatment of retinitis pigmentosa with vitamin A”, Digital Journal of Ophthalmology, 4(7), the disclosures of which are hereby incorporated by reference herein in their entireties).

Usher syndrome is inherited, which means that it is passed from parents to a child through genes. Each person inherits two copies of a gene, one from each parent. Sometimes genes are altered, or mutated. Mutated genes may cause cells to develop or act abnormally.

Usher syndrome is inherited as an autosomal recessive disorder. “Autosomal” means that men and women are equally likely to have the disorder and equally likely to pass it on to a child of either sex. “Recessive” means that the condition occurs only when a child inherits two copies of the same faulty gene, one from each parent. A person with one abnormal Usher gene does not have the disorder but is a carrier who has a 50 percent chance of passing on the abnormal gene to each child.

Diagnosis of Usher syndrome involves pertinent questions regarding the person's medical history and testing of hearing, balance, and vision. Early diagnosis is important, as it improves treatment success. An eye care specialist can use dilating drops to examine the retina for signs of retinitis pigmentosa. Visual field testing measures peripheral vision and can aid in diagnosis of Usher syndrome. An electroretinogram measures the electrical response of the eye's light-sensitive cells in the retina. Optical coherence tomography may be helpful to assess for macular cystic changes. Videonystagmography measures involuntary eye movements that could signify a balance issue. Audiology testing dertimes hearing sensitivity at a range of frequencies.

There are three types of Usher syndrome: Type I, Type II, and Type III. People with Usher syndrome III are not born deaf but experience a progressive loss of hearing, and roughly half have balance difficulties. For example, children with Usher syndrome type III have normal hearing at birth and normal to near-normal balance, which may decline with age. Decline in hearing and vision varies. Children with Usher syndrome type III often develop hearing loss by adolescence, requiring hearing aids by mid-to-late adulthood. Night blindness also usually begins during adolescence. Blind spots appear by the late teens to early twenties. Legal blindness also occurs by midlife.

Genetic testing may help diagnose Usher syndrome. For example, mutations in a CLRN1 gene have been linked to Usher syndrome III. CLRN1 encodes clarin-1, a protein important for the development and maintenance of the inner ear and retina. However, the protein's function and how mutations in clarin-1 cause hearing and vision loss, is poorly understand.

Presently, there is no cure for Usher syndrome. Treatment involves managing hearing, vision, and balance problems. Early diagnosis helps tailor educational programs that consider the severity of hearing and vision loss and a child's age and ability. Treatment and communication services may include hearing aids, assistive listening devices, cochlear implants, auditory (hearing) training, and/or learning American Sign Language. Independent-living training may include orientation and mobility training for balance problems, Braille instruction, and low-vision services.

Vitamin A may slow the progression of retinitis pigmentosa, according to results from a long-term clinical trial supported by the National Eye Institute and the Foundation Fighting Blindness. Based on the study, adults with a common form of retinitis pigmentosa may benefit from a daily supplement of 15,000 IU (international units) of the palmitate form of vitamin A.

### CLRN1

The CLRN1 gene encodes “clarin 1” (CLRN1), a protein that is expressed in hair cells of the inner ear (e.g., inner ear hair cells, outer ear hair cells) and in the retina.

For example, the CLRN1 gene encodes a protein that contains a cytosolic N-terminus, multiple helical transmembrane domains, and an endoplasmic reticulum membrane retention signal, TKGH, in the C-terminus. CLRN1 is thought to be necessary for hair cell and/eye cell function and associated with neural activation (see, e.g., Geng et al., “Usher syndrome IIIA gene clarin-1 is essential for hair cell function and associated neural activation” Hum Mol Genet. 2009 Aug. 1; 18(15):2748-60. doi: 10.1093/hmg/ddp210. Epub 2009 May 3, the contents of which are hereby incorporated by reference herein in its entirety; see also, e.g., Dinculescu et al., “AAV-mediated Clarin-1 expression in the mouse retina: implications for USH3A gene therapy” PLoS One. 2016; 11(2): e0148874, Published online 2016 Feb. 16, the contents of which are hereby incorporated by reference in its entirety).

The human CLRN1 gene is located on chromosome 3q25.1. It contains at least 8 (termed exons 0, 0b, 1, 1b, 2, 2b, 3a, and 3b) exons encompassing˜47 kilobases (kb) (see, e.g., Vastinsalo et al. (2011) Eur J Hum Genet 19(1): 30-35 which is incorporated in its entirety herein by reference; NCBI Accession No. NG_009168.1, which is incorporated herein by reference in its entirety). Multiple transcript variants encoding distinct isoforms have been identified for this gene (e.g., see NCBI Gene ID: 7401, which is incorporated herein by reference in its entirety).

Various mutations in the CLRN1 genes have been associated with Usher syndrome type III (see, e.g., Usher syndrome type IIIA (MIM #606397; Fields et al. (2002) Am J Hum Genet 71: 607-617, which is incorporated in its entirety herein by reference; and Joensuu et al. (2001) Am J Hum Genet 69: 673-684, which is incorporated in its entirety herein by reference) and retinitis pigmentosa (see, e.g., Khan et al. (2011) Ophthalmology 118: 1444-1448, which is incorporated in its entirety herein by reference). Usher syndrome type III-causing mutations have been predominantly found in exon 3 of CLRN1. Usher syndrome type III-deafness can be modeled by generating CLRN1-deficient mice (see, e.g., Geng et al. (2017) Sci Rep 7(1): 13480, which is incorporated in its entirety herein by reference). Exemplary mutations CLRN1-associated with Usher syndrome type III include: T528G, M120K, M44K, N48K, and C40G.

Exemplary mutations CLRN1-associated with retinitis pigmentosa include L154W and P31L (see, e.g., Khan et al. (2011) Ophthalmology 118: 1444-1448, which is incorporated in its entirety herein by reference).

Additional exemplary mutations in a CLRN1 gene that have been detected in subjects having hearing loss and methods of sequencing a nucleic acid encoding CLRN1 are described in, e.g., Fields et al. (2002) Am J Hum Genet 71: 607-617, Joensuu et al. (2001) Am J Hum Genet 69: 673-684, Adato et al. (2002) Europ J Hum Genet 10: 339-350, Aller et al. (2004), Clin Genet 66: 525-529, each of which is incorporated in its entirety herein by reference. Methods of detecting mutations in a gene are well-known in the art. Non-limiting examples of such techniques include: real-time polymerase chain reaction (RT-PCR), PCR, sequencing, Southern blotting, and Northern blotting.

Clarin 1 has been found in several areas of the body, including sensory cells in the inner ear called hair cells. These cells help transmit sound and motion signals to the brain. This protein is also active in the retina, which is the light-sensing tissue that lines the back of the eye. Although the function of clarin 1 has not been determined, studies suggest that it plays a role in communication between nerve cells (neurons) in the inner ear and in the retina. Clarin 1 may be important for the development and function of synapses, which are junctions between neurons where cell-to-cell communication occurs. Other names for the CLRN1 gene include USH3, USH3A, USH3A_Human, Usher syndrome 3A, and Usher syndrome type 3 protein.

Mutations in CLRN1 have been associated with hearing loss and deafness (see, e.g., Albert et al., Eur. J. Hum. Genet. 14:773-779, 2006; and Qing et al., Genet. Test Mol. Biomarkers 19(1):52-58, 2015, each of which is incorporated in its entirety herein by reference). Mutations in the CLRN1 gene alter the structure or function of clarin 1, disrupting its endogenous function. There are a few reported mutations in CLRN1 associated with hearing loss. For example, point mutations A123D, N48K, Y176X, and L54Phave been reported in USH3 patients (see, e.g., Isosomppi et al., “Disease-causing mutations in the CLRN1 gene alter normal CLRN1 protein trafficking to the plasma membrane” Molecular vision 15(191-121):1806-18, published September 2009, which is incorporated in its entirety herein by reference). Methods of detecting mutations in a gene are well-known in the art. Non-limiting examples of such techniques include: real-time polymerase chain reaction (RT-PCR), PCR, Sanger sequencing, next-generation sequencing, Southern blotting, and Northern blotting.

Mutations in the CLRN1 gene and encoded clarin 1 protein have been linked with Usher Syndrome Type III. Usher Syndrome Type III is an autosomal recessive disorder characterized by progressive sensorineural hearing loss, vestibular dysfunction, and retinitis pigmentosa Usher syndrome type III (USH3 [MIM #276902]) is unique among the clinical subtypes of Usher syndrome, in that it shows postlingual, progressive hearing loss and late onset of retinitis pigmentosa (RP), as well as a progressive loss of vestibular function (see, e.g., Kimberling W J, Orten D, Pieke-Dahl S (2000) Genetic heterogeneity of Usher syndrome. Adv Otorhinolaryngol 56:11-18, the contents of which is hereby incorporated by reference herein in its entirety). The disease locus was originally mapped to chromosome 3q25, between the markers WI-17533 and 486D12SP6, a region of ˜700 kb (see, e.g., Joensuu et al. 1996, which is incorporated in its entirety herein by reference). In a recent publication (see, e.g., Joensuu T, Blanco G, Pakarinen L, Sistonen P, Kaariainen H, Brown S, Chapelle A, Sankila E M (1996) Refined mapping of the Usher syndrome type III locus on chromosome 3, exclusion of candidate genes, and identification of the putative mouse homologous region. Genomics 38:255-263, the contents of which is hereby incorporated by reference herein in its entirety), the USH3 locus was assigned to a region of 250 kb between 107G19CA7 and D3S3625, by means of haplotype and linkage disequilibrium analyses in Finnish carriers of the putative founder mutation.

Wild-type CLRN1 is a glycoprotein localized to the plasma membrane in transfected BHK-21 cells. Mutant CLRN1 proteins are mislocalized. It has been suggested that part of the pathogenesis of USH3 may be associated with defective intracellular trafficking as well as decreased stability of mutant CLRN1 proteins (see, e.g., Isosomppi et al., “Disease-causing mutations in the CLRN1 gene alter normal CLRN1 protein trafficking to the plasma membrane” Molecular vision 15(191-121):1806-18, published September 2009, which is incorporated in its entirety herein by reference).

CLRN1 Polynucleotides

Among other things, the present disclosure provides polynucleotides, e.g., polynucleotides comprising a CLRN1 gene or characteristic portion thereof, as well as compositions including such polynucleotides and methods utilizing such polynucleotides and/or compositions.

In some embodiments, a polynucleotide comprising a CLRN1 gene or characteristic portion thereof can be DNA or RNA. In some embodiments, DNA can be genomic DNA or cDNA. In some embodiments, RNA can be an mRNA. In some embodiments, a polynucleotide comprises exons and/or introns of a CLRN1 gene.

In some embodiments, a gene product is expressed from a polynucleotide comprising a CLRN1 gene or characteristic portion thereof. In some embodiments, expression of such a polynucleotide can utilize one or more control elements (e.g., promoters, enhancers, splice sites, poly-adenylation sites, translation initiation sites, etc.). Thus, in some embodiments, a polynucleotide provided herein can include one or more control elements.

In some embodiments, a CLRN1 gene is a mammalian CLRN1 gene. In some embodiments, a CLRN1 gene is a murine CLRN1 gene. In some embodiments, a CLRN1 gene is a primate CLRN1 gene. In some embodiments, a CLRN1 gene is a human CLRN1 gene. In some embodiments, a CLRN1 gene is a human CLRN1 isoform. Human CLRN1 isoform variants are described in, e.g., Vastinsalo et al. (2011) Eur J Hum Genet 19(1): 30-35, which is hereby incorporated by reference herein in its entirety; NCBI Accession No. NG_009168.1, which is incorporated herein by reference in its entirety. An exemplary human CLRN1 cDNA sequence is or includes the sequence of SEQ ID NO: 1, SEQ ID NO: 2, SEQ ID NO: 3, or SEQ ID NO: 4. An exemplary human CLRN1 genomic DNA sequence can be found in SEQ ID NO: 5. An exemplary human CLRN1 cDNA sequence including untranslated regions is or includes the sequence of SEQ ID NO: 6, 7, 8, or 9.

Exemplary Human CLRN1 cDNA coding Sequence (Isoform A)
(SEQ ID NO: 1)
ATGCCAAGCCAACAGAAGAAAATCATTTTTTGCATGGCCGGAGTGTTCAGTTTTGCATGTGCCC
TCGGAGTTGTGACAGCCTTGGGGACACCGTTGTGGATCAAAGCCACTGTCCTCTGCAAAACGGG
AGCTCTGCTCGTCAATGCCTCAGGGCAGGAGCTGGACAAGTTTATGGGTGAAATGCAGTACGGG
CTTTTCCACGGAGAGGGTGTGAGGCAGTGTGGGTTGGGAGCAAGGCCCTTTCGGTTCTCATTTT
TTCCAGATTTGCTCAAAGCAATCCCAGTGAGCATCCACGTCAATGTCATTCTCTTCTCTGCCAT
CCTTATTGTGTTAACCATGGTGGGGACAGCCTTCTTCATGTACAATGCTTTTGGAAAACCTTTT
GAAACTCTGCATGGTCCCCTAGGGCTGTACCTTTTGAGCTTCATTTCAGGCTCCTGTGGCTGTC
TTGTCATGATATTGTTTGCCTCTGAAGTGAAAATCCATCACCTCTCAGAAAAAATTGCAAATTA
TAAAGAAGGGACTTATGTCTACAAAACGCAAAGTGAAAAATATACCACCTCATTCTGGGTCATT
TTCTTTTGCTTTTTTGTTCATTTTCTGAATGGGCTCCTAATACGACTTGCTGGATTTCAGTTCC
CTTTTGCAAAATCTAAAGACGCAGAAACAACTAATGTAGCTGCAGATCTAATGTAC
Exemplary Human CLRN1 cDNA coding Sequence (Isoform C)
(SEQ ID NO: 2)
ATGCAGGCCCTGCAGCAGCAACCAGTTTTTCCAGATTTGCTCAAAGCAATCCCAGTGAGCATCC
ACGTCAATGTCATTCTCTTCTCTGCCATCCTTATTGTGTTAACCATGGTGGGGACAGCCTTCTT
CATGTACAATGCTTTTGGAAAACCTTTTGAAACTCTGCATGGTCCCCTAGGGCTGTACCTTTTG
AGCTTCATTTCAGGCTCCTGTGGCTGTCTTGTCATGATATTGTTTGCCTCTGAAGTGAAAATCC
ATCACCTCTCAGAAAAAATTGCAAATTATAAAGAAGGGACTTATGTCTACAAAACGCAAAGTGA
AAAATATACCACCTCATTCTGGCTGACTAAAGGCCACAGCTGA
Exemplary Human CLRN1 cDNA coding Sequence (Isoform D)
(SEQ ID NO: 3)
ATGCCAAGCCAACAGAAGAAAATCATTTTTTGCATGGCCGGAGTGTTCAGTTTTGCATGTGCCC
TCGGAGTTGTGACAGCCTTGGGGACACCGTTGTGGATCAAAGCCACTGTCCTCTGCAAAACGGG
AGCTCTGCTCGTCAATGCCTCAGGGCAGGAGCTGGACAAGTTTATGGGTGAAATGCAGTACGGG
CTTTTCCACGGAGAGGGTGTGAGGCAGTGTGGGTTGGGAGCAAGGCCCTTTCGGTTCTCATTTT
TTCCAGATTTGCTCAAAGCAATCCCAGTGAGCATCCACGTCAATGTCATTCTCTTCTCTGCCAT
CCTTATTGTGTTAACCATGGTGGGGACAGCCTTCTTCATGTACAATGCTTTTGGAAAACCTTTT
GAAACTCTGCATGGTCCCCTAGGGCTGTACCTTTTGAGCTTCATTTCAGTTGCCCTTTGGCTGC
CAGCTACCAGGCACCAGGCTCAAGGCTCCTGTGGCTGTCTTGTCATGATATTGTTTGCCTCTGA
AGTGAAAATCCATCACCTCTCAGAAAAAATTGCAAATTATAAAGAAGGGACTTATGTCTACAAA
ACGCAAAGTGAAAAATATACCACCTCATTCTGGGTCATTTTCTTTTGCTTTTTTGTTCATTTTC
TGAATGGGCTCCTAATACGACTTGCTGGATTTCAGTTCCCTTTTGCAAAATCTAAAGACGCAGA
AACAACTAATGTAGCTGCAGATCTAATGTACTGA
Exemplary Human CLRN1 cDNA coding Sequence (Isoform E)
(SEQ ID NO: 4)
ATGCCAAGCCAACAGAAGAAAATCATTTTTTGCATGGCCGGAGTGTTCAGTTTTGCATGTGCCC
TCGGAGTTGTGACAGCCTTGGGGACACCGTTGTGGATCAAAGCCACTGTCCTCTGCAAAACGGG
AGCTCTGCTCGTCAATGCCTCAGGGCAGGAGCTGGACAAGTTTATGGGTGAAATGCAGTACGGG
CTTTTCCACGGAGAGGGTGTGAGGCAGTGTGGGTTGGGAGCAAGGCCCTTTCGGTTCTCATGCT
ATTTTCTTGACCCCTTCATGGGACTCCCAACAGGGGTACCCCATTTACTCAGCCTGCCCTGCTC
AACCTCTTGCAGGAGGGAGCACACGAGTGAACGAGTGCAGGAACCAGCTGGCTGCTTTAGTGCT
GTGAGGAGTAAACTCCATGCAGGCCCTGCAGCAGCAACCAGTTTTTCCAGATTTGCTCAAAGCA
ATCCCAGTGAGCATCCACGTCAATGTCATTCTCTTCTCTGCCATCCTTATTGTGTTAACCATGG
TGGGGACAGCCTTCTTCATGTACAATGCTTTTGGAAAACCTTTTGA
Exemplary Human CLRN1 cDNA coding Sequence (codon optimized)
(SEQ ID NO: 19)
ATGCCTAGCCAGCAGAAGAAAATCATCTTCTGCATGGCCGGCGTGTTCAGCTTCGCCTGTGCTC
TGGGAGTTGTGACAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAGATGCAGTACGGC
CTGTTCCATGGCGAAGGCGTCAGACAGTGTGGCCTGGGAGCCAGACCTTTCAGATTCAGCTTCT
TCCCAGACCTGCTGAAGGCTATCCCCGTGTCCATCCACGTGAACGTGATCCTGTTCAGCGCCAT
CCTGATCGTGCTGACAATGGTCGGAACCGCCTTCTTCATGTACAACGCCTTCGGCAAGCCCTTC
GAGACACTGCATGGACCTCTGGGCCTGTACCTGCTGAGCTTTATCAGCGGCAGCTGTGGCTGCC
TGGTCATGATTCTGTTCGCCAGCGAAGTGAAGATCCACCACCTGAGCGAGAAGATCGCCAACTA
CAAAGAGGGCACCTACGTCTACAAGACCCAGTCCGAGAAGTACACCACCAGCTTTTGGGTTATC
TTCTTCTGTTTCTTCGTGCACTTCCTGAACGGCCTGCTGATCAGACTGGCCGGCTTCCAGTTTC
CATTCGCCAAGAGCAAGGACGCCGAAACCACAAACGTGGCCGCCGATCTGATGTAC
Exemplary Human CLRN1 Genomic DNA Sequence
(SEQ ID NO: 5)
AGGAGATACTTGAAGGCAGTTTGAAAGACTTGTTTTACAGATTCTTAGTCCAAAGATTTCCAAT
TAGGGAGAAGAAGCAGCAGAAAAGGAGAAAAGCCAAGTATGAGTGATGATGAGGCCTTCATCTA
CTGACATTTAACCTGGCGAGAACCGTCGATGGTGAAGTTGCCTTTTCAGCTGGGAGCTGTCCGT
TCAGCTTCCGTAATAAATGCAGTCAAAGAGGCAGTCCCTTCCCATTGCTCACAAAGGTCTTGTT
TTTGAACCTCGCCCTCACAGAAGCCGTTTCTCATCATGCCAAGCCAACAGAAGAAAATCATTTT
TTGCATGGCCGGAGTGTTCAGTTTTGCATGTGCCCTCGGAGTTGTGACAGCCTTGGGGACACCG
TTGTGGATCAAAGCCACTGTCCTCTGCAAAACGGGAGCTCTGCTCGTCAATGCCTCAGGGCAGG
AGCTGGACAAGTTTATGGGTGAAATGCAGTACGGGCTTTTCCACGGAGAGGGTGTGAGGCAGTG
TGGGTTGGGAGCAAGGCCCTTTCGGTTCTCATGTAAGTAGCAATTGCATTTGAGTTATTTAATG
CTTTAGGCAGACTCTTCCCAGTGTTGCGAGGAATTATATTTGAGAATTTTGCCGTGTTTACTGC
AGGACTTTTTAAATCGGTGTGAACCATATGAAAAACCTATGACTCTGAGCAATTTCTTCTTCCT
AGTTTTTATTATTTTATACTTGCTTTTTATTATAATATAGAGTTAATTCATTGTTACATAATTA
AGGTTTTTGGAAATATTGGCAATTAAGATGCTTAAGTATTAATATTTATGTAAAAAATTATGGA
GTCTTTTTAAAAAAGTAAACTTGGGGAAATAGGAAAGCTGTAAAGAATGATCTTTATGCTTTTT
GTTCTTTATAAAAAGAACCAAGGTCATGGGCTCCGTATTTAACCAGGTTGCCACCTTTCTCATG
ATTTTGTTTCCTGCTCCCCACTCCCTCCCATTATTCCTGCTAAGACCTTTCCTGCTGCTAAATA
TTCAGTTTTCATTTTTAACTAATTTGGAATCATTTGGCTATAGAAATTTAAAATGATCTGCTGT
GCTAACTGGGAAAGAAATGGATGCCTATTTAGTATAGAACATTTTAAACTGATTGACCTGCAAA
TCATGTAGAGAATATGAGAGAGATTTTCTTGTTGTGATTTTTGTGAAATGGAAGTGTAATCCAC
AGTATTTATAACCTGTTTATCTTAAGAAGAGAATTTTTAAAAATTACCATGTGAATAGGCAACT
CATTAAATGAAAATTAATAGGAAGTCATTTGTTATATCTCTTACAACACACATTCAGAAGTTAT
TATTATTTCAGAAGGGCTGGTTTGGAACAACCTTATGAAGACACAGTCAGTAAATTACTGCATA
AATCACTCTTCAGGAAAGGAGGTTACCAACTGAAGCATTTAAAATGAATTATTATTTTGCCCAG
GTTTTTTTTTTCTTTCTAGTATAGGTAGAAGGCTAAATTAATTGAATTTATTATTAACATATGC
AGTGCCTAATTAAATTTCAGTGCTGGTCTATTTATATTTCTGCAACATTCCTTATATCTTCTTA
GCAGTCATTGGACACCAACCTTCAGCTCACATAGGTTACTAAGTGATATGAATTTTCATAGGGC
TCCAGAAAATTTCCAAGAATTGGTTGTTAGCTTTTTAATTGATGAAGTGGATACCAGTTCTTTT
CACTGAATGGCTTTTATTCATTAAGGTAATGGGGCTGTTAGAGTTGCTTAGTTTTCCTGGGGAA
GGGGAAGGAAGAAAACAAAGCAGAATGTCATGTGATATGCAACTGTATTAAAAAACCGAAAAGG
AAAAAAGTTGAGAGAGATGATTTAACCGTGAGTCACCGGCAGCCAAAGCGTGAGTAAAGCTTCT
CACAGATGAATTTAGACAAAAGCGGAGAAGGTACTGGTGAATTTTCTGGAGCCTTTACATTTTC
TACAGTGAAATGGAGATAAACTTTACTCATGCCATAGGACATGTTTCAAAACAATAATAAGATG
TTTTCTGAACACTTACTACATACTAAGCACTTTATATGCTTTGTCTCATTTAATCCTTACACAG
CCACATTCTTCTGGGGTTTAGCGAATGATTTTTGTGGTTGTGTCCTATGCTTGTCCTGTCTAAG
GATGAAGTTGTTCTAATTGGGTGCCCCTCCTTTTGCTTTCTGTGAGGACTTGCAGAACTGGTGG
GGTTTAAACAGTACCCTCACTTATCTCACAGAATTTCATTAGCTCCCAGATACCCCTGACATTC
TCCCCCTAGCCTAGTGAAGAAAATCTTCCATTTACTTGTTCATTCTGCAGTGACAGCTCCATCA
ATATACAATAGACTATACATATTAAGTGTACTGTATATACTATACATGTTAAAAATCTCATTCA
TTTTGGTGAGGCCCAGCTAAGAATACTTACAGTAGAGCTTTTTTTTTTTTCCTAAGCATAAAAG
TATCTTTTTCAATGCAGCATGAGACAGAGTTGGGAAAACCAAAATAAATAGATCCAATGGACTC
CCCAAAGAGGATAATATTCATTTAAATAAACACCCCTCTCAGTGTTAAAACTTTCTAATCAACA
TGCCTTTGGGACACATTGCACCCTCAAAGTTTACACTCCCATTGCAACGCAGCTTTGTGGTTCA
CGTTTTTTCCATTCAGAATGTCATTACCCTGTCAATGATGTTTCATCAACGTTTGCTTGGATGA
GAATCCTCTGATATTCTTCCTGATAGAAATGTATAAGCCCTGTTCATATAAATGAATAAAAGAT
CTAACCTTACTTTCTCAGTAGTGGCTTCCTTGGAGCAAAAAGCAGGGACCTCCAGAGAGCTCAG
GTGGATGACTCTTTTCTGTTTCTTCCAGAGCTCAACTTACAATTAGTGCACAATTCATTTCCCA
GAATGTCTTCTTTCTTATTGTGCCTTTAGAAAGTTATTAAGCAAACATTTGAATTCACAGAATC
TTACCAGTGTAAGAGGAATGGAAAAGGTAACTTATCAAGGTAACAATCACTTCGTGGCCAGTTT
TTTCGGCTCACTGCAACTACCCCTCCTGGGTTCAAGCGATTCTCCTGCCTCAGCCTCCCAAGTA
GCTGGGACTACAGGCGTGCACCACCATGCCCAGCTAATTTTTTTTTTGTATTTTTAGTAAAGAC
AGGGTTTCACCATGTGGGCCAGGCTGGTCTCATGGCAAGTTTTCTTTGTGTTGTCATGTTATTA
TCAATTAATAGGAATTTATATTTCAGTTCTGTTAGGTGGATAAACACTATTTTGCATACCTAAA
TGTTTCATTTATATCAGCACTGGCCAATAAAAATATACTATAAGCAGGCCGGGTGCAGTGGCTC
ACGCCTGTAATCCCAACTTTTGGGAGGCCAACACTTTGGGAGGACACAGGGTCAGGAGATCGAG
ACCATCCTGGCTAACCTGGTGAAATCCCGTCTCTACTAAAAATACAAAAAATTAGCCGGGCGTG
GCGGGGGCGCCTGTAGTCCCAGCTACTTGGGAGGCTGAGGCAGGAGAATGGCACGAACCCGGGA
GGTGGAGCTTGCAGTGAGCCGAGATCTCGCCACTGCACTCCAGCCTTGGTGACAGAGCAAGACT
CTGTCTCAAAAAAAAAAAAAAAAAAAAAAAAAAAAAAATATATATATATATATATATATTACAA
GCCACAAGCCACATATGTACTTTTAAATGTCCTAGTAGCCATATTAGAAAACAAAAGTAAAAAG
GAATAGATGAAATTAATTTTAATGATTTTTTTAAACCCAAGATATCCAAAATATTATCATTTTA
ACATATAATTAAAAATTTATTGAGATGTTTTACATTGTTTTTTTTTTTCTTGACGCTGTCTTGG
AAATCTAGAGTGTATTTTACATTTACATTACATCTAATTCAGTCTAGCCACATTTCACATGCTC
ATTTTTTTGTGTGTGGTTTTATGGAGCAGAGAGTTTAATAGGCAAGAAAGAAAAGAGAAGGCAG
AAGAAAATGGCTCCCCTGTACAGAGACGCGGGGGTGGGGCGCTCCAAAGCCAAAAGAGGAGGTC
CCTAAGTATGGTAGACACCAGCCAGGAATATATGCAGTGTCTGGAGGAGGGGATGTCTGATTTG
CATAGGGTCACATGCTCATTTTTATGGCTACTGTATTGGTCAGTACAGATTTAGATGGGGATTT
GCTCTGAACAAGTTGGTGATTGATGGTGTTCATATTTTAATTGAATTTTTCCTGGGTTCTGCTG
TACACTTGTATGTGTGTTAGTTTCATGTGCAATGCTTGTGTCACTTTTAAAACCCAGATATATG
GCATAACATGAGAATGAAAAATGGACCAGAAAAATAGTTTGGCAATGTAGTCATGTTTGTTCCT
ATTAAATGTTCCCTATTGACCACTCTATCTCTTTTAATTATAACAAGAATCTGCCCTGCCAGCA
TGCCCAGTTACGCTGGGAAAACTTCTGCCTCATTTACTCTGGCTGATTCTCATCCACTTATGGG
TCAGTGGTTCATTTTCTAGAGGTCACCAGCATTCATACCTAGCATACAATTTCATTCATTATAA
TGAAGGAATGTTTTCCCTTCAAAGAGACACAACTAGTGGGCTTAATTTTTCTTGATATGTCACC
TGTAAAATTTTAATGATGATGTTTAAACTCTAAATGTAGCCATCAAGACAAAAACTGCAAATTT
TGAGCCTCAGTGTGTGTGGGTGGGTTTCTGTTTCGGTAATTTGAAACATTGCAGAATATCATCA
AAATATGATACCCAAGAATCATATGGTATCAATCATTCCTAGATATACTGATCTATTCATTGCC
AAGATAGTTCAATGAGCTGGCAAAAACATATGGAATTATTTTCTTAAAATGTGAAAAATAAAAT
TTAACCAATCATGTATCACAGCTTGCAACTTTAGTCATACTTTGAAAAGCATTTTAATTTGGAC
CTCATGATTGAAAATTTATAAAAAGCTGAACAGAAATTAGTTCACTTCATATTTTAGAAAAGCA
GAGTTTCTTTACTAAATGAGGCATTTGACCCAAATTGGAGAGAAAATGTTGAAACTACTTCTGT
GAGCAAGCAGGTGGCTTCTCAAACACATGTTGGGATGAAATGGTTGGGCCTCAGGGTCTCAGTG
CCTGTCACTGAGAGTTGGCACTCTCTATCTCCATGGTCTCCTCCAAGTGTGACTCTTGTCTCTT
GCTGACCTGACCTGCCCCAAGTGACTCACTGGTCATGACCCTGCACACCTTGCGTCTCTCCTAT
CACCCTGCCGATGGCAGAGCTACAAAGGTCTTTGATGTAGCTCTGTCTGATATCCTGTGTTTCC
CCCTATGGTCTGTGTGGAAGCAGGTATGGGGGTGTGTAAAGGGGAAGCCTATGAAGTTCATCTG
CAAAGACTACCTGGTTAGGGGAGGAGAGGAAGAAGCTATATGCACCATTTCACCAGCAAGCATG
GGCTCTTCTGCCTTTTAGCTTAGGGGTCCTGTTGTCTAGTCTCACTCACCTATTAAAACAGTCC
AGCAAATAGAGGTTTTGTTTACCTCCCATTAAAAAAAAAGCAATTAATGGAATAGAAGATAATA
ATGTATGAGAAGCACTATTGTGAAAGAAAAACCTTCAACTTCTCTCAGCCAAATAATTGCTTCC
TCCTCTGTCCTTCCCAGACCTTGATGTTTGCTCTATTATTTCAAAACAACTATATTATAAATAT
TTGAGAATGTGTATTTCCCTGCAAGGAGATCTTAATCCCCAAGAAGGCAGGAGCTGTGTATTAT
TCATCCCAGTGCTCCTTCAAACCAGGGCCTCAGACAGTGCATGGCCCAAAGAGGTACTTAATAA
ACGTTTGGTAAACTGACACTATTGAAATTAAGCAACCTGGATTTGAGGTGGGTCTCTGCCACTC
ACAAGAGATTACGCTTTGAGAAAATTCCATCACTTCATTGATTTTCAGTTCTTGCATCTGTATA
TGGGAGACGATACTAGGTGATTTCTGACATCTCTCACCGTTTAAATGCTCTGTGATCTATACAA
CGAGGGGCTCGCTGTTCTAGACAAGTTCCTTCCAGCTTTACAGTTGCATAACCCTTCTAATCTT
AGTCACATGATGACTCCACTGACAGATTTTTGGCCACCATCATTAGACATGCTGAGTTACGTGT
GCCTTTGCTCTGATCCTCAAAACTCATGATTTTTAAAGTTTTCTGAAATATCTACCATTTATCA
GGATCCAGATGGATTTCATGACCAAAGTGGATGTTTCTTTTCTCTCCCATTACAATCTTTTACT
TTTTGTGTGGGAAATTGCATGTTAAAGAAAGGGAAATTGAAGAATGGGATGCTTTGGAATTCTG
GCAAGATGGATTAGTGGGTTCCAGAAAGTAGGGGCAGCCACAAATACCGAAATAAATGAGATCG
TATTATTGAGAAAGCACAAATGGAAGAAGGTCAAAAGCAAGAAGAAGCTGACATCCTGCTTCCT
CCAATTTTTGCTTTCTCTGTTTTTCCAAGAAACTCCTCTTCCAAGCCTTGCTGAAAAACTCCAC
TTTCCTAAATCTAACTTCTTAAACTGATAATGGCAAGAAGTTAGGAATGACCAATATGTTTAAC
ACTCCAAGAGTATTTGTTTTGTTTTGTTTTGAGACTGGGTCTCACTCTGTCGTCCAGCCTGGAG
TGCAGTGGTGTGATCACAGCTCACTGCAGCCTTGACCTCTCCTGCCCAAGGAATCCTCCCACCT
CAGCCTCCTGAGTAGAAGGGACCACAGGCATGTGCCACTACACCTGGCTAACTTTTAAAAAATT
TTTTTGTAGAGATGGGGTTGCCCAGGCAGGTCTCAAACTGCTGGGCTCAAGCAATCCTCCTGCA
TTGGCCTCCCAAAGTGCTGGAATTATGGGCATAAGCCACTACGCCTGACCTCTCCAAGGGCATT
CTTTACCCAGAAGAGGAACTTGGCAGAACTTATCCTCCAATTGGTGAGGAATATGGAGAAAATG
ACTTTAAGCAAAGGAACTTCTGGTTCTGCCTACCTAATCCAGAAAAAGAAGTTTTATTTCTCCC
TTCCCCTAGTAACTATCTTCCCATATTCACATAAAAAAGTACAGAATCAACATTGTTCAAGAAT
TATAATTTTACTTGTAAGCACATGTGCACACGCACACCCATATACCTTCCTTCCCTTTAAATCA
TCCCACACCCTAATAGTAGTAAAATCATTGACCCGAGCATACCTGGGAGAGGAAGAGGAGTCTG
ACAGGGGCAGGTTCTAAGTGGCACTCCTGGAACTTAACCCTGGTGTATATGAACTTTACCTATT
GAAGGATGACTCCTCAACTGTTCTCACAATTTGCTGCTCTGCTTTCTTTTCTAATTTCTGAAGG
TGACTCATCTTCCCCAAGGACTTTCAGACTTCTCAGAAGAAAAAAATATTGGGTGGGTCTCTGC
CACTGGCAAAAGATTAGACTTTGAGAATCATAAAAGTATATCAGTATATACTCATTAATATTGA
ATTACTATAATTAATATTATGATATTGATATAATGATAGAATGATATTGATAAAAGCAATATTC
AATAATGAATATTATTTCAGCTGCCCACTTATTGGGTGCCTCATAGGTGCCAGGCATTTTGTAT
GTATTATCTACAACCCTTACATGGGACATATTATGATGCTGTTTCTCTTGAAGAAATATGGAAA
CTGGAAACAGAGAGGTCACCACAATTTTCCAAAGTCACATAGCTAATAGGTAGCAGACTTGGGA
TTCAAATTCATATGCATATGGTAAATCATGCTCTTCCTCTGCTACATTTTGCCCCCTTAGAATA
TGAAAAAGGGATACAAAGAGATGAAGAAAATATGTAAGATTATCCTTCAATTTCACTATCTTTT
TTAAAGTTTTTTTTATTATACTTTAAATTCTGAAATACATGTGCAGAACATGCAGGTTTGTTAC
ATAGGTATACACGTGCCATGATGGTTTGTTGCACCCATCAACCTGTCATCTACATTAGGTATTT
TTCCTAATGCTATCCCTCCCCTAGTCCCCCACCCACCGACAAGCCCCGGTGTGTGATATTCCCC
TCCCTGTGTCCATGTGTTCTCATTGTTCAACTCCCACTTATGAGTGAGAACATGCGGTGTTTGG
TTTTCTGTTCCTGCGTCAGTTTGCTGAGAATGATGGTTTCCAGCTTCATTCATGTTCCTGCAAA
AGGCATGAACTCATTTTATGGCTGCATACCATTCTATGGTATACATGTGCCACATTTTCTTAAT
CTAGTCTATCATTGATGGGCATTTGGCTTAGTTCCAAGTCCTTGCTATTGTGAACAGTGCTGCA
ATAAACATATGTGGGCATATGTCTTTATAGTAGAATGATTTATAATCCTTTGGGTATAGACCCA
GTAATGGGATTGCTGGGTCAAATGGCATTTCTGGTTCTAGGTCCTTCAGGAATTGCCACACTGT
CTTCCACAATAGCTGAACTAGTTTACACTCCCACCAACAGTGTAAAAGCGTTCCTATTTCTCCA
CATCCTCTCCAACATCTGTTGCTTCCTGACTTTTTAATGATTGCCATTCTAATTGGAGTGAGAT
GGTATCTCAGTGTGGTTTTGATTAGCATTTCTTTAATGACAAGTGATGATGAGCTTTTTTTCAT
GTTTGTTGGCCGTATAAATGTCTTCTTTTGAGAAGTGTCCGTTCATATCCTTTGCCCACTTTTT
AACGGGGTTTTTTCTTGTAAATTTGTTTAACTTCCTTGTAGATTCTGGATATTAGTCCTTTGTC
AGATGGGTAGATTGCAAAAATTTTCTTCCATTCTGCAGGTTGCCCGTTCACTCTGATAATAGTT
TCTTTTGCTGCGCAGAAGTTTTTTTAGTTTAATTAGATCCCATTTGTCAATTTTGGCTTTTGTT
GCCATTGCTTTTGGTGTTTAGTCATGAAGTCTTTGCCCACGCCTATGTCCTGAATGGTAATGCC
TAGGTTTTCTTCTAGGATTTTTATGGTTTTAGGTCTTATGTTTAAATCTTTAATCCATCTTGAG
TTAATTTTTGTATAAGGTATAAGGAAGGGGTCCAGTTTAGTTTTCTGCATATGGCTAGCCAGTT
TTCCCAACACCATTTATTAAATAGGGAATCCTTTCCCCGTTGCTTGTTTTTGTCAGGTTTGTCA
AAGAGCAGATGGTTGTAGATGTGTGGCATTATTTCTGAGGCCTCTGTTCTGTTTCATTGGTCTC
TGTATCTGTTTTGATACAAGTACCATGCTGTTTTGGTTACTGTAGACTTGTAGTATAATTTGAA
GTCAGGTAGCGTAATACCTCCAGTTTTGTTCTTTTTGCTTAGGATTGTCTTGACTATTCAGGCT
CTTTTTTGGTTCCATATGAAATTTAAAGTAGTTTTTTCTAATTCTGTGAAGAAAGTCAATGGTA
GCTTGATGGGAAAAGCATTGAATCTATAAGTTACTTTGGGCAGTATGGCCATTTTCATGATATT
AATTCTTCCTATCCGTGAGCATGGAATGTTTTTCCATTTGTTTGTGTCCTTTCTTATTTCCTTG
AGCAGTAGTTTGTAGTTCTCCTTGAAGAGGTCCATCACATCCCTTGTAAGTTGTATTCCTAGGT
ATTTTGTTCTCTTTGTAGCAATTGTGAATGGAAGTTCACTCATAAGTTTGCTCTCTGTTTGTCT
GTTATTGGTGTATAGGAATGCTTGTGATTTTTGCACATTGATTTTGTATCCTGAGACTTTGCCG
AAGTTGCTTATCAGCTTAAGGAGATTTTGGGCTGAGACGACAGGGTTTTCTAAATATACAATCA
TCTCATCTGCAAACAGAGACAATTTGACTTCCTCTCTTCCTATTTGAATACGCTTTATTTCTTT
CTCTTGCCTGTTTGCCCTGGCCAGAACTTCCAATACTGTGTTGAATAGGAGTGTTCACCACCTA
TTTTAAGAATAGTATTGAAGCCTCACAAAAGCTGGTTCTCATGTAACCATCTGAGAATATTTGG
CCTTATGACTTGAATTCATTCATTGCCTTTTTATTTCACATTTTAGTGATCCTGATGTCTAAAT
CTTAATCTTTGATCCTTGCAAGGTAAAATAGCCAAGTCAAGCCTGTTTAATAATATTGGTTGAG
GAAGTCACATGCTTATGATCAATCTTTGGGTTATGTAATTATATTACCTTAATGTTGGCAGTTT
AGGTGTAAGGCAGAGATATCTGATCACATGTGTGGTTAGCTAATTTAAGATCACTGCCAACTAA
AATCTTCATGGTATGATCTTCAAAGTTAGCTACTTTGACCACAGCAATGATTTCACCACAGCAA
TTAACAAAATGGCAGACTCTTTCCTGAGGTGGCATGAACAGTTTTAAAACAAAGTCAAGGACCA
AAAGAAAAGCAGGCACATGGCATTTGATTCATTTCAAAAACTAGTATTGTATTAAGAGCCAAGG
GGATAGAATTGTAGCATCAATTAAAATCTTGTTTGAAAAAAAATAAAAACAAACGTCCATTTTT
ATCTCTCAAATATATTAGGGTTTTCATAAAGTTATAGGTGTATTTTTAAAAAAACAAAACTCAT
ATACATTAGACTGAAAAATTTGCCTGTCATCTCATCATGCAGCTAAATGCAATTGTCTATGGCG
AGATACACTCTTATTAGAGGTATGATAGCACTAACTAATAGCAAACTTTGTACCTGGTAGTCTA
ATTTATGCAGGGTTCATATTTCGCTCCCTCTCAGCATGCTGTAACTGTGGCAAAGCCATTCTCA
GGAGTTATTACCCCAACATAATCATACCCCTGTGGATTAGGAGCAGTTAAATGGGTCCTGTTAT
CAGAGACACATATGTGCCACAGCCGCTGTCATCCCTAAGCCACCGTGGGTGATTAAGACTCACT
GATGGGACTACCTCTGAATAGGCTTGAGTGAGGTGGATACACTTCAGCTGAGAGAAATTCAGGT
AAGGGGCTGAGAAAATCAGATTTTGAATGGTTTTATCATACCATCAGGTCTCCTTTTAAGTGCT
GGGGTCATGGATTTCATTCAACCTGACCACATAGCCTTTGGAAGCTTGGCTCAATACCTGAGTG
TGAGATTATGGGTGATATTAAAGGAGATGAATGCATTGAGCTGATGTCAGAGAATGTCTTTTAC
TGGATTTTCATAATGACTGGCTGCAGATGGGCTGAGGGGAAAGTCAGATCAAGAGCATTTCTGT
AAGAAGAAAGAAATCTTCCCTCTTATTCTCTTTCAAGAAAATGAATAGCTGAGCACCAAGAGGC
CAAATACTTTTTTAAAAAACACATCCTTTTATGTAGAGAAGGACAGGTTGAGACGAAAAACAGG
ACCTCTGAAACTGTCTTTATAGCTTTAATCTAGGAAGAAATTGCGGCACCATTGCTGACATCAT
TATCAGAGGCTGCCTTAGTTCTGAGAGCTTCACAGATGGCCTTTTCTGCATTTTACATCTGGCC
AGATGAAGGCAAAAAGGTTGATGAAACCAAAACTATTAGATCAGTGGTCCATAACTCTGGCTGC
ACTTTAGAATCATCTGGGGAGTCCTTAAAACTACTGATCCTGAGGCTCCATCCCAGACCAATTG
AATCAGGATCTCTGGGGGTGGGACCTGAGCATTGGAATGCTTTAAAAGTTCCTCAGGGACTCTA
ATGTGCAGCCAAGGCTGAGAATGACTGAGGTGGATGGTGGCCAAGACAGGTGAGGCCAAGAGTT
AGAAGCCCTTACTGTTAGGGAAGGCAGAAGCCACAAGGAGGAGGGGAGGGGGAAGGAGCAGTAT
TTAGCATTTCTTCCACACAGTTGGGGGGTTTTCTGATCAAAATTAGGCTGGGATCTTTCGCTTC
CATTTTCATGAAGGTCTTATGCTTTGTCCACAGCCACCTGGCCTCAGGGCAATGAGCAATCTGA
TTGATGAATTTTCAGTAAGAAACTGAGCACACTTGGCTCTCAGCCCCAGTGGCTTCCCCTGTTT
CCAAATCTGCCCACCAGTTACAGGAGCCTGCTCACCAACTGGTTGGGTTAAAGAAGTCGGCTCT
GTCCTTGGCAGGGAGGCCTTCAGCTGTCTGGCCCTGTCTGTGACTGCGTGGGTGAAGCTGCCTA
ATTTGGGGAACTTGATGGAAGATCTAAGCTATGTTCTCTAACAGTTTTATCAGAAATAAAGTTA
ACTTTTGACCTCCATCTGCCTGTCTCCTGTGGAAGGGCTCTGCTCCTTCCAAAGAGACTCTCAG
GGGTTCTCCTTAGAGGTGTGTTATCAGTCCAAAGATCATTTTAGACCAGCCATTACAGAGGATG
TCCAAGAAATTGCACAAGGGAAATAGAAGTAAGAATGAGAGCAATATATCAGATAGTAAGGAAA
GTATGTGACTGGTTTCAAATAAGTAATTAACCATAAATCCAAAGTTCCTGCATTAGTTATAGCA
ATAGTCATCCACCTGGTCAGGAAAAAAAAAGTAAAAGACTGAGCTGCAAGATAGAAAGTTTGCC
TGAATTCCACGTACTTCAAGGACTACTATGGAGGATTCCTTGGTCCCAATGCAGAGACATGTAC
TCTGACCACCCATGGGCCAAATCCCTCTTACCCACCCTCAGTGATTCCTCCTGGGACCTCACTA
CATTGAGTTCTTACATTCCCCACTCTTTTCGGGGGAAATATAACCCTTCTGCTCTTCTCATGAT
GTTAAAAATATTTAGACAATTACTTAAAATTTACAACAATCTTCCACATAAATGATCTCACTTA
TGTTTCTTGTGAGCTCTGTGATTTATTTCTATTATCATCTATGCTGATAGTTAAGGAAACTGAG
ATATCGAGACCAACATGCATAGTAAATGGCAAAACCAGATGAATTTTAAACTTTCCTAACTCCA
AAAGCCACATCCTGCCCAACCCGCCATGCTGCCGCTCAGTTAATGCCTGGCTGTTTGTCTCCCC
ATTGGCCCCTCTACCCATTGTGCTTTGATGGCACACTGTATTCCAACTGCCTGAGACTCCTGGT
TAATGCCATTACGTACAGACTTAGGTTGAATTTACTAGGATTTTTAAGATTTGTAGGATAAGAG
ATATGACTGTTAGACTGGAATCAGCAATAGATAAAAGGTTAACAAAGTTTCAGAATAAAATATA
ATAGAAAACCCCAGCAGATAGAAGTAAAATGAATGGTAAGACAACTGAAATGAAAGCCATACAT
TTAGAAATATCAAATAAAACACAGATTAATGGCAGACAATAAAGGAACATACTTAGCAGTTAGT
AAAAACACTTTTTACCTTATTCTTATTACCCTCCAGTAACCTTTTTTTTTTTTTTTTTTGAGAC
AAAGTCTCGCTGTGTCGTCCAGGCTGGAGGGTAGTGATGTGATCTCGGCTCACTGCAACTTCTG
CCTCTTGGGCTGAAGAGATTCTCCTGCCCCAGCCTCCTGAGTAGCTGGGACTATAGGGGCCCAT
CACTGCACCTGGCTAATTTTTGTATTTTTAGTAGAGACGGGGTTTCACCATGTTGGCCAGGCTG
GTCTTGAACTCCTGGCCTCAGGTGATCCACCCGCCTCAGCCTCCCAAAGTGCTGAGATTACAAG
CGTGAGCCACCAAGCCCGGCTGTAACCTATTGAAAATAACATTACTTGACATGTGAGACAAATT
ATTTGTAAGTTAAAGAGTTTATGTGCCTTCAATGCTCCCACCCTTCCCTCCCCTAAAAAGATTA
TTGAGTGCCCATGATGTGCCTAAGCCTTCTGGTAGACTCTGAGAATGTGAAGAGAGTTAGAGGA
TACTTGTTCAACAACCCAGAATCTAGCAGAAGTGTTTTGAAATGACCAGCCACTTGGGAGAGCT
ATGCTAGCATATCATTCAGGATGGGCTGGGTCATGTTTTAATAACAAGTAAGTTTGAACTTTAT
AGGTTTGAAACAAAAAAGATGTGTTTCTTGCTCACAGTTCATATTTTTTGGAGATTGGCTGGAG
CTTCATTCCAGGATACTGCCACCATCTGGAATGTTGACAGTTACCGTAACAGAGAAAGAGTTGT
GGAGGCTGTCACACTGGCAATTAAATTCGCTGACCTGGAACTGACACATGCCACCTCCACTTAT
ATTTTATTGGCCAAGGCTTGTTACACAGCCACATCTAACTTCAGAGAGGTCAAGATGAGCAAAT
CCTACCACGAGCTATAGATGGATGAGTAGCACTCATTATTATCACAATTATTTTATTTGTTATA
AAAACTCCAAGAAGGAGAGGTACTCTATGTGAGGAATAGGCATAGGAAAATCAGAGGGAAGTTC
TTAAAGATGAGCTAGATTTCACTAGATGGCATTGAAGAAACATTCCAAGTAAATGAACAGCATA
AGCAAATGCATGAAGAACTTATTGTGGTCCTAGTACAGACGATGCGTGAGAGTGTGGAGGAGGG
AAAAGGTAAGACTGGAGAGGAAGGCAGGAACCAGAACAGGACAGACTGTGTTCACTGTCAGGCA
GTTAAGTCTATCTTGTAGGCAACATGAAGCCTTTTAAGGAAGGCAAGCAGCTATGTGACAGGAC
AGAAGATGGATTGGAAGAAAATCAAAAGAGAAGTGGGGACCAGTCATAAGGCTCCTCCAATATT
TGGGGATCCAAACCAAAGCACTGGCAGAGAAATAGAAAGGAAGGGAAATATTTCCAAAATATTC
AAAACAGAAACCAAGAGAACTTGATGACAGAAGATGAACCCAGGTTTCTACTGAATGGACAGTT
GTTACATTCTCTGAGATAAGGAATACAGAAGGAAAAAGTTGAGAGGGAATATGAAATTATTTTT
AATCATGTTTAATTTGATCACTTGTGGAACATCAACCAGAGATGTCCATCAGTTACACCAATTT
GTAGGTTTTGAGAGAGGCCTGAGGTAGAGGCAGAGAAGTGGGAATCAGCAGATTAGCGGCAGTA
GTTGGAACCATAACTGAAGATGAGATTTCCCAGGGAGGGGAGTTGAACAGGAAGGTAGACAAAG
GCTGAGTGCAGTGGCTCATACCTGTAATCCCAGCACTTTGGGAGGCTGCAGTGGGTAGATCACA
AACAAGATCAGGAGTTCAAGACCAGCCTGGCCAACATGGTGAAACCCCGTCTCTACTAAAAAAA
CACAAAAATTAGCCGGGCGTGGTGGCACACACTTGTAATCCCAGCCTCAGGAGGCTGAGGCAGG
AGAATTGCTTGAACCCGGGAGGCAGAGGTTGCAGTGAGCCAAGATCGTGCCATTGCACTCCAGC
CTGGGCGACAGAGCGAGACTCCGTCCCCACCGACCCCCTAAAAAAGAAAGTAGACAAAGATGTG
TCCTAGAAAACACTAATATGAATGGGTAGAGAGGGGAGACTTGTCAAGGAGATCGAGGGGAGAG
TCAGTGAGGTGAAACCTTAGAGGATAGACCTTCACCAAGGAAGTAACAGTCAACAGGCCTAAAT
GCCACAGAGATGTCAAATGAGAGACACTGGAAATGGTTCTTTGAATATTACAGCCAGAACATCA
CAGGAGACCATTTCCAAAGCAGTCTTGATGAAGTGGTGGGGAAGGGAGGTCCCTGAAGGAGCTA
AGGAGGGACTGGGAGATCATGACCCAGAGATAAGTGTTGCAGGTATAAAAGGGAAAGACTGAGA
TCAGGAAATAGCCACAGGATCCCTAAGGCCAGGCTTCGGGTGGGGTGGTGTGTGTGTGTGTGTG
TGTGTGTGTGTGTGTGTATAAAATGGGAAGACCTTGAGTATAATTCTGTACTAAGAAGATAGAG
CAGTAGAGAGGAAAAGGCTGAAGACAGATGGGAGGGTAAATAGTGAATTAGAAAGGTCTGTGCA
AAGATGAGAAAGGATGAGTCTGAAAGCACAGGGAGAAGGGCCAGCCTTGGACAGGAGAAAACAT
TTCTTTCACTAAGAATAAAGGGAAGGTTGGGTATACACCACAAAGAAGGTGTAGATGGGTGGCA
ATGGAGTTCTGTCAAGTTGAGGGCATTCCATGATAGCCTCACCTTTCTCTGTGAAATGAGAGAG
TTTAGTTTACAAGATATTTCTGAGCACTTCATAAGCCAGGTTCATAGACTGAAAGAAGTGGAGG
TGGAGTGTGATAGGTCCTTAAGAATAGGGGAAGTTTGGAATATCTGACAAGGGACAGAGAGGAA
AAAACTAGAAAAGGCTTTGCAGAATGTGGGCCCACAGATCAGAGGCTAAGGGGTCCCCATTTGT
GCAGGAGAGTAAGGGCAGGGAGGCAAGGCTCACAGCCCAAAATATAGAGCCCCTCACCAAATGA
CTGCAGGAGGGCAGCTTTCCTATGAGAGCATCCCTATCACTGTTTTCACTCCGAGTCATTAACT
TACGACTTACTCAGCTCTGTTTCGTAATAGCAGACTCGAGTAATGAGGGTATGACAGCCTCTCT
CTGCATGCCAAGGTATGCAGCGTGGATTTCCTTTTTCGCTTTCTCTCTCCTGTGGCTTAGGTGC
CTTCTGTTCTGCTACCAGGATAGAGAACCCAGTGACTAGTTTCTTCTAGCTCTCTTTTTCTGAC
TAGGTATCTTGTCAGAAATTTCTGCTTACCAGACTTCATGGAGAGGGAATCAAGCTTTGAATCA
GGGTTGAAAAAGTAGAGCTTAATATATATATTACAAAATGCCACTCACGTTCTTGAGGTTACCT
TGTATCTATACCACAACTAGCATTCTTTTAGAAAGCACCATTACCGAAGTAATCCCTTTCCTGG
GAATTCACCCAAAAAGGTTATTCCCACTTATCCCCCATCTCCAAAATAAAAAAGAAAAATGTGT
GTGCTTAGAGATGTTCCTGGAAGCATGAGCTGTAATACTGAACCAATAGAAGACGAACTAACAG
ATTGCAGGGCATCCGTTTGGCAAAAAACTTATGCAGTTATCTAAATGATAGTTATGAAGACAAT
ATGTATAACACATTATATTCTGAGTGAAAAGAACAGAAGGTGATTTCAAAACTGCATTGGGATA
ATAGTAATATAGGAAATAGTGAGATGAACAAAGATTTCAAAGGAACAAAAATAAAGAAAAATTT
TGCTTTTTATATTGGTAGGCGTATGGGTGAAATTCCAATTTTAATTTTAATTTCATAGTTATAA
AATTGTTCATAAAAAAAGGTCCCCCATAGACAGTTGGGCTTTGGGACAAACTAACAGAAACAGA
GTAGGAAGAAAATTCATCTTCCTTCAATCCCCTTTCTCTGCTTAAAACAAAACAAAAAAGAGCT
TTGTCATGTTCAGGTGTGCAACGAATTCTTTTTCCAAATCTGGAACTTTACATCTGCTATTAAA
CAGGAGTCAGTTTCCATGTAACATGTTGACAATCCCCCAAGTGTGTTGGAATAATTTTTTTTAA
TGAGGAGATTTGAAATTCCATTTCAATTGCCAACCTGCCTCTTTCAACTTCTAAAAACAAAGTA
AAACAAAACAAAAACACACTGGGTCCTATCACCCCCTCTTGCTACTACTATTTTATCTCCATTG
CCCTGAATTCTTTCCAAACTTCTTTCCACCCAGCTTTGATTTGTTTTCAGTCGGGTTTATTGAG
GCATAATTTACGTACAGTAAAATTCATCCTTCTTAGATTTAGAGGTCTATGCATTTTGACTAAT
GCATATTGGCTTATAATGACTACCACAACAAAGATATAGAACACACCCATCACTCCCGGGTTCT
CCCTGTCCCTTTTTTGGTCCATCTCCTCTCCTACCCCCAACCCCTTGGCAACCACTGATCTGTT
TTCTGTCCTTATCATTTTGCTTTTTCCAGGATGTTGTATATAAGGAATCATGCAGCATGCAGCC
TCTCGAGTCTGACTTCTTCCAGTTAGCACAGTTATTTAAGATCTATCCGAGTTATTGTGAGTAG
CAGCATCTTTTTTATTGCTGACTAGTATTTCATCACATGGATGGGCCACAACTTGTTTATCTGT
TCACCTGTCAATGGATACTAAGTTGTTTCCAGTTTTTGGCAAATATGAATAAAGTAAACATTTG
CATACAGATTTTTGTGTGGACACATGTTTTCAATTCTCTTAGGTAAATACTGAGCAATGGGATT
GCTGGGTTATATGTTAAGTCTATGTTCAGTTTTCTAAGTTCTGAAACAATTGGATATCTATATG
CAAAAATACAAAATTAACCTTGACTCAAACTTGTACCATACACAAAAAATAACCTGAAAGAGAT
CACAGGTGTAAATGTAAACCTAGAACTAAAAACTTCAAGGAGAGAAACATAGCAGAAAATATTT
GTGACCTTGCATTAGGCAAAGACTTCTTAGATTTGACATTTGAAACATGATACATAAAAAATCT
TGATAAATTGGAGTTCATAAAAATAAGAACTACTCTTCAAAAGACACTGATAAGAGAATGAAAA
TACAAGCCACAGACAGAGAAAATATTTGTGAATTTCTTATCTGATAAAGGGTTTGTATCCAGCA
TGCATAAAGACTTTTCAAAACTCAATAATAAACAATCCAATAAGAAATGCACAAAAGATACAAA
CACATCATCGAAAATGACCTATGAAAGGCAAATAAGCCCACAAAAAAATTCTCAACATCATTAG
ACACTTGGGAAATGCAAATTAAAACAACACTGAGATAAACTACATATCTATTAAATGGCTACCA
CTTTAAAAACCTGTCAAGTGCCAGCCAGAATGTGGAACAAGTAGGACTCTCTTACATTGCTAGT
GGGAAGGTGAATGGTACAGCCACTTTGGAAAACACTCCGCAGCTTCTTATAGTTACACATAGGA
CCTGGGGGTAGAGGATGGATTGACTGTAAAGGGGCAAAACTTCCTGGTAGGGGTGGGGAAAGTT
TTTAGAAGGGTGAAATTGTTCTATATCTTGATTATTGTGGTGGTTACACAACTGTCTGCATTTG
TCAAAACTCACAGAACTGTACACTAAAAAGGTATATTTTTATGCTCTGCTAATTTTACTTTAAT
CTTAAAAATAGGAAGGAAAAAATAAAATCAATGCCACTGTGCGACTTTGGGCAAGTTACTTCAC
TTCTCTGTGCCTTGGTCTTTTGAAATCTATACATTAAGGATAAAATAATACCTTCCTCATACTG
TTAGAATTAAATGTGCTAATTTATGTTTTATATATATAAAGTACTTGGCCGGGTGTGGTGACTC
ACACCTGTAATCCCAGCACTGTGGGAGGCCGAGGTGGGCAGATCACTTGAGGACAGGAGTTCAA
GACTAGCCTGGTCAACATGGTGAAACCCTGTCTCTACTAAAAATACAAAAATTAGCTGGGCTTG
GTGGTGCGTGCTTGTAATCCCAGCTACTTGAGTGGCTGAGGTGGGAGGATCACTTGAACTCCAG
AGGTGGAGGCTGCAGTGGGCTCCACCCACTGCCTGGGTGACAGAGCTAGACTCCATCTCGAAGA
AAAAATAAGTACTTGAAACATAGCAAATGTTTTATAATTATTGGCTTTTTTTTCTTATTGTTAT
TACTTGCATTATTGCTGTTTGAAGAAGTTTGGTGATAATGGAGAGAAAAGGGCAATTAGGGGTC
TGGGATGGTTTAAGTATGAGGAGACCGAGACACATTGACTCAGAGTGAAGAAATCAAAGATAAG
AGAATGAAAGAAAGGGAGGGTAATTCTGAACGCACAGACAAAGTTATGTTACTAACGTGGCATC
GGCTGTGTGTTGTAATAAATAACTCCCTTTCACTTGTCAATAGCTAATCAAATACTTTCTGGAG
ACCAGAAGTGACTTGCTGGATCAATGACAACTCCTCCACAGATCAAAATGTTCAAATCCTTTTT
CTGTGTTGTAATCCTAAATCTAAAAGAACAGAGAGACCAAGCAAATCTACCTCCCAACATCATT
AAAGTGACAACTCTCAGTATTTATTTGAATGGTCTGCTCTCAGCTTCAACCAAGGAAAAGTCAA
ATTAGTGTTGGTCAGAAAACAGAAGGGTGTTACGAGAGTTCTGGCTGGTCATTACAGACTTGGG
GATTTTTGATTAAAAGAAGAAGAAGAAGAAGAAACCTGATAAAGTGTAAATATAGCAAGCAGGG
ATTAGTGTCCTGCTGGGTCATGTTCTCACAACAGTGAGAATTTCAGAGATTTCATAAGAATTAA
ACTGCTCCACATGACAATTTATTTTACCTTCTGGCTTTTCCAGGAGGCAAATCAGTGCAACTTC
TTTCTGCCTTTGTTTCAATTTGGTAACAACCCTCAATTTTAGGACAGGCTAAACCTAGCCACCC
TATCAGAGATGATGAAGTAGCCATCTTTTTAACAGGTGGGGAGATGAATGGAATCAGGGTTTGT
TTGTTTGTTTGTTTAATAACTGCTAGTAAAAACCAAGTCAATAGCTGACTGAGTGTAAGGGAGG
CTCCAGAAGGCAGGTTATTGTAGTATAGATGTGACTCGACTTATGATGATGTTACTTCCCGATA
AACCCGTCATAAGTTGAAATATCGTTAAATTGAAAATGCTTTTAATACACCGAATCTACCGAAC
ATCATAGCTTAGCTCAGCCTACGTTAAATGTGCTCAGGACGCACATTGCCTACAGCTGAGCCAA
ATCACCTGGCAACACAGGACACTGTAGAGTATCGGTTGCTGGCCCTTGTGATGCTGTGACTGAC
TGGGAGCTGCGCTTAGTGCCTCTACCCAGCATTGAGAGTTTCTTATCGCTTATTACTAGCCTGG
GAAAAGACCAAAATTCAAAACTCAAAGTGCGGTTTCTACCGAATGCTTATAACTTTCAGACCAT
CATGATGTTGAAAAATCGAACCATCGTAGGTTGGGATCCATCCTATAAGACGAGCTACACTGCC
GGAAGTGTAAGACTGCTATGCTGCCGGAAGATGGGGCATAGTGGACAACTGCAAGTCCTGACAA
CAGGAGGTCAGCATCTGCGACCTTTAACATCCACATTGACACTACCACAGTCTTCCAAACAGAG
CTGATGATATAGTTTGGATGTCGTCCCTGCCCAAATCTCATGTCGAATCGTAATCCCCAGTGTT
GGAGGTGGGGCCTGGTGGGAGGTGATTGGGTCATGGGGGCAGAGTTCTTATGAATGGTTTAGCA
CGGTCCCCCCTTGGTACTGTATAGTGAGTGAGTTCTCATGCGATCTGGTTGTTTAAAAGTGTGT
GGCACCTCCCCTCTCTCTCTTTCTCCTACTCTGGCCATGTGAAGTGTTGGCTCCCGCTTTGCCT
TCCACCATGATTGTTAAATTCCCAGAGGTCTCCCTAGAAGCTAATGCTGCCACGTACAGCCTGG
AGAACTGTGAGCCAATTAAACCTCATCTCTTTTTAAATTACCCAGTCTCAGGCCGGGCGCGGTG
ACTGACACCTGTAATCTCAGCACTTTGGGAGGCTCAGGCAGGAAGATGATTTGAGGTCAGGAGT
TCGAGACCAGCCTGGCCAACATGGTGAAACCCCATCTCTACTGAAAATATAAAAATTAGCCAGG
CATGGTGGCGGGTGCCTGTAATCCCAGCTACTTGGGAGGCTGAGGCAGGAGAATCGCTTGAACC
TGGGAGTCAGAGGTTGCAGAGAGCCAAAATGGAGCCACTGTACTCCAGCCTGGGCAATGGAGTG
AGACCCTGTCTCAAAAAATATATATATATTACCCAGACTCAGGTATTTCTTTACCTGAGACTAT
GAGAGAATGGACTAATATAGCTGAAGAATTTTATTTTATTTTTAAAAAACTTTTACGTTTGGGG
GTACCTGTAAAAGTCTGTTACATAGGTAAACTCCTGTCATGAGGATTTGTTGTACAGATTCTTT
CCTGCTCCTCCCCCTCCTCCCACCCTCCATCCTCAAGAAGATCCCAGTGTCTGTTGTTTCCTTC
TTTGTGTTCGTAAGTTCTCATCATGTAGCTCCCACGTATAAGTGAGAACATGCAGTATTTGGTT
TTCTGTCCCTGTGTTAGTTTGCTAAGGATGATAGCCTCCAACTCCATCTATCTTCCTGCAAAAG
ACATGATCTCATTCATTTTTATTGCTGCATAGTATTCCATGGTGTATATGTACCACATTTTCTT
TATCCAGTCTGTCATTGATGGGCATTTAGGTTGATTCTGTGTCTTCAGAATTGTGAATAGTGCT
GCAACGAACATTCGTGTGCTTGTGTCTTTATAGTAGAATGATTTCTATTCTTCTGGTAGTAATG
GGATTGCTGGGTCAAATGGTCGTTCTGCTTTTAGCTCTTTGCAGAATCACCATACTGCTTTCCA
CAGTGGTTGAACTAATTTACACTCCCACTAACAGTGTATAAGTGTTCCCTTTTCTCTGCAACCT
TGCCAGCCTCTGTTATCTTTTGACTTTTTAATAAAAACCATTCTAATTAGTGTGATGGTATTTC
ATTGTTGTTTTGATTTGCATTTCTCTAATGATCAGTGATGTTGAGCTTTTTTTCATGTTCGTTG
GCTGCAGGTACATCTTCTTTTGAAAAGTGTCTGCTCATGTCCTTTGCCCACTTTTTAATGGGGT
TGTTTTTCTCTTGTAAATTTAAGTTCCTCATAGATGCTGGGTATTAGACCTTTGTCAGATGTAT
AGCTTGCAAATATTTTCTCCCATTCTGTAGGTTGTCTGCTTACTCTTTTGATTGTTTCTTTTAC
CATGCAGAAGCTCCTAAGTTTAATTAGATCCCATTTGTCAATTTTTGCTTTTGTTGCAATTGCT
TTTGGTGTCTTTGTCATGAAATCTTTGCCAGGTCCTATGTCCAGAATGATATTGCCTAGGTTGT
CTTCTAGGGTTTTTATAGTTTTGGGTTTTACATTTAAATCTTTAATCCATCTTGAGTTGATTTT
TGTGTTTGGTGTAAGGAAGGGGTCCAGTTTCAATATTCTGCATATGGCTAGCCAGTTATCCCAG
CATTATTTATTGAGTAAGGAGTATCTCCTCCGTTGCTTGTTTTTCCCAGGTTTGTTGAAGATCA
GATGGTTGTAGGTGTGTGGCCTTATTTTGGGGCTCTCTATCCTGTTCATTTGGTCTATGTGCCT
GTTTTTGTACCAGTACCATTCTGTTTTGGTTACTGTAGCCCTATAGCATATTTCAAAGTTGGGT
AACATGATGCCTCCAGCTTTATTCTTTTTGCTTAGAATTACCTTGGCCATTTGGGCTCTTTTTG
GTACCATATGAAGTTTAAAATAGTTTTTTTTCTAGTTATGTGAAGAATGTCGTTGGTAATTTGA
TAGGAATAACATGTAATGATATTGATTCTTCCTATCCATGAGCATGGGATGTTTTTCCATTTGT
TTGTGTCTTCTCTGATTTCTTCAAGCAGTGTTTTGTAACTCATATTGTAGAGATTATTCACCTC
CTTGCTTAGCTGTATTCCTAGGTATTGTATTCTTTCTGTAGTAATTGTGAATGGGATTGCTTTT
CTGATTTGGCCCTCAGCTTGGTATTGTTGGTGTATAGGAATGCTAGTGATTTTTTGTATCCTGA
GACTTTGCTGAAGTTATTTATCAGCTGAAGGAGCTTTTGGGCTGAGACTAGGGGGTTTTTTAGA
TATAGAATCATGTTCTCTGCAAACAGATTTAGTTTGACTTCCTCTCTTCCTACTTGGATGCCCT
TTATTTCTTTCTCTTGCCTGATTTCCCTGGCCAGGACTTCCAGTACCATGTTGAATAGGCGTGG
TGAGAGAGGGCATTCTTGTCTTGTGCCAGTTTTCAGGGAGAATGCTTCCACCTTTTGCCCATTC
AGTACGATGTTTGTGGTGGTTTGTCATATATGGCTATTATTATTTTGAGGTGTGTTCCTTTAAT
ACCTAGTTTATTGACAGTTTTTAACATGAAGCAGTGTTTAATTTTATTAAAAGTCTTTTCTGCC
TCTGTTGAGATAGTCATGTGGCTTTTGTCTTTAGTTCTGTTTATGTGATGAATCACATTTGTTG
ATTTCCTTATGTTGGACCAACCTTGCATCCCAGGGATGAAGCCTACTTGATTGTGGTGGTTTAG
CTTTTTGATATACTACTGGATTCAGTTTGCAAGTATTTTGTTGAGGATTTTTGCATTGATGTTC
ATCACGGATATCGGCCTGAAGTTTCTTTTTTTGTTGTGTCTCTGTCAGGTTTTGGTATCAGAAT
GATGCTGGCCTCCTAGAATGAGTTGGGGAGGAGTTCCTCCTCCTCAATTTTTTTGGAATAGGTT
CTGTAGGAATGGTACCAGCTCTTCTTTATACATCTGGTAGAGTTTGGCTGTGAAGCCATCAGGT
CCTGGGATTTTTTAGTTGGTAGGGTATTTATTACTGATTCCCTAAATAGACCGATAATGATTTT
AGAAGTGGAGTCGGTTTTTTCCTGGTCCAGTCTTGGGAAGGTGTATGTATCCAGGAATTTATTT
AGCTCTTCTAGGTTTTCTAGTTTGTGTGCATATGGGTGTTCATAGTAGTTTCTGATGGTTGTTT
TTATTTCCGTGGGATCAGTGGTAACATTCTCTTCATCATTTCTTTTTTTTTTTTTTTTTTTTTT
TTTTGAGACGGAGTCTCGCTCTGTCGCCCAGGCTGGAGTGCAGTGGCGCGATCTCGGCTCACTG
CAAGCTCCGCCTCCCGGGTTCACGCCATTCTCCTGCCTCAGCCTCCCGAGTAGCTGGGACTACA
GGCGCCCGCTACCACGTCCGGCTAATTTTTTGTATTTTTAGTAGAGACGGGGTTTCACCGTGTT
AGCCAGGATGGTCTCGATCTCCTGACCTCGTGATCCGCCCGCCTCGGCCTCCCAAAGTGCTGGG
ATTACAGGCGTGAGCCACCGCGCCCGGCCCATTTCTAATTGTGTTTATTTGAATCCTCTCTCTT
CTCTTCTTTATTAGGCTAGCTAGTGGCCTATCTATCTTATTAATTTTTTCAAAAAACCAGCTCC
TGGATTTCTTGATCTTTTGAATGGTTTTTCATGTATCAATCCTTCAGTTCAGCTCTGATTTTGG
TTATTTCTTGTCTTGTGCTAGCTTTGGGGTTGACTTGTTCTTGCTTCTCTAATTCTTTCAGTTC
TGATGTTAGTTTGTTAGTTTGAGATCTAACTTTTTGATGTGGACATTTAGTGCTATAAATTTAA
CTCTTAACACTGCCTTAGCTGTGTCCCAGAGAGTCTGGTATGTTGTATCTTTGTTCTCATTAGT
TTGAAAAAACTTCTTGATTTCTGTCTTAATTTAATTATTTATCCAAAGTCATTCAGGAACATGT
TGTTTAATTTCCATGTAATTGCATGGTTTTGAGCGATTTTCTTAGTCTTGACTTCTATTTTTAT
TGTACCGTGGTCTGAGGGTGTTTGATATGACTTTGGTTCTTTTGCATTTGCTGAGGATTGTTTT
ATGTCCAATTATGTGGTTGATTTTAGAGTATGTGCCATGTGGTGATGAGAAGAATGTATATTCT
GTTGGTTTTGGGTACAGAGTTCTGTAGAGGTCTATTAGATCCATTTGGTCCAATGTTGAGTTCA
GATCCTGAATATCTTTGCTAATTTCCTGCCTCCATGATCTAATACTGTCAGTAAAGCACTGAAG
TCTCCTACTACTATTGTGTGGGAGTCTATGTCTCTTTATAGGTCTCTAAGAACTTGCTTTATGA
ATCTGGGTGCTTCTGTGTTGGATGCATATATATTTAGGATAGTTAGATCTTCTTGTTGAATTGA
ACCCTTTACCATTATGTAACGCCCTTCTTTGTCTTTTTTTTTCTTTGTTGGTTTGAAGTCTTCT
TTGTCTGAAATTAGGATTGCAACCCCTGCTTTTTTCTGTTTTCTGTTTGCTTGGTAGATTTTCC
TCCATCCCTTTATTTTGGACCTATGGGTGTCCTTACATATTTTATCTTTATCTATCCATCCAGC
CATCCAGCCATCCATTCATCCGTATCATTTTTAACCAATAAGGACTTTTAAAAGCGCAACCACA
ACACCATTAACATAACCAATAAAATCTATAACAATGATAAAATATCATCTAATACTCAGTCCAT
GTCCAATTTTCCCTCGCTATCTCAAAATCGTCTTCTTAGAAATGGTCTGTTCAAATGAGATCAC
ATGGACAGAGGAAGGCGAACCTCACACTGTGGGGACTGTTGTGGGGTGGGGGGAGGGGGGAGGG
ATAGCATTGGGAGATATACCTAATGCTAGATGACAAGTTAGTGGGTGCAGCGCACCAGCATGGC
ACATGTATATGTATGTAACTAACCTGCACAATGTGCACATGTACCCTAAAACTTAAAGTATAAT
AATAAAAAATAAAAATAAAAAAATAAAAAGTGAAAAAAGAAAAAAAAAAAAAAAAGAAATGGTC
TGTTCAAATCACAAACCAGATTCAGAAACAATAGCCATACATTACATTTTATTAATATGTCTCT
TAAATTTCTTTTAATCTATTACAGTCTTTGGAATTTTTATGTCTTCGTTTATCCTTCCAATTAT
TAAAAAAAAAGTATTTTTGTATTCATTGAATAGACAATGCTTGCAGAAAAGTAAAAAAAAAAAA
ATTTAGTACAAAAAGGTACATAGTGAGCTGTTCCTTAGTCTCCCTTCCCAGAAGCAATGTTACC
ACTTTTGTACAAATAGTCTCTGCCTAGACACACATGCCAGTCCCTAAGGTGGCTGTAACAAGGT
GGTTAAGAGTGAGAACATGAATTCAAATTCCTATTATGCCACTCACTAAGTATAAATCTTGGTC
ATGGTACATGCCTCTGTGCCTCAGTTTTTAATAATGGTACCTACCTCATAGGGCTGTTGAGAGA
ATTAAATCAGATAAGTGCTTAAATAACTATTAATATTTATTATTATTCACATTCCCTTTTGGCT
TTTTTCCCAAATAGCAGAGTGGTGCACATATGTCTTCATTTATTTGGCTTGTTTTTTCACCTCA
CATCACATTTTGATGAATAATTCCATACATGTTGTTATAGATTTGCTTCATTCTTTGTAATCAT
TGACTAATATTCCATTGTATGAATATGCTACTGCTAAACATGTACGTTATTTCCAACCTCTTAT
TATCAAGAAATGCTGCAATGAATATCCTTGTAATACTTTAGTGGATTCATGTGCAAAAATATTC
ATAGGATAAAATCCTGAAAGTTAAATTGCTGAGCTAAAGGGTATGTGCATTTTAATGCTTTATA
GATTGCCCAGCTGCCTCAAAGGAGGTTATAACAATTTACACTCCCAAGAAAAATGCACAAGGGT
CCCCATTTCCCCATACCCTAGCTAACACAGGATATTGCTAAATGCTTTCATCTTTGTAAACATG
ATGTATTGAAAATGGTATCTCAAAGTTTTAATGTGCATTTTTCTGATTGTGAGAAGATAAAGGA
AATGTAGTACAACTAAACATCAGCAGTCAAATGACCTGGCCATGACTCCTGAGTGAGGACACTG
GTAAACACCATCAGGATCCAAACACCTCTGTATTTACGAAGAGGATGCTCCCTATTGGATAGCA
CTAAGCTTATTTCATGTATGTACATATGTAGTTAGTTAATTACATCCAGCGGTGGCAAAGGGCT
TGTTCTGACCCAATGAAACTTTCTCTCCTGGCCCCCTTCCAGCATGTGGTCAGGAGTAGAGTGT
TGTGGCCATGAGGCATGCATTTGTACAGATGACTACTTACTCCTCCTTGAAACATTTTTTTCCA
TTTGCTTCCCTGCTGTCTCACTCATGGGTCTGCTCCTAATTCACAAATCACTCTTTTCCCAGTC
TTCTTGGTTGGGTTTTCTCCTCTTCTGTGCTTGTAGACATGGGGGAGCCCCAGGGCTTCTCTCT
TGAACTACAGCTTCTCCCTGGGTTCATCTCCTTGGGATGTCTGTTCCAATGGGTTTAAATACTA
GGGCTAGGACTAGGGAGAGGTCATTGAGGCACTTAGCACATAAAGTTTAAGGAAACATTCTTTA
TCAGGCCCATGCAAGTGCAGGACTGGCCCTGGAGGGTGACTACCACCTTACCTTTTCCACCCTA
GGCACCTTGTTTGCCTTACCCTAGCCCCAGTCCTCTTTAACACCCCAGTCTTCTCCCTGGACCT
CCAGAAACATAAATCCTATTTGACATTTCTACTTGGAGGTTTTAAGGTAACTCAAACGTAAAAT
ATCTAAGACAGAACTCTTGCATCACTTCCCATCCTGGGGCCCAAGCCTGTCTCTTCTACTAGTC
TATCTCAGTTAACAGCATCACCATTTATTCAGTTGCTCAGGACAAAAAATTTGAAGTAATCCTT
GACTCTTCTTTTTTTTTTTTTGAGACGGATTCTCACTCTGTTGCCCAGGCTGGAGTGCAGTGGT
GGGACCTTGGCTCACTGCAACCTCTGCCTCCTGGGTTCAAGCAATTCTCCTGCCTCAGTCTTCT
GACCTCGTGATCCACTCGCCTCGGCCTCCCAAAGTGCTGGGATTACAGGCGTGAGCCACCGCAC
CCGGCCTGGATTCTTTTTTTTTTTTTTAAACAAGTCCTATCTTCCATCTCCAAAATGTATCCCA
AATCTGACAACCTCTCCCACCGTAGGCCAGCCCCCATCTCTCCCCTCTGAAAATAGCCTCCCTT
AGATCTCTGGACATTTGTTCTTCCCCACCCCCTTGTGATCACTATTCAGCATTCAGAATGATCT
TTTAATATTATGAAGGAGACTGTGTTCCTCTCCTACTTAAAATTCTCTAGTGGCTTCCTAATAA
ATTTAGAATAAAAAGCCAACTCCTCGCCATGGTCACCAGGCCAGGATCCAGTGGGTACAACAAT
CTGTTCCCAGCACAATAATCCCACTTCTGCCTCCCCACCATTCACCAGGCTCCACTGCACTGGC
TCATTCCTGCCTCAGTTGTGCTTCTTTTCCCTGGAAAGTTCTTTCTGTAGATCTTTAAAGGGTG
ATTTCCTTCTCAACATTCAGGTGTCAGCTCGTTTCACTTTCCTGACCATGCCCATCCACTCAGA
GATCACTCAAAATCCCATTACCCTATTTTATTTCTCCATCATATGTATCACTATCTGAAACTAT
CTTGTTGTTGATGCAGGCTATTTTCTTGACCCCTTCATGGGACTCCCAACAGGGGTACCCCATT
TACTCAGCCTGCCCTGCTCAACCTCTTGCAGGAGGGAGCACACGAGTGAACGAGTGCAGGAACC
AGCTGGCTGCTTTAGTGCTGTGAGGAGTAAACTCCATGCAGGCCCTGCAGCAGCAACCAGGTAG
GGGTGCCTGCAACCCCAGGGCCCCAGAGGGTGTGTTACAATGCTCTCGTAGCTCTGCCATCTGT
GGACAGCAGTGTGTTGTCAGCTCAGTGGGCCCTTTGCTTCATCATGTAGGGTGGCTGCCCTCTG
CCTGTGAGGGCAAAGGGCCAGGGTGACAGTCTTTTTGGGTACCCACAATTTGTGCATCCTGAAT
TCTTGTTTGGTGCCCAAGAAGAATGGGGTCACACAGATGAACTGAAGGATGGTGAATGCAGAGA
ATTAGCAATGAAAGTGGCTCTCAGCAGAGAGAGAAGCTGAAAAGGGAATGGGAAGGGCAGGTCA
CTCTCCCCTGAAGTCAAGTCACATCTCTCCAATGTCCAGCCACCATCTCTGAAGTCAAGTTTCC
TCTCTCTGATGTCCAGCCACTTCTCCTCTCTACTGGCTGAGTCTGGGGTATTTATAGGCAGAGG
ATAGGTGGTGGGGCAGGCCATACATAATTTTGGAAAAGGCAACATTCTATTGGTAAAAAGACAT
TATTCATAAAGAACCAATTGGGAAAGAGCGGGCACACAGGGATGGAAGTTCTCACTTTGGGCTG
CAGGTTTCAGGCTTTTCAGCTCAAAAGTGAGGTTTTTCCAGGGACCTGCCCCGTCTGCCTAAAA
TTTCTACACTTCTGTCATTGATACCGCTGGATAACAGCTCTCCTATAAAGTTCAGGGGCTTAAA
ACAAAAATTTACTAATTCACCCGGACAGTTCTTGATAGGGTCTCTCCTAACTGTTGCAGTTACA
TAGCAACTGGGTTGGAGTCATCTTTTCTGGGCTTGACATCCAGGACAGCTTCTTCCCTTGTGTG
TCTGGTGCCTCAGTGCTCCTCCAGGCAGCCTTTCTCTCCAGAAGAGTAGCCTGGACTTCTTGGC
AACTCAAGCTTCCAAAAGAAAAAAAAGCAGAGACTGCTGGTTCTCTTATCAAACAGGCCTGGAA
CTGGCACAATGTACTTCTGCTGCCATATTCAGGAGGTCAAAGCAATCGAAGGCCAATCCAAGTT
CAAAGGCATTGGAGAAAAATGAGAAGTGTCACTTAAATGAGAAGTGACACATGCGTAAAAGGGG
GAAAAGCATTGATTGTGGCCATTTTTGGAGATAAGCTATCACGTTTATTTGTTTGTTTGCTTTC
TAATGGTCTGTCTTCTCCCATTAGGTTATAAGCTCTGTGAGACAACAGGAATCTTGTCCATCTT
GTTTTATGGCTCTACTTCCAACACCTAGAATAATGCCTGGCACATAGTAGGTGCTCAGTGAATA
ACTTAAGCACTTGATACATGTTTGGGGAACTAAAATGAACAGAATTAAACTTCCCCAGATTGGT
CCTGCCAGATTTGCTGATGCCAAGCATGCTGATGCCTCACCAGATGAAAGAAGCCCTAAAATGT
AGGGTTTCGCTTTCTCTGCAAACAAGAAAAACTTGCCCTGAACACAAAATCTAGAAATAGATTT
GGCGTGTTTTCTACATTGAAATATTTCCCGTAGTACCAGAAATTATTTTCCCACAGCTTTGTGC
TACATTAAAATATTGAAGTTGACTGAAAATATCTCCATTCTTTAATCTTGGTGTAGACTAGAAA
CAATTTTTTTGTAACAAAGTAAATATGAAAACTTCCTAATATTTGAACTCCCCAGATATCCCCA
GATATCTCCAAACTTAAAATATCATTGCAAGTTAAGATAAATTTTTTTAAATGACTACCGAGAA
AGGTCATTAAAGGCTTGGTTATTAAAATGTACAGATTTGGGTTATAAAGCCAGAACTTATTTGT
TTAAATCATTACATATGACCAAGCACAGAAAATAAATTACCTCAAATCTCCTCTTTGCTAATTT
TTACTGGTAAACTCTATAAAATGATCCTATCTTTAAACCTTTTTGTAAACCCCTTATAAGTTAG
TAAGTGAGAATGTATTCATCAGAAGGATTTTAGTGATGTTTGAAATTAAAAAAGAGAGATTTGA
TTTTTAAAATTATACTTGCAGACTACTGCTAATGAAACTTCTTCTAACCCTAGTTTTGTCTTAT
CTTCAGTTTTTCCAGATTTGCTCAAAGCAATCCCAGTGAGCATCCACGTCAATGTCATTCTCTT
CTCTGCCATCCTTATTGTGTTAACCATGGTGGGGACAGCCTTCTTCATGTACAATGCTTTTGGA
AAACCTTTTGAAACTCTGCATGGTCCCCTAGGGCTGTACCTTTTGAGCTTCATTTCAGGTAAGT
ACAAAATTCTACCTCTGAAGACAAATGTGCTTTTCAATATGTCAAAAAGACCGTCTACCTAAAT
ATAAAGTTATAATCTTAACATATATACATGGATGCACACTGTAGTATTATACATAATAACAAAA
AGTGTGGAAATACCACACTTGCTCAACAGTAGGGAATTCAATAAATACTTTATGGACATCTATA
TGAATAACTGTGATGCTGACATTAAATTATATTTTTGAAGATGTAATCAAGAGGATAAACGCTT
GTCTCAAAAAGTTACATGGGAAAAGCAGTATGTAAACTTATATAAACATTGTGAACCTAATTTG
ATTATATATATAAAATATAGGGAATATACATATAAAATACATATATGTATATATGGGGGCTATA
TATATATATATATGAAAGAGATAGATAGATAGATAGATAGATAGATAGATAGACAGACAGACAG
ACAATACAGACTCCAATCTGTTGGTCGTGGTTGTCTCTGACCCATGACACTATGGGGGACTTTT
ATTTTTGCTCATACTTTTCAATATTTCTTAGTGTTCAATAATGTGCTATTATTTATACATAATA
ATAAAAATAAATAAATGGCATCAAAAAAGAGTAAAGGGCCAGTGTTCCGCCCACATATGAGCAG
CCATATTCAAGCCTGTAGACACTTTGTGTAGCCTAATGCTAGGTGTATCTGGGCAAGGATAAAC
TCTAAAGCCAGAAATTAGTTCATCAATAAACATGTGCTACTCAATAGCTAGGGCTGATGGAAAA
GAATATAAAACCCAGTCTGTGCCAAATGGTGCTTACTATCTGCAAGTGGGAGAAGGAGAAAGAC
GAGAAAATGAAAAATGTGTGTATAATTTATATGTAGCTGTTCTGTAGGAGATCTCTGACTTCAC
CCCATTCTAACTTTGCAAAAAGATCCAACACTTTGTCAGATTCCTGGGAGGCAAGTAATTTTAT
TGATGGTTTCATGGAGGGATACAGAACGATAACAACTCACACAAAGCAAACAATGTAATGAAAA
TCTCTATTCGACTGTTTCTTTTTCTCCTGAAGTTGCCCTTTGGCTGCCAGCTACCAGGCACCAG
GCTCAAGGTACTTTCTTGCTCTTGACACTACTCCCTTCTCTCATACAATTCAACCCCAACCACA
AACGTGTATAGATCTCTCTCTCTATAAAACAAAGGCCTGTAGTTAACAGGAGGTCACTTGCAGT
GTAGCCTCTGTTCATTGTTACTTGTGCACACTGCTTAGGGTCTCACCCCATCCACATTCTGCTA
ATCACATTATTCACCCATCCAATGTAGATCTCTCCAGTGGAGATTCTGCTAATATTTTCTTTAG
ATTTGTCACAAGTATATAATACAGTTTTAAATTGTACAGATGATTATCCTATAACAGAAGAGTC
TAAGCCTTTTACATCTTTGTATCTCTAACGAAAGCATTCAGCATGAAGCTCTGTACACAGCAGA
CAATTCAATATGAATTTGCTGACTTGAAACAGCAAGCCTAGAAAGGAGATGTTAACTTGGTCAC
TTAGACAGAACAGGTTTCAGCAATCAGAATTCAGATGACATGGAACTGGTAGAACAGGCGCTTT
GAAGCAATAGGACATGAGCCAGTGAGGAGAGGGATGGAATATCATAAACAAAGGCCAAGGGCTT
TGCAATCAGAGCTGAAGAGCCAAGAGCACAGGCTCAGGGTGTGGGCAGACTGAATGAGAAAGTG
ATTCAATCACATGTGAAAGTCCAGATGAGAAGAGAGAGTTGGGATTACTTCTGCTCACCAAACA
TCCAAAACCAAACAGGTGGATCCGGGTGGTGTGCTGTTTTACTGATGACCATTACACAGAATTT
TAACAGAAGGAATGTAAAGCAGTGGTTCTCAAACTGGAGTTCCCAGATGAGCAGCGTCTGCATC
ATCTGGAAACCTGATAAAGCAGCAAATTCTCAGGCCCTACCCCAGACCCACTGAATCAGAAACT
TGGGGGTCTGGGGGAAGATGGCCATCTGTATTTTAACAATCTCCCTTCAGGAGATTCTGAGGCT
GGCTCAAGTTTGAACTACAGGTAGTTGGTTCAACAGGTGTTGGTGGACTGACAAACAAAAAGAG
ACTCCGAGGTAACTCCAAGATGGTAATGTCAGAAAGCAGCTACCACCCCTAGGGCTTGGGGGAA
CCAACAAAAGAGTTTGGCATTGCCAGAACCTAGAATCTTGAGGAGAGGCCCCAGAGCATTGTGT
CTCAGACCTTGAGGACTTGGCACTGGGACACCATGAGGGGTTTCTGGGTGTGGGAAAGGGCTGG
AAACTCCCCAGTTGCTGCCACCAGGGAGAACTACAAGTGAAGTGGAAGGTGTGGGCCTTTCTCC
CTTTTCTTTTCTCGTCTTCTCTCTCCCCCTGGTGCTCATGTTTGACAGAAAACAGCTGAAAAGG
CAGAACTAGTTTGGGGAGTCTTGACCTGGCATCATAAAGCAGAGAAAAGCAAAGCTGGAGTGAA
GGTGAGACACAACAGCTCATTAGCAGCAACAGCCGTCTAGCGCTCCAGCTTCTGAATGAAATTC
TGAAGAACAGCGCACTTGGAAGACAAATTATTTGACAGTTCTGACAGACGACCAAACTAACAGC
ATTTGAAAAGCAAGATGACTCAGAGAATACAGAATTTAATCCAAATCCCAGATCCTATCTCTGC
CTTTGGCCAGGCCTAATGCAAGGAGAACCTGAGCACACAAATATATGCAGGAATGATCAGGACC
TGTGCCTGCATTCTATTCTGTCTCACCCACCTTCAAATTTGTTGTAAAAACATGGGCTCAATAA
AGGTTTGTGAATCAGGGAAGGAAGAGAAGGGGAGAAAGGAAGGGAAGGAGCCAGCTCCAGATCT
GTGTCTTGCAGAGGATAAAGGCCAGTGTTTTTAGATCACCCAGTGTTTTTCTAAGCCCCCAATA
CTTATTTTGAAATATCAAAATGTTCAATAACTAAAAAAAAAACCGTTACAACAATAAAACATGT
TTGAGGGTCAGATGGACTGGCAGTTTGTGACCTCTGGGGATAAACAGGTCACTTTGGAATCACA
GACTTCCTCATTCCCCTTAAATCTCATATGGTACCCAGAAGCCCTTGGAACTTTGGAAGGTGTT
TATTCACAGTTGTAATGTCCATGCAGACCCTGGCTCTAAGACCCAATTGTGTAAGGGTAGGTTT
GTAGCCCTTATCCCAAACATTCTAAGTGTGAGCCAATGCGTCACACACTCAGAGGCCAGAGACT
GTATTGGGGTCCTTTATTTCACGTACGAGTCACATTCCATTAAGAGACCCCAGAAGTCAGCTCT
CTTCCACTGACTGGTTCTCTTCCCTTGTTTCTCTTGCCAATGTGTGCTGCCCAGGTGGCAGTGC
TCACTGTCAGCAGAGAAGAAAAATGCTTTCCTCCTTGGACCTCTTTTCTCTTTTTCTCCTCCCT
ACTCACATTCAGGTTCCCTAAGCTTCCCCGCTCCTTGTGCTGAAGTCATTCTATGGTCATTTCT
TCAACTGTCTACTTCCCTGCTGGATGGGCACCCAAGACTTGGCATCCTGGGGCATGTAGAAAGG
GGAAAGGGAAGGGAAGGGAAAAGAAGTCCTCCCAATTGTCTATCTGGACCTTTCCACACTGCCC
AGAGTACTGCTATGGGCATCTCCTTATGTCTCCCGATGTGGTGCATGCCAGACCCTGCAGGTAG
AAAAGGAAAGAAAGCAACCCATTGGACCAGGCCAGCAAAGGCTTCAGTCACACAGCTGGCTCAT
ACTTATGGCTTCATATTCTGTTGCCTCTTGAACCAGACATTTCTTCCACTCTCATAACCTCCAG
TTTAGCTCGTATTCCTCAGCATTCTCCATGTAATATTGTTGCATGAAACCCATGCAAGTCAGCC
AATTTGCTCTTTCTCATCTTGTCATTTATAAATTGATGCTGAGAAGTCTTTTTCCCAAGGTTTT
TAGTAATACCTTCATCATCCCCCATAGTTCATTTTGGGCAGTGATTCGCTTCTTTGACTGTACA
TTAGAATCATCTGAAGAACTTTCTAAAACTACTGATCTCAGGTCTCACACAACACTAATTAAAT
TATAGTCTCTGGTGGGGTAGGGCTTGGGCACTGCTATTTTACCTTAAGCTCTCTGGAGTCATTC
TAATTTGTAGCCAGTGCTAAGGGTTGCAATATAAGTGAATATATTTCACGTATTTGTCAAACAT
TGACTGAGTGCCCATTATGTGCCAGCCATTATAATAGGCACTGGTGATCCCACAGTGAATCAGG
CACACAATGCTGTCTTCACGGAGCTTGTTGTCTAGTGGGAGAGTCAAACAAAAGTGTATATCAA
TAATTAAGTGATTACAGATTGCAATAATTACAATAAGGGTGATAAACAGGTTGCTATAATATAC
AATAGTATTGCCTTTCACCAGACATTTCTTAAAGAGGTAAATCATCTATCAGACACCTTTTAAA
AATCTCATCTAATTTCAAAAGTGTACATAAATAATTAAGTGATTACAGTTTGCAATAATTACAA
TGAGGGTGATAAACAAGTTGCTATGAAAGAGAAAGATAGCAGAGATCTGGCTTTGAGATGGTGG
TCAGGGAAGACTGCTCCAATAGCTGAGTTCTAAAGCTAAGAAGGAACTGAGAACCTTCACAGAA
CGTCCCAAGTAGAAGAGAAAGCACACTGAAGACTCTAGGGAAAGAGGTCTGCTTGTTGTAGGAA
CCGAAAGAAGGCCAATGTGGCTAGGTGCTGGGTAGTGAGGGGAAATGGCACAAGGAAAAAATGA
GGTTAGAGAGATCAGTTGATACCAGTTAATGTTGGACCCTAAACATTAACCATGGTAAGTCTTT
TAGAATTGATTCTAATTGCAATGAAAACCTTTTGAAAGATTTTAAAAAGATATCTGATAGCTGA
TTTACCTCTCTAAGAAATGTCTGGTGAAAGACAATACCATCACATTGGAGATGGAAAAAGATGA
ATGGATTCTAAAAACATTCTGAAAGTACATTCAAAATGTTTTTCAGGTAGCTTATGCAACTAAT
AAATAGTGGTGGCATTCTGGGTAAGACAAGGGAGGAGCAGGCTTGCAGTTTAGGGCAAGAAAGG
GGTGGGGAGAAGAGCTCAGCACTAAAATCATGTGTTCCATTTGGGGCACATCGAGTCTGAGTTG
CTATGAGACCACCAAGTGGAGATGCCAAGTAAATAGTCAGTTACATGAATCTGGAGTTCAGTGA
AGAGGTCTAGAAGAAAGATGTATATTTGGGCATTATTCGGATATAGATATTATGTAAAGCAATA
AAATTGGATGAGATCACCTAGGGAGAGAATGCACATAGATAAAAACTGACCTAGGACCACTTCA
TGTCTAAAACACCAAAAGCAATGTCAACAAAAGCCAAAATTGACAAATGGGATCTAATTAAACT
AAAGAGCTTCTGCACAGCAAAAGAATCAGAGTGAACAGGCAACCCACAAAATGGAAGAAAATTT
TCACAACCTACTCATCTGACAAAGGGCTAATATCCAGAATCTACAGTGAACTCAAACAAATTTA
CAAGAAAAAAACAAACAACCCCATCAAAAAGTGGGTGAAGGACATGAACAGACACTTCTCAAAA
GAAGACATTTATGCAGCCAAAAAACACATGAAAAAATGCTCACCATCACTGGCCATCAGAGAAA
TGCAAATCAAAACCACAATGAGATACCATCTCACACCAGTTCAAATGGCAATCATTAAAAAGTC
AGGAAACAACAGGTGCTGGAGAGGATCTGGAGAAATAGGAACACTTTTACACTGTTGGTGGGAC
TATAAACTAGTTCAACCATTGGGGAAGTCATTGTGGCGATTCCTCAGGGATCTAGAACTAGAAA
TACCATTTGACCCAGCCATCCCATTACTGGGTATATACCCAAAGGACTATAAATCATGCTGCTA
TAAAGACACATGCACATGTATGTTTATTGCGGCACTATTCACAATAGCAAAGACTTGGAACCAA
CCTAAATGTCCAACAATGATAGACTGGATTAAGAAAATGTGGCACATATACACCATGGAATACT
ATGCAGCCATAAAAAATGATGAGTTCATGTCCTTTGTAGGGACATGGATGAAGCTGGAAACCAT
CATCCTCAGCAAACTATCGCAAGGACAAAAAACCAAACACCGCATGTTCTCACCCATAGGTGGG
AATTGAACGATGAGAACACATGGACACGGGAAGGGGAACATCACACACTGGGGACTGTTGTGGG
GTGGGAAGAGGGGGGAGGGATAGCATTAGGAGATATACCTAATGCTAAATGACGAGTTATTGGG
TGCAGCACACCAGCATGGCACATGTATACATATGTACCTAACCTGCACATTGAGCACACGTACC
CTAAAACTTAAAGTATAATAATAATAAAATAAAATAAAATAAAAAAACAAAAATTGATGTAGGA
CCAATTCCTGAAGAACACTGACAGTTAATTTTTTGGTTTAGGAGGAGGAGAAGCCAGCAAACGA
CACTGAGTAGCAATATCCAAAGAAAAAGAGGAAAAAGGAAAACTGGGAGATTATGAGTGTCCCA
GAGGGAATGTTTCAAGATTACCATCAGCAGTGAGCTTTGTGTAAAGGTGGCCTCCTGTAATAGA
GGTGCGGGCAGGAGAAGGCAGAATAGGGAAAAGGGGGTGAAAAAGCTTCCCTCAAGATTTATAA
TACAGTGGAAGAGAGAGACAGAGAGAGAGAAAGAGAAAGAGAGAGAGAGAACTTAAGGAGGTAG
AGGAAGAGAGAGAACCAAAAAAGAGGGAGCTGAGTATAGAAGCAATTAGATTCATAGTTTTTAG
TTGCGGCAGTGATATTTGAGTGGGGGCCTTTTATATATTCCATTCTAGGTGTTTCCCAGTTGAT
GGGAGAGGGTCTTACCTAGATCTGCATGTAAAAGGGAGTAGGCCAGCTGGCAGACTTGACATGG
ATCAGTGGTAGAACATCCTAGCAGTTCTGTGAATACTCTCTGAGAATGACATGAAAGGTATTGG
TTCAGGCCTTTTGGAGGTGATAAAAACCACCAAAATGTGGACTTATTGCAAATTGTATTTGTTA
CCATTTGCAATGATTATAATTATTCTCTTATATATAGGCTTTCTTACATATAGCTTCTCTTACA
CATAGCGTCATGAGTTATGCCTTCTCTTACATATAGTGTCTGCTATGAGTTATGCCAAGCAGGG
CAAACAAAATTGCTGCCTTTCTTTAAAAAGAGGACGCTCCTAGTATGGGCCTAATTAATTATGA
TAATTACAGCTATGGCATGGAACATAAGCACATTCATATACACAAAGACAATAAAATAAAGAAC
AGTTCAAAAACAGAACAGTTACATTATATATCAGTTTCAGTATGAATAATACCCTGGACTCTGA
AATATGTCTGGGGCACATTATTCTGTAATTGGTGGTGAAAAAAAATCTGCATCTTATCTCTACG
CCAATCCTTATGAAGGAGCTGTTTTTCAGGAGTTCGAGAAAGAGACACAGGGATGTCCAGTCAT
CAAAGCCGCAGAGCCTGAGGAAGAATAAAGGATTTGTGGCAGGAAAACCCAGTAATGAATGTAT
TCTGCTAGTTTCTCAGCATAGAACTTAGAAAAGAGGCCACAGAAGGAAAAGAGAAGTAATGTTA
GACAGCTGATGTTGGCAATGGGCAAAGAATTTCATTCTCATATGCAGGGCAGTGGCTAAAGGGG
CAGTGTTGTGAGGAATCACATTCCAGGTCATTCATGTCCAGGGTGTGGTAGGAGGACTGTGTTT
CATTTATTTTGTACATGGCCCAGCTATGACCTTGTGCAAGGAAATGCTTAATCATTTTCTATCA
CAGTTGCAAAGAGATTCTTATCCTACTCAGAATGTACGTCTTCTCTTCTGTTTCATTTACAGTC
ACAACCCAAGTCCTTGCTTTGACCTCCAAAGCACCACTTGATGTATCCACTTCAGAAACACACA
CAGACAGCTCACCTCTTTCTATCCCTTACCTTCCTCCCCTCTTCACTCAAACCACCTACTTCCT
TTGCATTCACTCTTCCTTCAGCCTGAAATAATCTTCCTCCAAATATCTACCTTCTCACTCCCTC
ACTTCTCTCAAGATACACTTAAATGTTATATTCCCTATGAGGCCTCCCCTGGCCATCCCTCCCC
AGCCTTCCTATCCCCCCTCTCTGCTTTATTTTATTCTCCTTAATATATATCACACTCTGATAAA
CCATTGAATGTACTTATCAAGTTATTGCCTCTCTCTCCCTTTCCATTGTCTCCATCACTGAGAG
CTCTGTGAAGAAAAGGATTTGTACCTGTTTCATTTAGTGCTGTACCCCCAGTTCCCACAACAGC
GCAACAGGCACTCAATAAATAGTTGTTGAATAAGTGAATAAAACAGAAGTAGCTGCATATTTTC
TGGTAACAAATGATATTCTTCTGAAAATGTCATATTTTCAGACATATTTCCGAAAATAAATTCA
AATTAGATAAACATTGTATTTTTAGACCATTTCTTCTTTGCATTAATCATCCTTCTCAATAATA
TAACATTTGTAAAACTTAGGTTAGATATGGGCTCTTCAACTTTCCATTACAGAAGATAAAGTGA
AAAGGCTAGACCCAATGGTGTTATTCCTTCATCTACATCTATCCTTTGGAAACACATGACCAAA
TTGCTTGCCATCACAATCTCAAAATCTACCCTTTGGTATTAACTCACTTCACTTGTCCCTCTGT
CCCTTTTTAGATGGTAGCCATCGGTCTCTGGAGCAGTGTAGAGTCAGAACAACTTCTATTTGGG
GAAGAAATCATTGCTGGTGACCTTACTTTCAATTACTAACTTTCTAGTGACATTTACATAATTT
TAGAGAAAATTAACACCTACACTTGTAAAGTTGTGGCTTTCCCACACCTATTTATCATCTCTCA
ATATTCCTTGAAAAGGAAATTATCAATTTATCATTCTATATTGGCAATGAAATGCCCCTAATAT
CTGTCACCTATAAGACAATTGAAGATGATGTGTTGAAAGCTTTCTGAAAATGCTGATCATTACT
TTAAATGGAATTGAAATTCCAGTTTATTATTTCCAAAAATATGATCTTACTGATCATAGGATAA
CATTTCATAACATTTCAGAGATTTCTTCCCCTTCGAGGAGCCAAACCCATAGGACCTCTGGACT
CCCACAGATCCTGGCAGGGAGTTCCCACTCATAAAAGCACAGGTGCCCTCAGAGTCATTCAGGG
ATGAAGAAGCAACCCTCATTGGCCATGTCCTACGTTCCCCATATAGTAAGGACTGGAGGAGACC
AGTGCTCAATTCTGCAGTCTCAGACAGCTGTCAGAGGAGAGTCATGAATGTGCAGTGTCTAGCA
CATTACAAACGTTTGTTAATTGACTGATCATTCATGGTGTGACAGCCCTATAACTCAGCTATCC
TATTCAGTCAGAAATTAACTCAGTAATCAAAGTCATTAAAAGGAAGAAAAAAAAAACCTACAGT
ACCAGACAGATGGTGGGGAAATCAGACAGATGAAAGGAAAAATGGCTGTAGGTCATTGAGTAAG
ACACTGGGCAGCAAAACCTGGGCCTTGTGCCTGGTTATACTCCACATTATAGCTCCAGCAAGGT
TTGGCAGGATTTCCACAGTCCTGGCTTATTCTAACCTTTCTTGGGAGCAGGAGCAGTGTTGGTC
AGATAGACAGACACATAAGGAATCTGTCCAACTGGCACCGTGTGAATTTGGGCTCTTGGTGTAC
ATGGATAACTGGGAAAAAGAGGAGAGAGACATGTAGGACTGATCCTACCGTTTGTGAAGTCTTG
GGCAAGAGTATGAATGAAAACCCACTTCTCTTCCCCTGCCTGGCTCCACTGCACACAGTAAAGA
GCCTCAAGCATAGGTGTGTGGACATTGCACCATGTATCCAAGCTCTGACCATGCCTCTTGAAAC
AGCTATTCCTCAGCCACCCTCTGACCATGGGAGGAATGACCCAGGAGAAATGACCACATAGGTC
TTGAAAATGGGCTCAGGGCTATTTACGAAGTCAATTCCGGGGTCCCAGGAGTATGGACTAAAAT
GTGAGTCAGGCATGCCAGATGGGTATGTTCTATTGACTTCAAGGATTCCTCATGCTGTGGGAAG
GAACCTCTCCAGAAGAGAGACAGAGCAGAACCCTCTAAATGTGGGGCACAAAGCAGGAGCCCCT
CTTGCTGGATTCAAAGGGTCATACTGGAAGAGTGTAGGTTGAGTCTTATTCTCACATCACTCAT
ATCACTTACACACTTCTTTTATAGCCTTAGCAGCCATGCCACAAAGAGAAACTCTTCATGCATA
TCTTTTGGTCCATAAGTCATAAATAGTTATCCTTGATCCCATGTCTTTTTTAGAGCCATGGACA
GAGAGAAGCAAAAATATACCAAGTTCACACTGAGTTGTCTCCCTTCATATCTCTTAGCAGTCAC
TGAAAGGTTATGAGACTCAGGCTGGGTTTCTATCCTCTGTCCCTGAAACGACAACGTTGACCTC
GTGATCAACCCTAGAATCCAGAGCAAGATCTCAGACTGTCCTCTCTACTACCAGACAGCACACT
TTGTTTTGGGGGCTGTGTGCTTGAAATATTAGCTATGGCAAAAGGCTTTGAGTCTTATGACACC
CCAAGTAACTTTTACTTTAGGAATTTGAAATACAGCCTTGCTGTAATGCTGTCTCCTTAACAAA
GCAGTACCTTTGAAATATTTAACAACTTGAAAAGGAAACCGAGCTTGAATTTTCCTTTCAGGTG
CTCAGGAAATAATGTTTCACTTCTGTCTGAAATTCACCATCTCCTCAGACAAAGAAGGCTCTTA
TGGTAAAAGGAATGGCATTTTCTCCACAATTTTCGAATAAAAGATAAAGAGAAAACAGCACTGC
AGCCTTTTTGTTAGGATCTAACAATAAAGAAATAATACGGTTTTGCCAGGGGAGAGCTCTGGTT
TTAAGCTCAGAATACAAAAATAGGCTGACAAAATTTTACAAAGGAATATTCTCAGCTACCACTC
TGAGGATGGTAGAAAGTGAAATTTCAAGAAAATTATATTATTTGATTATTTGATGATGATTAAG
CTGATTGGCCAGTCCTATGTGAAATTCTAAAGTAGAAGAAATGTGATGTTGTCTTTTCTGCTCA
CCTCTCCCCTCATTCCTACCCCCAAATCTCTGCCTCTACCCCAAACCCAGCCTGACCTTTGGGA
AAGGAATGGGGGCTGTCACTTGCACCGTAGCTCCTCCTGCCTGCAGTACTCTCCCCCCACAGTC
CAGCCTCCCTCTGCTCAGCTAACTTTTCCTATAGCTCCTTCTGGCTACTTCTAAGGAACCTTCC
ATAATGCTGCCCACCCTCTCAGTAACAGCCCCTCTACTATCCCTTGATTACATGCACTGTAATT
GGTTAGTAATTGATTTTATGTCCTCTGCCAAATTATACTCCATGAGAGCAAAAATCATGAGTAT
TATCTTTAATGTTTAAATCTCCACAACTATCCCCCATATAAGTCTAGAGAATAAATGAATGAGT
GAATTAATGAATAGAAACTCAAGCCATTATGTTGCCACTCCTAGGATATTTGGATTAACTTTAA
CTGAAGAGTAAAAAGCATTTACCTGTCCTAAAGGAGACATAAAATTAGTGGGAGAGTATTTGGA
GAAAAAAAAGACACTGTAATACATTCTTTTGTGTTGCCTACCCTGATGTAGTCAGGTGTCCCTG
ATATGGGGTGGGCTGAGGATTTGAAATAAAATACCTAATTTGACATTGTAAGTGGAGGAATCAG
GATTTGAAACCAAATTGGTTTGACCTAAATCAAGCTATTATGATAAGGACTTGCAAGAAAAAAG
GAATCCATAAAAACCATATGAATAGCTACCAATTAGTAAGCATTTATATGTGTCAATTACAAAG
CCAAATGCAAATCATGCTTTATTTATATTAGCCACCTTTTATAGATGAAGAAATTGAGACCTGA
ATATTAAAATTGCCTACTTTTACATAGTAAGTAAAGGAATCAGGGTTTGAACCCAAATTGGTTT
CACCTAAGGTAGAAAACCATCCCAGCAAGTCTCCTATTAACTGGAACCCTATTGTGGTGGCCTG
AGATATAACAGTAGCTGTGGAAGCGCTGTAGAGTCCTGGCCATCCTATGTGCTCCTGATCTGGT
CCCTCCTGCCACCTGCTTCTGCTCCCTGTGCCATCCACCCATCTGGAAGTCTCCCAGTGTCCAT
CTTCGGGGGAGACACTCACCAGAGTTTCCAGCTTCCAGCCAGTATGGAGTGCCCCTGTCCCACA
GCAATCTCACCGAAATCACAGCTACATCTGTTAAAATTAGGCTACCAATGAGTGATAGATGAGG
GGGAAAAATAATAATAGTGTACTAAACAAAACAAATGTTTATTTTTCTCACACATAAAAATCTA
GAGGTTGAAGTCCAGGGCTGGTCCAGAGGCTCCAAGGATCTGGGATTTAGACTCCCTCTTTCTT
GTTTTTCCACAGCATATGGCTTCCATTTCTGGGGCCACATTGGTCCAAAATGTATGCTGGGGCT
CCAGCCATTGCATCCATATTTCAGCCACAGGAAGGAGGAAGTGGGGAAGAAAGGACAGGCCCCT
AATACCTGTATAGTTCAAGAAGACTATCCCGCCCATACTTCCCAACCACCCTTAGTTGAACAAT
GCTGTCTTAATTCAAGACACTCACATGTCTAGCCAAAAATCTGAATTCTGTTACAAACAAGGAG
AATAGAGATGTGCGCCACCTCAATACCTCATCCATAGCTACCTTTTCCTTTGTGCAGCTGTGGC
CAAGTGAAAGCTGAAGGAGCTGTGGTAACCCTTCTGAAGGGAGGCTGGGGCCTTTCACAAGAGG
CTGCATGATTGACATTTATCCTGCATGGCCTGTGAAGTACAGAGAAATATTTTCTCTTGAAGCC
ACATCATAGCAGTGGCTGCTTTGTAGCCTGATTCCACCATTATGCCTTTAAAGTGCCTAGCAAT
TCAGCCTTCACATCATGCAAAGAGGAATATCTCCCAGTCTTTGTAAGATCAGCTTAATTCTAAC
CACCTCCTTACCTCCCACTGCACTCCTACACGCACACACAAATCTTCTTCACTCAGAGCAGAAC
CATAACCCAAGCCCTACCACCTAGAGACTGAAGAATCAGGCTCATGATTACAAATATGCAATAA
TTTTTTGTGTGGATAATGTCAATGGGGATGATGGTAAGAGAATTCCTTGGTTTACACATTGACC
CTCTTCCCTGTCCCTTACAATCAGGAAATATTTGTCCCAACACCTTGTTTCTTCTGTTGCAGGC
TCCTGTGGCTGTCTTGTCATGATATTGTTTGCCTCTGAAGTGAAAATCCATCACCTCTCAGAAA
AAATTGCAAATTATAAAGAAGGGACTTATGTCTACAAAACGCAAAGTGAAAAATATACCACCTC
ATTCTGGGTCATTTTCTTTTGCTTTTTTGTTCATTTTCTGAATGGGCTCCTAATACGACTTGCT
GGATTTCAGTTCCCTTTTGCAAAATCTAAAGACGCAGAAACAACTAATGTAGCTGCAGATCTAA
TGTACTGAAAGGCAAACCTTTCTATAATTTTACAAGGGAGTAGACTTGCTTTGGTCACTTTTAG
ATGTGGTTAATTTTGCATATCCTTTTAGTCTGCATATATTAAAGCATCAGGACCCTTCGTGACA
ATGTTTACAAATTACGTACTAAGGATACAGGCTGGAAAGTAAGGGAAGCAGAAGGAAGGCTTTG
AAAAGTTGTTTTATCTGGTGGGAAATTGCTTGACCCAGGTAGTCAAAGGCAGTTGACTAGAATC
GACAAATTGTTACTCCATATATATATATGTGTGTGTGTGTGTGTGTGTGTGTGTGTAAGATGTC
TTCCTATCAAAAAGATATCAAAGGCACATGGAATATATTTTAATAAAAACAAATAATATCTCTA
ATATATCCACACATTTGTTGCCAGATTTCAGAAAACTGAGCTGCAATCGCTTTCCTAAAACAGT
AGTGTATTAAATGAACATCTATAAAATGTATCAACACACATTTTAAAAAATTTGTTTAAAGTAT
ACTCTTAGGCCAGGCGTGGTGACTCACACCTGTAATTCCAGCACTTCAGGAGGCCAAGGTGGGA
AGATCATTTGAGTTCAGGAGTTCGAGTTACAGCCTGGGCAATAAAGTGAGACCCTGTCACTAAC
AAAATTAAAAAATAAAATAAATATAAAATATAGGCTTTAAAAAAGCATAGTCTTATTAACCATG
TCTGTTGGTCAAAATCTGCAAACTCTAAAAGAAGAAAAGAAGAAAAAACCAAGCTTAGGGTATT
TTTCCTCCCGTGCCTGAGTCCCAATTACATTCACGACAGTACTTTCAATGAACATAATTGTTAG
GACCACTGAGGAATCATGAAAAATGATCTCTGCTTAGTACATTTGATGCAAAATGACTTATTAG
GGGCTGTTTTTCTAGCTATAGTGTCTCGAGTACTAATATGCAATTATGAAAATTATATTAAATC
TGGGATTATGACGGTATCACTGTATCATCTTGGTCTTGTTCTGGCTGTCACCAAGCATGACCCA
GGTCAACTTTTTTTTTCCCCTGAATTACCCATCAAATTGATCTGCAGCTGACTAAAGGCCACAG
CTGAGCCTGGAACTGACCCTTCCTTCATCCTCAACCTGCTGTCCTCCAGAAAGCACCAAGGAAA
AAGCAGAGAATGACAGCAAACAGATCACTAGGCCTCTGACCACAGGTGCTGAGTACTCAGCAGC
CCTCATATAATAGGTTTGAAAGTACTCCTTAAAATAAAACACTGTTTCCCTTTGGAACTATTTA
CAAGGATGAAACAACCGTATACCTGAGAAATAACTTGCTCTGGTGTCAATTCGCTATTCGCCAG
CAGACATCAGAACACACCGAGTTTCCAGATGCTGGTTTTTCCCCTTAAATCAGGAAATACACCT
GGACAATTTCTAGAAGACTACAATTCAGTCTAGCCACAAAGGGGATTTTTTTTTTTTGGTAACA
GGCTAGAGCCCGGTTCTGTAAGTCTTTAGCTGAAATGGTCCAGTACAAAAGCACTGGAAATGAG
TGGGCTAGGAGGACAAGGACCGTCTCCTGCGTGAGGAGTTGGTTGGAGGTCCCCAAGGCCAGGT
ACCCCCTGCACTCTTATTGGATTCCTCTCTGTCTTCTTGGAGTTTTGAAAAACTCCTTCGAACA
CCAGGCTTTTTTCTTTAGAAAACAAGTCTCCAATCGTTCTCTGTTCCGTAGAAAGAGAAAGAAA
ACCTGGAGCAGCTGCTGAAAAATCTAATGAGGAACTAAGAGGCAAACCCACCA
Exemplary Human CLRN1 cDNA sequence (Isoform A) including untranslated
regions
(SEQ ID NO: 6)
ACAGAAGCCGTTTCTCATCATGCCAAGCCAACAGAAGAAAATCATTTTTTGCATGGCCGGAGTG
TTCAGTTTTGCATGTGCCCTCGGAGTTGTGACAGCCTTGGGGACACCGTTGTGGATCAAAGCCA
CTGTCCTCTGCAAAACGGGAGCTCTGCTCGTCAATGCCTCAGGGCAGGAGCTGGACAAGTTTAT
GGGTGAAATGCAGTACGGGCTTTTCCACGGAGAGGGTGTGAGGCAGTGTGGGTTGGGAGCAAGG
CCCTTTCGGTTCTCATTTTTTCCAGATTTGCTCAAAGCAATCCCAGTGAGCATCCACGTCAATG
TCATTCTCTTCTCTGCCATCCTTATTGTGTTAACCATGGTGGGGACAGCCTTCTTCATGTACAA
TGCTTTTGGAAAACCTTTTGAAACTCTGCATGGTCCCCTAGGGCTGTACCTTTTGAGCTTCATT
TCAGGCTCCTGTGGCTGTCTTGTCATGATATTGTTTGCCTCTGAAGTGAAAATCCATCACCTCT
CAGAAAAAATTGCAAATTATAAAGAAGGGACTTATGTCTACAAAACGCAAAGTGAAAAATATAC
CACCTCATTCTGGGTCATTTTCTTTTGCTTTTTTGTTCATTTTCTGAATGGGCTCCTAATACGA
CTTGCTGGATTTCAGTTCCCTTTTGCAAAATCTAAAGACGCAGAAACAACTAATGTAGCTGCAG
ATCTAATGTACTGAAAGGCAAACCTTTCTATAATTTTACAAGGGAGTAGACTTGCTTTGGTCAC
TTTTAGATGTGGTTAATTTTGCATATCCTTTTAGTCTGCATATATTAAAGCATCAGGACCCTTC
GTGACAATGTTTACAAATTACGTACTAAGGATACAGGCTGGAAAGTAAGGGAAGCAGAAGGAAG
GCTTTGAAAAGTTGTTTTATCTGGTGGGAAATTGCTTGACCCAGGTAGTCAAAGGCAGTTGACT
AGAATCGACAAATTGTTACTCCATATATATATATGTGTGTGTGTGTGTGTGTGTGTGTGTGTAA
GATGTCTTCCTATCAAAAAGATATCAAAGGCACATGGAATATATTTTAATAAAAACAAATAATA
TCTCTAATATATCCACACATTTGTTGCCAGATTTCAGAAAACTGAGCTGCAATCGCTTTCCTAA
AACAGTAGTGTATTAAATGAACATCTATAAAATGTATCAACACACATTTTAAAAAATTTGTTTA
AAGTATACTCTTAGGCCAGGCGTGGTGACTCACACCTGTAATTCCAGCACTTCAGGAGGCCAAG
GTGGGAAGATCATTTGAGTTCAGGAGTTCGAGTTACAGCCTGGGCAATAAAGTGAGACCCTGTC
ACTAACAAAATTAAAAAATAAAATAAATATAAAATATAGGCTTTAAAAAAGCATAGTCTTATTA
ACCATGTCTGTTGGTCAAAATCTGCAAACTCTAAAAGAAGAAAAGAAGAAAAAACCAAGCTTAG
GGTATTTTTCCTCCCGTGCCTGAGTCCCAATTACATTCACGACAGTACTTTCAATGAACATAAT
TGTTAGGACCACTGAGGAATCATGAAAAATGATCTCTGCTTAGTACATTTGATGCAAAATGACT
TATTAGGGGCTGTTTTTCTAGCTATAGTGTCTCGAGTACTAATATGCAATTATGAAAATTATAT
TAAATCTGGGATTATGACGGTATCACTGTATCATCTTGGTCTTGTTCTGGCTGTCACCAAGCAT
GACCCAGGTCAACTTTTTTTTTCCCCTGAATTACCCATCAAATTGATCTGCAGCTGACTAAAGG
CCACAGCTGAGCCTGGAACTGACCCTTCCTTCATCCTCAACCTGCTGTCCTCCAGAAAGCACCA
AGGAAAAAGCAGAGAATGACAGCAAACAGATCACTAGGCCTCTGACCACAGGTGCTGAGTACTC
AGCAGCCCTCATATAATAGGTTTGAAAGTACTCCTTAAAATAAAACACTGTTTCCCTTTGGAAC
TATTTACAAGGATGAAACAACCGTATACCTGAGAAATAACTTGCTCTGGTGTCAATTCGCTATT
CGCCAGCAGACATCAGAACACACCGAGTTTCCAGATGCT
Exemplary Human CLRN1 cDNA sequence (Isoform C) including untranslated
regions
(SEQ ID NO: 7)
TGCCTCCCCACCATTCACCAGGCTCCACTGCACTGGCTCATTCCTGCCTCAGTTGTGCTTCTTT
TCCCTGGAAAGTTCTTTCTGTAGATCTTTAAAGGGTGATTTCCTTCTCAACATTCAGGTGTCAG
CTCGTTTCACTTTCCTGACCATGCCCATCCACTCAGAGATCACTCAAAATCCCATTACCCTATT
TTATTTCTCCATCATATGTATCACTATCTGAAACTATCTTGTTGTTGATGCAGGCTATTTTCTT
GACCCCTTCATGGGACTCCCAACAGGGGTACCCCATTTACTCAGCCTGCCCTGCTCAACCTCTT
GCAGGAGGGAGCACACGAGTGAACGAGTGCAGGAACCAGCTGGCTGCTTTAGTGCTGTGAGGAG
TAAACTCCATGCAGGCCCTGCAGCAGCAACCAGTTTTTCCAGATTTGCTCAAAGCAATCCCAGT
GAGCATCCACGTCAATGTCATTCTCTTCTCTGCCATCCTTATTGTGTTAACCATGGTGGGGACA
GCCTTCTTCATGTACAATGCTTTTGGAAAACCTTTTGAAACTCTGCATGGTCCCCTAGGGCTGT
ACCTTTTGAGCTTCATTTCAGGCTCCTGTGGCTGTCTTGTCATGATATTGTTTGCCTCTGAAGT
GAAAATCCATCACCTCTCAGAAAAAATTGCAAATTATAAAGAAGGGACTTATGTCTACAAAACG
CAAAGTGAAAAATATACCACCTCATTCTGGCTGACTAAAGGCCACAGCTGAGCCTGGAACTGAC
CCTTCCTTCATCCTCAACCTGCTGTCCTCCAGAAAGCACCAAGGAAAAAGCAGAGAATGACAGC
AAACAGATCACTAGGCCTCTGACCACAGGTGCTGAGTACTCAGCAGCCCTCATATAATAGGTTT
GAAAGTACTCCTTAAAATAAAACACTGTTTCCCTTTGGAACTATTTACAAGGATGAAACAACCG
TATACCTGAGAAATAACTTGCTCTGGTGTCAATTCGCTATTCGCCAGCAGACATCAGAACACAC
CGAGTTTCCAGATGCTGGTTTTTCCCCTTAAATCAGGAAATACACCTGGACAATTTCTAGAAGA
CTACAATTCAGTCTAGCCACAAAGGGGATTTTTTTTTTTTGGTAACAGGCTAGAGCCCGGTTCT
GTAAGTCTTTAGCTGAAATGGTCCAGTACAAAAGCACTGGAAATGAGTGGGCTAGGAGGACAAG
GACCGTCTCCTGCGTGAGGAGTTGGTTGGAGGTCCCCAAGGCCAGGTACCCCCTGCACTCTTAT
TGGATTCCTCTCTGTCTTCTTGGAGTTTTGAAAAACTCCTTCGAACACCAGGCTTTTTTCTTTA
GAAAACAAGTCTCCAATCGTTCTCTGTTCCGTAGAAAGAGAAAGAAAACCTGGAGCAGCTGCTG
AAAAATCTAATGAGGAACTAAGAGGCAAACCCACCA
Exemplary Human CLRN1 cDNA sequence (Isoform D) including untranslated
regions
(SEQ ID NO: 8)
AGGAGATACTTGAAGGCAGTTTGAAAGACTTGTTTTACAGATTCTTAGTCCAAAGATTTCCAAT
TAGGGAGAAGAAGCAGCAGAAAAGGAGAAAAGCCAAGTATGAGTGATGATGAGGCCTTCATCTA
CTGACATTTAACCTGGCGAGAACCGTCGATGGTGAAGTTGCCTTTTCAGCTGGGAGCTGTCCGT
TCAGCTTCCGTAATAAATGCAGTCAAAGAGGCAGTCCCTTCCCATTGCTCACAAAGGTCTTGTT
TTTGAACCTCGCCCTCACAGAAGCCGTTTCTCATCATGCCAAGCCAACAGAAGAAAATCATTTT
TTGCATGGCCGGAGTGTTCAGTTTTGCATGTGCCCTCGGAGTTGTGACAGCCTTGGGGACACCG
TTGTGGATCAAAGCCACTGTCCTCTGCAAAACGGGAGCTCTGCTCGTCAATGCCTCAGGGCAGG
AGCTGGACAAGTTTATGGGTGAAATGCAGTACGGGCTTTTCCACGGAGAGGGTGTGAGGCAGTG
TGGGTTGGGAGCAAGGCCCTTTCGGTTCTCATTTTTTCCAGATTTGCTCAAAGCAATCCCAGTG
AGCATCCACGTCAATGTCATTCTCTTCTCTGCCATCCTTATTGTGTTAACCATGGTGGGGACAG
CCTTCTTCATGTACAATGCTTTTGGAAAACCTTTTGAAACTCTGCATGGTCCCCTAGGGCTGTA
CCTTTTGAGCTTCATTTCAGTTGCCCTTTGGCTGCCAGCTACCAGGCACCAGGCTCAAGGCTCC
TGTGGCTGTCTTGTCATGATATTGTTTGCCTCTGAAGTGAAAATCCATCACCTCTCAGAAAAAA
TTGCAAATTATAAAGAAGGGACTTATGTCTACAAAACGCAAAGTGAAAAATATACCACCTCATT
CTGGGTCATTTTCTTTTGCTTTTTTGTTCATTTTCTGAATGGGCTCCTAATACGACTTGCTGGA
TTTCAGTTCCCTTTTGCAAAATCTAAAGACGCAGAAACAACTAATGTAGCTGCAGATCTAATGT
ACTGAAAGGCAAACCTTTCTATAATTTTACAAGGGAGTAGACTTGCTTTGGTCACTTTTAGATG
TGGTTAATTTTGCATATCCTTTTAGTCTGCATATATTAAAGCATCAGGACCCTTCGTGACAATG
TTTACAAATTACGTACTAAGGATACAGGCTGGAAAGTAAGGGAAGCAGAAGGAAGGCTTTGAAA
AGTTGTTTTATCTGGTGGGAAATTGCTTGACCCAGGTAGTCAAAGGCAGTTGACTAGAATCGAC
AAATTGTTACTCCATATATATATATGTGTGTGTGTGTGTGTGTGTGTGTGTGTAAGATGTCTTC
CTATCAAAAAGATATCAAAGGCACATGGAATATATTTTAATAAAAACAAATAATATCTCTAATA
TATCCACACATTTGTTGCCAGATTTCAGAAAACTGAGCTGCAATCGCTTTCCTAAAACAGTAGT
GTATTAAATGAACATCTATAAAATGTATCAACACACATTTTAAAAAATTTGTTTAAAGTATACT
CTTAGGCCAGGCGTGGTGACTCACACCTGTAATTCCAGCACTTCAGGAGGCCAAGGTGGGAAGA
TCATTTGAGTTCAGGAGTTCGAGTTACAGCCTGGGCAATAAAGTGAGACCCTGTCACTAACAAA
ATTAAAAAATAAAATAAATATAAAATATAGGCTTTAAAAAAGCATAGTCTTATTAACCATGTCT
GTTGGTCAAAATCTGCAAACTCTAAAAGAAGAAAAGAAGAAAAAACCAAGCTTAGGGTATTTTT
CCTCCCGTGCCTGAGTCCCAATTACATTCACGACAGTACTTTCAATGAACATAATTGTTAGGAC
CACTGAGGAATCATGAAAAATGATCTCTGCTTAGTACATTTGATGCAAAATGACTTATTAGGGG
CTGTTTTTCTAGCTATAGTGTCTCGAGTACTAATATGCAATTATGAAAATTATATTAAATCTGG
GATTATGACGGTATCACTGTATCATCTTGGTCTTGTTCTGGCTGTCACCAAGCATGACCCAGGT
CAACTTTTTTTTTCCCCTGAATTACCCATCAAATTGATCTGCAGCTGACTAAAGGCCACAGCTG
AGCCTGGAACTGACCCTTCCTTCATCCTCAACCTGCTGTCCTCCAGAAAGCACCAAGGAAAAAG
CAGAGAATGACAGCAAACAGATCACTAGGCCTCTGACCACAGGTGCTGAGTACTCAGCAGCCCT
CATATAATAGGTTTGAAAGTACTCCTTAAAATAAAACACTGTTTCCCTTTGGAACTATTTACAA
GGATGAAACAACCGTATACCTGAGAAATAACTTGCTCTGGTGTCAATTCGCTATTCGCCAGCAG
ACATCAGAACACACCGAGTTTCCAGATGCT
Exemplary Human CLRN1 cDNA sequence (Isoform E) including untranslated
regions
(SEQ ID NO: 9)
ACAGAAGCCGTTTCTCATCATGCCAAGCCAACAGAAGAAAATCATTTTTTGCATGGCCGGAGTG
TTCAGTTTTGCATGTGCCCTCGGAGTTGTGACAGCCTTGGGGACACCGTTGTGGATCAAAGCCA
CTGTCCTCTGCAAAACGGGAGCTCTGCTCGTCAATGCCTCAGGGCAGGAGCTGGACAAGTTTAT
GGGTGAAATGCAGTACGGGCTTTTCCACGGAGAGGGTGTGAGGCAGTGTGGGTTGGGAGCAAGG
CCCTTTCGGTTCTCATGCTATTTTCTTGACCCCTTCATGGGACTCCCAACAGGGGTACCCCATT
TACTCAGCCTGCCCTGCTCAACCTCTTGCAGGAGGGAGCACACGAGTGAACGAGTGCAGGAACC
AGCTGGCTGCTTTAGTGCTGTGAGGAGTAAACTCCATGCAGGCCCTGCAGCAGCAACCAGTTTT
TCCAGATTTGCTCAAAGCAATCCCAGTGAGCATCCACGTCAATGTCATTCTCTTCTCTGCCATC
CTTATTGTGTTAACCATGGTGGGGACAGCCTTCTTCATGTACAATGCTTTTGGAAAACCTTTTG
AAACTCTGCATGGTCCCCTAGGGCTGTACCTTTTGAGCTTCATTTCAGGCTCCTGTGGCTGTCT
TGTCATGATATTGTTTGCCTCTGAAGTGAAAATCCATCACCTCTCAGAAAAAATTGCAAATTAT
AAAGAAGGGACTTATGTCTACAAAACGCAAAGTGAAAAATATACCACCTCATTCTGGGTCATTT
TCTTTTGCTTTTTTGTTCATTTTCTGAATGGGCTCCTAATACGACTTGCTGGATTTCAGTTCCC
TTTTGCAAAATCTAAAGACGCAGAAACAACTAATGTAGCTGCAGATCTAATGTACTGAAAGGCA
AACCTTTCTATAATTTTACAAGGGAGTAGACTTGCTTTGGTCACTTTTAGATGTGGTTAATTTT
GCATATCCTTTTAGTCTGCATATATTAAAGCATCAGGACCCTTCGTGACAATGTTTACAAATTA
CGTACTAAGGATACAGGCTGGAAAGTAAGGGAAGCAGAAGGAAGGCTTTGAAAAGTTGTTTTAT
CTGGTGGGAAATTGCTTGACCCAGGTAGTCAAAGGCAGTTGACTAGAATCGACAAATTGTTACT
CCATATATATATATGTGTGTGTGTGTGTGTGTGTGTGTGTGTAAGATGTCTTCCTATCAAAAAG
ATATCAAAGGCACATGGAATATATTTTAATAAAAACAAATAATATCTCTAATATATCCACACAT
TTGTTGCCAGATTTCAGAAAACTGAGCTGCAATCGCTTTCCTAAAACAGTAGTGTATTAAATGA
ACATCTATAAAATGTATCAACACACATTTTAAAAAATTTGTTTAAAGTATACTCTTAGGCCAGG
CGTGGTGACTCACACCTGTAATTCCAGCACTTCAGGAGGCCAAGGTGGGAAGATCATTTGAGTT
CAGGAGTTCGAGTTACAGCCTGGGCAATAAAGTGAGACCCTGTCACTAACAAAATTAAAAAATA
AAATAAATATAAAATATAGGCTTTAAAAAAGCATAGTCTTATTAACCATGTCTGTTGGTCAAAA
TCTGCAAACTCTAAAAGAAGAAAAGAAGAAAAAACCAAGCTTAGGGTATTTTTCCTCCCGTGCC
TGAGTCCCAATTACATTCACGACAGTACTTTCAATGAACATAATTGTTAGGACCACTGAGGAAT
CATGAAAAATGATCTCTGCTTAGTACATTTGATGCAAAATGACTTATTAGGGGCTGTTTTTCTA
GCTATAGTGTCTCGAGTACTAATATGCAATTATGAAAATTATATTAAATCTGGGATTATGACGG
TATCACTGTATCATCTTGGTCTTGTTCTGGCTGTCACCAAGCATGACCCAGGTCAACTTTTTTT
TTCCCCTGAATTACCCATCAAATTGATCTGCAGCTGACTAAAGGCCACAGCTGAGCCTGGAACT
GACCCTTCCTTCATCCTCAACCTGCTGTCCTCCAGAAAGCACCAAGGAAAAAGCAGAGAATGAC
AGCAAACAGATCACTAGGCCTCTGACCACAGGTGCTGAGTACTCAGCAGCCCTCATATAATAGG
TTTGAAAGTACTCCTTAAAATAAAACACTGTTTCCCTTTGGAACTATTTACAAGGATGAAACAA
CCGTATACCTGAGAAATAACTTGCTCTGGTGTCAATTCGCTATTCGCCAGCAGACATCAGAACA
CACCGAGTTTCCAGATGCT

The present disclosure recognizes that certain changes to a polynucleotide sequence will not impact its expression or a protein encoded by said polynucleotide. In some embodiments, a polynucleotide comprises a CLRN1 gene having one or more silent mutations. In some embodiments, the disclosure provides a polynucleotide that comprises a CLRN1 gene having one or more silent mutations, e.g., a CLRN1 gene having a sequence different from SEQ ID NO: 1, 2, 3, 4, 5, 6, or 7 but encoding the same amino acid sequence as a functional CLRN1 gene. In some embodiments, the disclosure provides a polynucleotide that comprises a CLRN1 gene having a sequence different from SEQ ID NO: 1, 2, 3, 4, 5, 6, or 7 that encodes an amino acid sequence including one or more mutations (e.g., a different amino acid sequence when compared to that produced from a functional CLRN1 gene), where the one or more mutations are conservative amino acid substitutions. In some embodiments, the disclosure provides a polynucleotide that comprises a CLRN1 gene having a sequence different from SEQ ID NO: 1, 2, 3, 4, 5, 6, or 7 that encodes an amino acid sequence including one or more mutations (e.g., a different amino acid sequence when compared to that produced from a functional CLRN1 gene), where the one or more mutations are not within a characteristic portion of a CLRN1 gene or an encoded clarin 1 protein. In some embodiments, a polynucleotide in accordance with the present disclosure comprises a CLRN1 gene that is at least 85%, at least 90%, at least 95%, at least 98%, or at least 99% identical to a sequence of SEQ ID NO: 1, 2, 3, 4, 5, 6, or 7. In some embodiments, a polynucleotide in accordance with the present disclosure comprises a CLRN1 gene that is identical to the sequence of SEQ ID NO: 1, 2, 3, 4, 5, 6, or 7. As can be appreciated in the art, SEQ ID NO: 1, 2, 3, 4, 5, 6, or 7 can be optimized (e.g., codon optimized) to achieve increased or optimal expression in an animal, e.g., a mammal, e.g., a human.

Polypeptides Encoded by CLRN1 Gene

Among other things, the present disclosure provides polypeptides encoded by a CLRN1 gene or characteristic portion thereof. In some embodiments, a CLRN1 gene is a mammalian CLRN1 gene. In some embodiments, a CLRN1 gene is a murine CLRN1 gene. In some embodiments, a CLRN1 gene is a primate CLRN1 gene. In some embodiments, a CLRN1 gene is a human CLRN1 gene.

In some embodiments, a polypeptide comprises a clarin 1 protein or characteristic portion thereof. In some embodiments, a clarin 1 protein or characteristic portion thereof is mammalian clarin 1 protein or characteristic portion thereof, e.g., primate clarin 1 protein or characteristic portion thereof. In some embodiments, a clarin 1 protein or characteristic portion thereof is a human clarin 1 protein or characteristic portion thereof.

In some embodiments, a polypeptide provided herein comprises post-translational modifications. In some embodiments, a clarin 1 protein or characteristic portion thereof provided herein comprises post-translational modifications. In some embodiments, post-translational modifications can comprise but is not limited to glycosylation (e.g., N-linked glycosylation, 0-linked glycosylation), phosphorylation, acetylation, amidation, hydroxylation, methylation, ubiquitylation, sulfation, and/or a combination thereof.

An exemplary human clarin 1 protein sequence is or includes the sequence of SEQ ID NO: 10, 11, 12, or 13. An exemplary human clarin 1 protein sequence with a c-terminal flag tag is or includes the sequence of SEQ ID NO: 14.

Exemplary Human Clarin 1 Protein Sequence (Isoform A)
(SEQ ID NO: 10)
MPSQQKKIIFCMAGVFSFACALGVVTALGTPLWIKATVLCKTGALLVNASGQELDKFMGEMQYG
LFHGEGVRQCGLGARPFRFSFFPDLLKAIPVSIHVNVILFSAILIVLTMVGTAFFMYNAFGKPF
ETLHGPLGLYLLSFISGSCGCLVMILFASEVKIHHLSEKIANYKEGTYVYKTQSEKYTTSFWVI
FFCFFVHFLNGLLIRLAGFQFPFAKSKDAETTNVAADLMY
Exemplary Human Clarin 1 Protein Sequence (Isoform C)
(SEQ ID NO: 11)
MQALQQQPVFPDLLKAIPVSIHVNVILFSAILIVLTMVGTAFFMYNAFGKPFETLHGPLGLYLL
SFISGSCGCLVMILFASEVKIHHLSEKIANYKEGTYVYKTQSEKYTTSFWLTKGHS
Exemplary Human Clarin 1 Protein Sequence (Isoform D)
(SEQ ID NO: 12)
MPSQQKKIIFCMAGVFSFACALGVVTALGTPLWIKATVLCKTGALLVNASGQELDKFMGEMQYG
LFHGEGVRQCGLGARPFRFSFFPDLLKAIPVSIHVNVILFSAILIVLTMVGTAFFMYNAFGKPF
ETLHGPLGLYLLSFISVALWLPATRHQAQGSCGCLVMILFASEVKIHHLSEKIANYKEGTYVYK
TQSEKYTTSFWVIFFCFFVHFLNGLLIRLAGFQFPFAKSKDAETTNVAADLMY
Exemplary Human Clarin 1 Protein Sequence (Isoform E)
(SEQ ID NO: 13)
MPSQQKKIIFCMAGVFSFACALGVVTALGTPLWIKATVLCKTGALLVNASGQELDKFMGEMQYG
LFHGEGVRQCGLGARPFRFSCYFLDPFMGLPTGVPHLLSLPCSTSCRREHTSERVQEPAGCFSA
VRSKLHAGPAAATSFSRFAQSNPSEHPRQCHSLLCHPYCVNHGGDSLLHVQCFWKTF
Exemplary Human Clarin 1 Protein Sequence (Isoform A) with C-terminal
Flag Tag
(SEQ ID NO: 14)
MPSQQKKIIFCMAGVFSFACALGVVTALGTPLWIKATVLCKTGALLVNASGQELDKFMGEMQYG
LFHGEGVRQCGLGARPFRFSFFPDLLKAIPVSIHVNVILFSAILIVLTMVGTAFFMYNAFGKPF
ETLHGPLGLYLLSFISGSCGCLVMILFASEVKIHHLSEKIANYKEGTYVYKTQSEKYTTSFWVI
FFCFFVHFLNGLLIRLAGFQFPFAKSKDAETTNVAADLMYGSRADYKDHDGDYKDHDIDYKDDD
DK

The present disclosure recognizes that certain mutations in an amino acid sequence of a polypeptide described herein (e.g., including clarin 1 or a characteristic portion thereof) will not impact the expression, folding, or activity of the polypeptide. In some embodiments, a polypeptide (e.g., including clarin 1 or a characteristic portion thereof) includes one or more mutations, where the one or more mutations are conservative amino acid substitutions. In some embodiments, a polypeptide in accordance with the present disclosure comprises a clarin 1 or a characteristic portion thereof that is at least 85%, at least 90%, at least 95%, at least 98%, or at least 99% identical to a sequence of SEQ ID NO: 8, 9, 10, or 11. In some embodiments, a polypeptide in accordance with the present disclosure comprises a clarin 1 or a characteristic portion thereof that is identical to the sequence of SEQ ID NO: 8, 9, 10, or 11. In some embodiments, a polypeptide in accordance with the present disclosure comprises a clarin 1 or a characteristic portion thereof that is at least 85%, at least 90%, at least 95%, at least 98%, or at least 99% identical to a sequence of SEQ ID NO: 14. In some embodiments, a polypeptide in accordance with the present disclosure comprises a clarin 1 protein or a characteristic portion thereof that is identical to the sequence of SEQ ID NO: 14.

### Constructs

Among other things, the present disclosure provides that some polynucleotides as described herein are polynucleotide constructs. Polynucleotide constructs according to the present disclosure include all those known in the art, including cosmids, plasmids (e.g., naked or contained in liposomes) and viral constructs (e.g., lentiviral, retroviral, adenoviral, and adeno-associated viral constructs) that incorporate a polynucleotide comprising a CLRN1 gene or characteristic portion thereof. Those of skill in the art will be capable of selecting suitable constructs, as well as cells, for making any of the polynucleotides described herein. In some embodiments, a construct is a plasmid (i.e., a circular DNA molecule that can autonomously replicate inside a cell). In some embodiments, a construct can be a cosmid (e.g., pWE or sCos series).

In some embodiments, a construct is a viral construct. In some embodiments, a viral construct is a lentivirus, retrovirus, adenovirus, or adeno-associated virus construct. In some embodiments, a construct is an adeno-associated virus (AAV) construct (see, e.g., Asokan et al., Mol. Ther. 20: 699-7080, 2012, which is incorporated in its entirety herein by reference). In some embodiments, a viral construct is an adenovirus construct. In some embodiments, a viral construct may also be based on or derived from an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral constructs for coding sequence delivery. Pseudotyped viruses may be formed by combining alphaviral envelope glycoproteins and retroviral capsids. Examples of alphaviral constructs can be found in U.S. Publication Nos. 20150050243, 20090305344, and 20060177819; constructs and methods of their making are incorporated herein by reference to each of the publications in its entirety.

Constructs provided herein can be of different sizes. In some embodiments, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some embodiments, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some embodiments, a construct is a viral construct and can have a total number of nucleotides of up to 10 kb. In some embodiments, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some embodiments, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb

In some embodiments, a construct is an adenovirus construct and can have a total number of nucleotides of up to 8 kb. In some embodiments, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can further include a control sequence, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or additional untranslated regions which may house pre- or post-transcriptional regulatory and/or control elements. In some embodiments, a promoter can be a native promoter, a constitutive promoter, an inducible promoter, and/or a tissue-specific promoter. Non-limiting examples of control sequences are described herein.

### Exemplary Construct Components

Inverted Terminal Repeat Sequences (ITRs)

AAV derived sequences of a construct typically comprises the cis-acting 5′ and 3′ ITRs (see, e.g., B. J. Carter, in “Handbook of Parvoviruses”, ed., P. Tijsser, CRC Press, pp. 155 168 (1990), which is incorporated in its entirety herein by reference). Generally, ITRs are able to form a hairpin. The ability to form a hairpin can contribute to an ITRs ability to self-prime, allowing primase-independent synthesis of a second DNA strand. ITRs can also aid in efficient encapsidation of an AAV construct in an AAV particle.

An rAAV particle (e.g., an AAV2/Anc80 particle) of the present disclosure can comprise a rAAV construct comprising a coding sequence (e.g., CLRN1 gene) and associated elements flanked by a 5′ and a 3′ AAV ITR sequences. In some embodiments, an ITR is or comprises about 145 nucleic acids. In some embodiments, all or substantially all of a sequence encoding an ITR is used. An AAV ITR sequence may be obtained from any known AAV, including presently identified mammalian AAV types. In some embodiments an ITR is an AAV2 ITR.

An example of a construct molecule employed in the present disclosure is a “cis-acting” construct containing a transgene, in which the selected transgene sequence and associated regulatory elements are flanked by 5′ or “left” and 3′ or “right” AAV ITR sequences. 5′ and left designations refer to a position of an ITR sequence relative to an entire construct, read left to right, in a sense direction. For example, in some embodiments, a 5′ or left ITR is an ITR that is closest to a promoter (as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some embodiments, a 3′ or right ITR is an ITR that is closest to a polyadenylation sequence (as opposed to a promoter sequence) for a given construct, when a construct is depicted in a sense orientation, linearly. ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to antisense direction. Further, it is well within the ability of one of skill in the art to transform a given sense ITR sequence (e.g., a 5′/left AAV ITR) into an antisense sequence (e.g., 3′/right ITR sequence). One of ordinary skill in the art would understand how to modify a given ITR sequence for use as either a 5′/left or 3′/right ITR, or an antisense version thereof.

For example, an ITR (e.g., a 5′ ITR) can have a sequence according to SEQ ID NO: 15, 17, 20, or 21. In some embodiments, an ITR (e.g., a 3′ ITR) can have a sequence according to SEQ ID NO: 16, 18, 20, or 22. In some embodiments, an ITR includes one or more modifications, e.g., truncations, deletions, substitutions or insertions, as is known in the art. In some embodiments, an ITR comprises fewer than 145 nucleotides, e.g., 127, 130, 134 or 141 nucleotides. For example, in some embodiments, an ITR comprises 110, 111, 112, 113, 114, 115, 116, 117, 118, 119, 120, 121, 122, 123,124, 125, 126, 127, 128, 129, 130, 131, 132, 133, 134, 135, 136, 137, 138, 139, 140, 141, 142, 143 144, or 145 nucleotides. In some embodiments, an ITR (e.g., a 5′ ITR) can have a sequence according to SEQ ID NO: 15. In some embodiments, an ITR (e.g., a 5′ ITR) can have a sequence according to SEQ ID NO: 17. In some embodiments, an ITR (e.g., a 5′ ITR) can have a sequence according to SEQ ID NO: 20. In some embodiments, an ITR (e.g., a 5′ ITR) can have a sequence according to SEQ ID NO: 21. In some embodiments, an ITR (e.g., a 3′ ITR) can have a sequence according to SEQ ID NO: 16. In some embodiments, an ITR (e.g., a 3′ ITR) can have a sequence according to SEQ ID NO: 18. In some embodiments, an ITR (e.g., a 3′ ITR) can have a sequence according to SEQ ID NO: 20. In some embodiments, an ITR (e.g., a 3′ ITR) can have a sequence according to SEQ ID NO: 22.

A non-limiting example of a 5′ AAV ITR sequence is SEQ ID NO: 15, 17, 20, or 21. A non-limiting example of a 3′ AAV ITR sequence is SEQ ID NO: 16, 18, 20, or 22. In some embodiments, rAAV constructs of the present disclosure comprise a 5′ AAV ITR and/or a 3′ AAV ITR. In some embodiments, an ITR (e.g., a 5′ ITR) can have a sequence according to SEQ ID NO: 17. In some embodiments, a 5′ AAV ITR sequence can have a sequence according to SEQ ID NO: 20. In some embodiments, a 5′ AAV ITR sequence can have a sequence according to SEQ ID NO: 21. In some embodiments, a 3′ AAV ITR sequence is SEQ ID NO: 16. In some embodiments, a 3′ AAV ITR sequence is SEQ ID NO: 18. In some embodiments, a 3′ AAV ITR sequence is SEQ ID NO: 20. In some embodiments, a 3′ AAV ITR sequence is SEQ ID NO: 22. In some embodiments, the 5′ and a 3′ AAV ITRs (e.g., SEQ ID NOs: 15, 17, or 21, or 16, 18, 20, or 22) flank a portion of a coding sequence, e.g., all or a portion of a CLRN1 gene (e.g., SEQ ID NO: 1, 2, 3, or 4). The ability to modify these ITR sequences is within the skill of the art. (see, e.g., texts such as Sambrook et al. “Molecular Cloning. A Laboratory Manual”, 2d ed., Cold Spring Harbor Laboratory, New York (1989); and K. Fisher et al., J Virol., 70:520 532 (1996), each of which is incorporated in its entirety herein by reference). In some embodiments, a 5′ ITR sequence is at least 85%, 90%, 95%, 98% or 99% identical to a 5′ ITR sequence represented by SEQ ID NO: 15, 17, 20, or 21. In some embodiments, a 3′ ITR sequence is at least 85%, 90%, 95%, 98% or 99% identical to a 3′ ITR sequence represented by SEQ ID NO: 16, 18, 20, or 22.

Exemplary 5′ AAV ITR
(SEQ ID NO: 15)
TTGGCCACTCCCTCTCTGCGCGCTCGCTCGCTCACTGAGGCCGCCCGGG
CAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCT
Exemplary 3′ AAV ITR
(SEQ ID NO: 16)
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAGAGAGGGAGTGGCCAA
Exemplary 5′ AAV ITR
(SEQ ID NO: 17)
CTGCGCGCTCGCTCGCTCACTGAGGCCGCCCGGGCGTCGGGCGACCTTT
GGTCGCCCGGCCTCAGTGAGCGAGCGAGCGCGCAGAGAGGGAGTGGCCA
ACTCCATCACTAGGGGTTCCT
Exemplary 3′ AAV ITR
(SEQ ID NO: 18)
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAG
Exemplary 5′ AAV ITR
(SEQ ID NO: 20)
CTGCGCGCTCGCTCGCTCACTGAGGCCGCCCGGGCAAAGCCCGGGCGTC
GGGCGACCTTTGGTCGCCCGGCCTCAGTGAGCGAGCGAGCGCGCAGAGA
GGGAGTGGCCAACTCCATCACTAGGGGTTCCT
Exemplary 5′ AAV ITR
(SEQ ID NO: 21)
TTGGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGCGA
GCGAGCGCGCAGAGAGGGAGTGGCCAACTCCATCACTAGGGGTTCCT
Exemplary 3′ AAV ITR
(SEQ ID NO: 22)
AGGAACCCCTAGTGATGGAGTTGGCCACTCCCTCTCTGCGCGCTCGCTC
GCTCACTGAGGCCGCCCGGGCAAAGCCCGGGCGTCGGGCGACCTTTGGT
CGCCCGGCCTCAGTGAGCGAGCGAGCGCGCAGAGAGGGAGTGGCCAA

Promoters

In some embodiments, a construct (e.g., an rAAV construct) comprises a promoter. The term “promoter” refers to a DNA sequence recognized by enzymes/proteins that can promote and/or initiate transcription of an operably linked gene (e.g., a CLRN1 gene). For example, a promoter typically refers to, e.g., a nucleotide sequence to which an RNA polymerase and/or any associated factor binds and from which it can initiate transcription. Thus, in some embodiments, a construct (e.g., an rAAV construct) comprises a promoter operably linked to one of the non-limiting example promoters described herein.

In some embodiments, a promoter is an inducible promoter, a constitutive promoter, a mammalian cell promoter, a viral promoter, a chimeric promoter, an engineered promoter, a tissue-specific promoter, or any other type of promoter known in the art. In some embodiments, a promoter is a RNA polymerase II promoter, such as a mammalian RNA polymerase II promoter. In some embodiments, a promoter is a RNA polymerase III promoter, including, but not limited to, a HI promoter, a human U6 promoter, a mouse U6 promoter, or a swine U6 promoter. A promoter will generally be one that is able to promote transcription in an inner ear cell or eye cell. In some embodiments, a promoter is a cochlea-specific promoter or a cochlea-oriented promoter. In some embodiments, a promoter is a hair cell specific promoter, or a supporting cell specific promoter.

A variety of promoters are known in the art, which can be used herein. Non-limiting examples of promoters that can be used herein include: human EF1α, human cytomegalovirus (CMV) (U.S. Pat. No. 5,168,062, which is incorporated in its entirety herein by reference), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g., human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2_{K} b, HSP70, proliferin, tumor necrosis factor, thyroid stimulating hormone a gene, immunoglobulin light chain, T-cell receptor, HLA DQa and DQ, interleukin-2 receptor, MHC class II, MHC class II HLA-DRa, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GALV) promoters. Additional examples of promoters are known in the art. See, e.g., Lodish, Molecular Cell Biology, Freeman and Company, New York 2007, each of which is incorporated in its entirety herein by reference. In some embodiments, a promoter is the CMV immediate early promoter. In some embodiments, the promoter is a CAG promoter or a CAG/CBA promoter. In some embodiments, the promoter comprises or consists of SEQ ID NO: 14. In some embodiments, a promoter comprises or consists of SEQ ID NO: 15. In certain embodiments, a promoter comprises a CMV/CBA enhancer/promoter construct exemplified in SEQ ID NO: 16. In certain embodiments, a promoter comprises a CMV/CBA enhancer/promoter construct exemplified in SEQ ID NO: 17. In certain embodiments, a promoter comprises a CAG promoter or CMV/CBA/SV-40 enhancer/promoter construct exemplified in SEQ ID NO: 43. In certain embodiments, a promoter comprises a CAG promoter or CMV/CBA/SV-40 enhancer/promoter construct exemplified in SEQ ID NO: 44. In some embodiments, a promoter sequence is at least 85%, 90%, 95%, 98% or 99% identical to the promoter sequences represented by SEQ ID NO: 14 or 15. In some embodiments, an enhancer-promoter sequence is at least 85%, 90%, 95%, 98% or 99% identical to enhancer-promoter sequence represented by SEQ ID NO: 16, 17, 43, or 44.

The term “constitutive” promoter refers to a nucleotide sequence that, when operably linked with a nucleic acid encoding a protein (e.g., a clarin 1 protein), causes RNA to be transcribed from the nucleic acid in a cell under most or all physiological conditions.

Examples of constitutive promoters include, without limitation, the retroviral Rous sarcoma virus (RSV) LTR promoter, the cytomegalovirus (CMV) promoter (see, e.g., Boshart et al, Cell 41:521-530, 1985, which is incorporated in its entirety herein by reference), the SV40 promoter, the dihydrofolate reductase promoter, the beta-actin promoter, the phosphoglycerol kinase (PGK) promoter, and the EF1-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art.

Examples of inducible promoters regulated by exogenously supplied compounds include the zinc-inducible sheep metallothionein (MT) promoter, the dexamethasone (Dex)-inducible mouse mammary tumor virus (MMTV) promoter, the T7 polymerase promoter system (WO 98/10088, which is incorporated in its entirety herein by reference); the ecdysone insect promoter (see, e.g., No et al, Proc. Natl. Acad Sci. US.A. 93:3346-3351, 1996, which is incorporated in its entirety herein by reference), the tetracycline-repressible system (see, e.g., Gossen et al, Proc. Natl. Acad Sci. US.A. 89:5547-5551, 1992, which is incorporated in its entirety herein by reference), the tetracycline-inducible system (see, e.g., Gossen et al, Science 268:1766-1769, 1995; and Harvey et al, Curr. Opin. Chem. Biol. 2:512-518, 1998, each of which is incorporated in their entirety herein by reference), the RU486-inducible system (see, e.g., Wang et al, Nat. Biotech. 15:239-243, 1997; and Wang et al, Gene Ther. 4:432-441, 1997, each of which is incorporated in their entirety herein by reference), and the rapamycin-inducible system (see, e.g., Magari et al. J Clin. Invest. 100:2865-2872, 1997, which is incorporated in its entirety herein by reference).

The term “tissue-specific” promoter refers to a promoter that is active only in certain specific cell types and/or tissues (e.g., transcription of a specific gene occurs only within cells expressing transcription regulatory and/or control proteins that bind to the tissue-specific promoter).

In some embodiments, regulatory and/or control sequences impart tissue-specific gene expression capabilities. In some cases, tissue-specific regulatory and/or control sequences bind tissue-specific transcription factors that induce transcription in a tissue-specific manner.

In some embodiments, a tissue-specific promoter is a cochlea-specific promoter. In some embodiments, a tissue-specific promoter is a cochlear hair cell-specific promoter. Non-limiting examples of cochlear hair cell-specific promoters include but are not limited to: a ATOH1 promoter, a POU4F3 promoter, a LHX3 promoter, a MYO7A promoter, a MYO6 promoter, a α9ACHR promoter, and a α10ACHR promoter. In some embodiments, a promoter is a cochlear hair cell-specific promoter such as a PRESTIN promoter or an ONCOMOD promoter. (See, e.g., Zheng et al., Nature 405:149-155, 2000; Tian et al. Dev. Dyn. 23 1: 199-203, 2004; and Ryan et al., Adv. Otorhinolaryngol. 66: 99-115, 2009, each of which is incorporated in their entirety herein by reference.)

In some embodiments, a tissue-specific promoter is an ear cell specific promoter. In some embodiments, a tissue-specific promoter is an inner ear cell specific promoter. Non-limiting examples of inner ear non-sensory cell-specific promoters include but are not limited to: GJB2, GJB6, SLC26A4, TECTA, DFNA5, COCH, NDP, SYN1, GFAP, PLP, TAK1, or SOX21. In some embodiments, a cochlear non-sensory cell specific promoter may be an inner ear supporting cell specific promoter. Non-limiting examples of inner ear supporting cell specific promoters include but are not limited to: SOX2, FGFR3, PROX1, GLAST1, LGR5, HES1, HES5, NOTCH1, JAG1, CDKN1A, CDKN1B, SOX10, P75, CD44, HEY2, LFNG, or S100b.

In some embodiments, a tissue-specific promoter is an eye cell specific promoter. Non-limiting examples of eye cell specific promoters include but are not limited to: RPE65, RLBP1, VMD2, IRBP, GNAT2, PR1.7, PR2.1, HB569, CAR, GRK1, RK, B-PDE, GRM6, Nefh, Tyh1, SYN, GFAP, or other opsin or rhodopsin promoters. Non-limiting examples of eye cell specific promoters and exemplary contents describing such promoters are as follows:

Promoter in Retina Gene Therapy
RPE

| | | |
|---|---|---|
| Retinal Pigment Epithelium 65 | RPE | Bainbridge et al., NEJM 2008, Bainbridge et |
| | | al., NEJM 2015, each of which are hereby |
| | | incorporated by reference herein in its |
| | | entirety |
| Retinaldehyde-binding protein 1 | RLBP1 | Choi et al., Mol Ther Methods Clin Dev |
| | | 2015, which hereby is incorporated by |
| | | reference herein in its entirety |
| Vitelliform Macular Dystrophy | VMD2 | Esumi et al, JBC 2004, which is hereby |
| | | incorporated by reference herein in its |
| | | entirety |

Photoreceptors

| | | |
|---|---|---|
| Interphotoreceptor retinoid- | IRBP | Dyka et al., Adv Exp Med Biol 2014, which |
| binding protein | | is hereby incorporated by reference herein in |
| | | its entirety |
| Transducin alpha-subunit | GNAT2 | Dyka et al., Adv Exp Med Biol 2014, which |
| | | is hereby incorporated by reference herein in |
| | | its entirety |
| Red/Green opsin | PR1.7, | Alexander et al. Nat Med 2007, Komaromy |
| | PR2.1 | et al., Hum Mol Genet 2010, Ye et al., Hum |
| | | Gene Ther 2016, each of which is hereby |
| | | incorporated by reference herein in its |
| | | entirety |
| S opsin | HB569 | Glushakova et al., Invest Opthalmol Vis Sci |
| | | 2006, which is hereby incorporated by |
| | | reference herein in its entirety |
| Cone arrestin | CAR | Dyka et al., Adv Exp Med Biol 2014, which |
| | | is hereby incorporated by reference herein in |
| | | its entirety |
| G-coupled receptor kinase | GRK1 | Beltran et al., Gene Ther 2010which is |
| Rhodopsin kinase promoter | RK | hereby incorporated by reference herein in |
| | | its entirety |
| | | Khani et al., Invest Opthalmol Vis Sci 2007, |
| | | which is hereby incorporated by reference |
| | | herein in its entirety |
| GMP phosphodiesterase β- | B-PDE | Ogueta et al., Invest Opthalmol Vis Sci |
| subunit | | 2000, which is hereby incorporated by |
| | | reference herein in its entirety |

Bipolar Cells

| | | |
|---|---|---|
| Glutamate Receptor 6 | GRM6 | Cronin et al., EMBO Mol Med 2014, van |
| | | Wyk et al., PLoS Biol 2015, Hulliger et al., |
| | | Mol Ther: Methods Clin Dev 2020, each of |
| | | which is hereby incorporated by reference |
| | | herein in its entirety |

Ganglion Cells

| | | |
|---|---|---|
| Neurofilament Heavy | Nefh | Hanlon et al., Front Neurosci 2017, which is |
| | | hereby incorporated by reference herein in |
| | | its entirety |
| Thy1 surface antigen | Tyh1 | Alic et al., Neurosci Lett 2016, which is |
| | | hereby incorporated by reference herein in |
| | | its entirety |
| Synapsin | SYN | Gaub et al., PNAS 2014, which is hereby |
| | | incorporated by reference herein in its |
| | | entirety |

Muller Cells

| | | |
|---|---|---|
| Glial Fibrillary Acidic Protein | GFAP | Kuzmanivic et al., Invest Opthalmol Vis Sci |
| | | 2003, which is hereby incorporated by |
| | | reference herein in its entirety |

In some embodiments, provided AAV constructs comprise a promoter sequence selected from a CAG, a CBA, a CMV, or a CB7 promoter. In some embodiments of any of the therapeutic compositions described herein, the first or sole AAV construct further includes at least one promoter sequence selected from Cochlea and/or inner ear specific promoters.

In certain embodiments, a promoter is an CBA promoter as set forth in SEQ ID NO: 23. In some embodiments, an enhancer-promoter sequence is at least 85%, 90%, 95%, 98% or 99% identical to enhancer-promoter sequence represented by SEQ ID NO: 23.

Exemplary CBA promoter
(SEQ ID NO: 23)
GTCGAGGTGAGCCCCACGTTCTGCTTCACTCTCCCCATCTCCCCCCCCT
CCCCACCCCCAATTTTGTATTTATTTATTTTTTAATTATTTTGTGCAGC
GATGGGGGCGGGGGGGGGGGGGGGGCGCGCGCCAGGCGGGGCGGGGCGG
GGCGAGGGGGGGGGCGGGGCGAGGCGGAGAGGTGCGGCGGCAGCCAATC
AGAGCGGCGCGCTCCGAAAGTTTCCTTTTATGGCGAGGCGGCGGCGGCG
GCGGCCCTATAAAAAGCGAAGCGCGCGGCGGGCG
Exemplary CBA promoter
(SEQ ID NO: 24)
GTCGAGGTGAGCCCCACGTTCTGCTTCACTCTCCCCATCTCCCCCCCCT
CCCCACCCCCAATTTTGTATTTATTTATTTTTTAATTATTTTGTGCAGC
GATGGGGGCGGGGGGGGGGGGGGCGCGCGCCAGGCGGGGCGGGGCGGGG
CGAGGGGCGGGGCGGGGCGAGGCGGAGAGGTGCGGCGGCAGCCAATCAG
AGCGGCGCGCTCCGAAAGTTTCCTTTTATGGCGAGGCGGCGGCGGCGGC
GGCCCTATAAAAAGCGAAGCGCGCGGCGGGCG

In certain embodiments, a promoter is an CMV/CBA enhancer/promoter as set forth in SEQ ID NO: 25. In some embodiments, an enhancer-promoter sequence is at least 85%, 90%, 95%, 98% or 99% identical to enhancer-promoter sequence represented by SEQ ID NO: 25.

Exemplary CMV/CBA enhancer/promoter
(SEQ ID NO: 25)
GACATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGGTC
GAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGGGGGGGGCGCGCGCC
AGGCGGGGCGGGGGCGGGGCGAGGGGCGGGGGGGGCGAGGCGGAGAGGTGCGGCGGCAGCCAAT
CAGAGCGGCGCGCTCCGAAAGTTTCCTTTTATGGCGAGGCGGCGGCGGCGGCGGCCCTATAAAA
AGCGAAGCGCGCGGCGGGCG
Exemplary CMV/CBA enhancer/promoter
(SEQ ID NO: 26)
GACATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGGTC
GAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGCGCGCGCCAG
GCGGGGCGGGGCGGGGCGAGGGGCGGGGCGGGGCGAGGCGGAGAGGTGCGGCGGCAGCCAATCA
GAGCGGCGCGCTCCGAAAGTTTCCTTTTATGGCGAGGCGGCGGCGGCGGCGGCCCTATAAAAAG
CGAAGCGCGCGGCGGGCG
Exemplary CAG enhancer/promoter
(SEQ ID NO: 27)
GACATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGGTC
GAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGGGGGGGGCGCGCGCC
AGGCGGGGCGGGGCGGGGCGAGGGGCGGGGCGGGGCGAGGCGGAGAGGTGCGGCGGCAGCCAAT
CAGAGCGGCGCGCTCCGAAAGTTTCCTTTTATGGCGAGGCGGCGGCGGCGGCGGCCCTATAAAA
AGCGAAGCGCGCGGCGGGCGGGAGTCGCTGCGTTGCCTTCGCCCCGTGCCCCGCTCCGCGCCGC
CTCGCGCCGCCCGCCCCGGCTCTGACTGACCGCGTTACTCCCACAGGTGAGCGGGCGGGACGGC
CCTTCTCCTCCGGGCTGTAATTAGCGCTTGGTTTAATGACGGCTCGTTTCTTTTCTGTGGCTGC
GTGAAAGCCTTAAAGGGCTCCGGGAGGGCCCTTTGTGCGGGGGGGAGCGGCTCGGGGGGTGCGT
GCGTGTGTGTGTGCGTGGGGAGCGCCGCGTGCGGCCCGCGCTGCCCGGCGGCTGTGAGCGCTGC
GGGCGCGGCGCGGGGCTTTGTGCGCTCCGCGTGTGCGCGAGGGGAGCGCGGCCGGGGGCGGTGC
CCCGCGGTGCGGGGGGGCTGCGAGGGGAACAAAGGCTGCGTGCGGGGTGTGTGCGTGGGGGGGT
GAGCAGGGGGTGTGGGCGCGGCGGTCGGGCTGTAACCCCCCCCTGCACCCCCCTCCCCGAGTTG
CTGAGCACGGCCCGGCTTCGGGTGCGGGGCTCCGTGCGGGGCGTGGCGCGGGGCTCGCCGTGCC
GGGCGGGGGGTGGCGGCAGGTGGGGGTGCCGGGGGGGGGGGGGCCGCCTCGGGCCGGGGAGGGC
TCGGGGGAGGGGCGCGGCGGCCCCCGGAGCGCCGGCGGCTGTCGAGGCGCGGCGAGCCGCAGCC
ATTGCCTTTTATGGTAATCGTGCGAGAGGGCGCAGGGACTTCCTTTGTCCCAAATCTGTGCGGA
GCCGAAATCTGGGAGGCGCCGCCGCACCCCCTCTAGCGGGCGCGGGGCGAAGCGGTGCGGCGCC
GGCAGGAAGGAAATGGGCGGGGAGGGCCTTCGTGCGTCGCCGCGCCGCCGTCCCCTTCTCCCTC
TCCAGCCTCGGGGCTGTCCGCGGGGGGACGGCTGCCTTCGGGGGGGACGGGGCAGGGCGGGGTT
CGGCTTCTGGCGTGTGACCGGCGGCTCTAGAGCCTCTGCTAACCATGTTCATGCCTTCTTCTTT
TTCCTACAG
Exemplary CAG enhancer/promoter
(SEQ ID NO: 28)
GACATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGGTC
GAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGCGCGCGCCAG
GCGGGGCGGGGCGGGGCGAGGGGCGGGGCGGGGCGAGGCGGAGAGGTGCGGCGGCAGCCAATCA
GAGCGGCGCGCTCCGAAAGTTTCCTTTTATGGCGAGGCGGCGGCGGCGGCGGCCCTATAAAAAG
CGAAGCGCGCGGCGGGCGGGAGTCGCTGCGTTGCCTTCGCCCCGTGCCCCGCTCCGCGCCGCCT
CGCGCCGCCCGCCCCGGCTCTGACTGACCGCGTTACTCCCACAGGTGAGCGGGCGGGACGGCCC
TTCTCCTCCGGGCTGTAATTAGCGCTTGGTTTAATGACGGCTCGTTTCTTTTCTGTGGCTGCGT
GAAAGCCTTAAAGGGCTCCGGGAGGGCCCTTTGTGCGGGGGGGAGCGGCTCGGGGGGTGCGTGC
GTGTGTGTGTGCGTGGGGAGCGCCGCGTGCGGCCCGCGCTGCCCGGCGGCTGTGAGCGCTGCGG
GCGCGGCGCGGGGCTTTGTGCGCTCCGCGTGTGCGCGAGGGGAGCGCGGCCGGGGGCGGTGCCC
CGCGGTGCGGGGGGGCTGCGAGGGGAACAAAGGCTGCGTGCGGGGTGTGTGCGTGGGGGGGTGA
GCAGGGGGTGTGGGCGCGGCGGTCGGGCTGTAACCCCCCCCTGCACCCCCCTCCCCGAGTTGCT
GAGCACGGCCCGGCTTCGGGTGCGGGGCTCCGTGCGGGGCGTGGCGCGGGGCTCGCCGTGCCGG
GCGGGGGGTGGCGGCAGGTGGGGGTGCCGGGCGGGGGGGGGCCGCCTCGGGCCGGGGAGGGCTC
GGGGGAGGGGCGCGGCGGCCCCCGGAGCGCCGGCGGCTGTCGAGGCGCGGCGAGCCGCAGCCAT
TGCCTTTTATGGTAATCGTGCGAGAGGGCGCAGGGACTTCCTTTGTCCCAAATCTGTGCGGAGC
CGAAATCTGGGAGGCGCCGCCGCACCCCCTCTAGCGGGCGCGGGGCGAAGCGGTGCGGCGCCGG
CAGGAAGGAAATGGGGGGGGAGGGCCTTCGTGCGTCGCCGCGCCGCCGTCCCCTTCTCCCTCTC
CAGCCTCGGGGCTGTCCGCGGGGGGACGGCTGCCTTCGGGGGGGACGGGGCAGGGGGGGGTTCG
GCTTCTGGCGTGTGACCGGCGGCTCTAGAGCCTCTGCTAACCATGTTCATGCCTTCTTCTTTTT
CCTACAG

In certain embodiments, a promoter is an endogenous human ATOH1 enhancer-promoter as set forth in SEQ ID NO: 29. In some embodiments, an enhancer-promoter sequence is at least 85%, 90%, 95%, 98% or 99% identical to enhancer-promoter sequence represented by SEQ ID NO: 29.

Exemplary Human ATOH1 enhancer-promoter
(SEQ ID NO: 29)
CTATGGAGTTTGCATAACAAACGTTTGGCAGCTCGCTCTCTTACACTCCATTAACAAGCTGTAA
CATATAGCTGCAGGTTGCTATAATCTCATTAATATTTTGGAAACTTGAATATTGAGTATTTCTG
AGTGCTCATTCCCCATATGCCAGCCACTTCTGCCATGCTGACTGGTTCCTTTCTCTCCATTATT
AGCAATTAGCTTCTTACCTTCCAAAGTCAGATCCAAGGTATCCAAGATACTAGCAAAGGAATCA
ACTATGTGTGCAAGTTAAGCATGCTTAATATCACCCAAACAAACAAAGAGGCAGCATTTCTTAA
AGTAATGAAGATAGATAAATCGGGTTAGTCCTTTGCGACACTGCTGGTGCTTTCTAGAGTTTTA
TATATTTTAAGCAGCTTGCTTTATATTCTGTCTTTGCCTCCCACCCCACCAGCACTTTTATTTG
TGGAGGGTTTTGGCTCGCCACACTTTGGGAAACTTATTTGATTTCACGGAGAGCTGAAGGAAGA
TCATTTTTGGCAACAGACAAGTTTAAACACGATTTCTATGGGACATTGCTAACTGGGGCCCCTA
AGGAGAAAGGGGAAACTGAGCGGAGAATGGGTTAAATCCTTGGAAGCAGGGGAGAGGCAGGGGA
GGAGAGAAGTCGGAGGAGTATAAAGAAAAGGACAGGAACCAAGAAGCGTGGGGGTGGTTTGCCG
TAATGTGAGTGTTTCTTAATTAGAGAACGGTTGACAATAGAGGGTCTGGCAGAGGCTCCTGGCC
GCGGTGCGGAGCGTCTGGAGCGGAGCACGCGCTGTCAGCTGGTGAGCGCACTCTCCTTTCAGGC
AGCTCCCCGGGGAGCTGTGCGGCCACATTTAACACCATCATCACCCCTCCCCGGCCTCCTCAAC
CTCGGCCTCCTCCTCGTCGACAGCCTTCCTTGGCCCCCACCAGCAGAGCTCACAGTAGCGAGCG
TCTCTCGCCGTCTCCCGCACTCGGCCGGGGCCTCTCTCCTCCCCCAGCTGCGCAGCGGGAGCCG
CCACTGCCCACTGCACCTCCCAGCAACCAGCCCAGCACGCAAAGAAGCTGCGCAAAGTTAAAGC
CAAGCAATGCCAAGGGGAGGGGAAGCTGGAGGCGGGCTTTGAGTGGCTTCTGGGCGCCTGGCGG
GTCCAGAATCGCCCAGAGCCGCCCGCGGTCGTGCACATCTGACCCGAGTCAGCTTGGGCACCAG
CCGAGAGCCGGCTCCGCACCGCTCCCGCACCCCAGCCGCCGGGGTGGTGACACACACCGGAGTC
GAATTACAGCCCTGCAATTAACATATGAATCTGACGAATTTAAAAGAAGGAAAAAAAAAAAAAA
ACCTGAGCAGGCTTGGGAGTCCTCTGCACACAAGAACTTTTCTCGGGGTGTAAAAACTCTTTGA
TTGGCTGCTCGCACGCGCCTGCCCGCGCCCTCCATTGGCTGAGAAGACACGCGACCGGCGCGAG
GAGGGGGTTGGGAGAGGAGCGGGGGGAGACTGAGTGGCGCGTGCCGCTTTTTAAAGGGGCGCAG
CGCCTTCAGCAACCGGAGAAGCATAGTTGCACGCGACCTGGTGTGTGATCTCCGAGTGGGTGGG
GGAGGGTCGAGGAGGGAAAAAAAAATAAGACGTTGCAGAAGAGACCCGGAAAGGGCCTTTTTTT
TGGTTGAGCTGGTGTCCCAGTGCTGCCTCCGATCCTGAGCCTCCGAGCCTTTGCAGTGCAA

In certain embodiments, a promoter is an endogenous human SLC26A4 immediate promoter as set forth in SEQ ID NO: 30 or 31. In certain embodiments, a promoter is an endogenous human SLC26A4 enhancer-promoter as set forth in SEQ ID NO: 32, 33 or 34. In some embodiments, an enhancer-promoter sequence is at least 85%, 90%, 95%, 98% or 99% identical to a promoter or enhancer-promoter sequence represented by SEQ ID NO: 30, 31, 32, 33, or 34. In certain embodiments, a promoter is a human SLC26A4 endogenous enhancer-promoter sequence comprised within SEQ ID NO: 32, 33, or 34.

Exemplary Human SLC26A4 immediate promoter
(SEQ ID NO: 30)
CTGCCTTCTGAGAGCGCTATAAAGGCAGCGGAAGGGTAGTCCGCGGGGCATTCCGGGCGG
Exemplary Human SLC26A4 immediate promoter
(SEQ ID NO: 31)
CTCTAGGCGGGCTCTGCTCTTCTTTAAGGAGTCCCACAGGGCCTGGCCCGCCCCTGACCT
Exemplary Human SLC26A4 enhancer-promoter
(SEQ ID NO: 32)
TAAAGAGTTGTGAGTTGTGTAGGTGAGTTGCCATGGAGCTACAAATATGAGTTGATATTCTGAA
ATCCTAGACAGCCATCTCCAAGGTTAAGAAAAATCCTTATGCACTCACTTGCAAAGATATCCAC
AGCATGCTCTTAATGGAGAAAAACAAAGCCTTAGATCAAATATGTAAAGTAATTTTTAGTTTTT
TGAAAAGGTATGTTTGGGCTATAGATAAATCTGTTCAAAAAACATGAGAGAAGATAATAATGGT
TGAAAGGAGACACAGTGCTTGCCCTCAAGAAGTTTTTGTCTAGTGAGGGAGAGAGAACTTGTAT
GTAAATAAAATTGTGTTACTAAGGTAGATAGTGAGAAGTAACTTAAGAGAGGATCAGATAAGGT
ATTAAGAGAATACAGAAAAGGGTCTGGATTAATTCTGAACAGCATCAAAGAATGTTCTTGCAAG
AGATAGTGTTTTCACCAGATCTTGAAGGTATGGATGAGGGTATACAGAGTGAGTATATTCAGAT
TCTACTTTAAAACAAATACTTTCCTCTGTTGTAGTGGAGTTGAGCTATACATCCAACAATAATG
AAAAAATACACGCATATATACATATATGGAGAGAGATACATATTTTAGTACATGTAGCAATTGA
TTAATAAATGTACAGTTTAAGTCGCATGCAAAACCTTGGAGTGATAGCAAACTTCATTGTAGGA
TGTTTAGCAGCATCTCTGGTCTCTACTCACTAGATCCCAATAGCATCTCCCTAGGTGTGACAAC
CAAAAATGTCTCCAGGCATTGACCTCTGGAGGCAAAAAAAGCCCTTTATTAAGAACCAGTGGTA
TACATAAGTAAAACATACACAAGAGATTCCTCCCCTCTTCTCTGTATGTGAATAAAAATTGCAA
AGTTCATGACCTGGATTTTCCTTTTAGGTTTCTTCTTTAGTGGTTCTTAACTTCATTGGGTGAA
GTAAGCCTTTGAAGATCTGTTGAAAGCTGTTGACTCATTCACTTCTCAGGAAAACGCACATGCT
GACTACCATTTCAGAGAATTTGCATCAGGGTTCTCTGGGGAGGAGTTCTGAGTTCTGTTTCCAG
GAGCTCGTAGAATTGTCATGGTCTGCATATGCAAGGCAGGTGGATTACGGAAGGTTGATGTACA
GAGGTCTGTATTTTGGAGCCTCTTCTGTATTTACTTCAGAACACTAACAATCAGGCGAGAATGT
TCTGGTTTATCAAACCCTTCCTTCTGCCTTTCATCTTAACCATGCATTAGTTTTAACAAAGTTC
ATCCCAACAGAAGACAAAACACTGATGAGGTAGGATAGCTCCAGCTCCTCCTCCCTCTCTTCTA
GTCTTGATTTCCATGTAGTCCAGTTTATTCCTTCCCTGATTGTCCAGGAGAATGAGAAAAAGAA
AAAACAGAGTCTAGTGGGTAAGAAAGGGCCACCTGGACGGCTTGATTTGGATTGTGAAATAAAA
CACACACACATGCACACGTAGAATAAGTGGCTAAAATCTGAGTAAATCGTGAACTCTCTGTATC
CTCCACCCATTGAATACTCCTAAAAGACTTTCTAGAAATTCAAGGACTTATTAATATAGAAACC
TGGCCATTGTTCCTCTTCTCCTCCCCATGTGGTATGAGAGCACCTGTGGCAGGCTCCCAGAGAC
CACGGACCTCTTCCTCTAGGCGGGCTCTGCTCTTCTTTAAGGAGTCCCACAGGGCCTGGCCCGC
CCCTGACCTCGCAACCCTTGAGATTAGTAACGGGATGAGTGAGGATCCGGGTGGCCCCTGCGTG
GCAGCCAGTAAGAGTCTCAGCCTTCCCGGTTCGGGAAAGGGGAAGAATGCAGGAGGGGTAGGAT
TTCTTTCCTGATAGGATCGGTTGGGAAAGACCGCAGCCTGTGTGTGTCTTTCCCTTCGACCAAG
GTGTCTGTTGCTCCGTAAATAAAACGTCCCACTGCCTTCTGAGAGCGCTATAAAGGCAGCGGAA
GGGTAGTCCGCGGGGC
Exemplary Human SLC26A4 enhancer-promoter
(SEQ ID NO: 33)
GGCTGCTCGGAAAACAGGACGAGGGGAGAGACTTGCTCAATAAGCTGAAAGTTCTGCCCCCGAG
AGGGCTGCGACAGCTGCTGGAATGTGCCTGCAGCGTCCGCCTCTTGGGGACCCGCGGAGCGCGC
CCTGACGGTTCCACGCCTGGCCCGGGGGTCTGCACCTCTCCTCCAGTGCGCACCTGGAGCTGCG
TCCCGGGTCAGGTGCGGGGAGGGAGGGAATCTCAGTGTCCCCTTCCAGCCTTGCAAGCGCCTTT
GGCCCCTGCCCCAGCCCCTCGGTTTGGGGGAGATTTCAGAACGCGGACAGCGCCCTGGCTGCGG
GCCATAGGGGACTGGGTGGAACTCGGGAAGCCCCCAGAGCAGGGGCTTACTCGCTTCAAGTTTG
GGGAACCCCGGGCAGCGGGTGCAGGCCACGAGACCCGAAGGTTCTCAGGTGCCCCCCTGCAGGC
TGGCCGTGCGCGCCGTGGGGCGCTTGTCGCGAGCGCCGAGGGCTGCAGGACGCGGACCAGACTC
GCGGTGCAGGGGGGCCTGGCTGCAGCTAACAGGTGATCCCGTTCTTTCTGTTCCTCGCTCTTCC
CCTCCGATCGTCCTCGCTTACCGCGTGTCCTCCCTCCTCGCTGTCCTCTGGCTCGCAGGTCATG
GCAGCGCCAGGCGGCAGGTCGGAGCCGCCGCAGCTCCCCGAGTACAGCTGCAGCTACATGGTGT
CGCGGCCGGTCTACAGCGAGCTCGCTTTCCAGCAACAGCACGAGCGGCGCCTGCAGGAGCGCAA
GACGCTGCGGGAGAGCCTGGCCAAGTGCTGCAGGTAGCGGCCGCGCGGGCCTGCGTAGAGAGAA
GCGGAGCGGGGCGTCCACGCCTTGGGGAGGGAAGGGCGTCCCCAGCGGGCGAGAGTGGGGTGCG
GGCGGCGGAGCCCCTGGGCGCCAGCTGCTTCTCCCAGAGGCCCGACTTTCGGTCTCCGGTCCTC
CACGCCGCCCTTCTGGTGGGAGGGTGGCTCCATCAGTCTCGGGCCCGAAATGAACTTACCTGGG
AAACTCGCCTTTGGGGAGAGTGGGTTCTAGGAGCCCCGTCTCTCTTTTTCCTCTCTGAAGGAAA
CTTGGAGTGCCTCTTGGGGTACAGTGGGTCCCTGTTGCCTTCTTGGGAGCTTGTTTAAATGAAA
TGAATAGGGAAACCCAGCTCTTGACCAGGAGGAGTCCTTGAAACACTCAAGCTAAGTAGGCGGG
CTACCATTCAGTTAGAGACCAGGATGCAAGCTAGAACCCAGGGGAGCGCGGGGTGTGCCAAGTA
CTTCATCAGCAGGCTGTGGGACCCCTGGGGAAAGCCACCCTCAGTCTCTAAACCCAAACATGCC
GTAACTAGATGTCACAAACATAAAGAAATTAGAGTTTCTAAAACCTTTCATTATAG
Exemplary Human SLC26A4 enhancer-promoter
(SEQ ID NO: 34)
CGGAAGGTTGATGTACAGAGGTCTGTATTTTGGAGCCTCTTCTGTATTTACTTCAGAACACTAA
CAATCAGGCGAGAATGTTCTGGTTTATCAAACCCTTCCTTCTGCCTTTCATCTTAACCATGCAT
TAGTTTTAACAAAGTTCATCCCAACAGAAGACAAAACACTGATGAGGTAGGATAGCTCCAGCTC
CTCCTCCCTCTCTTCTAGTCTTGATTTCCATGTAGTCCAGTTTATTCCTTCCCTGATTGTCCAG
GAGAATGAGAAAAAGAAAAAACAGAGTCTAGTGGGTAAGAAAGGGCCACCTGGACGGCTTGATT
TGGATTGTGAAATAAAACACACACACATGCACACGTAGAATAAGTGGCTAAAATCTGAGTAAAT
CGTGAACTCTCTGTATCCTCCACCCATTGAATACTCCTAAAAGACTTTCTAGAAATTCAAGGAC
TTATTAATATAGAAACCTGGCCATTGTTCCTCTTCTCCTCCCCATGTGGTATGAGAGCACCTGT
GGCAGGCTCCCAGAGACCACGGACCTCTTCCTCTAGGCGGGCTCTGCTCTTCTTTAAGGAGTCC
CACAGGGCCTGGCCCGCCCCTGACCTCGCAACCCTTGAGATTAGTAACGGGATGAGTGAGGATC
CGGGTGGCCCCTGCGTGGCAGCCAGTAAGAGTCTCAGCCTTCCCGGTTCGGGAAAGGGGAAGAA
TGCAGGAGGGGTAGGATTTCTTTCCTGATAGGATCGGTTGGGAAAGACCGCAGCCTGTGTGTGT
CTTTCCCTTCGACCAAGGTGTCTGTTGCTCCGTAAATAAAACGTCCCACTGCCTTCTGAGAGCG
CTATAAAGGCAGCGGAAGGGTAGTCCGCGGGGCATTCCGGGCGGGGCGCGAGCAGAGACAGGTG
AGTT

In certain embodiments, a promoter is a human LGR5 enhancer-promoter as set forth in SEQ ID NO: 35. In some embodiments, an enhancer-promoter sequence is at least 85%, 90%, 95%, 98% or 99% identical to enhancer-promoter sequence represented by SEQ ID NO: 35. In some embodiments, a promoter is a human LGR5 endogenous enhancer-promoter sequence comprised within SEQ ID NO: 35.

Exemplary Human LGR5 enhancer-promoter
(SEQ ID NO: 35)
AGGGCTATTTGTACCTCAACGAGGGCTTCTCTCCAAGAAAGCCCTGAATCCTTTTCCTCCTTTT
TCCTGCAGATTCACTATAGGACACTTTTTGAAGCAAGAGCATGCATTTTCCCCCTGGCGCTCTG
CAGCGGTTCTCAGAGCCCAGTGTCACTCACATAGGTGGGACTGCTCTCAGTTCAGAGAGCGCTG
GGACACTTAAGATGAAAAGTCCCTGGAAGTTAGCAAACAGCCATCTGTCACTCTGGCATCGATT
TACTAAAAGTGACTTCTAGGGTATTCTAAACCACTTTTAAAAAACAAATGAGTCACTTCGACTT
CCTCACCCCGCAAGAGATAGGAAGGCAGCAGTGGAGTGCTCGCTCAGGAGCTGTATTTGTTTAG
CGATTAGCCTAGAGCTTTGATTTTAGGGCAAAAGCGAGCCAGACAGTGCGGCAGACGTAAGGAT
CAAAAAGGCCACCTATCATTCGCCGGGGACGCCTGCCTCCTTACCCTGATAACGTAACTATTTC
TCTGCATAGGATTTTAGTTTTTGTGTTTTTGTTTTGTTTTATTCTGTTTAATCACTTCAAGTAT
CTCATCCATTATTTGAAGCGGGCTCGGAGGAAACGTGCCGCATCCTCCAGTCCTTGTGCGTCTG
TTTAGGTCTCTCCGAAGCAGGTCCCTCTCGACTCTTAGATCTGGGTCTCCAGCACGCATGAAGG
GGTAAGGGTGGGGGGGTCCCCTATTCCGGCGCGCGGCGTTGAGCACTGAATCTTCCAGGCGGAG
GCTCAGTGGGAGCGCCGAGAACTCGCCAGTACCGCGCGCTGCCTGCTGCCTGCTGCCTCCCAGC
CCAGGACTTGGGAAAGGAGGGAGGGGACAAGTGGAGGGAAAGTGGGGCCGGGCGGGGGGTGCCT
GGGAAGCCAGGCTGCGCTGACGTCACTGGGCGCGCAATTCGGGCTGGAGCGCTTTAAAAAACGA
GCGTGCAAGCAGAGATGCTGCTCCACACCGCTCAGGCCGCGAGCAGCAGCAAGGCGCACCGCCA
CTGTCGCCGCTGCAGCCAGGGCTGCTCCGAAGGCCGGCGTGGCGGCAACCGGCACCTCTGTCCC
CGCCGCGCTTCTCCTCGCCGCCCACGCCGTGGGGTCAGGAACGCGGCGTCTGGCGCTGCAGACG
CCCGCTGAGTTGCAGAAGCCCACGGAGCGGCGCCCGGCGCGCCACGGCCCGTAGCAGTCCGGTG
CTGCTCTCCGCCCGCGTCCGGCTCGTGGCCCCCTACTTCGGGCACCGACCGGT

In certain embodiments, a promoter is a human SYN1 enhancer-promoter as set forth in SEQ ID NO: 36. In some embodiments, an enhancer-promoter sequence is at least 85%, 90%, 95%, 98% or 99% identical to enhancer-promoter sequence represented by SEQ ID NO: 36. In some embodiments, a promoter is a human SYN1 endogenous enhancer-promoter sequence comprised within SEQ ID NO: 36.

Exemplary Human SYN1 enhancer-promoter
(SEQ ID NO: 36)
TGCGTATGAGTGCAAGTGGGTTTTAGGACCAGGATGAGGCGGGGTGGGGGTGCCTACCTGACGA
CCGACCCCGACCCACTGGACAAGCACCCAACCCCCATTCCCCAAATTGCGCATCCCCTATCAGA
GAGGGGGAGGGGAAACAGGATGCGGCGAGGCGCGTGCGCACTGCCAGCTTCAGCACCGCGGACA
GTGCCTTCGCCCCCGCCTGGCGGCGCGCGCCACCGCCGCCTCAGCACTGAAGGCGCGCTGACGT
CACTCGCCGGTCCCCCGCAAACTCCCCTTCCCGGCCACCTTGGTCGCGTCCGCGCCGCCGCCGG
CCCAGCCGGACCGCACCACGCGAGGCGCGAGATAGGGGGGCACGGGCGCGACCATCTGCGCTGC
GGCGCCGGCGACTCAGCGCTGCCTCAGTCTGCGGTGGGCAGCGGAGGAGTCGTGTCGTGCCTGA
GAGCGCAGTCGAGAA

In certain embodiments, a promoter is a human GFAP enhancer-promoter as set forth in SEQ ID NO: 37. In some embodiments, an enhancer-promoter sequence is at least 85%, 90%, 95%, 98% or 99% identical to enhancer-promoter sequence represented by SEQ ID NO: 37. In some embodiments, a promoter is a human GFAP endogenous enhancer-promoter sequence comprised within SEQ ID NO: 37.

Exemplary Human GFAP enhancer-promoter
(SEQ ID NO: 37)
CCCACCTCCCTCTCTGTGCTGGGACTCACAGAGGGAGACCTCAGGAGGCAGTCTGTCCATCACA
TGTCCAAATGCAGAGCATACCCTGGGCTGGGCGCAGTGGCGCACAACTGTAATTCCAGCACTTT
GGGAGGCTGATGTGGAAGGATCACTTGAGCCCAGAAGTTCTAGACCAGCCTGGGCAACATGGCA
AGACCCTATCTCTACAAAAAAAGTTAAAAAATCAGCCACGTGTGGTGACACACACCTGTAGTCC
CAGCTATTCAGGAGGCTGAGGTGAGGGGATCACTTAAGGCTGGGAGGTTGAGGCTGCAGTGAGT
CGTGGTTGCGCCACTGCACTCCAGCCTGGGCAACAGTGAGACCCTGTCTCAAAAGACAAAAAAA
AAAAAAAAAAAAAAAAGAACATATCCTGGTGTGGAGTAGGGGACGCTGCTCTGACAGAGGCTCG
GGGGCCTGAGCTGGCTCTGTGAGCTGGGGAGGAGGCAGACAGCCAGGCCTTGTCTGCAAGCAGA
CCTGGCAGCATTGGGCTGGCCGCCCCCCAGGGCCTCCTCTTCATGCCCAGTGAATGACTCACCT
TGGCACAGACACAATGTTCGGGGTGGGCACAGTGCCTGCTTCCCGCCGCACCCCAGCCCCCCTC
AAATGCCTTCCGAGAAGCCCATTGAGCAGGGGGCTTGCATTGCACCCCAGCCTGACAGCCTGGC
ATCTTGGGATAAAAGCAGCACAGCCCCCTAGGGGCTGCCCTTGCTGTGTGGCGCCACCGGCGGT
GGAGAACAAGGCTCTATTCAGCCTGTGCCCAGGAAAGGGGATCAGGGGATGCCCAGGCATGGAC
AGTGGGTGGCAGGGGGGGAGAGGAGGGCTGTCTGCTTCCCAGAAGTCCAAGGACACAAATGGGT
GAGGGGACTGGGCAGGGTTCTGACCCTGTGGGACCAGAGTGGAGGGCGTAGATGGACCTGAAGT
CTCCAGGGACAACAGGGCCCAGGTCTCAGGCTCCTAGTTGGGCCCAGTGGCTCCAGCGTTTCCA
AACCCATCCATCCCCAGAGGTTCTTCCCATCTCTCCAGGCTGATGTGTGGGAACTCGAGGAAAT
AAATCTCCAGTGGGAGACGGAGGGGTGGCCAGGGAAACGGGGCGCTGCAGGAATAAAGACGAGC
CAGCACAGCCAGCTCATGTGTAACGGCTTTGTGGAGCTGTCAAGGCCTGGTCTCTGGGAGAGAG
GCACAGGGAGGCCAGACAAGGAAGGGGTGACCTGGAGGGACAGATCCAGGGGCTAAAGTCCTGA
TAAGGCAAGAGAGTGCCGGCCCCCTCTTGCCCTATCAGGACCTCCACTGCCACATAGAGGCCAT
GATTGACCCTTAGACAAAGGGCTGGTGTCCAATCCCAGCCCCCAGCCCCAGAACTCCAGGGAAT
GAATGGGCAGAGAGCAGGAATGTGGGACATCTGTGTTCAAGGGAAGGACTCCAGGAGTCTGCTG
GGAATGAGGCCTAGTAGGAAATGAGGTGGCCCTTGAGGGTACAGAACAGGTTCATTCTTCGCCA
AATTCCCAGCACCTTGCAGGCACTTACAGCTGAGTGAGATAATGCCTGGGTTATGAAATCAAAA
AGTTGGAAAGCAGGTCAGAGGTCATCTGGTACAGCCCTTCCTTCCCTTTTTTTTTTTTTTTTTT
GTGAGACAAGGTCTCTCTCTGTTGCCCAGGCTGGAGTGGCGCAAACACAGCTCACTGCAGCCTC
AACCTACTGGGCTCAAGCAATCCTCCAGCCTCAGCCTCCCAAAGTGCTGGGATTACAAGCATGA
GCCACCCCACTCAGCCCTTTCCTTCCTTTTTAATTGATGCATAATAATTGTAAGTATTCATCAT
GGTCCAACCAACCCTTTCTTGACCCACCTTCCTAGAGAGAGGGTCCTCTTGCTTCAGCGGTCAG
GGCCCCAGACCCATGGTCTGGCTCCAGGTACCACCTGCCTCATGCAGGAGTTGGCGTGCCCAGG
AAGCTCTGCCTCTGGGCACAGTGACCTCAGTGGGGTGAGGGGAGCTCTCCCCATAGCTGGGCTG
CGGCCCAACCCCACCCCCTCAGGCTATGCCAGGGGGTGTTGCCAGGGGCACCCGGGCATCGCCA
GTCTAGCCCACTCCTTCATAAAGCCCTCGCATCCCAGGAGCGAGCAGAGCCAGAGCAGGTTGGA
GAGGAGACGCATCACCTCCGCTGCTCGC

Enhancers

In some instances, a construct can include an enhancer sequence. The term “enhancer” refers to a nucleotide sequence that can increase the level of transcription of a nucleic acid encoding a protein of interest (e.g., a clarin 1 protein). Enhancer sequences (generally 50-1500 bp in length) generally increase the level of transcription by providing additional binding sites for transcription-associated proteins (e.g., transcription factors). In some embodiments, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and/or a SV40 enhancer. In some embodiments, a construct comprises a CMV enhancer exemplified by SEQ ID NO: 38. In some embodiments, an enhancer sequence is at least 85%, 90%, 95%, 98% or 99% identical to the enhancer sequence represented by SEQ ID NO: 38. In some embodiments, an SV-40 derived enhancer is the SV-40 T intron sequence, which is exemplified by SEQ ID NO: 39. In some embodiments, a an enhancer sequence is at least 85%, 90%, 95%, 98% or 99% identical to the enhancer sequence represented by SEQ ID NO: 39.

Exemplary CMV enhancer
(SEQ ID NO: 38)
GACATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGG
Exemplary SV-40 synthetic intron
(SEQ ID NO: 39)
GGAGTCGCTGCGTTGCCTTCGCCCCGTGCCCCGCTCCGCGCCGCCTCGCGCCGCCCGCCCCGGC
TCTGACTGACCGCGTTACTCCCACAGGTGAGCGGGCGGGACGGCCCTTCTCCTCCGGGCTGTAA
TTAGCGCTTGGTTTAATGACGGCTCGTTTCTTTTCTGTGGCTGCGTGAAAGCCTTAAAGGGCTC
CGGGAGGGCCCTTTGTGCGGGGGGGAGCGGCTCGGGGGGTGCGTGCGTGTGTGTGTGCGTGGGG
AGCGCCGCGTGCGGCCCGCGCTGCCCGGCGGCTGTGAGCGCTGCGGGCGCGGCGCGGGGCTTTG
TGCGCTCCGCGTGTGCGCGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAGCAGGGGGTGTGGGCGCG
GCGGTCGGGCTGTAACCCCCCCCTGCACCCCCCTCCCCGAGTTGCTGAGCACGGCCCGGCTTCG
GGTGCGGGGCTCCGTGCGGGGCGTGGCGCGGGGCTCGCCGTGCCGGGCGGGGGGTGGCGGCAGG
TGGGGGTGCCGGGCGGGGGGGGGCCGCCTCGGGCCGGGGAGGGCTCGGGGGAGGGGCGCGGCGG
CCCCCGGAGCGCCGGCGGCTGTCGAGGCGCGGCGAGCCGCAGCCATTGCCTTTTATGGTAATCG
TGCGAGAGGGCGCAGGGACTTCCTTTGTCCCAAATCTGTGCGGAGCCGAAATCTGGGAGGCGCC
GCCGCACCCCCTCTAGCGGGCGCGGGGCGAAGCGGTGCGGCGCCGGCAGGAAGGAAATGGGCGG
GGAGGGCCTTCGTGCGTCGCCGCGCCGCCGTCCCCTTCTCCCTCTCCAGCCTCGGGGCTGTCCG
CGGGGGGACGGCTGCCTTCGGGGGGGACGGGGCAGGGCGGGGTTCGGCTTCTGGCGTGTGACCG
GCGGCTCTAGAGCCTCTGCTAACCATGTTCATGCCTTCTTCTTTTTCCTACAG

Flanking Untranslated Regions, 5′ UTRs and 3′ UTRs

In some embodiments, any of the constructs described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, compositions, kits, or methods as described herein to enhance or otherwise modulate the expression of a clarin 1 protein.

Natural 5′ UTRs include a sequence that plays a role in translation initiation. in some embodiments, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. The 5′ UTRs have also been known to form secondary structures that are involved in elongation factor binding.

In some embodiments, a 5′ UTR is included in any of the constructs described herein. Non-limiting examples of 5′ UTRs, including those from the following genes: albumin, serum amyloid A, Apolipoprotein A/B/E, transferrin, alpha fetoprotein, erythropoietin, and Factor VIII, can be used to enhance expression of a nucleic acid molecule, such as an mRNA.

In some embodiments, a 5′ UTR from an mRNA that is transcribed by a cell in the cochlea can be included in any of the constructs, compositions, kits, and methods described herein. In some embodiments, a 5′ UTR is derived from the endogenous CLRN1 gene loci and may include all or part of the endogenous sequence exemplified by SEQ ID NO: 40. In some embodiments, a 5′ UTR sequence is at least 85%, 90%, 95%, 98% or 99% identical to the 5′ UTR sequence represented by SEQ ID NO: 40.

3′ UTRs are found immediately 3′ to the stop codon of the gene of interest. In some embodiments, a 3′ UTR from an mRNA that is transcribed by a cell in the cochlea can be included in any of the constructs, compositions, kits, and methods described herein. In some embodiments, a 3′ UTR is derived from the endogenous CLRN1 gene loci and may include all or part of the endogenous sequence exemplified by SEQ ID NO: 41. In some embodiments, a 3′ UTR sequence is at least 85%, 90%, 95%, 98% or 99% identical to the 3′ UTR sequence represented by SEQ ID NO: 41.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (see, e.g., Chen et al., Mal. Cell. Biol. 15:5777-5788, 1995; Chen et al., Mal. Cell Biol. 15:2010-2018, 1995, each of which is incorporated herein by reference in its entirety): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif, two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

In some embodiments, the introduction, removal, or modification of 3′ UTR AREs can be used to modulate the stability of an mRNA encoding a clarin 1 protein. In other embodiments, AREs can be removed or mutated to increase the intracellular stability and thus increase translation and production of a clarin 1 protein.

In other embodiments, non-ARE sequences may be incorporated into the 5′ or 3′ UTRs. In some embodiments, introns or portions of intron sequences may be incorporated into the flanking regions of the polynucleotides in any of the constructs, compositions, kits, and methods provided herein. Incorporation of intronic sequences may increase protein production as well as mRNA levels.

Exemplary 5′ UTR Sequence
(SEQ ID NO: 40)
AGGAGATACTTGAAGGCAGTTTGAAAGACTTGTTTTACAGATTCTTAGTCCAAAGATTTCCAAT
TAGGGAGAAGAAGCAGCAGAAAAGGAGAAAAGCCAAGTATGAGTGATGATGAGGCCTTCATCTA
CTGACATTTAACCTGGCGAGAACCGTCGATGGTGAAGTTGCCTTTTCAGCTGGGAGCTGTCCGT
TCAGCTTCCGTAATAAATGCAGTCAAAGAGGCAGTCCCTTCCCATTGCTCACAAAGGTCTTGTT
TTTGAACCTCGCCCTCACAGAAGCCGTTTCTCATC
Exemplary 3′ UTR Sequence
(SEQ ID NO: 41)
AAGGCAAACCTTTCTATAATTTTACAAGGGAGTAGACTTGCTTTGGTCACTTTTAGATGTGGTT
AATTTTGCATATCCTTTTAGTCTGCATATATTAAAGCATCAGGACCCTTCGTGACAATGTTTAC
AAATTACGTACTAAGGATACAGGCTGGAAAGTAAGGGAAGCAGAAGGAAGGCTTTGAAAAGTTG
TTTTATCTGGTGGGAAATTGCTTGACCCAGGTAGTCAAAGGCAGTTGACTAGAATCGACAAATT
GTTACTCCATATATATATATGTGTGTGTGTGTGTGTGTGTGTGTGTGTAAGATGTCTTCCTATC
AAAAAGATATCAAAGGCACATGGAATATATTTTAATAAAAACAAATAATATCTCTAATATATCC
ACACATTTGTTGCCAGATTTCAGAAAACTGAGCTGCAATCGCTTTCCTAAAACAGTAGTGTATT
AAATGAACATCTATAAAATGTATCAACACACATTTTAAAAAATTTGTTTAAAGTATACTCTTAG
GCCAGGCGTGGTGACTCACACCTGTAATTCCAGCACTTCAGGAGGCCAAGGTGGGAAGATCATT
TGAGTTCAGGAGTTCGAGTTACAGCCTGGGCAATAAAGTGAGACCCTGTCACTAACAAAATTAA
AAAATAAAATAAATATAAAATATAGGCTTTAAAAAAGCATAGTCTTATTAACCATGTCTGTTGG
TCAAAATCTGCAAACTCTAAAAGAAGAAAAGAAGAAAAAACCAACGTTAGGGTATTTTTCCTCC
CGTGCCTGAGTCCCAATTACATTCACGACAGTACTTTCAATGAACATAATTGTTAGGACCACTG
AGGAATCATGAAAAATGATCTCTGCTTAGTACATTTGATGCAAAATGACTTATTAGGGGCTGTT
TTTCTAGCTATAGTGTCTCGAGTACTAATATGCAATTATGAAAATTATATTAAATCTGGGATTA
TGACGGTATCACTGTATCATCTTGGTCTTGTTCTGGCTGTCACCAAGCATGACCCAGGTCAACT
TTTTTTTTCCCCTGAATTACCCATCAAATTGATCTGCAGCTGACTAAAGGCCACAGCTGAGCCT
GGAACTGACCCTTCCTTCATCCTCAACCTGCTGTCCTCCAGAAAGCACCAAGGAAAAAGCAGAG
AATGACAGCAAACAGATCACTAGGCCTCTGACCACAGGTGCTGAGTACTCAGCAGCCCTCATAT
AATAGGTTTGAAAGTACTCCTTAAAATAAAACACTGTTTCCCTTTGGAACTATTTACAAGGATG
AAACAACCGTATACCTGAGAAATAACTTGCTCTGGTGTCAATTCGCTATTCGCCAGCAGACATC
AGAACACACCGAGTTTCCAGATGCT

Internal Ribosome Entry Sites (IRES)

In some embodiments, a construct encoding a clarin 1 protein can include an internal ribosome entry site (IRES). An IRES forms a complex secondary structure that allows translation initiation to occur from any position with an mRNA immediately downstream from where the IRES is located (see, e.g., Pelletier and Sonenberg, Mal. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV) (see e.g., Alberts, Molecular Biology of the Cell, Garland Science, 2002; and Hellen et al., Genes Dev. 15(13):1593-612, 2001, each of which is incorporated in its entirety herein by reference).

In some embodiments, the IRES sequence that is incorporated into a construct that encodes a clarin 1 protein, or a C-terminal portion of a clarin 1 protein is the foot and mouth disease virus (FMDV) 2A sequence. The Foot and Mouth Disease Virus 2A sequence is a small peptide (approximately 18 amino acids in length) that has been shown to mediate the cleavage of polyproteins (see, e.g., Ryan, M D et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999, each of which is incorporated in its entirety herein by reference). The cleavage activity of the 2A sequence has previously been demonstrated in artificial systems including plasmids and gene therapy constructs (AAV and retroviruses) (see, e.g., Ryan et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy 11: 1921-1931, 2000; and Klump et al., Gene Therapy 8:811-817, 2001, each of which is incorporated in its entirety herein by reference).

An IRES can be utilized in an AAV construct. In some embodiments, a construct encoding the C-terminal portion of the clarin 1 protein can include a polynucleotide internal ribosome entry site (IRES). In some embodiments, an IRES can be part of a composition comprising more than one construct. In some embodiments, an IRES is used to produce more than one polypeptide from a single gene transcript.

Splice Sites

In some embodiments, any of the constructs provided herein can include splice donor and/or splice acceptor sequences, which are functional during RNA processing occurring during transcription. In some embodiments, splice sites are involved in trans-splicing.

Exemplary splice donor intron
(SEQ ID NO: SEQ ID NO: 42)
GTAAGTATCAAGGTTACAAGACAGGTTTAAGGAGACCAATAGAAACTGGGCTTGTCGAGACAGA
GAAGACTCTTGCGTTTCT
Exemplary splice acceptor intron
(SEQ ID NO: SEQ ID NO: 43)
GATAGGCACCTATTGGTCTTACTGACATCCACTTTGCCTTTCTCTCCACAG

Polyadenylation Sequences

In some embodiments, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference in its entirety). A poly(A) tail confers mRNA stability and transferability (see, e.g., Molecular Biology of the Cell, Third Edition by B. Alberts et al., Garland Publishing, 1994, which is incorporated herein by reference in its entirety). In some embodiments, a poly(A) signal sequence is positioned 3′ to the coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some embodiments, a poly(A) tail is added onto transcripts that contain a specific sequence, e.g., a poly(A) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bGH) (see, e.g., Woychik et al., Proc. Natl. Acad Sci. US.A. 81(13):3944-3948, 1984; U.S. Pat. No. 5,122,458, each of which is incorporated herein by reference in its entirety), mouse-β-globin, mouse-α-globin (see, e.g., Orkin et al., EMBO J 4(2):453-456, 1985; Thein et al., Blood 71(2):313-319, 1988, each of which is incorporated herein by reference in its entirety), human collagen, polyoma virus (see, e.g., Batt et al., Mal. Cell Biol. 15(9):4783-4790, 1995, which is incorporated herein by reference in its entirety), the Herpes simplex virus thymidine kinase gene (HSV TK), IgG heavy-chain gene polyadenylation signal (US 2006/0040354, which is incorporated herein by reference in its entirety), human growth hormone (hGH) (see, e.g., Szymanski et al., Mal. Therapy 15(7):1340-1347, 2007, which is incorporated herein by reference in its entirety), the group consisting of SV40 poly(A) site, such as the SV40 late and early poly(A) site (see, e.g., Schek et al., Mal. Cell Biol. 12(12):5386-5393, 1992, which is incorporated herein by reference in its entirety).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference in its entirety).

In some embodiments, a poly(A) signal sequence can be a synthetic polyadenylation site (see, e.g., the pCl-neo expression construct of Promega that is based on Levitt el al, Genes Dev. 3(7):1019-1025, 1989, which is incorporated herein by reference in its entirety). In some embodiments, a poly(A) signal sequence is the polyadenylation signal of soluble neuropilin-1 (sNRP) (AAATAAAATACGAAATG) (see, e.g., WO 05/073384, which is incorporated herein by reference in its entirety). In some embodiments, a poly(A) signal sequence comprises or consists of the SV40 poly(A) site. In some embodiments, a poly(A) signal comprises or consists of SEQ ID NO: 45. In some embodiments, a poly(A) signal sequence comprises or consists of bGHpA. In some embodiments, a poly(A) signal comprises or consists of SEQ ID NO: 44. Additional examples of poly(A) signal sequences are known in the art. In some embodiments, a poly(A) sequence is at least 85%, 90%, 95%, 98% or 99% identical to the poly(A) sequence represented by SEQ ID NO: 44 or 45.

Exemplary bGH poly(A) signal sequence
(SEQ ID NO: 44)
CTGTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGAAGACAATA
GCAGGCATGCTGGGGATGCGGTGGGCTCTATGG
Exemplary SV40 poly(A) signal sequence
(SEQ ID NO: 45)
AACTTGTTTATTGCAGCTTATAATGGTTACAAATAAAGCAATAGCATCACAAATTTCACAAATA
AAGCATTTTTTTCACTGCATTCTAGTTGTGGTTTGTCCAAACTCATCAATGTATCTTA

Additional Sequences

In some embodiments, constructs of the present disclosure may comprise a T2A element or sequence. In some embodiments, constructs of the present disclosure may include one or more cloning sites. In some such embodiments, cloning sites may not be fully removed prior to manufacturing for administration to a subject. In some embodiments, cloning sites may have functional roles including as linker sequences, or as portions of a Kozak site. As will be appreciated by those skilled in the art, cloning sites may vary significantly in primary sequence while retaining their desired function. In some embodiments, constructs may contain any combination of cloning sites, exemplary cloning sites are represented by SEQ ID NO: 46-56.

Exemplary cloning site A
(SEQ ID NO: 46)
TTGTCGACGCGGCCGCACGCGT
Exemplary cloning site B
(SEQ ID NO: 47)
CTCCTGGGCAACGTGCTGGTTATTGTGACCGGTCGCTAGCCACC
Exemplary cloning site C
(SEQ ID NO: 48)
TAAGAGCTCGCTGATCAGCCTCGA
Exemplary cloning site D
(SEQ ID NO: 49)
AAGCTTGAATTCAGCTGACGTGCCTCGGACCGTCCTAGG
Exemplary cloning site E
(SEQ ID NO: 50)
GCGGCCGCACGCGT
Exemplary cloning site F
(SEQ ID NO: 51)
CTCCTGGGCAACGTGCTGGTTATTGTGACCGGTGCCACC
Exemplary cloning site G
(SEQ ID NO: 52)
TAAGAGCTCGCTGATCAGCCTCGA
Exemplary cloning site H
(SEQ ID NO: 53)
AAGCTTGAATTCAGCTGACGTGCCTCGGACCGCT
Exemplary cloning site I
(SEQ ID NO: 54)
CTCCTGGGCAACGTGCTGGTTATTGTGACCGGT
Exemplary cloning site J
(SEQ ID NO: 55)
GCCACC
Exemplary cloning site K
(SEQ ID NO: 56)
TAAGAGCTC

Destabilization Domains

In some embodiments, any of the constructs provided herein can optionally include a sequence encoding a destabilizing domain (“a destabilizing sequence”) for temporal control of protein expression. Non-limiting examples of destabilizing sequences include sequences encoding a FK506 sequence, a dihydrofolate reductase (DHFR) sequence, or other exemplary destabilizing sequences.

In the absence of a stabilizing ligand, a protein sequence operatively linked to a destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

Additional examples of destabilizing sequences are known in the art. In some embodiments, the destabilizing sequence is a FK506- and rapamycin-binding protein (FKBP12) sequence, and the stabilizing ligand is Shield-1 (Shldl) (see, e.g., Banaszynski et al. (2012) Cell 126(5): 995-1004, which is incorporated in its entirety herein by reference). In some embodiments, a destabilizing sequence is a DHFR sequence, and a stabilizing ligand is trimethoprim (TMP) (see, e.g., Iwamoto et al. (2010) Chem Biol 17:981-988, which is incorporated in its entirety herein by reference).

In some embodiments, a destabilizing sequence is a FKBP12 sequence, and a presence of an AAV construct carrying the FKBP12 gene in a subject cell (e.g., a supporting cochlear outer hair cell) is detected by western blotting. In some embodiments, a destabilizing sequence can be used to verify the temporally-specific activity of any of the AAV constructs described herein.

Exemplary DHFR destabilizing amino acid sequence
(SEQ ID NO: 57)
MISLIAALAVDYVIGMENAMPWNLPADLAWFKRNTLNKPVIMGRHTWESIGRPLPGRKNIILSS
QPSTDDRVTWVKSVDEAIAACGDVPEIMVIGGGRVIEQFLPKAQKLYLTHIDAEVEGDTHEPDY
EPDDWESVFSEFHDADAQNSHSYCFEILERR
Exemplary DHFR destabilizing nucleotide sequence
(SEQ ID NO: 58)
GGTACCATCAGTCTGATTGCGGCGTTAGCGGTAGATTACGTTATCGGCATGGAAAACGCCATGC
CGTGGAACCTGCCTGCCGATCTCGCCTGGTTTAAACGCAACACCTTAAATAAACCCGTGATTAT
GGGCCGCCATACCTGGGAATCAATCGGTCGTCCGTTGCCAGGACGCAAAAATATTATCCTCAGC
AGTCAACCGAGTACGGACGATCGCGTAACGTGGGTGAAGTCGGTGGATGAAGCCATCGCGGCGT
GTGGTGACGTACCAGAAATCATGGTGATTGGCGGCGGTCGCGTTATTGAACAGTTCTTGCCAAA
AGCGCAAAAACTGTATCTGACGCATATCGACGCAGAAGTGGAAGGCGACACCCATTTCCCGGAT
TACGAGCCGGATGACTGGGAATCGGTATTCAGCGAATTCCACGATGCTGATGCGCAGAACTCTC
ACAGCTATTGCTTTGAGATTCTGGAGCGGCGATAA
Exemplary destabilizing domain
(SEQ ID NO: 59)
ATCAGTCTGATTGCGGCGTTAGCGGTAGATTACGTTATCGGCATGGAAAACGCCATGCCGTGGA
ACCTGCCTGCCGATCTCGCCTGGTTTAAACGCAACACCTTAAATAAACCCGTGATTATGGGCCG
CCATACCTGGGAATCAATCGGTCGTCCGTTGCCAGGACGCAAAAATATTATCCTCAGCAGTCAA
CCGAGTACGGACGATCGCGTAACGTGGGTGAAGTCGGTGGATGAAGCCATCGCGGCGTGTGGTG
ACGTACCAGAAATCATGGTGATTGGCGGCGGTCGCGTTATTGAACAGTTCTTGCCAAAAGCGCA
AAAACTGTATCTGACGCATATCGACGCAGAAGTGGAAGGCGACACCCATTTCCCGGATTACGAG
CCGGATGACTGGGAATCGGTATTCAGCGAATTCCACGATGCTGATGCGCAGAACTCTCACAGCT
ATTGCTTTGAGATTCTGGAGCGGCGA
Exemplary FKBP12 destabilizing peptide amino acid sequence
(SEQ ID NO: 60)
MGVEKQVIRPGNGPKPAPGQTVTVHCTGFGKDGDLSQKFWSTKDEGQKPFSFQIGKGAVIKGWD
EGVIGMQIGEVARLRCSSDYAYGAGGFPAWGIQPNSVLDFEIEVLSVQ

Reporter Sequences or Elements

In some embodiments, constructs provided herein can optionally include a sequence encoding a reporter polypeptide and/or protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with control elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, a reporter sequence is the LacZ gene, and the presence of a construct carrying the LacZ gene in a mammalian cell (e.g., a cochlear hair cell) is detected by assays for beta-galactosidase activity. When the reporter is a fluorescent protein (e.g., green fluorescent protein) or luciferase, the presence of a construct carrying the fluorescent protein or luciferase in a mammalian cell (e.g., a cochlear hair cell) may be measured by fluorescent techniques (e.g., fluorescent microscopy or FACS) or light production in a luminometer (e.g., a spectrophotometer or an IVIS imaging instrument). In some embodiments, a reporter sequence can be used to verify the tissue-specific targeting capabilities and tissue-specific promoter regulatory and/or control activity of any of the constructs described herein.

In some embodiments, a reporter sequence is a FLAG tag (e.g., a 3×FLAG tag), and the presence of a construct carrying the FLAG tag in a mammalian cell (e.g., an inner ear cell, e.g., a cochlear hair or supporting cell, e.g., an eye cell) is detected by protein binding or detection assays (e.g., Western blots, immunohistochemistry, radioimmunoassay (RIA), mass spectrometry). An exemplary 3×FLAG tag sequence is provided as SEQ ID NO: 61.

Exemplary 3xFLAG tag sequence
(SEQ ID NO: 61)
GGATCCCGGGCTGACTACAAAGACCATGACGGTGATTATAAAGATCAT
GACATCGACTACAAGGATGACGATGACAAG

### AAV Particles

Among other things, the present disclosure provides AAV particles that comprise a construct encoding a CLRN1 gene or characteristic portion thereof described herein, and a capsid described herein. In some embodiments, AAV particles can be described as having a serotype, which is a description of the construct strain and the capsid strain. For example, in some embodiments an AAV particle may be described as AAV2, wherein the particle has an AAV2 capsid and a construct that comprises characteristic AAV2 Inverted Terminal Repeats (ITRs). In some embodiments, an AAV particle may be described as a pseudotype, wherein the capsid and construct are derived from different AAV strains, for example, AAV2/9 would refer to an AAV particle that comprises a construct utilizing the AAV2 ITRs and an AAV9 capsid.

AAV Construct

The present disclosure provides polynucleotide constructs that comprise a CLRN1 gene or characteristic portion thereof. In some embodiments described herein, a polynucleotide comprising a CLRN1 gene or characteristic portion thereof can be included in an AAV particle.

In some embodiments, a polynucleotide construct comprises one or more components derived from or modified from a naturally occurring AAV genomic construct. In some embodiments, a sequence derived from an AAV construct is an AAV1 construct, an AAV2 construct, an AAV3 construct, an AAV4 construct, an AAV5 construct, an AAV6 construct, an AAV7 construct, an AAV8 construct, an AAV9 construct, an AAV2.7m8 construct, an AAV8BP2 construct, an AAV293 construct, or AAV Anc80 construct. In some embodiments, an rAAV Anc80 capsid is an rAAV Anc80L65 capsid. Additional exemplary AAV constructs that can be used herein are known in the art (see, e.g., Kanaan et al., Mol. Ther. Nucleic Acids 8:184-197, 2017; Li et al., Mol. Ther. 16(7): 1252-1260, 2008; Adachi et al., Nat. Commun. 5: 3075, 2014; Isgrig et al., Nat. Commun. 10(1): 427, 2019; and Gao et al., J. Virol. 78(12): 6381-6388, 2004; each of which is incorporated in its entirety herein by reference).

In some embodiments, provided constructs comprise coding sequence, e.g., a CLRN1 gene or a characteristic portion thereof, one or more regulatory and/or control sequences, and optionally 5′ and 3′ AAV derived inverted terminal repeats (ITRs). In some embodiments wherein a 5′ and 3′ AAV derived ITR is utilized, the polynucleotide construct may be referred to as a recombinant AAV (rAAV) construct. In some embodiments, provided rAAV constructs are packaged into an AAV capsid to form an AAV particle.

In some embodiments, AAV derived sequences (which are comprised in a polynucleotide construct) typically include the cis-acting 5′ and 3′ ITR sequences (see, e.g., B. J. Carter, in “Handbook of Parvoviruses,” ed., P. Tijsser, CRC Press, pp. 155 168, 1990, which is incorporated herein by reference in its entirety). Typical AAV2-derived ITR sequences are about 145 nucleotides in length. In some embodiments, at least 80% of a typical ITR sequence (e.g., at least 85%, at least 90%, or at least 95%) is incorporated into a construct provided herein. The ability to modify these ITR sequences is within the skill of the art. (see, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996, each of which is incorporated in its entirety by reference). In some embodiments, any of the coding sequences and/or constructs described herein are flanked by 5′ and 3′ AAV ITR sequences. The AAV ITR sequences may be obtained from any known AAV, including presently identified AAV types.

In some embodiments, polynucleotide constructs described in accordance with this disclosure and in a pattern known to the art (see, e.g., Asokan et al., Mol. Ther. 20: 699-7080, 2012, which is incorporated herein by reference in its entirety) are typically comprised of, a coding sequence or a portion thereof, at least one and/or control sequence, and optionally 5′ and 3′ AAV inverted terminal repeats (ITRs). In some embodiments, provided constructs can be packaged into a capsid to create an AAV particle. An AAV particle may be delivered to a selected target cell. In some embodiments, provided constructs comprise an additional optional coding sequence that is a nucleic acid sequence (e.g., inhibitory nucleic acid sequence), heterologous to the construct sequences, which encodes a polypeptide, protein, functional RNA molecule (e.g., miRNA, miRNA inhibitor) or other gene product, of interest. In some embodiments, a nucleic acid coding sequence is operatively linked to and/or control components in a manner that permits coding sequence transcription, translation, and/or expression in a cell of a target tissue.

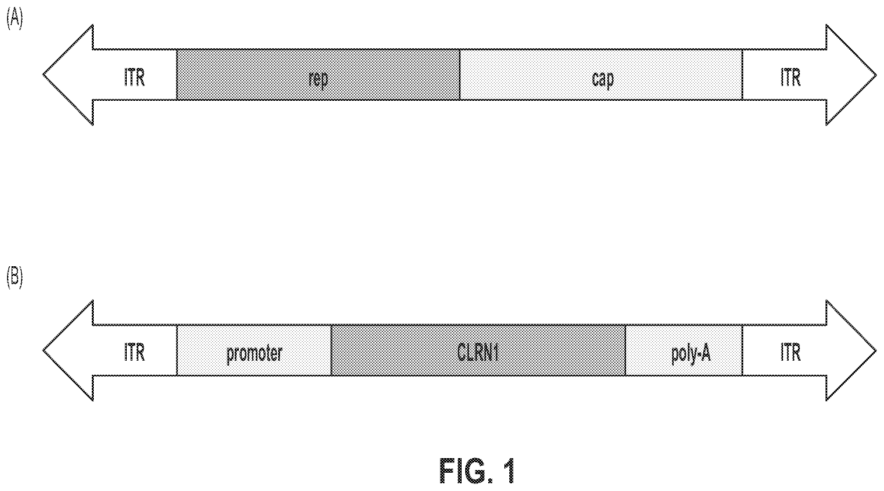

As shown in FIG. 1A, an unmodified AAV endogenous genome includes two open reading frames, “cap” and “rep,” which are flanked by ITRs. As shown in FIG. 1B, exemplary rAAV constructs similarly include ITRs flanking a coding region, e.g., a coding sequence (e.g., a CLRN1 gene). In some embodiments, an rAAV construct also comprises conventional control elements that are operably linked to the coding sequence in a manner that permits its transcription, translation and/or expression in a cell transfected with the plasmid construct or infected with the virus produced by the disclosure. In some embodiments, an rAAV construct optionally comprises a promoter (shown in FIG. 1B), an enhancer, an untranslated region (e.g., a 5′ UTR, 3′ UTR), a Kozak sequence, an internal ribosomal entry site (IRES), splicing sites (e.g., an acceptor site, a donor site), a polyadenylation site (shown in FIG. 1B), or any combination thereof. Such additional elements are described further herein.

In some embodiments, a construct is an rAAV construct. In some embodiments, an rAAV construct can include at least 500 bp, at least 1 kb, at least 1.5 kb, at least 2 kb, at least 2.5 kb, at least 3 kb, at least 3.5 kb, at least 4 kb, or at least 4.5 kb. In some embodiments, an AAV construct can include at most 7.5 kb, at most 7 kb, at most 6.5 kb, at most 6 kb, at most 5.5 kb, at most 5 kb, at most 4.5 kb, at most 4 kb, at most 3.5 kb, at most 3 kb, or at most 2.5 kb. In some embodiments, an AAV construct can include about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

Any of the constructs described herein can further include regulatory and/or control sequences, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or any combination thereof. In some embodiments, a promoter can be a native promoter, a constitutive promoter, an inducible promoter, and/or a tissue-specific promoter. Non-limiting examples of control sequences are described herein.

AAV Capsids

The present disclosure provides one or more polynucleotide constructs packaged into an AAV capsid. In some embodiments, an AAV capsid is from or derived from an AAV capsid of an AAV2, 3, 4, 5, 6, 7, 8, 9, 10, rh8, rh10, rh39, rh43 or Anc80 serotype, or one or more hybrids thereof. In some embodiments, an AAV capsid is from an AAV ancestral serotype. In some embodiments, an AAV capsid is an ancestral (Anc) AAV capsid. An Anc capsid is created from a construct sequence that is constructed using evolutionary probabilities and evolutionary modeling to determine a probable ancestral sequence. Thus, an Anc capsid/construct sequence is not known to have existed in nature. For example, in some embodiments, an AAV capsid is an Anc80 capsid (e.g., an Anc80L65 capsid).

In some embodiments, an AAV capsid is created using a template nucleotide coding sequence comprising SEQ ID NO: 62. In some embodiments, the capsid comprises a polypeptide represented by SEQ ID NO: 63. In some embodiments, the capsid comprises a polypeptide with at least 85%, 90%, 95%, 98% or 99% sequence identity to the polypeptide represented by SEQ ID NO: 63.

As provided herein, any combination of AAV capsids and AAV constructs (e.g., comprising AAV ITRs) may be used in recombinant AAV (rAAV) particles of the present disclosure. For example, wild type or variant AAV2 ITRs and Anc80 capsid, wild type or variant AAV2 ITRs and AAV6 capsid, etc. In some embodiments of the present disclosure, an AAV particle is wholly comprised of AAV2 components (e.g., capsid and ITRs are AAV2 serotype). In some embodiments, an AAV particle is an AAV2/6, AAV2/8 or AAV2/9 particle (e.g., an AAV6, AAV8 or AAV9 capsid with an AAV construct having AAV2 ITRs). In some embodiments of the present disclosure, an AAV particle is an AAV2/Anc80 particle that comprises an Anc80 capsid (e.g., comprising a polypeptide of SEQ ID NO: 63) that encapsidates an AAV construct with AAV2 ITRs (e.g., SEQ ID NOs: 15-22) flanking a portion of a coding sequence, for example, a CLRN1 gene or characteristic portion thereof (e.g., SEQ ID NO: 1, 2, 3, or 4). Other AAV particles are known in the art and are described in, e.g., Sharma et al., Brain Res Bull. 2010 Feb. 15; 81(2-3): 273, which is incorporated in its entirety herein by reference. In some embodiments, a capsid sequence is at least 85%, 90%, 95%, 98% or 99% identical to a capsid nucleotide or amino acid sequence represented by SEQ ID NO: 62 or 63, respectively.

In some embodiments, a platform delivery approach disclosed herein combines a library of synthetic AAV capsids, known as ancestral AAV (AAVAnc) capsids that recreate the evolutionary lineage of current naturally occurring viruses. In some embodiments, these AAV capsids are coupled with a novel, minimally invasive administration procedure to deliver product candidates directly to the cochlea. In some embodiment, a delivery approach utilizes an AAV Anc80 capsid variant from this library (e.g., Anc80L65). In some embodiments, such a capsid is utilized to create an rAAV particle, wherein such a particle is created through the addition of a construct as described herein, e.g., a construct comprising CLRN1 cDNA encoding a CLRN1 protein as described herein, to create an rAAV Anc80-CLRN1.

In some embodiments, a composition disclosed herein comprises an AAV Anc80 capsid, which is a rationally designed, synthetic AAV capsid whose sequence was inferred by ancestral sequence reconstruction. Ancestral sequence reconstruction uses available sequence information from naturally occurring adeno-associated viruses and, as a result of phylogenetic and statistical prediction, identifies the ancestral state of a sequence at various intermediary evolutionary nodes. During the creation of AAV Anc80, nine nodes were reconstructed, and in silico derived sequences across the AAV lineage were synthesized de novo and characterized. This led to the identification of the Anc80 Library node (Anc80Lib), the putative ancestor of the widely studied AAV serotypes 1, 2, 8, and 9. Anc80Lib protein sequences were subsequently reverse-translated and generated by gene synthesis, and individual clones were evaluated in isolation for packaging, infectivity, and biological properties. In some embodiments, and based on these results, AAV Anc80, the 65th Anc80Lib clone (Anc80L65), was selected for further characterization. An AAV Anc80 capsid variant has a distinctive composition; although the sequences of AAV8 and AAV2 differ by only approximately 9% and 12%, respectively, from AAV Anc80, structural modeling of AAV Anc80 has shown that around 20% of its particle external surface is divergent from known circulating AAVs in a manner that is distributed across the capsid surface (see, e.g., Zinn 2015, incorporated herein in its entirety by reference).

In some embodiments, AAV Anc80's performance as a gene therapy particle in-vivo has been characterized and rAAV particles comprising AAV Anc80 have demonstrated a potential to act as a broadly applicable gene therapy particle. In some embodiments, studies conducted in mice and non-human primates (NHPs) have shown that AAV Anc80 has a similar transduction efficiency to AAV8 when targeting the liver after intravenous injection, without obvious signs of systemic toxicity (see, e.g., Zinn 2015; Murillo 2019, each of which is incorporated herein in its entirety by reference). In addition, in some embodiments, AAV Anc80 has shown tropism for and efficient transduction of the mouse anterior segment of the eye (Wang 2017, incorporated herein in its entirety by reference), mouse and NHP retina (see, e.g., Zinn 2015; Carvalho 2018, each of which is incorporated herein in its entirety by reference), mouse skeletal muscle (see, e.g., Zinn 2015, incorporated herein in its entirety by reference), mouse central nervous system (CNS) by systemic and intraparenchymal delivery (Hudry 2018, incorporated herein in its entirety by reference), and murine kidney (see, e.g., Ikeda 2018, incorporated herein in its entirety by reference).

In some embodiments, compositions as described herein (e.g., comprising rAAV-CLRN1) comprise an AAV Anc80 capsid. In some embodiments, an rAAV Anc80 capsid is an rAAV Anc80L65 capsid. In some embodiments, AAV Anc80 capsid demonstrates high transduction efficiency for cochlear and vestibular cells. In some embodiments, AAV Anc80 capsid demonstrates high transduction efficiency for eye cells.

AAV Anc80 is a rationally designed AAV capsid whose sequence was inferred by ancestral sequence reconstruction (see, e.g., Zinn 2015, incorporated herein in its entirety by reference). Ancestral sequence reconstruction uses available sequence information from naturally occurring AAVs and, as a result of phylogenetic and statistical prediction, identifies the ancestral state of a sequence at various intermediary evolutionary nodes. As described in the literature, de novo synthesis and characterization of in silico derived sequences across the AAV lineage led to identification of the Anc80 Library (Anc80Lib) node, the putative ancestor of the widely studied AAV serotypes 1, 2, 8, and 9. Subsequent evaluation of Anc80Lib sequences led to the further characterization of AAV Anc80, the 65th Anc80Lib clone (Anc80L65). These studies indicated that the AAV Anc80 capsid variant has a distinctive composition with a divergent external surface particle distribution which yields a stable and functional AAV variant with a similar transduction efficiency to AAV8 (see, e.g., Zinn 2015, incorporated herein in its entirety by reference). The first reported use of AAV Anc80 in the mammalian inner ear revealed a high transduction efficiency in cochlear and vestibular hair cells (see, e.g., Landegger 2017, incorporated herein in its entirety by reference). Multiple subsequent, independent investigations have confirmed the increased cochlear and vestibular cell transduction efficiency of AAV Anc80 relative to other AAV serotypes; in mice of various ages (see, e.g., Landegger 2017; Tao 2018; Yoshimura 2018; and Omichi 2020, each of which is incorporated herein in its entirety by reference) and in non-human primates (see, e.g., Andres-Mateos 2019, incorporated herein in its entirety by reference), AAV Anc80 has a higher transduction efficiency and broader tropism compared to a number of other AAV capsids.

Gene therapy using AAV particles is a promising therapeutic modality for inner ear disorders for several reasons, such as: (1) the inner ear, which contains the auditory and vestibular sensory epithelia, has modified immune surveillance, similar to that in the central nervous system (Fujioka 2014, incorporated herein in its entirety by reference); (2) the sensory and supporting cells of the cochlear organ of Corti are post-mitotic, allowing for the possibility of long-term expression following a single administration of AAV; and (3) the aggregate clinical experience with rAAV delivery in both adults and children, via multiple routes of administration, suggests a strong safety profile for AAV as a delivery vehicle, particularly in localized delivery and/or at low to moderate doses.

Beginning with initial clinical trials more than two decades ago, rAAV particles have been administered to hundreds of participants in dozens of clinical trials at doses of up to approximately 1E15 vg or more for systemic administration (see, e.g., Flotte 1996; Flotte 2013; Parente 2018; and Wang 2019, each of which is incorporated herein in its entirety by reference). The number of trials in which AAV particles have been used for in-vivo gene transfer has steadily increased. The safety profile, together with the high efficiency of transduction of a broad range of target tissues, has established rAAV particles as the platform of choice for in-vivo gene therapy (see, e.g., Wang 2019, incorporated herein in its entirety by reference). Successful application of the rAAV technology has been achieved in the clinic for a variety of conditions, including coagulation disorders, inherited blindness, and neurodegenerative diseases (see, e.g., Colella 2018; and Wang 2019, each of which is incorporated herein in its entirety by reference).

An rAAV particle product (alipogene tiparvovec; Glybera®) was first approved by the European Medicines Agency (EMA) for treatment of lipoprotein lipase deficiency in 2012. Subsequently, two rAAV products, voretigene neparvovec-rzyl (Luxturna®) for the treatment of confirmed biallelic RPE65 mutation-associated retinal dystrophy and onasemnogene abeparvovec-xioi (Zolgensma®) for the treatment of spinal muscular atrophy (SMA) with biallelic mutations in the SMN1 gene, were approved by the U.S. Food and Drug Administration (FDA) in 2017 and 2019, respectively; voretigene neparvovec-rzyl (Luxturna®) was also approved by the EMA for the treatment of loss of vision due to inherited retinal dystrophy, when the disease is caused by mutations in the gene RPE65.

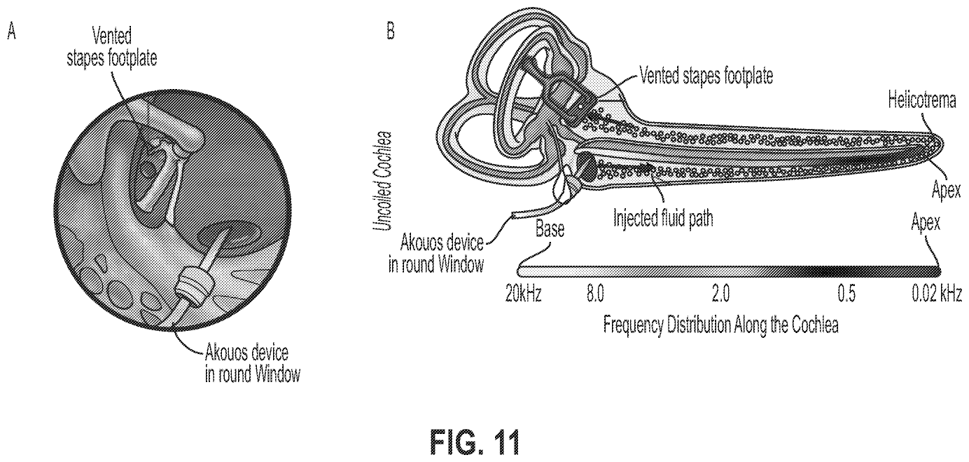

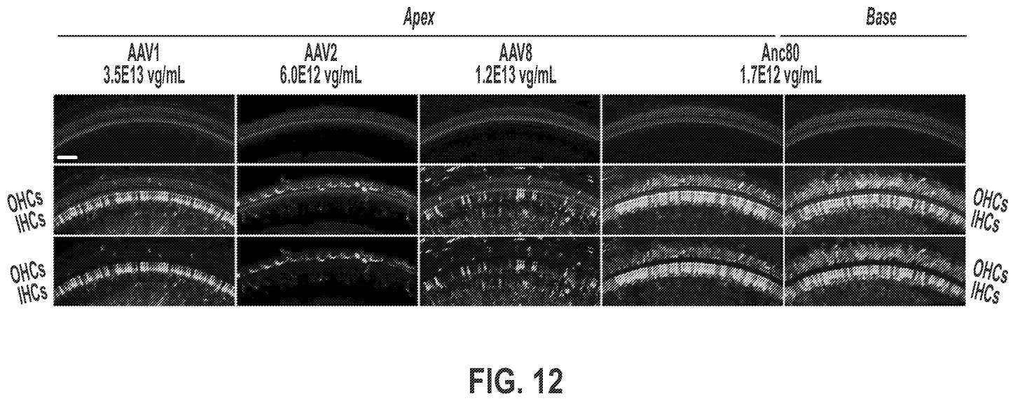

In some embodiments, drugs and biologics, including rAAV particles, can reach many target cells in the inner ear by delivering them into the perilymph. Perilymph is a fluid very similar in composition to (see, e.g., Lysaght 2011, incorporated herein in its entirety by reference), and in diffusional continuity with, cerebrospinal fluid (CSF). Perilymph bathes most of the sensory, neural, and supporting cells of the cochlea and of the vestibular system, housed in the bony labyrinth of the inner ear (FIGS. 11 and 12). In some embodiments, perilymphatic space of cochlea, to which a composition disclosed herein, e.g., rAAV-CLRN1, is delivered, comprises two scalae, or passages: scala tympani and scala vestibuli, which are continuous with one another at the apex of the cochlear spiral via the helicotrema. Many cells of the inner ear are in fluid continuity with perilymph through interstitial spaces in the tissue.

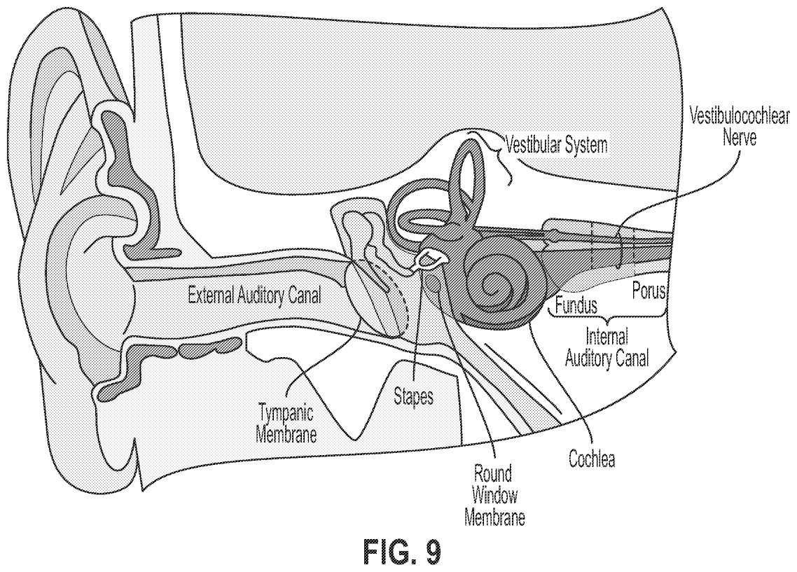

In some embodiments, also disclosed herein is a sterile, one-time use delivery device for intracochlear administration, to deliver a composition disclosed herein, e.g., rAAV-CLRN1 to perilymph fluid of inner ear through a round window membrane with a vent located in a stapes footplate. In some embodiments, in this intracochlear administration approach, a composition disclosed herein, e.g., rAAV-CLRN1, will be administered into the scala tympani through the round window membrane, with a vent in a stapes footplate within the oval window, such that composition is perfused through scala tympani, then through scala vestibuli via connection at the helicotrema, and follows the fluid path to a vent in a stapes footplate (FIG. 11). In some embodiments, presence of a vent distinct from the injection port allows for more even distribution of drug along the length of the cochlea and prevents the deleterious build-up of additional fluid pressure within the inner ear. In some embodiments, as evidenced by transduction of vestibular cells using this dual-fenestrae, injection plus venting technique, this delivery approach also permits diffusion of a composition disclosed herein, e.g., injectate, to a vestibular system. In some embodiments, the entire process can be accomplished in a subject with a relatively nontraumatic approach through an external auditory canal; see FIG. 9, and the Examples for additional information regarding the surgical administration procedure.

A number of studies on AAV Anc80 transduction in mice have been published. Different types of viral vectors (e.g., adenoviral vector, herpes simplex viral vectors) have been considered for gene delivery to the inner ear in animal models (Chen 2001; Wenzel 2007; Husseman 2009, each of which is incorporated herein in its entirety by reference); however, rAAV particles appear to be a promising tool for gene delivery directly to the cochlea given the acceptable safety profile and the long-lasting transgene expression, including recovery of auditory, cochlear, and vestibular function in knock-out and knock-in mouse models (see, e.g., Akil 2012; Kim 2016; Pan 2017; Akil 2019; Al-Moyed 2019; György 2019, each of which is incorporated herein in its entirety by reference). Several AAV serotypes have been delivered into the inner ear, using different surgical approaches and doses, in both neonatal and adult mice (see, e.g., Akil 2012; Askew 2015; Chien 2016; Landegger 2017; Suzuki 2017; Tao 2018; Yoshimura 2018; Akil 2019; Al-Moyed 2019; György 2019; Kim 2019; Omichi 2020, each of which is incorporated herein in its entirety by reference). Transduction efficiency, as assessed by GFP expression in different cell types of the cochlear and vestibular organs, differs depending on the mouse postnatal age, method use to deliver the particle, and serotype or capsid variant evaluated.

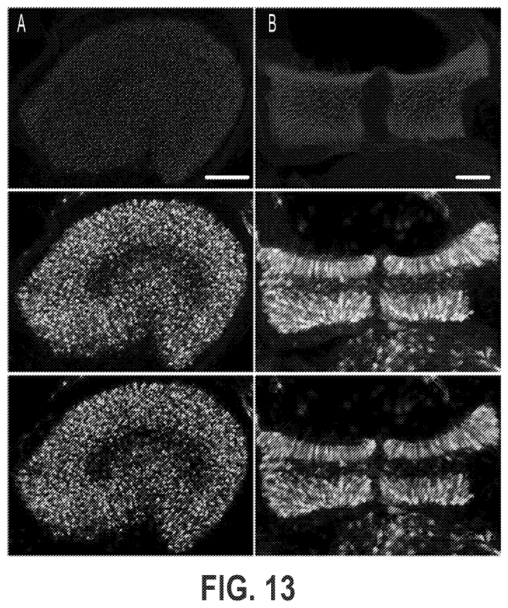

In some embodiments, AAV Anc80 variant has shown high efficiency targeting of cochlear and vestibular sensory cells (hair cells) and accessory cells of cochlear and vestibular organs in neonatal and adult mice compared with other AAV capsids (see, e.g., Landegger 2017; Suzuki 2017; Omichi 2020, each of which is incorporated herein in its entirety by reference). Anc80 neonatal tropism and gene transfer efficiency was evaluated in-vivo in C57BL/6 mice injected at postnatal day 1 (P1) by round-window administration (see, e.g., Landegger 2017, incorporated herein in its entirety by reference). Consistent with prior studies, AAV2, AAV6, and AAV8 targeted a low percentage of IHCs, and AAV1 was able to transduce IHCs with higher efficiency, but OHC transduction was minimal; in contrast, AAV Anc80 (1.7E9 vg/cochlea) was able to transduce around 100% of IHCs and ˜90% of OHCs (FIG. 12) without any deleterious effects to cochlear or auditory function (see, e.g., Landegger 2017, incorporated herein in its entirety by reference). In some embodiments, in the vestibular system, AAV Anc80 transduced type I and type II hair cells of the utricle as well as cells of the semicircular canal cristae (FIG. 13), without impacting vestibular function (see, e.g., Landegger 2017, incorporated herein in its entirety by reference).

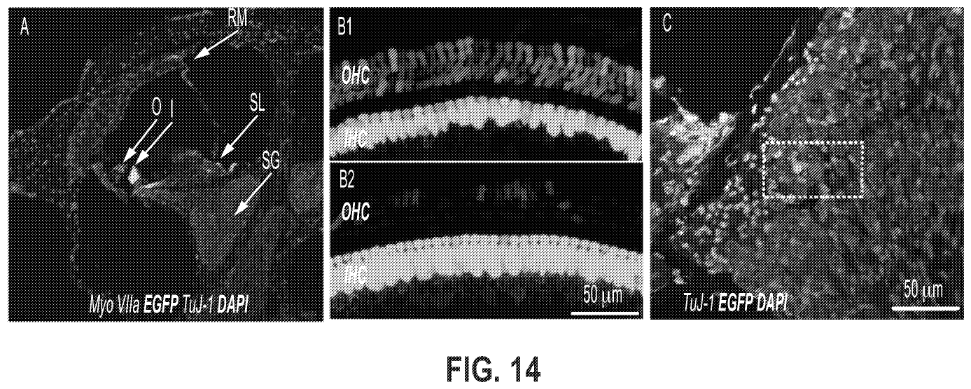

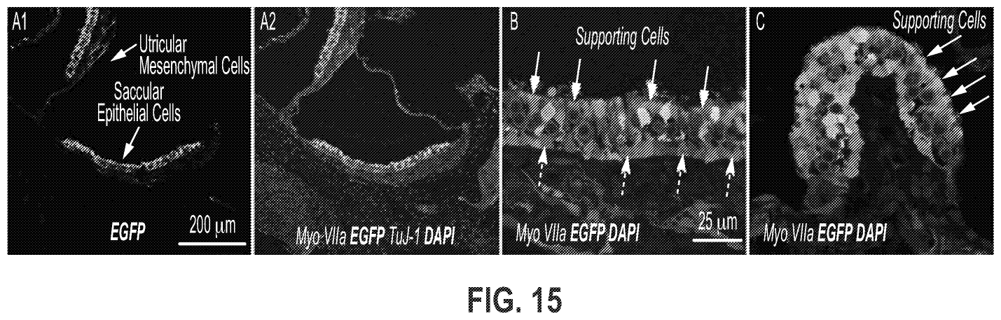

Using a different route of administration via the posterior semicircular canal, AAV Anc80 tropism and gene transfer efficiency was evaluated in adult (7 wks) CBA/CaJ mice (see, e.g., Suzuki 2017, incorporated herein in its entirety by reference). AAV Anc80 (9.6E8 vg/cochlea) targeted sensory and accessory cells of the cochlea, including approximately 100% of IHCs throughout the cochlear length as well as a significant fraction of OHCs, cells of the spiral limbus and Reissner's membrane, and cells of the cochlear modiolus (e.g., spiral ganglion neurons and satellite glial cells) (FIG. 14) while maintaining normal cochlear and auditory function (see, e.g., Suzuki 2017, incorporated herein in its entirety by reference). Multiple cell types of the vestibular system were also transduced, including a subset of hair cells and virtually 100% of supporting cells in the utricle and semicircular canal cristae, as well as the saccule (FIG. 15), all without deleterious effects on vestibular function (see, e.g., Suzuki 2017, incorporated herein in its entirety by reference).

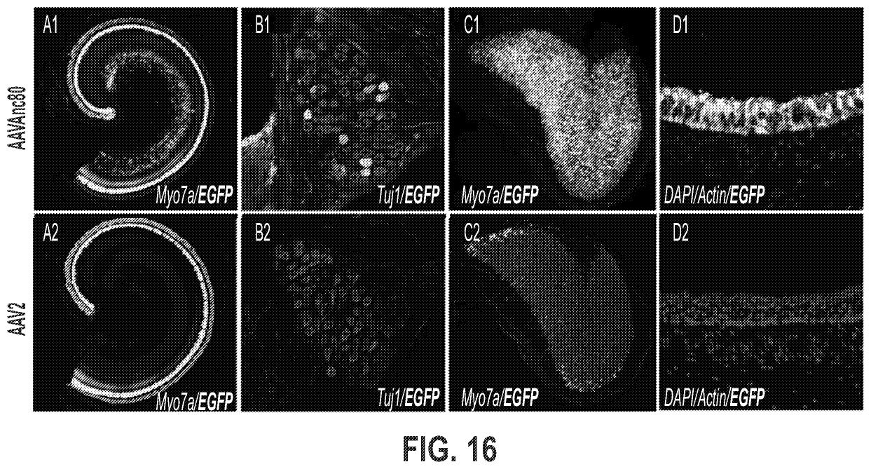

More recently, AAV Anc80 in-vivo transduction in adult (4 wks) C3H/FeJ mice was evaluated using a route of delivery utilized herein (via round window membrane delivery with posterior semicircular canal fenestration) and directly compared to transduction by naturally occurring serotypes AAV1, AAV2, AAV8, and AAV9 (see, e.g., Omichi 2020, incorporated herein in its entirety by reference). All particles produced some degree of transduction without deleterious effects to auditory function, as demonstrated by control-like (uninjected) ABR thresholds. AAV Anc80 (5.5E9 vg/cochlea) transduced virtually 100% of IHCs along the cochlear length, and approximately 27 to 66% of OHCs depending on cochlear location (FIG. 16). Despite a slightly higher transduction efficiency for OHCs by AAV2 (3.68E9 vg/cochlea) compared to AAV Anc80, AAV Anc80 maintained a significantly broader tropism than AAV2 as evidenced by eGFP-positive hair cells of the saccule and spiral ganglion neurons in the cochlea; in these same cell types, AAV2 produced little-to-no transduction (FIG. 16) (see, e.g., Omichi 2020, incorporated herein in its entirety by reference).

In some embodiments, the ability of AAV Anc80 to target a wide range of inner ear cell types, including cochlear IHCs and OHCs, supporting cells, cells of the cochlear spiral ganglion, vestibular hair cells of utricle, saccule, and crista ampularis, and cochlear and vestibular supporting/accessory cells, in neonatal to adult mice, suggests, e.g., that AAV Anc80 could facilitate development of gene therapy approaches for disorders of the inner ear.

Exemplary AAV Anc80 Capsid DNA Sequence
(SEQ ID NO: 62)
ATGGCTGCCGATGGTTATCTTCCAGATTGGCTCGAGGACAACCTCTCTGAGGGCATTCGCGAGT
GGTGGGACTTGAAACCTGGAGCCCCGAAACCCAAAGCCAACCAGCAAAAGCAGGACGACGGCCG
GGGTCTGGTGCTTCCTGGCTACAAGTACCTCGGACCCTTCAACGGACTCGACAAGGGGGAGCCC
GTCAACGCGGCGGACGCAGCGGCCCTCGAGCACGACAAGGCCTACGACCAGCAGCTCAAAGCGG
GTGACAATCCGTACCTGCGGTATAACCACGCCGACGCCGAGTTTCAGGAGCGTCTGCAAGAAGA
TACGTCTTTTGGGGGCAACCTCGGGCGAGCAGTCTTCCAGGCCAAGAAGCGGGTTCTCGAACCT
CTCGGTCTGGTTGAGGAAGGCGCTAAGACGGCTCCTGGAAAGAAGAGACCGGTAGAGCAATCAC
CCCAGGAACCAGACTCCTCTTCGGGCATCGGCAAGAAAGGCCAGCAGCCCGCGAAGAAGAGACT
CAACTTTGGGCAGACAGGCGACTCAGAGTCAGTGCCCGACCCTCAACCACTCGGAGAACCCCCC
GCAGCCCCCTCTGGTGTGGGATCTAATACAATGGCAGCAGGCGGTGGCGCTCCAATGGCAGACA
ATAACGAAGGCGCCGACGGAGTGGGTAACGCCTCAGGAAATTGGCATTGCGATTCCACATGGCT
GGGCGACAGAGTCATCACCACCAGCACCCGAACCTGGGCCCTCCCCACCTACAACAACCACCTC
TACAAGCAAATCTCCAGCCAATCGGGAGCAAGCACCAACGACAACACCTACTTCGGCTACAGCA
CCCCCTGGGGGTATTTTGACTTTAACAGATTCCACTGCCACTTCTCACCACGTGACTGGCAGCG
ACTCATCAACAACAACTGGGGATTCCGGCCCAAGAGACTCAACTTCAAGCTCTTCAACATCCAG
GTCAAGGAGGTCACGACGAATGATGGCACCACGACCATCGCCAATAACCTTACCAGCACGGTTC
AGGTCTTTACGGACTCGGAATACCAGCTCCCGTACGTCCTCGGCTCTGCGCACCAGGGCTGCCT
GCCTCCGTTCCCGGCGGACGTCTTCATGATTCCTCAGTACGGGTACCTGACTCTGAACAATGGC
AGTCAGGCCGTGGGCCGTTCCTCCTTCTACTGCCTGGAGTACTTTCCTTCTCAAATGCTGAGAA
CGGGCAACAACTTTGAGTTCAGCTACACGTTTGAGGACGTGCCTTTTCACAGCAGCTACGCGCA
CAGCCAAAGCCTGGACCGGCTGATGAACCCCCTCATCGACCAGTACCTGTACTACCTGTCTCGG
ACTCAGACCACGAGTGGTACCGCAGGAAATCGGACGTTGCAATTTTCTCAGGCCGGGCCTAGTA
GCATGGCGAATCAGGCCAAAAACTGGCTACCCGGGCCCTGCTACCGGCAGCAACGCGTCTCCAA
GACAGCGAATCAAAATAACAACAGCAACTTTGCCTGGACCGGTGCCACCAAGTATCATCTGAAT
GGCAGAGACTCTCTGGTAAATCCCGGTCCCGCTATGGCAACCCACAAGGACGACGAAGACAAAT
TTTTTCCGATGAGCGGAGTCTTAATATTTGGGAAACAGGGAGCTGGAAATAGCAACGTGGACCT
TGACAACGTTATGATAACCAGTGAGGAAGAAATTAAAACCACCAACCCAGTGGCCACAGAACAG
TACGGCACGGTGGCCACTAACCTGCAATCGTCAAACACCGCTCCTGCTACAGGGACCGTCAACA
GTCAAGGAGCCTTACCTGGCATGGTCTGGCAGAACCGGGACGTGTACCTGCAGGGTCCTATCTG
GGCCAAGATTCCTCACACGGACGGACACTTTCATCCCTCGCCGCTGATGGGAGGCTTTGGACTG
AAACACCCGCCTCCTCAGATCCTGATTAAGAATACACCTGTTCCCGCGAATCCTCCAACTACCT
TCAGTCCAGCTAAGTTTGCGTCGTTCATCACGCAGTACAGCACCGGACAGGTCAGCGTGGAAAT
TGAATGGGAGCTGCAGAAAGAAAACAGCAAACGCTGGAACCCAGAGATTCAATACACTTCCAAC
TACAACAAATCTACAAATGTGGACTTTGCTGTTGACACAAATGGCGTTTATTCTGAGCCTCGCC
CCATCGGCACCCGTTACCTCACCCGTAATCTG
Exemplary AAV Anc80L65 Capsid Amino Acid Sequence
(SEQ ID NO: 63)
MAADGYLPDWLEDNLSEGIREWWDLKPGAPKPKANQQKQDDGRGLVLPGYKYLGPFNGLDKGEP
VNAADAAALEHDKAYDQQLKAGDNPYLRYNHADAEFQERLQEDTSFGGNLGRAVFQAKKRVLEP
LGLVEEGAKTAPGKKRPVEQSPQEPDSSSGIGKKGQQPAKKRLNFGQTGDSESVPDPQPLGEPP
AAPSGVGSNTMAAGGGAPMADNNEGADGVGNASGNWHCDSTWLGDRVITTSTRTWALPTYNNHL
YKQISSQSGASTNDNTYFGYSTPWGYFDFNRFHCHFSPRDWQRLINNNWGFRPKRLNFKLFNIQ
VKEVTTNDGITTIANNLTSTVQVFTDSEYQLPYVLGSAHQGCLPPFPADVFMIPQYGYLTLNNG
SQAVGRSSFYCLEYFPSQMLRTGNNFEFSYTFEDVPFHSSYAHSQSLDRLMNPLIDQYLYYLSR
TQTTSGTAGNRTLQFSQAGPSSMANQAKNWLPGPCYRQQRVSKTANQNNNSNFAWTGATKYHLN
GRDSLVNPGPAMATHKDDEDKFFPMSGVLIFGKQGAGNSNVDLDNVMITSEEEIKTTNPVATEQ
YGTVATNLQSSNTAPATGTVNSQGALPGMVWQNRDVYLQGPIWAKIPHTDGHFHPSPLMGGFGL
KHPPPQILIKNTPVPANPPTTFSPAKFASFITQYSTGQVSVEIEWELQKENSKRWNPEIQYTSN
YNKSTNVDFAVDINGVYSEPRPIGTRYLTRNL*
Exemplary AAV Anc80 Scaffold Amino Acid Sequence
(SEQ ID NO: XX)
MAADGYLPDWLEDNLSEGIREWWDLKPGAPKPKANQQKQDDGRGLVLPGYKYLGPFNGLDKGEP
VNAADAAALEHDKAYDQQLKAGDNPYLRYNHADAEFQERLQEDTSFGGNLGRAVFQAKKRVLEP
LGLVEEGAKTAPGKKRPVEQSPQEPDSSSGIGKKGQQPAX₁KRLNFGQTGDSESVPDPQPLGEPP
AAPSGVGSNTMX₂AGGGAPMADNNEGADGVGNASGNWHCDSTWLGDRVITTSTRTWALPTYNNHL
YKQISSQSGX₃STNDNTYFGYSTPWGYFDFNRFHCHFSPRDWQRLINNNWGFRPKX₄LNFKLFNI
QVKEVTTNDGTTTIANNLTSTVQVFTDSEYQLPYVLGSAHQGCLPPFPADVFMIPQYGYLTLNN
GSQAVGRSSFYCLEYFPSQMLRTGNNFX₅FSYTFEDVPFHSSYAHSQSLDRLMNPLIDQYLYYLS
RTQTTSGTAGNRX₆LQFSQAGPSSMANQAKNWLPGPCYRQQRVSKTX₇NQNNNSNFAWTGATKYH
LNGRDSLVNPGPAMATHKDDEDKFFPMSGVLIFGKQGAGNSNVDLDNVMITX₈EEEIKTTNPVAT
EX₉YGTVATNLQSX₁₀NTAPATGTVNSQGALPGMVWQX₁₁RDVYLQGPIWAKIPHTDGHFHPSPLM
GGFGLKHPPPQILIKNTPVPANPPTTFSPAKFASFITQYSTGQVSVEIEWELQKENSKRWNPEI
QYTSNYNKSTNVDFAVDINGVYSEPRPIGTRYLTRNL

| Reference | Amino Acid |
|---|---|
| X₁ | K or R |
| X₂ | A or S |
| X₃ | A or G |
| X₄ | R or k |
| X₅ | E or Q |
| X₆ | T or E |
| X₇ | A or T |
| X₈ | S or N |
| X₉ | Q or E |
| X₁₀ | S or A |
| X₁₁ | N or D |

### Compositions

Among other things, the present disclosure provides compositions. In some embodiments, a composition comprises a construct as described herein. In some embodiments, a composition comprises one or more constructs as described herein. In some embodiments, a composition comprises a plurality of constructs as described herein. In some embodiments, when more than one construct is included in the composition, the constructs are each different.

In some embodiments, a composition comprises an AAV particle as described herein. In some embodiments, a composition comprises one or more AAV particles as described herein. In some embodiments, a composition comprises a plurality of AAV particles. In come embodiments, when more than one AAV particle is included in the composition, the AAV particles are each different.

In some embodiments, a composition comprises clarin 1 protein. In some embodiments, a composition comprises a cell.

In some embodiments, a composition is or comprises a pharmaceutical composition.

Single AAV Construct Compositions

In some embodiments, the present disclosure provides compositions or systems comprising AAV particles comprised of a single construct. In some such embodiments, a single construct may deliver a polynucleotide that encodes a functional (e.g., wild type or otherwise functional, e.g., codon optimized) copy of a CLRN1 gene. In some embodiments, a construct is or comprises an rAAV construct. In some embodiments described herein, a single rAAV construct is capable of expressing a full-length CLRN1 messenger RNA or a characteristic protein thereof in a target cell (e.g., an inner ear cell, e.g., an eye cell). In some embodiments, a single construct (e.g., any of the constructs described herein) can include a sequence encoding a functional clarin 1 protein (e.g., any construct that generates functional clarin 1 protein). In some embodiments, a single construct (e.g., any of the constructs described herein) can include a sequence encoding a functional clarin 1 protein (e.g., any construct that generates functional clarin 1 protein) and optionally additional polypeptide sequences (e.g., regulatory sequences, and/or reporter sequences).

In some embodiments, a single construct composition or system may comprise any or all of the exemplary construct components described herein. In some embodiments, an exemplary single construct is represented by SEQ ID NO: 64. In some embodiments, an exemplary single construct is at least 85%, 90%, 95%, 98% or 99% identical to the sequence represented by SEQ ID NO: 64. One skilled in the art would recognize that constructs may undergo additional modifications including codon-optimization, introduction of novel but functionally equivalent (e.g., silent mutations), addition of reporter sequences, and/or other routine modification.

In some embodiments, a single construct composition or system may comprise any or all of the exemplary construct components described herein. In some embodiments, an exemplary single construct is represented by SEQ ID NO: 68. In some embodiments, an exemplary single construct is at least 85%, 90%, 95%, 98% or 99% identical to the sequence represented by SEQ ID NO: 68. One skilled in the art would recognize that constructs may undergo additional modifications including codon-optimization, introduction of novel but functionally equivalent (e.g., silent mutations), addition of reporter sequences, and/or other routine modifications.

In some embodiments, an exemplary construct comprises: a5′ITR exemplified by SEQ ID NO: 21, optionally a cloning site exemplified by SEQ ID NO: 46, a CMV enhancer exemplified by SEQ ID NO: 38, a CBA promoter exemplified by SEQ ID NO: 23, a chimeric intron exemplified by SEQ ID NO: 39, optionally a cloning site exemplified by SEQ ID NO: 54, optionally a 5′ UTR exemplified by SEQ ID NO: 40, a CLRN1 coding region exemplified by SEQ ID NO: 1, optionally a 3′ UTR exemplified by SEQ ID NO: 41, a poly(A) site exemplified by SEQ ID NO: 44, optionally a cloning site exemplified by SEQ ID NO: 49, and a 3′ ITR exemplified by SEQ ID NO: 22 (see below Table 1).

TABLE 1
Exemplary Construct and Components

| | | | Position in Exemplary | |
|---|---|---|---|---|
| Components | | | Single Construct sequence | |
| (5′ to 3′ order) | Size (nt) | Origins and Notes | (SEQ ID NO: 64) | SEQ ID NO. |
| 5′ ITR | 145 | Wildtype AAV2 ITR | 1-145 | 21 |
| | | (AF043303.1) | | |
| Cloning site | 22 | N/A (SalI/NotI/MluI) | 146-167 | 46 |
| CMV enhancer | 381 | NCBI K03104.1 | 168-548 | 38 |
| | | (140 . . . 520) | | |
| CBA promoter | 279 | NCBI X00182.1 | 549-827 | 23 |
| | | (268 . . . 543) | | |
| Chimeric intron | 1013 | NCBI X00182.1 | 828-1840 | 39 |
| | | (544 . . . 1503) NCBI | | |
| | | V00882.1 (1250 . . . 1317); | | |
| | | XbaI cloning site | | |
| Cloning site | 33 | N/A; AgeI | 1841-1873 | 54 |
| 5′ UTR | 291 | NCBI NG_009168.1 | 1874-2164 | 40 |
| | | (5001-5291) | | |
| CLRN1 | 696 | NCBI NM_174878.3 | 2171-2866 | 1 |
| | | (20-715) | | |
| 3′ UTR | 1369 | NCBI NG_009168.1 | 2876-4244 | 41 |
| | | (50065-51433) | | |
| bGHpA | 225 | NCBI M57764.1 | 4245-4469 | 44 |
| | | (2326-2550) | | |
| Cloning site | 39 | N/A; | 4470-4508 | 49 |
| | | HindIII/EcoRI/PvuII/ | | |
| | | RsrII/AvrII | | |
| 3′ ITR | 145 | Wildtype AAV2 ITR | 4509-4653 | 22 |
| | | (AF043303.1) | | |

In some embodiments, an exemplary construct comprises: a5′ITR exemplified by SEQ ID NO: 21, optionally a cloning site exemplified by SEQ ID NO: 46, a CMV enhancer exemplified by SEQ ID NO: 38, a CBA promoter exemplified by SEQ ID NO: 23, a chimeric intron exemplified by SEQ ID NO: 39, optionally a cloning site exemplified by SEQ ID NO: 54, optionally a 5′ UTR exemplified by SEQ ID NO: 40, a CLRN1 coding region exemplified by SEQ ID NO: 19, optionally a 3′ UTR exemplified by SEQ ID NO: 41, a poly(A) site exemplified by SEQ ID NO: 44, optionally a cloning site exemplified by SEQ ID NO: 49, and a 3′ ITR exemplified by SEQ ID NO: 22 (see below Table 2).

TABLE 2
Exemplary Construct and Components

| | | | Position in Exemplary | |
|---|---|---|---|---|
| Components | | | Single Construct sequence | |
| (5′ to 3′ order) | Size (nt) | Origins and Notes | (SEQ ID NO: 68) | SEQ ID NO. |
| 5′ ITR | 145 | Wildtype AAV2 ITR | 1-145 | 21 |
| | | (AF043303.1) | | |
| Cloning site | 22 | N/A (SalI/NotI/MluI) | 146-167 | 46 |
| CMV enhancer | 381 | NCBI K03104.1 | 168-548 | 38 |
| | | (140 . . . 520) | | |
| CBA promoter | 279 | NCBI X00182.1 | 549-827 | 23 |
| | | (268 . . . 543) | | |
| Chimeric intron | 1013 | NCBI X00182.1 | 828-1840 | 39 |
| | | (544 . . . 1503) NCBI | | |
| | | V00882.1 (1250 . . . 1317); | | |
| | | XbaI cloning site | | |
| Cloning site | 33 | N/A; AgeI | 1841-1873 | 54 |
| 5′ UTR | 291 | NCBI NG_009168.1 | 1874-2164 | 40 |
| | | (5001-5291) | | |
| CLRN1 | 696 | NCBI NM_174878.3 | 2171-2866 | 19 |
| | | (20-715) | | |
| 3′ UTR | 1369 | NCBI NG_009168.1 | 2876-4244 | 41 |
| | | (50065-51433) | | |
| bGHpA | 225 | NCBI M57764.1 | 4245-4469 | 44 |
| | | (2326-2550) | | |
| Cloning site | 39 | N/A; | 4470-4508 | 49 |
| | | HindIII/EcoRI/PvuII/ | | |
| | | RsrII/A vrII | | |
| 3′ ITR | 145 | Wildtype AAV2 ITR | 4509-4653 | 22 |
| | | (AF043303.1) | | |

Exemplary Single Construct sequence 1
(SEQ ID NO: 64)
TTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGAGAGGGAGTGGCCAACTC
CATCACTAGGGGTTCCTTTGTCGACGCGGCCGCACGCGTGACATTGATTATTGACTAGTTATTA
ATAGTAATCAATTACGGGGTCATTAGTTCATAGCCCATATATGGAGTTCCGCGTTACATAACTT
ACGGTAAATGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGACTATTTACGGTA
AACTGCCCACTTGGCAGTACATCAAGTGTATCATATGCCAAGTACGCCCCCTATTGACGTCAAT
GACGGTAAATGGCCCGCCTGGCATTATGCCCAGTACATGACCTTATGGGACTTTCCTACTTGGC
AGTACATCTACGTATTAGTCATCGCTATTACCATGGGTCGAGGTGAGCCCCACGTTCTGCTTCA
CTCTCCCCATCTCCCCCCCCTCCCCACCCCCAATTTTGTATTTATTTATTTTTTAATTATTTTG
TGCAGCGATGGGGGCGGGGGGGGGGGGGGGGCGCGCGCCAGGCGGGGCGGGGCGGGGCGAGGGG
CGGGGCGGGGCGAGGCGGAGAGGTGCGGCGGCAGCCAATCAGAGCGGCGCGCTCCGAAAGTTTC
CTTTTATGGCGAGGCGGCGGCGGCGGCGGCCCTATAAAAAGCGAAGCGCGCGGCGGGCGGGAGT
CGCTGCGTTGCCTTCGCCCCGTGCCCCGCTCCGCGCCGCCTCGCGCCGCCCGCCCCGGCTCTGA
CTGACCGCGTTACTCCCACAGGTGAGCGGGCGGGACGGCCCTTCTCCTCCGGGCTGTAATTAGC
GCTTGGTTTAATGACGGCTCGTTTCTTTTCTGTGGCTGCGTGAAAGCCTTAAAGGGCTCCGGGA
GGGCCCTTTGTGCGGGGGGGAGCGGCTCGGGGGGTGCGTGCGTGTGTGTGTGCGTGGGGAGCGC
CGCGTGCGGCCCGCGCTGCCCGGCGGCTGTGAGCGCTGCGGGCGCGGCGCGGGGCTTTGTGCGC
TCCGCGTGTGCGCGAGGGGAGCGCGGCCGGGGGGGGTGCCCCGCGGTGCGGGGGGGCTGCGAGG
GGAACAAAGGCTGCGTGCGGGGTGTGTGCGTGGGGGGGTGAGCAGGGGGTGTGGGCGCGGCGGT
CGGGCTGTAACCCCCCCCTGCACCCCCCTCCCCGAGTTGCTGAGCACGGCCCGGCTTCGGGTGC
GGGGCTCCGTGCGGGGCGTGGCGCGGGGCTCGCCGTGCCGGGCGGGGGGTGGCGGCAGGTGGGG
GTGCCGGGCGGGGCGGGGCCGCCTCGGGCCGGGGAGGGCTCGGGGGAGGGGCGCGGCGGCCCCC
GGAGCGCCGGCGGCTGTCGAGGCGCGGCGAGCCGCAGCCATTGCCTTTTATGGTAATCGTGCGA
GAGGGCGCAGGGACTTCCTTTGTCCCAAATCTGTGCGGAGCCGAAATCTGGGAGGCGCCGCCGC
ACCCCCTCTAGCGGGCGCGGGGCGAAGCGGTGCGGCGCCGGCAGGAAGGAAATGGGGGGGGAGG
GCCTTCGTGCGTCGCCGCGCCGCCGTCCCCTTCTCCCTCTCCAGCCTCGGGGCTGTCCGCGGGG
GGACGGCTGCCTTCGGGGGGGACGGGGCAGGGGGGGGTTCGGCTTCTGGCGTGTGACCGGCGGC
TCTAGAGCCTCTGCTAACCATGTTCATGCCTTCTTCTTTTTCCTACAGCTCCTGGGCAACGTGC
TGGTTATTGTGACCGGTAGGAGATACTTGAAGGCAGTTTGAAAGACTTGTTTTACAGATTCTTA
GTCCAAAGATTTCCAATTAGGGAGAAGAAGCAGCAGAAAAGGAGAAAAGCCAAGTATGAGTGAT
GATGAGGCCTTCATCTACTGACATTTAACCTGGCGAGAACCGTCGATGGTGAAGTTGCCTTTTC
AGCTGGGAGCTGTCCGTTCAGCTTCCGTAATAAATGCAGTCAAAGAGGCAGTCCCTTCCCATTG
CTCACAAAGGTCTTGTTTTTGAACCTCGCCCTCACAGAAGCCGTTTCTCATCGCCACCATGCCA
AGCCAACAGAAGAAAATCATTTTTTGCATGGCCGGAGTGTTCAGTTTTGCATGTGCCCTCGGAG
TTGTGACAGCCTTGGGGACACCGTTGTGGATCAAAGCCACTGTCCTCTGCAAAACGGGAGCTCT
GCTCGTCAATGCCTCAGGGCAGGAGCTGGACAAGTTTATGGGTGAAATGCAGTACGGGCTTTTC
CACGGAGAGGGTGTGAGGCAGTGTGGGTTGGGAGCAAGGCCCTTTCGGTTCTCATTTTTTCCAG
ATTTGCTCAAAGCAATCCCAGTGAGCATCCACGTCAATGTCATTCTCTTCTCTGCCATCCTTAT
TGTGTTAACCATGGTGGGGACAGCCTTCTTCATGTACAATGCTTTTGGAAAACCTTTTGAAACT
CTGCATGGTCCCCTAGGGCTGTACCTTTTGAGCTTCATTTCAGGCTCCTGTGGCTGTCTTGTCA
TGATATTGTTTGCCTCTGAAGTGAAAATCCATCACCTCTCAGAAAAAATTGCAAATTATAAAGA
AGGGACTTATGTCTACAAAACGCAAAGTGAAAAATATACCACCTCATTCTGGGTCATTTTCTTT
TGCTTTTTTGTTCATTTTCTGAATGGGCTCCTAATACGACTTGCTGGATTTCAGTTCCCTTTTG
CAAAATCTAAAGACGCAGAAACAACTAATGTAGCTGCAGATCTAATGTACTAAGAGCTCAAGGC
AAACCTTTCTATAATTTTACAAGGGAGTAGACTTGCTTTGGTCACTTTTAGATGTGGTTAATTT
TGCATATCCTTTTAGTCTGCATATATTAAAGCATCAGGACCCTTCGTGACAATGTTTACAAATT
ACGTACTAAGGATACAGGCTGGAAAGTAAGGGAAGCAGAAGGAAGGCTTTGAAAAGTTGTTTTA
TCTGGTGGGAAATTGCTTGACCCAGGTAGTCAAAGGCAGTTGACTAGAATCGACAAATTGTTAC
TCCATATATATATATGTGTGTGTGTGTGTGTGTGTGTGTGTGTAAGATGTCTTCCTATCAAAAA
GATATCAAAGGCACATGGAATATATTTTAATAAAAACAAATAATATCTCTAATATATCCACACA
TTTGTTGCCAGATTTCAGAAAACTGAGCTGCAATCGCTTTCCTAAAACAGTAGTGTATTAAATG
AACATCTATAAAATGTATCAACACACATTTTAAAAAATTTGTTTAAAGTATACTCTTAGGCCAG
GCGTGGTGACTCACACCTGTAATTCCAGCACTTCAGGAGGCCAAGGTGGGAAGATCATTTGAGT
TCAGGAGTTCGAGTTACAGCCTGGGCAATAAAGTGAGACCCTGTCACTAACAAAATTAAAAAAT
AAAATAAATATAAAATATAGGCTTTAAAAAAGCATAGTCTTATTAACCATGTCTGTTGGTCAAA
ATCTGCAAACTCTAAAAGAAGAAAAGAAGAAAAAACCAACGTTAGGGTATTTTTCCTCCCGTGC
CTGAGTCCCAATTACATTCACGACAGTACTTTCAATGAACATAATTGTTAGGACCACTGAGGAA
TCATGAAAAATGATCTCTGCTTAGTACATTTGATGCAAAATGACTTATTAGGGGCTGTTTTTCT
AGCTATAGTGTCTCGAGTACTAATATGCAATTATGAAAATTATATTAAATCTGGGATTATGACG
GTATCACTGTATCATCTTGGTCTTGTTCTGGCTGTCACCAAGCATGACCCAGGTCAACTTTTTT
TTTCCCCTGAATTACCCATCAAATTGATCTGCAGCTGACTAAAGGCCACAGCTGAGCCTGGAAC
TGACCCTTCCTTCATCCTCAACCTGCTGTCCTCCAGAAAGCACCAAGGAAAAAGCAGAGAATGA
CAGCAAACAGATCACTAGGCCTCTGACCACAGGTGCTGAGTACTCAGCAGCCCTCATATAATAG
GTTTGAAAGTACTCCTTAAAATAAAACACTGTTTCCCTTTGGAACTATTTACAAGGATGAAACA
ACCGTATACCTGAGAAATAACTTGCTCTGGTGTCAATTCGCTATTCGCCAGCAGACATCAGAAC
ACACCGAGTTTCCAGATGCTCTGTGCCTTCTAGTTGCCAGCCATCTGTTGTTTGCCCCTCCCCC
GTGCCTTCCTTGACCCTGGAAGGTGCCACTCCCACTGTCCTTTCCTAATAAAATGAGGAAATTG
CATCGCATTGTCTGAGTAGGTGTCATTCTATTCTGGGGGGTGGGGTGGGGCAGGACAGCAAGGG
GGAGGATTGGGAAGACAATAGCAGGCATGCTGGGGATGCGGTGGGCTCTATGGAAGCTTGAATT
CAGCTGACGTGCCTCGGACCGTCCTAGGAGGAACCCCTAGTGATGGAGTTGGCCACTCCCTCTC
TGCGCGCTCGCTCGCTCACTGAGGCCGCCCGGGCAAAGCCCGGGCGTCGGGCGACCTTTGGTCG
CCCGGCCTCAGTGAGCGAGCGAGCGCGCAGAGAGGGAGTGGCCAA
Exemplary Single Construct sequence 2
(SEQ ID NO: 68)
TTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGAGAGGGAGTGGCCAACTC
CATCACTAGGGGTTCCTTTGTCGACGCGGCCGCACGCGTGACATTGATTATTGACTAGTTATTA
ATAGTAATCAATTACGGGGTCATTAGTTCATAGCCCATATATGGAGTTCCGCGTTACATAACTT
ACGGTAAATGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGACTATTTACGGTA
AACTGCCCACTTGGCAGTACATCAAGTGTATCATATGCCAAGTACGCCCCCTATTGACGTCAAT
GACGGTAAATGGCCCGCCTGGCATTATGCCCAGTACATGACCTTATGGGACTTTCCTACTTGGC
AGTACATCTACGTATTAGTCATCGCTATTACCATGGGTCGAGGTGAGCCCCACGTTCTGCTTCA
CTCTCCCCATCTCCCCCCCCTCCCCACCCCCAATTTTGTATTTATTTATTTTTTAATTATTTTG
TGCAGCGATGGGGGGGGGGGGGGGGGGGGGGGGCGCGCCAGGCGGGGCGGGGCGGGGCGAGGGG
CGGGGCGGGGCGAGGCGGAGAGGTGCGGCGGCAGCCAATCAGAGCGGCGCGCTCCGAAAGTTTC
CTTTTATGGCGAGGCGGCGGCGGCGGCGGCCCTATAAAAAGCGAAGCGCGCGGCGGGCGGGAGT
CGCTGCGTTGCCTTCGCCCCGTGCCCCGCTCCGCGCCGCCTCGCGCCGCCCGCCCCGGCTCTGA
CTGACCGCGTTACTCCCACAGGTGAGCGGGCGGGACGGCCCTTCTCCTCCGGGCTGTAATTAGC
GCTTGGTTTAATGACGGCTCGTTTCTTTTCTGTGGCTGCGTGAAAGCCTTAAAGGGCTCCGGGA
GGGCCCTTTGTGCGGGGGGGAGCGGCTCGGGGGGTGCGTGCGTGTGTGTGTGCGTGGGGAGCGC
CGCGTGCGGCCCGCGCTGCCCGGCGGCTGTGAGCGCTGCGGGCGCGGCGCGGGGCTTTGTGCGC
TCCGCGTGTGCGCGAGGGGAGCGCGGCCGGGGGCGGTGCCCCGCGGTGCGGGGGGGCTGCGAGG
GGAACAAAGGCTGCGTGCGGGGTGTGTGCGTGGGGGGGTGAGCAGGGGGTGTGGGCGCGGCGGT
CGGGCTGTAACCCCCCCCTGCACCCCCCTCCCCGAGTTGCTGAGCACGGCCCGGCTTCGGGTGC
GGGGCTCCGTGCGGGGCGTGGCGCGGGGCTCGCCGTGCCGGGCGGGGGGGGGCGGCAGGTGGGG
GTGCCGGGCGGGGGGGGGCCGCCTCGGGCCGGGGAGGGCTCGGGGGAGGGGCGCGGCGGCCCCC
GGAGCGCCGGCGGCTGTCGAGGCGCGGCGAGCCGCAGCCATTGCCTTTTATGGTAATCGTGCGA
GAGGGCGCAGGGACTTCCTTTGTCCCAAATCTGTGCGGAGCCGAAATCTGGGAGGCGCCGCCGC
ACCCCCTCTAGCGGGCGCGGGGCGAAGCGGTGCGGCGCCGGCAGGAAGGAAATGGGGGGGGAGG
GCCTTCGTGCGTCGCCGCGCCGCCGTCCCCTTCTCCCTCTCCAGCCTCGGGGCTGTCCGCGGGG
GGACGGCTGCCTTCGGGGGGGACGGGGCAGGGCGGGGTTCGGCTTCTGGCGTGTGACCGGCGGC
TCTAGAGCCTCTGCTAACCATGTTCATGCCTTCTTCTTTTTCCTACAGCTCCTGGGCAACGTGC
TGGTTATTGTGACCGGTAGGAGATACTTGAAGGCAGTTTGAAAGACTTGTTTTACAGATTCTTA
GTCCAAAGATTTCCAATTAGGGAGAAGAAGCAGCAGAAAAGGAGAAAAGCCAAGTATGAGTGAT
GATGAGGCCTTCATCTACTGACATTTAACCTGGCGAGAACCGTCGATGGTGAAGTTGCCTTTTC
AGCTGGGAGCTGTCCGTTCAGCTTCCGTAATAAATGCAGTCAAAGAGGCAGTCCCTTCCCATTG
CTCACAAAGGTCTTGTTTTTGAACCTCGCCCTCACAGAAGCCGTTTCTCATCGCCACCATGCCT
AGCCAGCAGAAGAAAATCATCTTCTGCATGGCCGGCGTGTTCAGCTTCGCCTGTGCTCTGGGAG
TTGTGACAGCCCTGGGAACCCCTCTGTGGATCAAAGCCACAGTGCTGTGCAAGACAGGCGCCCT
GCTGGTTAATGCCTCTGGCCAAGAGCTGGACAAGTTCATGGGCGAGATGCAGTACGGCCTGTTC
CATGGCGAAGGCGTCAGACAGTGTGGCCTGGGAGCCAGACCTTTCAGATTCAGCTTCTTCCCAG
ACCTGCTGAAGGCTATCCCCGTGTCCATCCACGTGAACGTGATCCTGTTCAGCGCCATCCTGAT
CGTGCTGACAATGGTCGGAACCGCCTTCTTCATGTACAACGCCTTCGGCAAGCCCTTCGAGACA
CTGCATGGACCTCTGGGCCTGTACCTGCTGAGCTTTATCAGCGGCAGCTGTGGCTGCCTGGTCA
TGATTCTGTTCGCCAGCGAAGTGAAGATCCACCACCTGAGCGAGAAGATCGCCAACTACAAAGA
GGGCACCTACGTCTACAAGACCCAGTCCGAGAAGTACACCACCAGCTTTTGGGTTATCTTCTTC
TGTTTCTTCGTGCACTTCCTGAACGGCCTGCTGATCAGACTGGCCGGCTTCCAGTTTCCATTCG
CCAAGAGCAAGGACGCCGAAACCACAAACGTGGCCGCCGATCTGATGTACTAAGAGCTCAAGGC
AAACCTTTCTATAATTTTACAAGGGAGTAGACTTGCTTTGGTCACTTTTAGATGTGGTTAATTT
TGCATATCCTTTTAGTCTGCATATATTAAAGCATCAGGACCCTTCGTGACAATGTTTACAAATT
ACGTACTAAGGATACAGGCTGGAAAGTAAGGGAAGCAGAAGGAAGGCTTTGAAAAGTTGTTTTA
TCTGGTGGGAAATTGCTTGACCCAGGTAGTCAAAGGCAGTTGACTAGAATCGACAAATTGTTAC
TCCATATATATATATGTGTGTGTGTGTGTGTGTGTGTGTGTGTAAGATGTCTTCCTATCAAAAA
GATATCAAAGGCACATGGAATATATTTTAATAAAAACAAATAATATCTCTAATATATCCACACA
TTTGTTGCCAGATTTCAGAAAACTGAGCTGCAATCGCTTTCCTAAAACAGTAGTGTATTAAATG
AACATCTATAAAATGTATCAACACACATTTTAAAAAATTTGTTTAAAGTATACTCTTAGGCCAG
GCGTGGTGACTCACACCTGTAATTCCAGCACTTCAGGAGGCCAAGGTGGGAAGATCATTTGAGT
TCAGGAGTTCGAGTTACAGCCTGGGCAATAAAGTGAGACCCTGTCACTAACAAAATTAAAAAAT
AAAATAAATATAAAATATAGGCTTTAAAAAAGCATAGTCTTATTAACCATGTCTGTTGGTCAAA
ATCTGCAAACTCTAAAAGAAGAAAAGAAGAAAAAACCAACGTTAGGGTATTTTTCCTCCCGTGC
CTGAGTCCCAATTACATTCACGACAGTACTTTCAATGAACATAATTGTTAGGACCACTGAGGAA
TCATGAAAAATGATCTCTGCTTAGTACATTTGATGCAAAATGACTTATTAGGGGCTGTTTTTCT
AGCTATAGTGTCTCGAGTACTAATATGCAATTATGAAAATTATATTAAATCTGGGATTATGACG
GTATCACTGTATCATCTTGGTCTTGTTCTGGCTGTCACCAAGCATGACCCAGGTCAACTTTTTT
TTTCCCCTGAATTACCCATCAAATTGATCTGCAGCTGACTAAAGGCCACAGCTGAGCCTGGAAC
TGACCCTTCCTTCATCCTCAACCTGCTGTCCTCCAGAAAGCACCAAGGAAAAAGCAGAGAATGA
CAGCAAACAGATCACTAGGCCTCTGACCACAGGTGCTGAGTACTCAGCAGCCCTCATATAATAG
GTTTGAAAGTACTCCTTAAAATAAAACACTGTTTCCCTTTGGAACTATTTACAAGGATGAAACA
ACCGTATACCTGAGAAATAACTTGCTCTGGTGTCAATTCGCTATTCGCCAGCAGACATCAGAAC
ACACCGAGTTTCCAGATGCTCTGTGCCTTCTAGTTGCCAGCCATCTGTTGTTTGCCCCTCCCCC
GTGCCTTCCTTGACCCTGGAAGGTGCCACTCCCACTGTCCTTTCCTAATAAAATGAGGAAATTG
CATCGCATTGTCTGAGTAGGTGTCATTCTATTCTGGGGGGTGGGGTGGGGCAGGACAGCAAGGG
GGAGGATTGGGAAGACAATAGCAGGCATGCTGGGGATGCGGTGGGCTCTATGGAAGCTTGAATT
CAGCTGACGTGCCTCGGACCGTCCTAGGAGGAACCCCTAGTGATGGAGTTGGCCACTCCCTCTC
TGCGCGCTCGCTCGCTCACTGAGGCCGCCCGGGCAAAGCCCGGGCGTCGGGCGACCTTTGGTCG
CCCGGCCTCAGTGAGCGAGCGAGCGCGCAGAGAGGGAGTGGCCAA

Multiple AAV Construct Compositions

The present disclosure recognizes that some coding sequences encoding a protein (e.g., clarin 1 protein) may be delivered by dividing the coding sequence into multiple portions, which are each included in a different construct. In some embodiments, provided herein are compositions or systems comprising at least two different constructs (e.g., two, three, four, five, or six). In some embodiments, each of the at least two different constructs includes a coding sequence that encodes a different portion of a coding region (e.g., encoding a target protein, e.g., an inner ear target protein, e.g., an eye target protein, e.g., e.g., a clarin 1 protein), each of the encoded portions being at least 10 amino acids (e.g., at least about 10 amino acids, at least about 20 amino acids, at least about 30 amino acids, at least about 60 amino acids, at least about 70 amino acids, at least about 80 amino acids, at least about 90 amino acids, at least about 100 amino acids, at least about 110 amino acids, at least about 120 amino acids, at least about 130 amino acids, at least about 140 amino acids, at least about 150 amino acids, at least about 160 amino acids, at least about 170 amino acids, at least about 180 amino acids, at least about 190 amino acids, at least about 200 amino acids, at least about 210 amino acids, at least about 220 amino acids, at least about 230 amino acids, at least about 240 amino acids, at least about 250 amino acids, at least about 260 amino acids, at least about 270 amino acids, at least about 280 amino acids, at least about 290 amino acids, at least about 300 amino acids, at least about 310 amino acids, at least about 320 amino acids, at least about 330 amino acids, at least about 340 amino acids, at least about 350 amino acids, at least about 360 amino acids, at least about 370 amino acids, at least about 380 amino acids, at least about 390 amino acids, at least about 400 amino acids, at least about 410 amino acids, at least about 420 amino acids, at least about 430 amino acids, at least about 440 amino acids, at least about 450 amino acids, at least about 460 amino acids, at least about 470 amino acids, at least about 480 amino acids, at least about 490 amino acids, at least about 500 amino acids, at least about 510 amino acids, at least about 520 amino acids, at least about 530 amino acids, at least about 540 amino acids, at least about 550 amino acids, at least about 560 amino acids, at least about 570 amino acids, at least about 580 amino acids, at least about 590 amino acids, at least about 600 amino acids, at least about 610 amino acids, at least about 620 amino acids, at least about 630 amino acids, at least about 640 amino acids, at least about 650 amino acids, at least about 660 amino acids, at least about 670 amino acids, at least about 680 amino acids, at least about 690 amino acids, at least about 700 amino acids, at least about 710 amino acids, at least about 720 amino acids, at least about 730 amino acids, at least about 740 amino acids, at least about 750 amino acids, at least about 760 amino acids, at least about 770 amino acids, at least about 780 amino acids, at least about 790 amino acids, at least about 800 amino acids, at least about 810 amino acids, or at least about 820 amino acids) where the amino acid sequence of each of the encoded portions may optionally partially overlap with the amino acid sequence of a different one of the encoded portions; no single construct of the at least two different constructs encodes the active target protein; and, when introduced into a subject cell (e.g., an animal cell, e.g., a primate cell, e.g., a human cell), the at least two different constructs undergo homologous recombination with each other, where the recombined nucleic acid encodes an active target protein (e.g., a gene product encoded by a CLRN1 gene or a characteristic portion thereof). In some embodiments, one of the nucleic acid constructs can include a coding sequence that encodes a portion of a target protein (e.g., an inner ear target protein, e.g., a clarin 1 protein), where the encoded portion is at most about 820 amino acids (e.g., at most about 10 amino acids, at most about 20 amino acids, at most about 30 amino acids, at most about 60 amino acids, at most about 70 amino acids, at most about 80 amino acids, at most about 90 amino acids, at most about 100 amino acids, at most about 110 amino acids, at most about 120 amino acids, at most about 130 amino acids, at most about 140 amino acids, at most about 150 amino acids, at most about 160 amino acids, at most about 170 amino acids, at most about 180 amino acids, at most about 190 amino acids, at most about 200 amino acids, at most about 210 amino acids, at most about 220 amino acids, at most about 230 amino acids, at most about 240 amino acids, at most about 250 amino acids, at most about 260 amino acids, at most about 270 amino acids, at most about 280 amino acids, at most about 290 amino acids, at most about 300 amino acids, at most about 310 amino acids, at most about 320 amino acids, at most about 330 amino acids, at most about 340 amino acids, at most about 350 amino acids, at most about 360 amino acids, at most about 370 amino acids, at most about 380 amino acids, at most about 390 amino acids, at most about 400 amino acids, at most about 410 amino acids, at most about 420 amino acids, at most about 430 amino acids, at most about 440 amino acids, at most about 450 amino acids, at most about 460 amino acids, at most about 470 amino acids, at most about 480 amino acids, at most about 490 amino acids, at most about 500 amino acids, at most about 510 amino acids, at most about 520 amino acids, at most about 530 amino acids, at most about 540 amino acids, at most about 550 amino acids, at most about 560 amino acids, at most about 570 amino acids, at most about 580 amino acids, at most about 590 amino acids, at most about 600 amino acids, at most about 610 amino acids, at most about 620 amino acids, at most about 630 amino acids, at most about 640 amino acids, at most about 650 amino acids, at most about 660 amino acids, at most about 670 amino acids, at most about 680 amino acids, at most about 690 amino acids, at most about 700 amino acids, at most about 710 amino acids, at most about 720 amino acids, at most about 730 amino acids, at most about 740 amino acids, at most about 750 amino acids, at most about 760 amino acids, at most about 770 amino acids, at most about 780 amino acids, at most about 790 amino acids, at most about 800 amino acids, at most about 810 amino acids, or at most about 820 amino acids).

In some embodiments, at least one of the constructs includes a nucleotide sequence spanning two neighboring exons of target genomic DNA (e.g., an inner ear target genomic DNA, e.g., CLRN1 genomic DNA), and lacks the intronic sequence that naturally occurs between the two neighboring exons.

In some embodiments, an amino acid sequence of an encoded portion of each of the constructs does not overlap, even in part, with an amino acid sequence of a different one of the encoded portions. In some embodiments, an amino acid sequence of an encoded portion of a construct partially overlaps with an amino acid sequence of an encoded portion of a different construct. In some embodiments, an amino acid sequence of an encoded portion of each construct partially overlaps with an amino acid sequence of an encoded portion of at least one different construct. In some embodiments, an overlapping amino acid sequence is between about 10 amino acid residues to about 300 amino acids, or any of the subranges of this range (e.g., about 10 amino acids, about 20 amino acids, about 30 amino acids, about 60 amino acids, about 70 amino acids, about 80 amino acids, about 90 amino acids, about 100 amino acids, about 110 amino acids, about 120 amino acids, about 130 amino acids, about 140 amino acids, about 150 amino acids, about 160 amino acids, about 170 amino acids, about 180 amino acids, about 190 amino acids, about 200 amino acids, about 210 amino acids, about 220 amino acids, about 230 amino acids, about 240 amino acids, about 250 amino acids, about 260 amino acids, about 270 amino acids, about 280 amino acids, about 290 amino acids, about 300 amino acids, about 310 amino acids, about 320 amino acids, about 330 amino acids, about 340 amino acids, about 350 amino acids, about 360 amino acids, about 370 amino acids, about 380 amino acids, about 390 amino acids, about 400 amino acids, about 410 amino acids, about 420 amino acids, about 430 amino acids, about 440 amino acids, about 450 amino acids, about 460 amino acids, about 470 amino acids, about 480 amino acids, about 490 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, about 600 amino acids, about 610 amino acids, about 620 amino acids, about 630 amino acids, about 640 amino acids, about 650 amino acids, about 660 amino acids, about 670 amino acids, about 680 amino acids, about 690 amino acids, about 700 amino acids, about 710 amino acids, about 720 amino acids, about 730 amino acids, about 740 amino acids, about 750 amino acids, about 760 amino acids, about 770 amino acids, about 780 amino acids, about 790 amino acids, about 800 amino acids, about 810 amino acids, or about 820 amino acids in length).

In some examples, a desired gene product (e.g., a therapeutic gene product) is encoded by at least two different constructs. In some embodiments, each of at least two different constructs includes a different segment of an intron, where the intron includes a nucleotide sequence of an intron that is present in a target genomic DNA (e.g., an inner ear cell target genomic DNA or an eye cell target genomic DNA (e.g., CLRN1 genomic DNA) (e.g., any of the exemplary introns in SEQ ID NO: 5 described herein). In some embodiments, different intron segments overlap. In some embodiments, different intron segments overlap in sequence by at most about 12,000 nucleotides (e.g., at most about 100 nucleotides, at most about 200 nucleotides, at most about 300 nucleotides, at most about 600 nucleotides, at most about 700 nucleotides, at most about 800 nucleotides, at most about 900 nucleotides, at most about 1,000 nucleotides, at most about 1,100 nucleotides, at most about 1,200 nucleotides, at most about 1,300 nucleotides, at most about 1,400 nucleotides, at most about 1,500 nucleotides, at most about 1,600 nucleotides, at most about 1,700 nucleotides, at most about 1,800 nucleotides, at most about 1,900 nucleotides, at most about 2,000 nucleotides, at most about 2,100 nucleotides, at most about 2,200 nucleotides, at most about 2,300 nucleotides, at most about 2,400 nucleotides, at most about 2,500 nucleotides, at most about 2,600 nucleotides, at most about 2,700 nucleotides, at most about 2,800 nucleotides, at most about 2,900 nucleotides, at most about 3,000 nucleotides, at most about 3,100 nucleotides, at most about 3,200 nucleotides, at most about 3,300 nucleotides, at most about 3,400 nucleotides, at most about 3,500 nucleotides, at most about 3,600 nucleotides, at most about 3,700 nucleotides, at most about 3,800 nucleotides, at most about 3,900 nucleotides, at most about 4,000 nucleotides, at most about 4,100 nucleotides, at most about 4,200 nucleotides, at most about 4,300 nucleotides, at most about 4,400 nucleotides, at most about 4,500 nucleotides, at most about 4,600 nucleotides, at most about 4,700 nucleotides, at most about 4,800 nucleotides, at most about 4,900 nucleotides, at most about 5,000 nucleotides, at most about 5,100 nucleotides, at most about 5,200 nucleotides, at most about 5,300 nucleotides, at most about 5,400 nucleotides, at most about 5,500 nucleotides, at most about 5,600 nucleotides, at most about 5,700 nucleotides, at most about 5,800 nucleotides, at most about 5,900 nucleotides, at most about 6,000 nucleotides, at most about 6,100 nucleotides, at most about 6,200 nucleotides, at most about 6,300 nucleotides, at most about 6,400 nucleotides, at most about 6,500 nucleotides, at most about 6,600 nucleotides, at most about 6,700 nucleotides, at most about 6,800 nucleotides, at most about 6,900 nucleotides, at most about 7,000 nucleotides, at most about 7,100 nucleotides, at most about 7,200 nucleotides, at most about 7,300 nucleotides, at most about 7,400 nucleotides, at most about 7,500 nucleotides, at most about 7,600 nucleotides, at most about 7,700 nucleotides, at most about 7,800 nucleotides, at most about 7,900 nucleotides, at most about 8,000 nucleotides, at most about 8,100 nucleotides, at most about 8,200 nucleotides, at most about 8,300 nucleotides, at most about 8,400 nucleotides, at most about 8,500 nucleotides, at most about 8,600 nucleotides, at most about 8,700 nucleotides, at most about 8,800 nucleotides, at most about 8,900 nucleotides, at most about 9,000 nucleotides, at most about 9,100 nucleotides, at most about 9,200 nucleotides, at most about 9,300 nucleotides, at most about 9,400 nucleotides, at most about 9,500 nucleotides, at most about 9,600 nucleotides, at most about 9,700 nucleotides, at most about 9,800 nucleotides, at most about 9,900 nucleotides, at most about 10,000 nucleotides, at most about 10,100 nucleotides, at most about 10,200 nucleotides, at most about 10,300 nucleotides, at most about 10,400 nucleotides, at most about 10,500 nucleotides, at most about 10,600 nucleotides, at most about 10,700 nucleotides, at most about 10,800 nucleotides, at most about 10,900 nucleotides, at most about 11,000 nucleotides, at most about 11,100 nucleotides, at most about 11,200 nucleotides, at most about 11,300 nucleotides, at most about 11,400 nucleotides, at most about 11,500 nucleotides, at most about 11,600 nucleotides, at most about 11,700 nucleotides, at most about 11,800 nucleotides, at most about 11,900 nucleotides, or at most about 12,000 nucleotides) in length. In some embodiments, the overlapping nucleotide sequence in any two of the different constructs can include part or all of one or more exons of a target gene (e.g., an inner ear cell target gene or an eye cell target gene (e.g., a CLRN1 gene) (e.g., any one or more of the exemplary exons in SEQ ID NO: 5 described herein).

In some embodiments, a composition or system is or comprises two, three, four, or five different constructs. In compositions where the number of different constructs in the composition is two, the first of the two different constructs can include a coding sequence that encodes an N-terminal portion of a protein (e.g., clarin 1 protein), which may be referred to as a lead portion, a first construct, or a 5′ portion (e.g., an N-terminal portion of an inner ear cell protein, e.g., an N-terminal portion of an eye cell protein, e.g., an N-terminal portion of a clarin 1 protein). In some examples, an N-terminal portion of the target gene is at least about 10 amino acids (e.g., at least about 10 amino acids, at least about 20 amino acids, at least about 30 amino acids, at least about 60 amino acids, at least about 70 amino acids, at least about 80 amino acids, at least about 90 amino acids, at least about 100 amino acids, at least about 110 amino acids, at least about 120 amino acids, at least about 130 amino acids, at least about 140 amino acids, at least about 150 amino acids, at least about 160 amino acids, at least about 170 amino acids, at least about 180 amino acids, at least about 190 amino acids, at least about 200 amino acids, at least about 210 amino acids, at least about 220 amino acids, at least about 230 amino acids, at least about 240 amino acids, at least about 250 amino acids, at least about 260 amino acids, at least about 270 amino acids, at least about 280 amino acids, at least about 290 amino acids, at least about 300 amino acids, at least about 310 amino acids, at least about 320 amino acids, at least about 330 amino acids, at least about 340 amino acids, at least about 350 amino acids, at least about 360 amino acids, at least about 370 amino acids, at least about 380 amino acids, at least about 390 amino acids, at least about 400 amino acids, at least about 410 amino acids, at least about 420 amino acids, at least about 430 amino acids, at least about 440 amino acids, at least about 450 amino acids, at least about 460 amino acids, at least about 470 amino acids, at least about 480 amino acids, at least about 490 amino acids, at least about 500 amino acids, at least about 510 amino acids, at least about 520 amino acids, at least about 530 amino acids, at least about 540 amino acids, at least about 550 amino acids, at least about 560 amino acids, at least about 570 amino acids, at least about 580 amino acids, at least about 590 amino acids, at least about 600 amino acids, at least about 610 amino acids, at least about 620 amino acids, at least about 630 amino acids, at least about 640 amino acids, at least about 650 amino acids, at least about 660 amino acids, at least about 670 amino acids, at least about 680 amino acids, at least about 690 amino acids, at least about 700 amino acids, at least about 710 amino acids, at least about 720 amino acids, at least about 730 amino acids, at least about 740 amino acids, at least about 750 amino acids, at least about 760 amino acids, at least about 770 amino acids, at least about 780 amino acids, at least about 790 amino acids, at least about 800 amino acids, at least about 810 amino acids, or at least about 820 amino acids) in length. In some examples, a first construct includes one or both of a promoter (e.g., any of the promoters described herein or known in the art) and a Kozak sequence (e.g., any of the exemplary Kozak sequences described herein or known in the art). In some examples, a first construct includes a promoter that is an inducible promoter, a constitutive promoter, or a tissue-specific promoter. In some examples, a second of the two different constructs includes a coding sequence that encodes a C-terminal portion of the protein, which may be referred to as a terminal portion, a second construct, or a 3′ portion (e.g., a C-terminal portion of an inner ear cell target protein, e.g., a C-terminal portion of an eye cell target protein, e.g., a C-terminal portion of a clarin 1 protein). In some examples, a C-terminal portion of the target protein is at least about 10 amino acids (e.g., at least about 10 amino acids, at least about 20 amino acids, at least about 30 amino acids, at least about 60 amino acids, at least about 70 amino acids, at least about 80 amino acids, at least about 90 amino acids, at least about 100 amino acids, at least about 110 amino acids, at least about 120 amino acids, at least about 130 amino acids, at least about 140 amino acids, at least about 150 amino acids, at least about 160 amino acids, at least about 170 amino acids, at least about 180 amino acids, at least about 190 amino acids, at least about 200 amino acids, at least about 210 amino acids, at least about 220 amino acids, at least about 230 amino acids, at least about 240 amino acids, at least about 250 amino acids, at least about 260 amino acids, at least about 270 amino acids, at least about 280 amino acids, at least about 290 amino acids, at least about 300 amino acids, at least about 310 amino acids, at least about 320 amino acids, at least about 330 amino acids, at least about 340 amino acids, at least about 350 amino acids, at least about 360 amino acids, at least about 370 amino acids, at least about 380 amino acids, at least about 390 amino acids, at least about 400 amino acids, at least about 410 amino acids, at least about 420 amino acids, at least about 430 amino acids, at least about 440 amino acids, at least about 450 amino acids, at least about 460 amino acids, at least about 470 amino acids, at least about 480 amino acids, at least about 490 amino acids, at least about 500 amino acids, at least about 510 amino acids, at least about 520 amino acids, at least about 530 amino acids, at least about 540 amino acids, at least about 550 amino acids, at least about 560 amino acids, at least about 570 amino acids, at least about 580 amino acids, at least about 590 amino acids, at least about 600 amino acids, at least about 610 amino acids, at least about 620 amino acids, at least about 630 amino acids, at least about 640 amino acids, at least about 650 amino acids, at least about 660 amino acids, at least about 670 amino acids, at least about 680 amino acids, at least about 690 amino acids, at least about 700 amino acids, at least about 710 amino acids, at least about 720 amino acids, at least about 730 amino acids, at least about 740 amino acids, at least about 750 amino acids, at least about 760 amino acids, at least about 770 amino acids, at least about 780 amino acids, at least about 790 amino acids, at least about 800 amino acids, at least about 810 amino acids, or at least about 820 amino acids) in length. In some examples, a second construct further includes a poly(A) sequence.

In some examples where the number of different constructs in the composition is two, an N-terminal portion encoded by one of the two constructs can include a portion including amino acid position 1 to about amino acid position 820, or any subrange of this range (e.g., amino acid 1 to at least about amino acid 10, amino acid 1 to at least about amino acid 20, amino acid 1 to at least about amino acid 30, amino acid 1 to at least about amino acid 60, amino acid 1 to at least about amino acid 70, amino acid 1 to at least about amino acid 80, amino acid 1 to at least about amino acid 90, amino acid 1 to at least about amino acid 100, amino acid 1 to at least about amino acid 110, amino acid 1 to at least about amino acid 120, amino acid 1 to at least about amino acid 130, amino acid 1 to at least about amino acid 140, amino acid 1 to at least about amino acid 150, amino acid 1 to at least about amino acid 160, amino acid 1 to at least about amino acid 170, amino acid 1 to at least about amino acid 180, amino acid 1 to at least about amino acid 190, amino acid 1 to at least about amino acid 200, amino acid 1 to at least about amino acid 210, amino acid 1 to at least about amino acid 220, amino acid 1 to at least about amino acid 230, amino acid 1 to at least about amino acid 240, amino acid 1 to at least about amino acid 250, amino acid 1 to at least about amino acid 260, amino acid 1 to at least about amino acid 270, amino acid 1 to at least about amino acid 280, amino acid 1 to at least about amino acid 290, amino acid 1 to at least about amino acid 300, amino acid 1 to at least about amino acid 310, amino acid 1 to at least about amino acid 320, amino acid 1 to at least about amino acid 330, amino acid 1 to at least about amino acid 340, amino acid 1 to at least about amino acid 350, amino acid 1 to at least about amino acid 360, amino acid 1 to at least about amino acid 370, amino acid 1 to at least about amino acid 380, amino acid 1 to at least about amino acid 390, amino acid 1 to at least about amino acid 400, amino acid 1 to at least about amino acid 410, amino acid 1 to at least about amino acid 420, amino acid 1 to at least about amino acid 430, amino acid 1 to at least about amino acid 440, amino acid 1 to at least about amino acid 450, amino acid 1 to at least about amino acid 460, amino acid 1 to at least about amino acid 470, amino acid 1 to at least about amino acid 480, amino acid 1 to at least about amino acid 490, amino acid 1 to at least about amino acid 500, amino acid 1 to at least about amino acid 510, amino acid 1 to at least about amino acid 520, amino acid 1 to at least about amino acid 530, amino acid 1 to at least about amino acid 540, amino acid 1 to at least about amino acid 550, amino acid 1 to at least about amino acid 560, amino acid 1 to at least about amino acid 570, amino acid 1 to at least about amino acid 580, amino acid 1 to at least about amino acid 590, amino acid 1 to at least about amino acid 600, amino acid 1 to at least about amino acid 610, amino acid 1 to at least about amino acid 620, amino acid 1 to at least about amino acid 630, amino acid 1 to at least about amino acid 640, amino acid 1 to at least about amino acid 650, amino acid 1 to at least about amino acid 660, amino acid 1 to at least about amino acid 670, amino acid 1 to at least about amino acid 680, amino acid 1 to at least about amino acid 690, amino acid 1 to at least about amino acid 700, amino acid 1 to at least about amino acid 710, amino acid 1 to at least about amino acid 720, amino acid 1 to at least about amino acid 730, amino acid 1 to at least about amino acid 740, amino acid 1 to at least about amino acid 750, amino acid 1 to at least about amino acid 760, amino acid 1 to at least about amino acid 770, amino acid 1 to at least about amino acid 780, amino acid 1 to at least about amino acid 790, amino acid 1 to at least about amino acid 800, amino acid 1 to at least about amino acid 810, or amino acid 1 to at least about amino acid 820) of an inner ear cell target protein or an eye cell target protein (e.g., SEQ ID NO: 10, 11, 12, 13, or 14). In some examples where the number of different constructs in the composition is two, an N-terminal portion of the precursor inner ear cell target protein or eye cell target protein can include a portion including at most amino acid position 1 to amino acid position 820 or any subrange of this range (e.g., amino acid 1 to at most about amino acid 10, amino acid 1 to at most about amino acid 20, amino acid 1 to at most about amino acid 30, amino acid 1 to at most about amino acid 60, amino acid 1 to at most about amino acid 70, amino acid 1 to at most about amino acid 80, amino acid 1 to at most about amino acid 90, amino acid 1 to at most about amino acid 100, amino acid 1 to at most about amino acid 110, amino acid 1 to at most about amino acid 120, amino acid 1 to at most about amino acid 130, amino acid 1 to at most about amino acid 140, amino acid 1 to at most about amino acid 150, amino acid 1 to at most about amino acid 160, amino acid 1 to at most about amino acid 170, amino acid 1 to at most about amino acid 180, amino acid 1 to at most about amino acid 190, amino acid 1 to at most about amino acid 200, amino acid 1 to at most about amino acid 210, amino acid 1 to at most about amino acid 220, amino acid 1 to at most about amino acid 230, amino acid 1 to at most about amino acid 240, amino acid 1 to at most about amino acid 250, amino acid 1 to at most about amino acid 260, amino acid 1 to at most about amino acid 270, amino acid 1 to at most about amino acid 280, amino acid 1 to at most about amino acid 290, amino acid 1 to at most about amino acid 300, amino acid 1 to at most about amino acid 310, amino acid 1 to at most about amino acid 320, amino acid 1 to at most about amino acid 330, amino acid 1 to at most about amino acid 340, amino acid 1 to at most about amino acid 350, amino acid 1 to at most about amino acid 360, amino acid 1 to at most about amino acid 370, amino acid 1 to at most about amino acid 380, amino acid 1 to at most about amino acid 390, amino acid 1 to at most about amino acid 400, amino acid 1 to at most about amino acid 410, amino acid 1 to at most about amino acid 420, amino acid 1 to at most about amino acid 430, amino acid 1 to at most about amino acid 440, amino acid 1 to at most about amino acid 450, amino acid 1 to at most about amino acid 460, amino acid 1 to at most about amino acid 470, amino acid 1 to at most about amino acid 480, amino acid 1 to at most about amino acid 490, amino acid 1 to at most about amino acid 500, amino acid 1 to at most about amino acid 510, amino acid 1 to at most about amino acid 520, amino acid 1 to at most about amino acid 530, amino acid 1 to at most about amino acid 540, amino acid 1 to at most about amino acid 550, amino acid 1 to at most about amino acid 560, amino acid 1 to at most about amino acid 570, amino acid 1 to at most about amino acid 580, amino acid 1 to at most about amino acid 590, amino acid 1 to at most about amino acid 600, amino acid 1 to at most about amino acid 610, amino acid 1 to at most about amino acid 620, amino acid 1 to at most about amino acid 630, amino acid 1 to at most about amino acid 640, amino acid 1 to at most about amino acid 650, amino acid 1 to at most about amino acid 660, amino acid 1 to at most about amino acid 670, amino acid 1 to at most about amino acid 680, amino acid 1 to at most about amino acid 690, amino acid 1 to at most about amino acid 700, amino acid 1 to at most about amino acid 710, amino acid 1 to at most about amino acid 720, amino acid 1 to at most about amino acid 730, amino acid 1 to at most about amino acid 740, amino acid 1 to at most about amino acid 750, amino acid 1 to at most about amino acid 760, amino acid 1 to at most about amino acid 770, amino acid 1 to at most about amino acid 780, amino acid 1 to at most about amino acid 790, amino acid 1 to at most about amino acid 800, amino acid 1 to at most about amino acid 810, or amino acid 1 to at most about amino acid 820) of an inner ear cell target protein or an eye cell target protein (e.g., SEQ ID NO: 10, 11, 12, 13, or 14)

In some examples where the number of different constructs in the composition is two, a C-terminal portion encoded by one of the two constructs can include a portion including the final amino acid (e.g., about amino acid position 820) to about amino acid position 1, or any subrange of this range (e.g., amino acid 820 to at least about amino acid 10, amino acid 820 to at least about amino acid 20, amino acid 820 to at least about amino acid 30, amino acid 820 to at least about amino acid 60, amino acid 820 to at least about amino acid 70, amino acid 820 to at least about amino acid 80, amino acid 820 to at least about amino acid 90, amino acid 820 to at least about amino acid 100, amino acid 820 to at least about amino acid 110, amino acid 820 to at least about amino acid 120, amino acid 820 to at least about amino acid 130, amino acid 820 to at least about amino acid 140, amino acid 820 to at least about amino acid 150, amino acid 820 to at least about amino acid 160, amino acid 820 to at least about amino acid 170, amino acid 820 to at least about amino acid 180, amino acid 820 to at least about amino acid 190, amino acid 820 to at least about amino acid 200, amino acid 820 to at least about amino acid 210, amino acid 820 to at least about amino acid 220, amino acid 820 to at least about amino acid 230, amino acid 820 to at least about amino acid 240, amino acid 820 to at least about amino acid 250, amino acid 820 to at least about amino acid 260, amino acid 820 to at least about amino acid 270, amino acid 820 to at least about amino acid 280, amino acid 820 to at least about amino acid 290, amino acid 820 to at least about amino acid 300, amino acid 820 to at least about amino acid 310, amino acid 820 to at least about amino acid 320, amino acid 820 to at least about amino acid 330, amino acid 820 to at least about amino acid 340, amino acid 820 to at least about amino acid 350, amino acid 820 to at least about amino acid 360, amino acid 820 to at least about amino acid 370, amino acid 820 to at least about amino acid 380, amino acid 820 to at least about amino acid 390, amino acid 820 to at least about amino acid 400, amino acid 820 to at least about amino acid 410, amino acid 820 to at least about amino acid 420, amino acid 820 to at least about amino acid 430, amino acid 820 to at least about amino acid 440, amino acid 820 to at least about amino acid 450, amino acid 820 to at least about amino acid 460, amino acid 820 to at least about amino acid 470, amino acid 820 to at least about amino acid 480, amino acid 820 to at least about amino acid 490, amino acid 820 to at least about amino acid 500, amino acid 820 to at least about amino acid 510, amino acid 820 to at least about amino acid 520, amino acid 820 to at least about amino acid 530, amino acid 820 to at least about amino acid 540, amino acid 820 to at least about amino acid 550, amino acid 820 to at least about amino acid 560, amino acid 820 to at least about amino acid 570, amino acid 820 to at least about amino acid 580, amino acid 820 to at least about amino acid 590, amino acid 820 to at least about amino acid 600, amino acid 820 to at least about amino acid 610, amino acid 820 to at least about amino acid 620, amino acid 820 to at least about amino acid 630, amino acid 820 to at least about amino acid 640, amino acid 820 to at least about amino acid 650, amino acid 820 to at least about amino acid 660, amino acid 820 to at least about amino acid 670, amino acid 820 to at least about amino acid 680, amino acid 820 to at least about amino acid 690, amino acid 820 to at least about amino acid 700, amino acid 820 to at least about amino acid 710, amino acid 820 to at least about amino acid 720, amino acid 820 to at least about amino acid 730, amino acid 820 to at least about amino acid 740, amino acid 820 to at least about amino acid 750, amino acid 820 to at least about amino acid 760, amino acid 820 to at least about amino acid 770, amino acid 820 to at least about amino acid 780, amino acid 820 to at least about amino acid 790, amino acid 820 to at least about amino acid 800, amino acid 820 to at least about amino acid 810, or amino acid 820 to at least about amino acid 820) of an inner ear cell target protein or an eye cell target protein (e.g., SEQ ID NO: 10, 11, 12, 13, or 14). In some examples where the number of different constructs in the composition is two, a C-terminal portion of the precursor inner ear cell target protein or an eye cell target protein can include a portion including the final amino acid (e.g., about amino acid position 820) to at most about amino acid position 1, or any subrange of this range (e.g., amino acid 820 to at most about amino acid 10, amino acid 820 to at most about amino acid 20, amino acid 820 to at most about amino acid 30, amino acid 820 to at most about amino acid 60, amino acid 820 to at most about amino acid 70, amino acid 820 to at most about amino acid 80, amino acid 820 to at most about amino acid 90, amino acid 820 to at most about amino acid 100, amino acid 820 to at most about amino acid 110, amino acid 820 to at most about amino acid 120, amino acid 820 to at most about amino acid 130, amino acid 820 to at most about amino acid 140, amino acid 820 to at most about amino acid 150, amino acid 820 to at most about amino acid 160, amino acid 820 to at most about amino acid 170, amino acid 820 to at most about amino acid 180, amino acid 820 to at most about amino acid 190, amino acid 820 to at most about amino acid 200, amino acid 820 to at most about amino acid 210, amino acid 820 to at most about amino acid 220, amino acid 820 to at most about amino acid 230, amino acid 820 to at most about amino acid 240, amino acid 820 to at most about amino acid 250, amino acid 820 to at most about amino acid 260, amino acid 820 to at most about amino acid 270, amino acid 820 to at most about amino acid 280, amino acid 820 to at most about amino acid 290, amino acid 820 to at most about amino acid 300, amino acid 820 to at most about amino acid 310, amino acid 820 to at most about amino acid 320, amino acid 820 to at most about amino acid 330, amino acid 820 to at most about amino acid 340, amino acid 820 to at most about amino acid 350, amino acid 820 to at most about amino acid 360, amino acid 820 to at most about amino acid 370, amino acid 820 to at most about amino acid 380, amino acid 820 to at most about amino acid 390, amino acid 820 to at most about amino acid 400, amino acid 820 to at most about amino acid 410, amino acid 820 to at most about amino acid 420, amino acid 820 to at most about amino acid 430, amino acid 820 to at most about amino acid 440, amino acid 820 to at most about amino acid 450, amino acid 820 to at most about amino acid 460, amino acid 820 to at most about amino acid 470, amino acid 820 to at most about amino acid 480, amino acid 820 to at most about amino acid 490, amino acid 820 to at most about amino acid 500, amino acid 820 to at most about amino acid 510, amino acid 820 to at most about amino acid 520, amino acid 820 to at most about amino acid 530, amino acid 820 to at most about amino acid 540, amino acid 820 to at most about amino acid 550, amino acid 820 to at most about amino acid 560, amino acid 820 to at most about amino acid 570, amino acid 820 to at most about amino acid 580, amino acid 820 to at most about amino acid 590, amino acid 820 to at most about amino acid 600, amino acid 820 to at most about amino acid 610, amino acid 820 to at most about amino acid 620, amino acid 820 to at most about amino acid 630, amino acid 820 to at most about amino acid 640, amino acid 820 to at most about amino acid 650, amino acid 820 to at most about amino acid 660, amino acid 820 to at most about amino acid 670, amino acid 820 to at most about amino acid 680, amino acid 820 to at most about amino acid 690, amino acid 820 to at most about amino acid 700, amino acid 820 to at most about amino acid 710, amino acid 820 to at most about amino acid 720, amino acid 820 to at most about amino acid 730, amino acid 820 to at most about amino acid 740, amino acid 820 to at most about amino acid 750, amino acid 820 to at most about amino acid 760, amino acid 820 to at most about amino acid 770, amino acid 820 to at most about amino acid 780, amino acid 820 to at most about amino acid 790, amino acid 820 to at most about amino acid 800, amino acid 820 to at most about amino acid 810, or amino acid 820 to at most about amino acid 820, or any length sequence there between of an inner ear cell target protein or an eye cell target protein (e.g., SEQ ID NO: 10, 11, 12, 13, or 14).

In some embodiments, splice sites are involved in trans-splicing. In some embodiments, a splice donor site (see, e.g., Trapani et al. EMBO Mol. Med. 6(2):194-211, 2014, which is incorporated in its entirety herein by reference) follows the coding sequence in the N-terminal construct. In the C-terminal construct, a splice acceptor site may be subcloned just before the coding sequence for CLRN1. In some embodiments, within the coding sequence, a silent mutation can be introduced, generating an additional site for restriction digestion.

In some embodiments, any of the constructs provided herein can be included in a composition suitable for administration to an animal for the amelioration of symptoms associated with syndromic and/or nonsyndromic hearing loss.

In some embodiments, any of the constructs provided herein can be included in a composition suitable for administration to an animal for the amelioration of symptoms associated with vision loss.

Pharmaceutical Compositions

Among other things, the present disclosure provides pharmaceutical compositions. In some embodiments compositions provided herein are suitable for administration to an animal for the amelioration of symptoms associated with syndromic and/or nonsyndromic hearing loss. In some embodiments compositions provided herein are suitable for administration to an animal for the amelioration of symptoms associated with vision loss.

In some embodiments, pharmaceutical compositions of the present disclosure may comprise, e.g., a polynucleotide, e.g., one or more constructs, as described herein. In some embodiments, a pharmaceutical composition may comprise one or more AAV particles, e.g., one or more rAAV construct encapsulated by one or more AAV serotype capsids, as described herein.

In some embodiments, a pharmaceutical composition comprises one or more pharmaceutically or physiologically acceptable carriers, diluents or excipients. As used herein, the term “pharmaceutically acceptable carrier” includes solvents, dispersion media, coatings, antibacterial agents, antifungal agents, and the like that are compatible with pharmaceutical administration. Supplementary active compounds can also be incorporated into any of the compositions described herein. Such compositions may include one or more buffers, such as neutral-buffered saline, phosphate-buffered saline, and the like; one or more carbohydrates, such as glucose, mannose, sucrose, and dextran; mannitol; one or more proteins, polypeptides, or amino acids, such as glycine; one or more antioxidants; one or more chelating agents, such as EDTA or glutathione; and/or one or more preservatives. In some embodiments, formulations are in a dosage forms, such as injectable solutions, injectable gels, drug-release capsules, and the like.

In some embodiments, compositions of the present disclosure are formulated for intravenous administration. In some embodiments compositions of the present disclosure are formulated for intra-cochlear administration. In some embodiments, a therapeutic composition is formulated to comprise a lipid nanoparticle, a polymeric nanoparticle, a mini-circle DNA and/or a CELiD DNA.

In some embodiments, a therapeutic composition is formulated to comprise a synthetic perilymph solution. For example, in some embodiments, a synthetic perilymph solution includes 20-200 mM NaCl; 1-5 mM KCl; 0.1-10 mM CaCl₂); 1-10 mM glucose; and 2-50 mM HEPES, with a pH between about 6 and about 9. In some embodiments, a therapeutic composition is formulated to comprise a physiologically suitable solution. For example, in some embodiments, a physiologically suitable solution comprises commercially available 1×PBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% Pluronic Acid F68). In some embodiments, alternative pluronic acids are utilized. In some embodiments, alternative ion concentrations are utilized.

In some embodiments, any of the pharmaceutical compositions described herein may further comprise one or more agents that promote the entry of a nucleic acid or any of the constructs described herein into a mammalian cell (e.g., a liposome or cationic lipid). In some embodiments, any of the constructs described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif.), formulations from Mirus Bio (Madison, Wis.) and Roche Madison (Madison, Wis.), PhaseRX polymer formulations such as, without limitation, SMARTT POLYMER TECHNOLOGY® (PhaseRX, Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly (lactic-co-glycolic acid) (PLGA) polymers, RONDEL™ (RNAi/Oligonucleotide Nanoparticle Delivery) polymers (Arrowhead Research Corporation, Pasadena, Calif.), and pH responsive co-block polymers, such as, but not limited to, those produced by PhaseRX (Seattle, Wash.). Many of these polymers have demonstrated efficacy in delivering oligonucleotides in vivo into a mammalian cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007, each of which is incorporated in its entirety herein by reference).

In some embodiments, a composition includes a pharmaceutically acceptable carrier (e.g., phosphate buffered saline, saline, or bacteriostatic water). Upon formulation, solutions will be administered in a manner compatible with a dosage formulation and in such amount as is therapeutically effective. Formulations are easily administered in a variety of dosage forms such as injectable solutions, injectable gels, drug-release capsules, and the like.

In some embodiments, a composition provided herein can be, e.g., formulated to be compatible with their intended route of administration. A non-limiting example of an intended route of administration is local administration (e.g., intra-cochlear administration).

In some embodiments, a delivery approach as disclosed herein comprises a synthetic AAV capsid (e.g., AAV Anc80) for transduction of inner ear cells, and/or a device for targeted delivery directly to the cochlea. In certain embodiments, the present disclosure provides methods and compositions suitable for transduction of inner ear cells. In some embodiments, transduction of inner ear cells may enable long-lasting expression of CLRN1 protein in the cochlea with minimal systemic exposure.

In some embodiments, a delivery approach as disclosed herein comprises a synthetic AAV capsid (e.g., AAV Anc80) for transduction of eye cells, and/or a device for targeted delivery directly to the eye. In certain embodiments, the present disclosure provides methods and compositions suitable for transduction of eye cells. In some embodiments, transduction of eye cells may enable long-lasting expression of CLRN1 protein in the eye with minimal systemic exposure.

In some embodiments, a provided composition comprises one nucleic acid construct. In some embodiments, a provided composition comprises two or more different constructs. In some embodiments, a composition that include a single nucleic acid construct comprising a coding sequence that encodes a clarin 1 protein and/or a functional characteristic portion thereof. In some embodiments, compositions comprise a single nucleic acid construct comprising a coding sequence that encodes a clarin 1 protein and/or a functional characteristic portion thereof, which, when introduced into a mammalian cell, that coding sequence is integrated into the genome of the mammalian cell. In some embodiments, a composition comprising at least two different constructs, e.g., constructs comprise coding sequences that encode a different portion of a clarin 1 protein, the constructs can be combined to generate a sequence encoding an active clarin 1 protein (e.g., a full-length clarin 1 protein) in a mammalian cell, and thereby treat associated syndromic or nonsyndromic sensorineural hearing loss in a subject in need thereof.

Also provided are kits including any of the compositions described herein. In some embodiments, a kit can include a solid composition (e.g., a lyophilized composition including the at least two different constructs described herein) and a liquid for solubilizing the lyophilized composition. In some embodiments, a kit can include a pre-loaded syringe including any of the compositions described herein.

In some embodiments, the kit includes a vial comprising any of the compositions described herein (e.g., formulated as an aqueous composition, e.g., an aqueous pharmaceutical composition).

In some embodiments, the kits can include instructions for performing any of the methods described herein.

### Genetically Modified Cells

The present disclosure also provides a cell (e.g., an animal cell, e.g., a mammalian cell, e.g., a primate cell, e.g., a human cell) that includes any of the nucleic acids, constructs or compositions described herein. In some embodiments, an animal cell is a human cell (e.g., a human supporting cell or a human hair cell). In other embodiments, an animal cell is a non-human mammal (e.g., Simian cell, Felidae cell, Canidae cell etc.). A person skilled in the art will appreciate that the nucleic acids and constructs described herein can be introduced into any animal cell (e.g., the supporting or hair cells of any animal suitable for veterinary intervention). Non-limiting examples of constructs and methods for introducing constructs into animal cells are described herein.

In some embodiments, an animal cell can be any cell of the inner ear, including hair and/or supporting cells. Non-limiting examples such cells include: Hensen's cells, Deiters cells, cells of the endolymphatic sac and duct, transitional cells in the saccule, utricle, and ampulla, inner and outer hair cells, spiral ligament cells, spiral ganglion cells, spiral prominence cells, external saccule cells, marginal cells, intermediate cells, basal cells, inner pillar cells, outer pillar cells, Claudius cells, inner border cells, inner phalangeal cells, or cells of the stria vascularis.

In some embodiments, an animal cell is a specialized cell of the cochlea. In some embodiments, an animal cell is a hair cell. In some embodiments, an animal cell is a cochlear inner hair cell or a cochlear outer hair cell. In some embodiments, an animal cell is a cochlear inner hair cell. In some embodiments, an animal cell is a cochlear outer hair cell.

In some embodiments, an animal cell can be any cell of the eye, including supporting cells. Non-limiting examples of such cells include: rod cells, cone cells, pigmented cells, horizontal cells, bipolar cells, amacrine cells, ganglion cells, photoreceptor cells, Muller cells, or retina cells.

In some embodiments, an animal cell is a specialized cell of the eye. In some embodiments, an animal cell is an eye cell. In some embodiments, an animal cell is a retinal cell. In.

In some embodiments, an animal cell is in vitro. In some embodiments, an animal cell is of a cell type which is endogenously present in an animal, e.g., in a primate and/or human. In some embodiments, an animal cell is an autologous cell obtained from an animal and cultured ex vivo.

### Methods

Hearing Loss

Among other things, the present disclosure provides methods. In some embodiments, a method comprises introducing a composition as described herein into the inner ear (e.g., a cochlea) of a subject. For example, provided herein are methods that in some embodiments include administering to an inner ear (e.g., cochlea) of a subject (e.g., an animal, e.g., a mammal, e.g., a primate, e.g., a human) a therapeutically effective amount of any composition described herein. In some embodiments of any of these methods, the subject has been previously identified as having a defective inner ear cell target gene (e.g., a supporting and/or hearing cell target gene having a mutation that results in a decrease in the expression and/or activity of a supporting and/or hearing cell target protein encoded by the gene). Some embodiments of any of these methods further include, prior to the introducing or administering step, determining that the subject has a defective inner ear cell target gene. Some embodiments of any of these methods can further include detecting a mutation in an inner ear cell target gene in a subject. Some embodiments of any of the methods can further include identifying or diagnosing a subject as having nonsyndromic or syndromic sensorineural hearing loss.

In some embodiments, provided herein are methods of correcting an inner ear cell target gene defect (e.g., a defect in CLRN1) in an inner ear of a subject, e.g., an animal, e.g., a mammal, e.g., a primate, e.g., a human. In some embodiments, methods include administering to the inner ear of a subject a therapeutically effective amount of any of the compositions described herein, where the administering repairs and or ameliorates the inner ear cell target gene defect in any cell subset of the inner ear of a subject. In some embodiments, the inner ear target cell may be a sensory cell, e.g., a hair cell, and/or a non-sensory cell, e.g., a supporting cell, and/or all or any subset of inner ear cells.

Also provided herein are methods of increasing the expression level of an inner ear cell target protein in any subset of inner ear cells of a subject (e.g., an animal, e.g., a mammal, e.g., a primate, e.g., a human,) that include: administering to the inner ear of the subject a therapeutically effective amount of any of the compositions described herein, where the administering results in an increase in the expression level of the inner ear cell target protein (e.g., clarin 1 protein) in any cell subset of the inner ear of a subject. In some embodiments, the inner ear target cell may be a sensory cell, e.g., a hair cell, and/or a non-sensory cell, e.g., a supporting cell, and/or all or any subset of inner ear cells.

Also provided herein are methods of treating hearing loss, e.g., nonsyndromic sensorineural hearing loss or syndromic sensorineural hearing loss, in a subject (e.g., an animal, e.g., a mammal, e.g., a primate, e.g., a human) identified as having a defective inner ear cell target gene that include: administering to the inner ear of the subject a therapeutically effective amount of any of the compositions described herein. In some embodiments of any of the methods provided herein, the subject has Usher syndrome type III.

Also provided herein are methods of restoring synapses and/or preserving spiral ganglion nerves in a subject identified or diagnosed as having an inner ear disorder that include: administering to the inner ear of the subject a therapeutically effective amount of any of the compositions described herein.

Also provided herein are methods comprising administering a composition disclosed herein, e.g., rAAV-CLRN1, for the treatment of a subject, e.g., mammal, e.g., human, e.g., patient, with Usher syndrome type III. In some embodiments, a composition disclosed herein is delivered via surgical delivery, e.g., to the cochlea.

Also provided herein are methods of reducing the size of, and/or restoring the vestibular aqueduct to an appropriate size. Also provided herein are methods of restoring endolymphatic pH to an appropriate and/or acceptable level in a subject identified or diagnosed as having an inner ear disorder that include: administering to the inner ear of the subject a therapeutically effective amount of any of the compositions described herein.

Also provided herein are methods that include administering to an inner ear of a subject a therapeutically effective amount of any of the compositions described herein.

Also provided herein are surgical methods for treatment of hearing loss (e.g., nonsyndromic sensorineural hearing loss or syndromic sensorineural hearing loss). In some embodiments, the methods include the steps of: introducing into a cochlea of a subject a first incision at a first incision point; and administering intra-cochlearly a therapeutically effective amount of any of the compositions provided herein. In some embodiments, the composition is administered to the subject at the first incision point. In some embodiments, the composition is administered to the subject into or through the first incision.

In some embodiments of any of the methods described herein, any composition described herein is administered to the subject into or through the cochlea oval window membrane. In some embodiments of any of the methods described herein, any of the compositions described herein is administered to the subject into or through the cochlea round window membrane. In some embodiments of any of the methods described herein, the composition is administered using a medical device capable of creating a plurality of incisions in the round window membrane. In some embodiments, the medical device includes a plurality of micro-needles. In some embodiments, the medical device includes a plurality of micro-needles including a generally circular first aspect, where each micro-needle has a diameter of at least about 10 microns. In some embodiments, the medical device includes a base and/or a reservoir capable of holding the composition. In some embodiments, the medical device includes a plurality of hollow micro-needles individually including a lumen capable of transferring the composition. In some embodiments, the medical device includes a means for generating at least a partial vacuum.

In some embodiments, technologies of the present disclosure are used to treat subjects with or at risk of hearing loss. For example, in some embodiments, a subject has an autosomal recessive hearing loss attributed to at least one pathogenic variant of CLRN1. It will be understood by those in the art that many different mutations in CLRN1 can result in a pathogenic variant. In some such embodiments, a pathogenic variant causes or is at risk of causing hearing loss.

In some embodiments, a subject experiencing hearing loss will be evaluated to determine if and where one or more mutations may exist that may cause hearing loss. In some such embodiments, the status of CLRN1 gene products or function (e.g., via protein or sequencing analyses) will be evaluated. In some embodiments of any of the methods described herein, the subject or animal is a mammal, in some embodiments the mammal is a domestic animal, a farm animal, a zoo animal, a non-human primate, or a human. In some embodiments of any of the methods described herein, the animal, subject, or mammal is an adult, a teenager, a juvenile, a child, a toddler, an infant, or a newborn. In some embodiments of any of the methods described herein, the animal, subject, or mammal is 1-5, 1-10, 1-20, 1-30, 1-40, 1-50, 1-60, 1-70, 1-80, 1-90, 1-100, 1-110, 2-5, 2-10, 10-20, 20-30, 30-40, 40-50, 50-60, 60-70, 70-80, 80-90, 90-100, 100-110, 10-30, 10-40, 10-50, 10-60, 10-70, 10-80, 10-90, 10-100, 10-110, 20-40, 20-50, 20-60, 20-70, 20-80, 20-90, 20-100, 20-110, 30-50, 30-60, 30-70, 30-80, 30-90, 30-100, 40-60, 40-70, 40-80, 40-90, 40-100, 50-70, 50-80, 50-90, 50-100, 60-80, 60-90, 60-100, 70-90, 70-100, 70-110, 80-100, 80-110, or 90-110 years of age. In some embodiments of any of the methods described herein, the subject or mammal is 1, 2, 3, 4, 5, 6, 7, 8, 9, 10, or 11 months of age.

In some embodiments of any of the methods described herein, the methods result in improvement in hearing (e.g., any of the metrics for determining improvement in hearing described herein) in a subject in need thereof for at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 100 days, at least 105 days, at least 110 days, at least 115 days, at least 120 days, at least 5 months, at least 6 months, at least 7 months, at least 8 months, at least 9 months, at least 10 months, at least 11 months, or at least 12 months.

In some embodiments a subject (e.g., an animal, e.g., a mammal, e.g., a human) has or is at risk of developing syndromic or nonsyndromic sensorineural hearing loss. In some embodiments a subject (e.g., an animal, e.g., a mammal, e.g., a human) has been previously identified as having a mutation in a CLRN1 gene. In some embodiments a subject (e.g., an animal, e.g., a mammal, e.g., a human) has any of the mutations in a CLRN1 gene that are described herein or are known in the art to be associated with syndromic or nonsyndromic sensorineural hearing loss.

In some embodiments, a subject (e.g., an animal, e.g., a mammal, e.g., a human) has been identified as being a carrier of a mutation in a CLRN1 gene (e.g., via genetic testing). In some embodiments, a subject (e.g., an animal, e.g., a mammal, e.g., a human) has been identified as having a mutation in a CLRN1 gene and has been diagnosed with syndromic or nonsyndromic sensorineural hearing loss. In some embodiments, a subject (e.g., an animal, e.g., a mammal, e.g., a human) has been identified as having syndromic or nonsyndromic sensorineural hearing loss.

In some embodiments, a subject (e.g., an animal, e.g., a mammal, e.g., a human) has been identified as being at risk of hearing loss (e.g., at risk of being a carrier of a gene mutation, e.g., a CLRN1 mutation). In some such embodiments, a subject (e.g., an animal, e.g., a mammal, e.g., a human) may have certain risk factors of hearing loss or risk of hearing loss (e.g., known parental carrier, afflicted sibling, or symptoms of hearing loss). In some such embodiments, a subject (e.g., an animal, e.g., a mammal, e.g., a human) has been identified as being a carrier of a mutation in a CLRN1 gene (e.g., via genetic testing) that has not previously been identified (i.e., is not a published or otherwise known variant of CLRN1). In some such embodiments, identified mutations may be novel (i.e., not previously described in the literature), and methods of treatment for a subject suffering from or susceptible to hearing loss will be personalized to the mutation(s) of the particular patient.

In some embodiments, successful treatment of syndromic or nonsyndromic sensorineural hearing loss can be determined in a subject using any of the conventional functional hearing tests known in the art. Non-limiting examples of functional hearing tests are various types of audiometric assays (e.g., pure-tone testing, speech testing, test of the middle ear, auditory brainstem response, and otoacoustic emissions).

In some embodiments of any method provided herein, two or more doses of any composition described herein are introduced or administered into a cochlea of a subject. Some embodiments of any of these methods can include introducing or administering a first dose of a composition into a cochlea of a subject, assessing hearing function of the subject following introduction or administration of a first dose, and administering an additional dose of a composition into the cochlea of the subject found not to have a hearing function within a normal range (e.g., as determined using any test for hearing known in the art).

In some embodiments of any method provided herein, the composition can be formulated for intra-cochlear administration. In some embodiments of any of the methods described herein, the compositions described herein can be administered via intra-cochlear administration or local administration. In some embodiments of any of the methods described herein, the compositions are administered through the use of a medical device (e.g., any of the exemplary medical devices described herein).

In some embodiments, intra-cochlear administration can be performed using any of the methods described herein or known in the art. For example, in some embodiments, a composition can be administered or introduced into the cochlea using the following surgical technique: first using visualization with a 0 degree, 2.5-mm rigid endoscope, the external auditory canal is cleared and a round knife is used to sharply delineate an approximately 5-mm tympanomeatal flap. The tympanomeatal flap is then elevated and the middle ear is entered posteriorly. The chorda tympani nerve is identified and divided, and a curette is used to remove the scutal bone, exposing the round window membrane. To enhance apical distribution of the administered or introduced composition, a surgical laser may be used to make a small 2-mm fenestration in the oval window to allow for perilymph displacement during trans-round window membrane infusion of the composition. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the composition. The device is then withdrawn and the round window and stapes foot plate are sealed with a gelfoam patch.

In some embodiments of any method provided herein, a subject has or is at risk of developing syndromic or nonsyndromic sensorineural hearing loss. In some embodiments of any method provided herein, a subject has been previously identified as having a mutation in an inner ear cell target gene, a gene which may be expressed in supporting cells and/or hair cells.

In some embodiments of any method provided herein, a subject has been identified as being a carrier of a mutation in an inner ear cell target gene (e.g., via genetic testing). In some embodiments of any method provided herein, a subject has been identified as having a mutation in an inner ear cell target gene and has been diagnosed with hearing loss (e.g., nonsyndromic sensorineural hearing loss or syndromic sensorineural hearing loss, e.g., Pendred syndrome or DFNB4). In some embodiments of any of the methods described herein, the subject has been identified as having hearing loss (e.g., nonsyndromic sensorineural hearing loss or syndromic sensorineural hearing loss). In some embodiments, successful treatment of hearing loss (e.g., nonsyndromic sensorineural hearing loss or syndromic sensorineural hearing loss) can be determined in a subject using any of the conventional functional hearing tests known in the art. Non-limiting examples of functional hearing tests include various types of audiometric assays (e.g., pure-tone testing, speech testing, test of the middle ear, auditory brainstem response, and otoacoustic emissions).

In some embodiments, a subject cell is in vitro. In some embodiments, a subject cell is originally obtained from a subject and is cultured ex vivo. In some embodiments, a subject cell has previously been determined to have a defective inner ear cell target gene. In some embodiments, a subject cell has previously been determined to have a defective hair cell target gene. In some embodiments, a subject cell has previously been determined to have a defective supporting cell target gene.

In some embodiments of these methods, following treatment e.g., one or two or more administrations of compositions described herein, there is an increase in expression of an active inner ear cell target protein (e.g., clarin 1 protein). In some embodiments, an increase in expression of an active inner ear target protein as described herein (e.g., clarin 1 protein) is relative to a control level, e.g., as compared to the level of expression of an inner ear cell target protein prior to introduction of the compositions comprising any construct(s) as described herein.

Methods of detecting expression and/or activity of a target protein (e.g., clarin 1 protein) are known in the art. In some embodiments, a level of expression of an inner ear cell target protein can be detected directly (e.g., detecting inner ear cell target protein or target mRNA. Non-limiting examples of techniques that can be used to detect expression and/or activity of a target RNA or protein (e.g., a CLRN1 gene product and/or clarin 1 protein or functional characteristic portion thereof) directly include: real-time PCR, Western blotting, immunoprecipitation, immunohistochemistry, mass spectrometry, or immunofluorescence. In some embodiments, expression of an inner ear cell target protein can be detected indirectly (e.g., through functional hearing tests).

In some embodiments, safety and tolerability of a composition disclosed herein can be assessed in hearing loss. In some embodiments, safety and tolerability of a composition disclosed herein, e.g., rAAV-CLRN1, can be assessed in a subject disclosed herein.

Administration

Provided herein are therapeutic delivery systems for treating hearing loss (e.g., nonsyndromic sensorineural hearing loss or syndromic sensorineural hearing loss). In one aspect, a therapeutic delivery system includes: i) a medical device capable of creating one or a plurality of incisions in a round window membrane of an inner ear of a subject in need thereof, and ii) an effective dose of a composition (e.g., any of the compositions described herein). In some embodiments, a medical device includes a plurality of micro-needles.

Also provided herein are surgical methods for treatment of hearing loss (e.g., nonsyndromic sensorineural hearing loss or syndromic sensorineural hearing loss). In some embodiments, a method the steps of: introducing into a cochlea of a subject a first incision at a first incision point; and administering intra-cochlearly a therapeutically effective amount of any of the compositions provided herein. In some embodiments, a composition is administered to a subject at the first incision point. In some embodiments, a composition is administered to a subject into or through the first incision.

In some embodiments of any method provided herein, any of the compositions described herein is administered to the subject into or through the cochlea oval window membrane. In some embodiments of any method provided herein, any of the compositions described herein is administered to the subject into or through the cochlea round window membrane. In some embodiments of any method provided herein, the composition is administered using a medical device capable of creating a plurality of incisions in the round window membrane. In some embodiments, a medical device includes a plurality of micro-needles. In some embodiments, a medical device includes a plurality of micro-needles including a generally circular first aspect, where each micro-needle has a diameter of at least about 10 microns. In some embodiments, a medical device includes a base and/or a reservoir capable of holding a composition. In some embodiments, a medical device includes a plurality of hollow micro-needles individually including a lumen capable of transferring a composition. In some embodiments, a medical device includes a means for generating at least a partial vacuum.

In some embodiments, any of the methods disclosed herein comprise a dose-escalation study to assess safety and tolerability in subjects, e.g., mammals, e.g., humans, e.g., patients, with hearing loss. In some embodiments, a composition disclosed herein, e.g., rAAV-CLRN1, is administered at a dosing regimen disclosed herein. In some embodiments, the dosing regimen comprises either unilateral or bilateral intracochlear administrations of a dose, e.g., as described herein, of a composition disclosed herein, e.g., rAAV-CLRN1. In some embodiments, the dosing regimen comprises delivery in a volume of at least 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per cochlea. In some embodiments, the dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, or at most 0.05 mL per cochlea. In some embodiments, the dosing regimen comprises delivery in a volume of about 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per cochlea, depending on the population. In some embodiments, the dosing regimen comprises delivery in a volume of at least 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per cochlea. In some embodiments, the dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, or at most 0.05 mL per cochlea. In some embodiments, the dosing regimen comprises delivery in a volume of about 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per cochlea, depending on the population.

In some embodiments, a method disclosed herein evaluates the safety and tolerability of escalating doses of a composition disclosed herein, e.g., rAAV-CLRN1, administered via intracochlear injection to a subject, e.g., 18 to 80 years of age, with hearing loss.

In some embodiments, a method disclosed herein evaluates the safety and tolerability of escalating doses of a composition disclosed herein, e.g., rAAV-CLRN1, administered via intraocular injection to a subject, e.g., 18 to 80 years of age, with hearing loss.

In some embodiments, any of the methods disclosed herein comprise an evaluation of the safety and tolerability of a composition disclosed herein, e.g., rAAV-CLRN1. In some embodiments, evaluation of the efficacy of a composition disclosed herein, e.g., rAAV-CLRN1 to treat hearing loss, is performed in a randomized, controlled setting (using a concurrent, non-intervention observation arm).

In some embodiments, any of the methods disclosed herein comprise an evaluation of the safety and tolerability of a composition disclosed herein, e.g., rAAV-CLRN1. In some embodiments, evaluation of the efficacy of a composition disclosed herein, e.g., rAAV-CLRN1 to treat vision loss, is performed in a randomized, controlled setting (using a concurrent, non-intervention observation arm).

Devices and Surgical Methods

The present disclosure provides, among other things technologies (e.g., systems, methods, devices, etc.) that may be used, in some embodiments, for treating deafness and other hearing-associated diseases, disorders and conditions. Examples of such technologies are also included in, e.g., WO2017223193 and WO2019084145, each of which is herein incorporated by reference in its entirety. In some embodiments, for example, the present disclosure provides therapeutic delivery systems for treating hearing loss (e.g., nonsyndromic sensorineural hearing loss). In some such embodiments, a therapeutic delivery system may include: (i) a medical device capable of creating one or a plurality of incisions in a round window membrane of an inner ear of a subject, and (ii) an effective dose of a composition (e.g., any compositions described herein). In some embodiments, a medical device includes a plurality of micro-needles.

AAV constructs are capable of constituting a full-length auditory polypeptide messenger RNA in a target cell of an inner ear. In some embodiments, the present disclosure provides a means for performing a surgical method, a method including the steps of: administering intra-cochlear to a human subject in need thereof an effective dose of a therapeutic composition of the present disclosure. A therapeutic composition is capable of being administered by using a medical device including: a) means for creating one or a plurality of incisions in a round window membrane, and b) an effective dose of a therapeutic composition.

The present disclosure provides, among other things, surgical methods for treatment (e.g., prevention, reversal, mitigation, attenuation) of hearing loss. In one aspect, methods include the steps of: introducing into a cochlea of a human subject a first incision at a first incision point; and administering intra-cochlear an effective dose of a therapeutic composition (e.g., any compositions described herein) as provided herein. In one embodiment, a therapeutic composition (e.g., any compositions described herein) is administered to a subject at a first incision point. In some embodiments, a therapeutic composition is administered to a subject into or through a first incision. In one embodiment, a therapeutic composition is administered to a subject into or through a cochlea oval window membrane. In one embodiment, a therapeutic composition is administered to a subject into or through a cochlea round window membrane.

For example, in some embodiments, a therapeutic composition is administered using a medical device capable of creating a plurality of incisions in a round window membrane. In some embodiments, a medical device includes a plurality of micro-needles. In some embodiments, a medical device includes a plurality of micro-needles including a generally circular first aspect, wherein each micro-needle includes a diameter of at least about 10 microns. In some embodiments, a medical device includes a base and/or a reservoir capable of holding a therapeutic composition. In some embodiments, a medical device includes a plurality of hollow micro-needles individually including a lumen capable of transferring a therapeutic composition. In some embodiments, a medical device includes a means for generating at least a partial vacuum.

Method of Introduction into Cochlea

The present disclosure provides, among other things, a method of introducing into a cochlea of a mammal (e.g., a human) a therapeutically effective amount of any compositions or systems as described herein. Also provided are methods of increasing expression of a functional CLRN1 protein in a cell (e.g., a hair cell, e.g., an outer hair cell, e.g., an inner ear cell, e.g., a spinal ganglion neuron (SGN)) in a cochlea of a mammal (e.g., a human) that include introducing into a cochlea of a subject a therapeutically effective amount of any compositions described herein.

Also provided are methods of treating hearing loss in a subject (e.g., a human) identified as having a defective (i.e., non-functional) CLRN1 gene product. In some such embodiments, methods include administering a therapeutically effective amount of any compositions described herein into a cochlea of a subject. In some embodiments, methods of treating may further comprise administering a cochlear implant to a subject (e.g., at substantially the same time as any compositions described herein are administered to a subject).

In some embodiments, a method of treating comprises administering two or more doses of any compositions described herein. In some such embodiments, compositions are introduced or administered into a cochlea of a mammal or subject. In some embodiments a method of treating comprises introducing or administering a first dose of a composition into a cochlea of a subject, assessing hearing function of a subject following introducing or administering of a first dose, and administering at least one additional dose of a composition into a cochlea of a subject if a subject is found not to have a hearing function within a normal range (e.g., as determined using any test for hearing known in the art).

In some embodiments, a method of treatment comprises intra-cochlear administration. In some such embodiments of any methods described herein, compositions are administered through use of a medical device (e.g., any exemplary medical devices described herein). In some embodiments, intra-cochlear administration can be performed as described herein or known in the art. For example, in some embodiments, a composition can be administered or introduced into a cochlea using the following surgical technique: first using visualization with a 0 degree, 2.5-mm rigid endoscope, an external auditory canal is cleared and a round knife is used to sharply delineate an approximately 5-mm tympanomeatal flap. A tympanomeatal flap is then elevated and the middle ear is entered posteriorly. The chorda tympani nerve is identified and divided, and a currette is used to remove the scutal bone, exposing the round window membrane. To enhance apical distribution of the administered or introduced composition, a surgical laser may be used to make a small 2-mm fenestration in the oval window to allow for perilymph displacement during trans-round window membrane infusion of the composition. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the composition. The device is then withdrawn and the round window and stapes foot plate are sealed with a gelfoam patch. One of skill in the art will understand that other variations or methods of intra-cochlear administration are available. In some embodiments, any such acceptable methods may be used to deliver one or more compositions and/or treat one or more subjects as described herein.

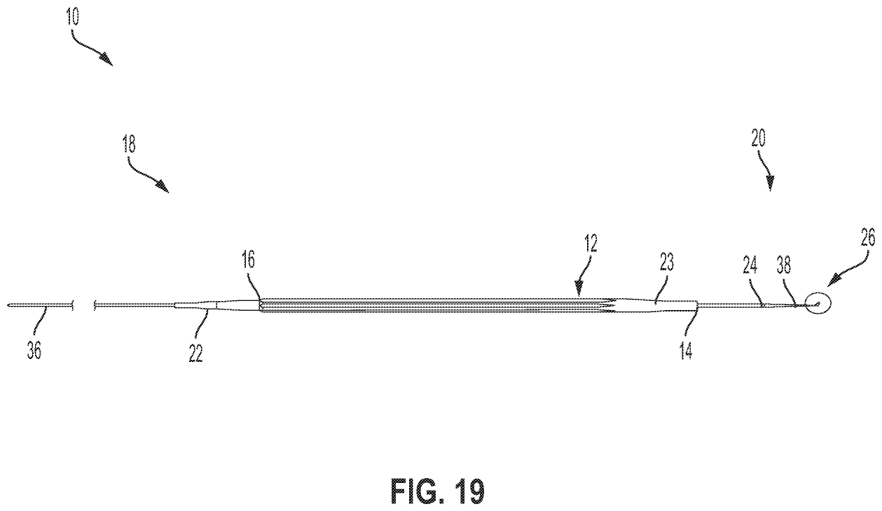

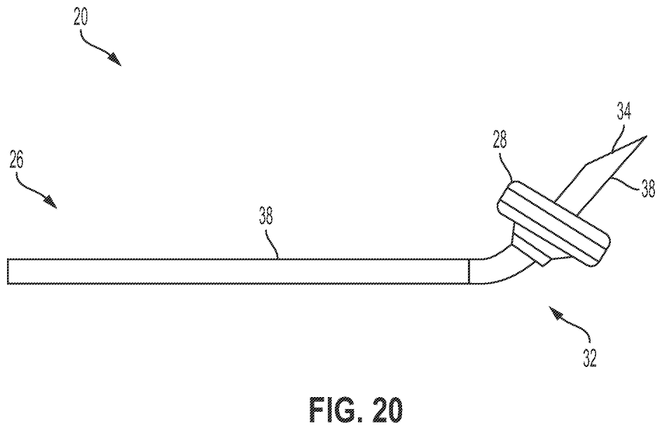

In some embodiments, an exemplary device for use in any of the methods disclosed herein is described in FIGS. 19-22. FIG. 19 illustrates an exemplary device 10 for delivering fluid to an inner ear. Device 10 includes a knurled handle 12, and a distal handle adhesive 14 (for example, an epoxy such as Loctite 4014) that couples to a telescoping hypotube needle support 24. The knurled handle 12 (or handle portion) may include kurling features and/or grooves to enhance the grip. The knurled handle 12 (or handle portion) may be from about 5 mm to about 15 mm thick or from about 5 mm to about 12 mm thick, or from about 6 mm to about 10 mm thick, or from about 6 mm to about 9 mm thick, or from about 7 mm to about 8 mm thick. The knurled handle 12 (or handle portion) may be hollow such that fluid may pass through the device 10 during use. The device 10 may also include a proximal handle adhesive 16 at a proximal end 18 of the knurled handle 12, a needle sub-assembly 26 (shown in FIG. 20) with stopper 28 (shown in FIG. 20) at a distal end 20 of the device 10, and a strain relief feature 22. Strain relief feature 22 may be composed of a Santoprene material, a Pebax material, a polyurethane material, a silicone material, a nylon material, and/or a thermoplastic elastomer. The telescoping hypotube needle support 24 surrounds and supports a bent needle 38 (shown in FIG. 20) disposed therewithin.

Referring still to FIG. 20, the stopper 28 may be composed of a thermoplastic material or plastic polymer (such as a UV-cured polymer), as well as other suitable materials, and may be used to prevent the bent needle 38 from being inserted too far into the ear canal (for example, to prevent insertion of bent needle 38 into the lateral wall or other inner ear structure). Device 10 also may include a tapered portion 23 disposed between the knurled handle 12 and the distal handle adhesive 14 that is coupled to the telescoping hypotube needle support 24. The knurled handle 12 (or handle portion) may include the tapered portion 23 at the distal end of the handle portion 12. Device 10 may also include tubing 36 fluidly connected to the proximal end 16 the device 10 and acts as a fluid inlet line connecting the device to upstream components (for example, a pump, a syringe, and/or upstream components which, in some embodiments, may be coupled to a control system and/or power supply (not shown)). In some embodiments, the bent needle 38 (shown in FIG. 20) extends from the distal end 20, through the telescoping hypotube needle support 24, through the tapered portion 23, through the knurled handle 12, and through the strain relief feature 22 and fluidly connects directly to the tubing 36. In other embodiments, the bent needle 38 fluidly connects with the hollow interior of the knurled handle (for example, via the telescoping hypotube needle support 24) which in turn fluidly connects at a proximal end 16 with tubing 36. In embodiments where the bent needle 38 does not extend all the way through the interior of the device 10, the contact area (for example, between overlapping nested hypotubes 42), the tolerances, and/or sealants between interfacing components must be sufficient to prevent therapeutic fluid from leaking out of the device 10 (which operates at a relatively low pressure (for example, from about 1 Pascal to about 50 Pa, or from about 2 Pa to about 20 Pa, or from about 3 Pa to about 10 Pa)).

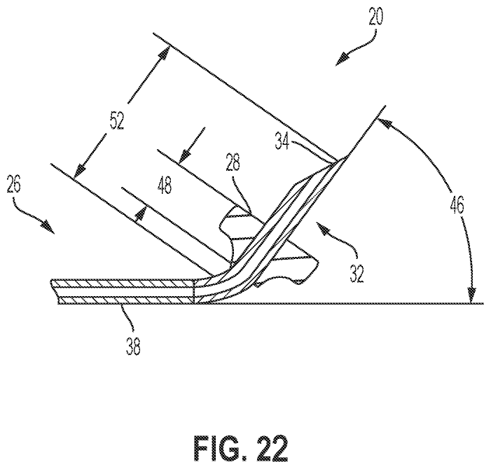

FIG. 20 illustrates a sideview of the bent needle sub-assembly 26, according to aspects of the present disclosed embodiments. Bent needle sub-assembly 26 includes a needle 38 that has a bent portion 32. Bent needle sub-assembly 26 may also include a stopper 28 coupled to the bent portion 32. The bent portion 32 includes an angled tip 34 at the distal end 20 of the device 10 for piercing a membrane of the ear (for example, the RWM). The needle 38, bent portion 32, and angled top 34 are hollow such that fluid may flow therethrough. The angle 46 (as shown in FIG. 22) of the bent portion 32 may vary. A stopper 28 geometry may be cylindrical, disk-shaped, annulus-shaped, dome-shaped, and/or other suitable shapes. Stopper 28 may be molded into place onto bent portion 32. For example, stopper 28 may be positioned concentrically around the bent portion 32 using adhesives or compression fitting. Examples of adhesives include an UV cure adhesive (such as Dymax 203A-CTH-F-T), elastomer adhesives, thermoset adhesives (such as epoxy or polyurethane), or emulsion adhesives (such as polyvinyl acetate). Stopper 28 fits concentrically around the bent portion 32 such that angled tip 34 is inserted into the ear at a desired insertion depth. The bent needle 38 may be formed from a straight needle using incremental forming, as well as other suitable techniques.

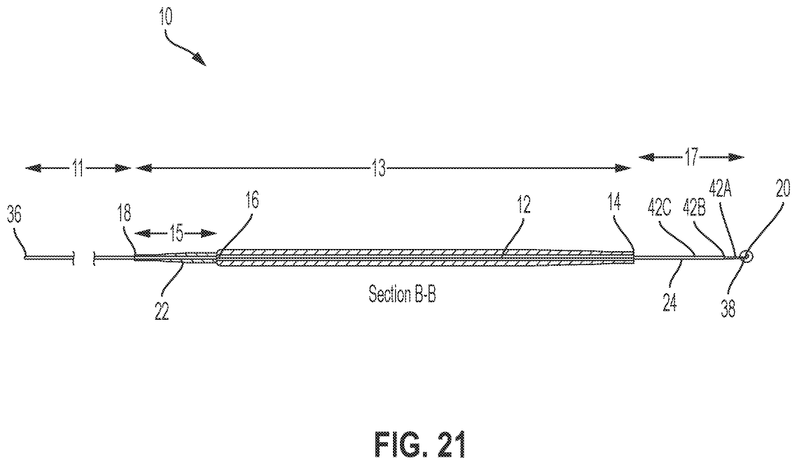

FIG. 21 illustrates a perspective view of exemplary device 10 for delivering fluid to an inner ear. Tubing 36 may be from about 1300 mm in length (dimension 11 in FIG. 21) to about 1600 mm, or from about 1400 mm to about 1500 mm, or from about 1430 mm to about 1450 mm. Strain release feature 22 may be from about 25 mm to about 30 mm in length (dimension 15 in FIG. 21), or from about 20 mm to about 35 mm in length. Handle 12 may be about 155.4 mm in length (dimension 13 in FIG. 21), or from about 150 mm to about 160 mm, or from about 140 mm to about 170 mm. The telescoping hypotube needle support 24 may have two or more nested hypotubes, for example three nested hypotubes 42A, 42B, and 42C, or four nested hypotubes 42A, 42B, 42C, and 42D. The total length of hypotubes 42A, 42B, 42C and tip assembly 26 (dimension 17 in FIG. 21) may be from about 25 mm to about 45 mm, or from about 30 mm to about 40 mm, or about 35 mm. In addition, telescoping hypotube needle support 24 may have a length of about 36 mm, or from about 25 mm to about 45 mm, or form about 30 mm to about 40 mm. The three nested hypotubes 42A, 42B, and 42C each may have a length of 3.5 mm, 8.0 mm, and 19.8 mm, respectively, plus or minus about 20%. The inner-most nested hypotube (or most narrow portion) of the telescoping hypotube needle support 24 may be concentrically disposed around needle 38.

FIG. 22 illustrates a perspective view of bent needle sub-assembly 26 coupled to the distal end 20 of device 10, according to aspects of the present disclosed embodiments. As shown in FIG. 22, bent needle sub-assembly 26 may include a needle 38 coupled to a bent portion 32. In other embodiments, the bent needle 38 may be a single needle (for example, a straight needle that is then bent such that it includes the desired angle 46). Needle 38 may be a 33-gauge needle, or may include a gauge from about 32 to about 34, or from about 31 to 35. At finer gauges, care must be taken to ensure tubing 36 is not kinked or damaged. Needle 38 may be attached to handle 12 for safe and accurate placement of needle 38 into the inner ear. As shown in FIG. 22, bent needle sub-assembly 26 may also include a stopper 28 disposed around bent portion 32. FIG. 22 also shows that bent portion 32 may include an angled tip 34 for piercing a membrane of the ear (for example, the RWM). Stopper 28 may have a height 48 of about 0.5 mm, or from about 0.4 mm to about 0.6 mm, or from about 0.3 mm to about 0.7 mm. Bent portion 32 may have a length 52 of about 1.45 mm, or from about 1.35 mm to about 1.55 mm, or from about 1.2 mm to about 1.7 mm. In other embodiments, the bent portion 32 may have a length greater than 2.0 mm such that the distance between the distal end of the stopper 28 and the distal end of the angled tip 34 is from about 0.5 mm to about 1.7 mm, or from about 0.6 mm to about 1.5 mm, or from about 0.7 mm to about 1.3 mm, or from about 0.8 mm to about 1.2 mm. FIG. 22 shows that stopper 28 may have a geometry that is cylindrical, disk-shaped, and/or dome-shaped. A person of ordinary skill will appreciate that other geometries could be used.

In some embodiments, a delivery approach as disclosed herein comprises a synthetic AAV capsid (e.g., AAV Anc80) for transduction of inner ear cells, and/or a device for targeted delivery directly to the cochlea. In certain embodiments, the present disclosure provides methods and compositions suitable for transduction of inner ear cells.

In some embodiments of any of the methods described herein, any composition described herein is administered to the subject into or through the cochlea oval window membrane. In some embodiments of any of the methods described herein, any of the compositions described herein is administered to the subject into or through the cochlea round window membrane. In some embodiments of any of the methods described herein, the composition is administered using a medical device capable of creating a plurality of incisions in the round window membrane. In some embodiments, the medical device includes a plurality of micro-needles. In some embodiments, the medical device includes a plurality of micro-needles including a generally circular first aspect, where each micro-needle has a diameter of at least about 10 microns. In some embodiments, the medical device includes a base and/or a reservoir capable of holding the composition. In some embodiments, the medical device includes a plurality of hollow micro-needles individually including a lumen capable of transferring the composition. In some embodiments, the medical device includes a means for generating at least a partial vacuum.

In some embodiments, also disclosed herein is a sterile, one-time use delivery device for intracochlear administration, to deliver a composition disclosed herein, e.g., rAAV-CLRN1 to perilymph fluid of inner ear through a round window membrane with a vent located in a stapes footplate. In some embodiments, in this intracochlear administration approach, a composition disclosed herein, e.g., e.g., rAAV-CLRN1 can be administered into the scala tympani through the round window membrane, with a vent in a stapes footplate within the oval window, such that composition is perfused through scala tympani, then through scala vestibuli via connection at the helicotrema, and follows the fluid path to a vent in a stapes footplate (FIGS. 11A-11B).

Methods of Treating a Subject

The present disclosure provides, among other things, that technologies described herein may be used to treat an underlying disease and/or symptoms in a subject suffering from or at risk of Usher syndrome type III characterized by hearing loss.

In some embodiments, a method comprises administering a construct (e.g., an rAAV construct) described herein, a particle (e.g., an rAAV particle), or a composition described herein to a subject. In some embodiments, a method is a method of treatment. In some embodiments, a subject is a subject suffering from or at risk of Usher syndrome type III characterized by hearing loss.

In some embodiments, administering a construct (e.g., an rAAV construct) described herein, a particle (e.g., an rAAV particle), or a composition described herein to a subject may alleviate and/or ameliorate one or more symptoms associated with Usher syndrome type III characterized by hearing loss. Symptoms can include, for example, hearing loss, degeneration of hair cells, alteration of biochemical milieu of inner ear fluids, elevated intralabyrinthine protein, endolymphatic hydrops, cochlear aperture obstruction, intralabryinthine hemorrhage, disruption of cochlear vascular supply, tinnitus, dizziness, intractable headache, facial neuropathy, trigeminal neuropathy, facial paralysis, facial paresthesia, hydrocephalus, cerebellar herniation, and/or death.

In some embodiments, Usher syndrome type III characterized by hearing loss is associated with a gene mutation (e.g., a deletion mutation, a frameshift mutation, a nonsense mutation, a hypomorphic mutation, a hypermorphic mutation, a neomorphic mutation, aberrant over expression, aberrant under expression, etc.). In some embodiments, a subject suffering from or at risk of Usher syndrome type III characterized by hearing loss may have a mutation in a CLRN1 gene, which may be characterized as described herein.

In some embodiments, a subject is genetically and/or symptomatically characterized prior to, during, and/or after treatment with technologies described herein (e.g. real-time PCR, quantitative real-time PCR, Western blotting, immunoprecipitation, immunohistochemistry, mass spectrometry, or immunofluorescence, indirect phenotypic determination of expression of a gene and/or protein (e.g., through functional hearing tests, ABRs, DPOAEs, etc.), etc.). In some embodiments, a subject suffering from or at risk of Usher syndrome type III characterized by hearing loss may have their associated disease state characterized through tissue sampling (e.g., comprising one or more inner ear cells, e.g., comprising one or more hair cells and/or one or more supporting cells). In some embodiments, tissues are evaluated via morphological analysis to determine morphology of hair cells and/or support cells before, during, and/or after administration of any technologies (e.g., methodologies, e.g., compositions, e.g., compositions comprising constructs, and/or particles, etc.) as described herein. In some such embodiments, standard immunohistochemical or histological analyses may be performed. In some embodiments, if cells are used in-vitro or ex-vivo, additional immunocytochemical or immunohistochemical analyses may be performed. In some embodiments, one or more assays of one or more proteins or transcripts (e.g., western blot, ELISA, polymerase chain reactions) may be performed on one or more samples from a subject or in-vitro cell populations.

In some embodiments, administering a construct (e.g., an rAAV construct) described herein, a particle (e.g., an rAAV particle), or a composition described herein to a subject improves a patients immunohistochemical evaluation (e.g., tests as described above) when compared to immunohistochemical tests performed prior to treatment with technologies described herein or when compared to a control population.

Treating a Subject to Improve Symptoms of Usher Syndrome Type III

In some embodiments, a subject suffering from or at risk of Usher syndrome type III characterized by hearing loss may receive a treatment regime that is characterized by hearing function. In some embodiments, functionality of a treatment regime is characterized through hearing function, wherein such hearing function is determined in an individual using auditory brainstem response measurements (ABR) before, after, and/or during treatment with compositions and methods described herein. In some embodiments, functionality of a treatment regime is characterized through hearing function, wherein such hearing function is determined in an individual by measuring distortion product optoacoustic emissions (DPOAEs) before, after, and/or during treatment with compositions and methods described herein. In some such embodiments, hearing measurements are taken from one or both ears of a subject. In some such embodiments, recordings are compared to prior recordings for the same subject and/or known thresholds on such response measurements used to define, e.g., hearing loss versus acceptable hearing ranges to be defined as normal hearing. In some embodiments, a subject has ABR and/or DPOAE measurements recorded prior to receiving any treatment. In some embodiments, a subject treated with one or more technologies described herein will have improvements on ABR and/or DPOAE measurements after treatment as compared to before treatment. In some embodiments, ABR and/or DPOAE measurements are taken after treatment is administered and at regular follow-up intervals post-treatment. In some embodiments, treatment with technologies described herein improve a patients test evaluation (e.g., tests as described above) when compared to tests performed prior to treatment with technologies described herein or when compared to a control population.

Evaluating Hearing Loss and Recovery

In some embodiments, hearing function is determined using auditory brainstem response measurements (ABR). A decrease in an ABR threshold compared to a reference, the presence (e.g., detection) of an ABR threshold, and/or a normal ABR morphology indicate improved hearing. In some embodiments, hearing is tested by measuring distortion product optoacoustic emissions (DPOAEs). A decrease in an DPOAE threshold compared to a reference, the presence (e.g., detection) of an DPOAE threshold, and/or a normal DPOAE morphology indicate improved hearing. In some such embodiments, measurements are taken from one or both ears of a subject. In some such embodiments, recordings are compared to prior recordings for the same subject and/or known thresholds on such response measurements used to define, e.g., hearing loss versus acceptable hearing ranges to be defined as normal hearing. In some embodiments, a subject has ABR and/or DPOAE measurements recorded prior to receiving any treatment. In some embodiments, a subject treated with one or more technologies described herein will have improvements on ABR and/or DPOAE measurements after treatment as compared to before treatment. In some embodiments, ABR and/or DPOAE measurements are taken after treatment is administered and at regular follow-up intervals post-treatment.

In some embodiments, hearing function is determined using speech pattern recognition or is determined by a speech therapist. In some embodiments, hearing function is determined by pure tone testing. In some embodiments, hearing function is determined by bone conduction testing. In some embodiments, hearing function is determined by acoustic reflex testing. In some embodiments hearing function is determined by tympanometry. In some embodiments, hearing function is determined by any combination of hearing analysis known in the art. In some such embodiments, measurements are taken holistically, and/or from one or both ears of a subject. In some such embodiments, recordings and/or professional analysis are compared to prior recordings and/or analysis for the same subject and/or known thresholds on such response measurements used to define, e.g., hearing loss versus acceptable hearing ranges to be defined as normal hearing. In some embodiments, a subject has speech pattern recognition, pure tone testing, bone conduction testing, acoustic reflex testing and/or tympanometry measurements and/or analysis conducted prior to receiving any treatment. In some embodiments a subject treated with one or more technologies described herein will have improvements on speech pattern recognition, pure tone testing, bone conduction testing, acoustic reflex testing and/or tympanometry measurements after treatment as compared to before treatment. In some embodiments, speech pattern recognition, pure tone testing, bone conduction testing, acoustic reflex testing and/or tympanometry measurements are taken after treatment is administered and at regular follow-up intervals post-treatment.

Methods of Characterizing a Disease State

The term “mutation in a CLRN1 gene” refers to a modification in a known consensus functional CLRN1 gene that results in the production of a clarin 1 protein having one or more of: a deletion in one or more amino acids, one or more amino acid substitutions, and one or more amino acid insertions as compared to the consensus functional clarin 1 protein, and/or results in a decrease in the expressed level of the encoded clarin 1 protein in a mammalian cell as compared to the expressed level of the encoded clarin 1 protein in a mammalian cell not having a mutation. In some embodiments, a mutation can result in the production of a clarin 1 protein having a deletion in one or more amino acids (e.g., 2, 3, 4, 5, 6, 7, 8, 9, 10, 11, 12, 13, 14, 15 16, 17, 18, 19, 20, or more amino acids). In some embodiments, the mutation can result in a frameshift in the CLRN1 gene. The term “frameshift” is known in the art to encompass any mutation in a coding sequence that results in a shift in the reading frame of the coding sequence. In some embodiments, a frameshift can result in a nonfunctional protein. In some embodiments, a point mutation can be a nonsense mutation (i.e., result in a premature stop codon in an exon of the gene). A nonsense mutation can result in the production of a truncated protein (as compared to a corresponding consensus functional protein) that may or may not be functional. In some embodiments, the mutation can result in the loss (or a decrease in the level) of expression of CLRN1 mRNA or clarin 1 protein or both the mRNA and protein. In some embodiments, the mutation can result in the production of an altered clarin 1 protein having a loss or decrease in one or more biological activities (functions) as compared to a consensus functional clarin 1 protein.

In some embodiments, the mutation is an insertion of one or more nucleotides into a CLRN1 gene. In some embodiments, the mutation is in a regulatory and/or control sequence of the clarin 1 gene, i.e., a portion of the gene that is not coding sequence. In some embodiments, a mutation in a regulatory and/or control sequence may be in a promoter or enhancer region and prevent or reduce the proper transcription of the CLRN1 gene. In some embodiments, a mutation is in a known heterologous gene known to interact with a clarin 1 protein, or the CLRN1 gene.

Methods of genotyping and/or detecting expression or activity of CLRN1 mRNA and/or clarin 1 protein are known in the art (see, e.g., Ito et al., World J Otorhinolaryngol. 2013 May 28; 3(2): 26-34, and Roesch et al., Int J Mol Sci. 2018 January; 19(1): 209, each of which is incorporated in its entirety herein by reference). In some embodiments, level of expression of CLRN1 mRNA or clarin 1 protein may be detected directly (e.g., detecting clarin 1 protein, detecting CLRN1 mRNA etc.). Non-limiting examples of techniques that can be used to detect expression and/or activity of CLRN1 directly include, e.g., real-time PCR, quantitative real-time PCR, Western blotting, immunoprecipitation, immunohistochemistry, mass spectrometry, or immunofluorescence. In some embodiments, expression of CLRN1 and/or clarin 1 protein can be detected indirectly (e.g., through functional hearing tests, ABRs, DPOAEs, etc.).

In some embodiments, tissue samples (e.g., comprising one or more inner ear cells, e.g., comprising one or more hair cells and/or one or more supporting cells) may be evaluated via morphological analysis to determine morphology of eye cells and/or hair cells and/or support cells before and after administration of any agents (e.g., compositions, e.g., compositions comprising constructs, and/or particles, etc.) as described herein. In some such embodiments, standard immunohistochemical or histological analyses may be performed. In some embodiments, if cells are used in vitro or ex vivo, additional immunocytochemical or immunohistochemical analyses may be performed. In some embodiments, one or more assays of one or more proteins or transcripts (e.g., western blot, ELISA, polymerase chain reactions) may be performed on one or more samples from a subject or in vitro cell populations.

Evaluating Hearing Loss, Tinnitus, Dizziness, and Symptom Recovery

In some embodiments, hearing function is determined in an individual using auditory brainstem response measurements (ABR) before, after, and/or during treatment with compositions and methods described herein. In some embodiments, hearing function is determined in an individual by measuring distortion product optoacoustic emissions (DPOAEs) before, after, and/or during treatment with compositions and methods described herein. In some such embodiments, measurements are taken from one or both ears of a subject. In some such embodiments, recordings are compared to prior recordings for the same subject and/or known thresholds on such response measurements used to define, e.g., hearing loss versus acceptable hearing ranges to be defined as normal hearing. In some embodiments, a subject has ABR and/or DPOAE measurements recorded prior to receiving any treatment. In some embodiments, a subject treated with one or more technologies described herein will have improvements on ABR and/or DPOAE measurements after treatment as compared to before treatment. In some embodiments, ABR and/or DPOAE measurements are taken after treatment is administered and at regular follow-up intervals post-treatment.

In some embodiments, hearing function is determined using speech pattern recognition or is determined by a speech therapist. In some embodiments, hearing function is determined by pure tone testing. In some embodiments, hearing function is determined by bone conduction testing. In some embodiments, hearing function is determined by acoustic reflex testing. In some embodiments hearing function is determined by tympanometry. In some embodiments, hearing function is determined by any combination of hearing analysis known in the art. In some such embodiments, measurements are taken holistically, and/or from one or both ears of a subject. In some such embodiments, recordings and/or professional analysis are compared to prior recordings and/or analysis for the same subject and/or known thresholds on such response measurements used to define, e.g., hearing loss versus acceptable hearing ranges to be defined as normal hearing. In some embodiments, a subject has speech pattern recognition, pure tone testing, bone conduction testing, acoustic reflex testing and/or tympanometry measurements and/or analysis conducted prior to receiving any treatment. In some embodiments a subject treated with one or more technologies described herein will have improvements on speech pattern recognition, pure tone testing, bone conduction testing, acoustic reflex testing and/or tympanometry measurements after treatment as compared to before treatment. In some embodiments, speech pattern recognition, pure tone testing, bone conduction testing, acoustic reflex testing and/or tympanometry measurements are taken after treatment is administered and at regular follow-up intervals post-treatment.

In some embodiments, any of the methods disclosed herein comprise behavioral audiometry evaluation. In some embodiments, behavioral audiometry evaluation comprises pure-tone audiometry with air and bone curves with appropriate masking, Speech audiometry, Words in quiet, or words in noise. In some embodiments, behavioral audiometry evaluation comprises electrophysiologic audiometry by auditory brainstem response testing. In some embodiments, behavioral audiometry evaluation comprises standardized questionnaires: HHIA: Hearing Handicap Inventory for Adults, DHI: Dizziness Handicap Inventory, THI: Tinnitus Handicap Inventory, PANQOL: Penn Acoustic Neuroma Quality of Life (QoL).

In some embodiments of any of the methods disclosed herein, safety and efficacy may be monitored by evaluation of otologic, vestibular, and systemic adverse events, as well as hematology, clinical chemistry, and/or urinalysis parameters. In some embodiments of any of the methods disclosed herein, additional parameters to be assessed will include CLRN1 protein levels in blood and construct DNA in ear swabs, nasal swabs, saliva, and blood. In some embodiments of any of the methods disclosed herein, blood may also be collected for evaluation of potential humoral immune responses to the capsid and transgene product.

The ABR test measures whether the animal's cochlea, cochlear nerve, and brainstem responds to each sound stimulus and is often used as a measure of the health of the ear. This same basic test is commonly used to test hearing of newborn humans in hospitals and it is a standard hearing test used in lab animals. In some embodiments, an ABR test involves injecting mice with an IP dose of ketamine/xylazine anesthetic to minimize movement and muscle artifacts and to aid in the placement of measurement electrodes. In some embodiments, an ABR test (when done with the DPOAE at the same time, and in both ears) takes approximately 45 minutes to complete, so a booster dose of anesthetic is sometimes required. In some embodiments, an initial dose of anesthetic consists of ketamine (100 mg/kg) and xylazine (10 mg/kg) IP and if needed, a ketamine only booster which consists of ¼-½ of the original ketamine dose. In some embodiments, no redosing of xylazine is performed based on veterinarian recommendations. In some embodiments, transdermal recording electrodes through the skin surface are placed at three standard locations (at vertex of the skull equidistant between the ears, over the mastoid behind the pinna on the test-ear, and over the mastoid behind the pinna for the opposite ear for the grounding electrode). In some embodiments, stimuli consists of low (8 kHz), middle (16 kHz), and high (32 kHz) frequency pure tone pip stimuli (0.1 ms rise fall, 1.5 ms duration) from 10-80 dB SPL in ascending 5-dB steps. In some embodiments, stimuli will be presented at a rate of 30/sec, and 512 artifact-free averages are acquired at each stimulus level. In some embodiments, ABRs were collected at 2-3 separate time points, depending upon the group; baseline and at terminal week 4-6 with a subset of animals also tested at 3 weeks post-surgery. In some embodiments, in addition to measuring the lowest intensity of each stimulus frequency that the animal's brainstem could reliably process (threshold), the amplitude of Wave I in response to suprathreshold stimuli can be measured in order to assess the integrity of the afferent flow of information from the cochlear hair cells to the auditory nerve. In some embodiments, ABR thresholds can provide important information about the lowest level of sound that an animal's ear passes along to and is processed by the brainstem, the suprathreshold responses in the amplitude of the ABR Wave I has increasingly been used as a proxy for the integrity of the ribbon synapse connection between the base of the inner hair cells and the auditory nerve dendrites.

DPOAEs are sounds created by movement of the cochlear outer hair cells and are non-invasively measured in the ear canal with a transducer & microphone combination. In some embodiments, a size of evoked DPOAEs is a useful measure of outer hair cell function. This same basic test is also commonly used to test hearing of newborn humans in hospitals and it is a standard hearing test used in lab animals. In some embodiments, two primary tones (f1 and f2) are presented to an ear producing mechanical vibrations that causes pressure changes in cochlear fluids at stimulus and distortion frequencies. In some embodiments, these pressure changes drive the ear in reverse, activating the middle ear and then eardrum to produce sound in the ear canal. In some embodiments, DPOAEs are collected at the same test frequencies used in ABRs (8, 16, 32 kHz) and while under anesthesia for ABRs. In some embodiments, an f2 is centered at 8, 16, and 32 kHz while an f1=f2*0.8+10 dB. In some embodiments, at each frequency, tones are presented from 10-80 dB SPL in 5-dB ascending increments. In some embodiments, DPOAEs assess outer hair cell function and are therefore typically used as a suprathreshold assessment of the strength of the response, measured as an amplitude of the distortion product emission response. In some embodiments, all three test frequencies are used. In some embodiments, reliable distortion product responses may not be obtainable for each frequency tested, in such cases, analyses may be done as appropriate.

Evaluating CLRN1 Protein Concentration in Biological Samples

In some embodiments, methods described herein include evaluating CLRN1 protein concentrations in one or more biological samples form an individual before, during, and/or after treatment with compositions described herein.

In some embodiments of these methods, following treatment e.g., one or two or more administrations of compositions described herein, there is an increase in expression of a CLRN1 protein. In some embodiments, an increase in expression of an active CLRN1 protein as described herein when compared relative to a control level, e.g., as compared to the level of expression of a CLRN1 protein prior to introduction of the compositions comprising any construct(s) as described herein.

Methods of detecting expression and/or activity of a target RNA and/or protein are known in the art. In some embodiments, a level of expression of an inner ear cell target protein can be detected directly (e.g., detecting inner ear cell target protein or target mRNA). Non-limiting examples of techniques that can be used to detect expression and/or activity of a target RNA or protein directly include: real-time PCR, Western blotting, immunoprecipitation, immunohistochemistry, mass spectrometry, or immunofluorescence. In some embodiments, expression of an inner ear cell target protein can be detected indirectly (e.g., through functional hearing tests).

In some embodiments, biodistribution and/or shedding analysis of rAAV particles is performed. In some embodiments, disclosed herein is a composition comprising an rAAV-CLRN1 replication-defective, rAAV particle designed to deliver a cDNA, e.g., to express a CLRN1 protein. In some embodiments, an rAAV particle as described herein is used to treat a subject, e.g., a human, e.g., a patient, with Usher syndrome type III. In some embodiments, a composition disclosed herein is administered via an intracochlear route. In some embodiments, a composition disclosed herein is administered at a low dose of rAAV particles (e.g., as measured by vector genome qPCR analysis). In some embodiments, a composition disclosed herein is administered to a localized area of the body. In some embodiments, a composition disclosed herein, is expected to result in limited vascular spread and systemic exposure. In some embodiments, a composition disclosed herein results in higher levels of construct sequences in the cochlea. In some embodiments, a composition disclosed herein results in lower but detectable levels in most other tissues and fluids collected. In some embodiments, levels of construct sequences were generally decreased overall by six months. In some embodiments, levels of construct sequences decrease over time in blood samples, e.g., one month following intracochlear administration of a composition disclosed herein.

In some embodiments of any of the methods disclosed herein, relevant fluids for biodistribution and shedding (e.g., blood, serum, urine, saliva, nasal and ear swabs, and CSF fluid) are collected and/or evaluated. In some embodiments of any of the methods disclosed herein, non-target tissues are collected and/or evaluated. In some embodiments, capsid variant is expected to determine tropism. In some embodiments, a composition disclosed herein comprises a capsid variant, e.g., AAV Anc80 capsid variant. In some embodiments, delivery of a composition disclosed herein comprising a capsid variant, e.g., via the same route of administration, in the same particle formulation, and/or at equivalent or lower particle doses, is not expected to result in differences in biodistribution and shedding patterns.

In some embodiments, a composition disclosed herein, e.g., rAAV-CLRN1, is dosed at a level less than about 2E12 total vg/cochlea. In some embodiments, a dose of a composition disclosed herein is a function of, e.g., limitations of volume and rAAV particle concentration.

In contrast, clinical trials using rAAV particles have delivered more than 1E14 vg/kg via systemic routes of administration, in some cases to participants younger than 6 months of age (AveXis 2019, incorporated herein in its entirety by reference). Other localized deliveries with relatively low doses of rAAV particles have not reported extensive biodistribution beyond the target area, e.g., throughout the body, or extensive rAAV particle shedding (excretion; generally measuring underlying construct DNA concentration through methods such as qPCR). For example, low levels of distribution beyond the ocular target area (specifically in the optic nerve of the particle-injected eye, optic chiasm, spleen and liver, and sporadically in the lymph nodes of study animals) have been reported for Luxturna® delivered bilaterally at a dose of 7.5E11 vg/eye. Similarly, in the Phase 3 clinical trial, rAAV particle was shed transiently and at low levels in tears of 45% of the participants; it was detected, also at low levels, in serum (but not whole blood) samples of 10% of the participants in the days immediately following subretinal administration of Luxturna® delivered bilaterally at a dose of 1.5E11 vg/eye (see, e.g., Russell 2017; Spark Therapeutics 2017, each of which is incorporated herein in its entirety by reference).

In some embodiments, any of the methods disclosed herein comprise evaluating a distribution of rAAV particle sequences in blood (e.g., serum and whole blood) over a time course following unilateral administration to the cochlea using, e.g., a validated qPCR method. In some embodiments, additional specimens (e.g., external auditory canal swabs, nasal swabs, saliva, and urine) will be collected for evaluation of shedding from the subject. In some embodiments, a specimen is collected from a subject until at least three consecutive negative samples are obtained.

In some embodiments, proteins correlating with hearing loss are measured before, during, and/or after treatment with compositions and/or methods described herein. In certain embodiments, such hearing loss associated proteins include: μ-Crystallin (CRYM), low density lipoprotein receptor-related protein 2 (LRP2), immunoglobulin (Ig) γ-4 chain C region, Ig κ-chain C region, complement C3, immunoglobulin heavy constant γ3, and/or chemokine receptor-4 (CXCR4).

In some embodiments, immunogenicity to AAV capsids and/or particles are measured. Immunogenicity to AAV capsids and/or particles delivered to localized areas, and in relatively low doses compared to systemic applications, have generally not yielded specific patterns of immune responses; importantly, responses observed through both humoral and cell-mediated immunological monitoring (e.g., through enzyme-linked immunosorbent assay [ELISA]/neutralizing antibody [NAb] and enzyme-linked immunosorbent spot [ELISPOT] assays, respectively) have predominantly been without clinical correlate for route(s) of administration (ROA) that afford some immunological protection (e.g., direct administration to the brain).

In some embodiments of any of the methods disclosed herein, an intracochlear ROA, e.g., in species with a non-patent cochlear aqueduct (e.g., NHPs and humans), is expected to provide a similar level of protection. In some embodiments of any of the methods disclosed herein, a subject will receive a short, peri-operative course of an immunomodulatory regimen, e.g., systemic oral corticosteroids, for approximately 17 days, beginning 3 days before administration of a compositions disclosed herein, e.g., rAAV-CLRN1. In some embodiments, the immunomodulatory regimen reduces inflammation related to the surgical administration procedure. In some embodiments, the immunomodulatory regimen can also further reduce the potential for an immune reaction to either a capsid (e.g., AAV Anc80) or the underlying construct (e.g., a transgene product, e.g., a CLRN1 protein).

In some embodiments, any method disclosed herein further comprise evaluating humoral immunity (e.g., antibody responses) in response to administration of a composition disclosed herein. In some embodiments, effect of pre-existing immunity, measured e.g., by serum NAb levels, on the transduction of a compositions disclosed herein when delivered via the intracochlear ROA is evaluated. In some embodiments, pre-existing NAb levels do not inhibit transduction of AAV particles delivered by an intracochlear route of administration. In some embodiments, any method disclosed herein further comprise evaluating serum for potential systemic humoral responses to both the AAV capsid and/or the transgene product (e.g., a protein). In some embodiments, responsive to the evaluation of systemic humoral responses, a treatment interval for bilateral intracochlear administration of a composition disclosed herein, e.g., rAAV-CLRN1 can be developed.

In some embodiments, any method disclosed herein does not result in cytotoxic T cell responses, e.g., to either an AAV particle, capsid, and/or construct (e.g. underlying transgene) product from rAAV particles delivered via a localized route of administration (ROA), such as intracochlear administration. For example, the labeling for Luxturna® notes that no subject had a clinically significant, cytotoxic T cell response (Spark Therapeutics 2017, incorporated herein in its entirety by reference); isolated positive interferon-gamma (IFN-gamma) ELISPOT assay results were obtained during the clinical development program (Bennett 2012, incorporated herein in its entirety by reference), but the significance of these isolated results is unknown, as no clinical inflammatory response was observed and no dose limiting toxicity was seen in the clinical program.

In certain embodiments, pharmacokinetics of any of the compositions or products of compositions described herein are measured and collected. In some embodiments of any of the methods disclosed herein, a composition disclosed herein is administered locally. In some embodiments, local delivery of a composition disclosed herein results in a decrease in likelihood of any one or more detrimental off-target effects. In some embodiments, local delivery of a composition disclosed herein does not results in any detrimental off-target effects. In some embodiments of any of the methods disclosed herein, a subject is followed-up by monitoring CLRN1 protein in serum (e.g., using an electrochemiluminescence assay), vital signs, urinalysis, and/or clinical chemistry. In some embodiments, monitoring of a subject administered a composition disclosed herein allows for early intervention and/or minimization of any off-target effects.

In some embodiments, following administration of a composition as described herein, serum can be collected and analyzed for CLRN1 protein measurement. In some embodiments, such measurements can take place prior to composition administration (Baseline), at week 2 following administration, and monthly for an appropriate duration (e.g., 3 months, 6 months, 1 year, 2 years, 3 years, 4 years, or greater than 5 years). In some embodiments, CLRN1 protein will not be detected in an individual's serum at baseline, or at any timepoint post-administration in individuals that received intracochlear delivery of either vehicle or a low dose of rAAV-CLRN1 particles described herein. In some embodiments, an individual receiving a composition as described herein through a method as described herein at a higher dose of rAAV-CLRN1, CLRN1 protein may be detected in serum at levels either below the limit of detection or quantification or at levels below the reported biologically active range (11 ng/mL to 27 ng/mL [Genentech 2017 which is incorporated in its entirety herein by reference]).

In some embodiments of any method disclosed herein, a method comprises collection and/or evaluation of serum for presence of a CLRN1 protein using, (e.g., an electrochemiluminescence assay as described herein).

Production Methods

AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012), each of which is incorporated in its entirety herein by reference). Methods for generating and using AAV constructs are described, for example, in U.S. Pat. Nos. 5,139,941, 4,797,368 and PCT filing application US2019/060328, each of which is incorporated in its entirety herein by reference.

Methods for obtaining viral constructs are known in the art. For example, to produce AAV constructs, the methods typically involve culturing a host cell which contains a nucleic acid sequence encoding an AAV capsid protein or fragment thereof; a functional rep gene; a recombinant AAV construct composed of AAV inverted terminal repeats (ITRs) and a coding sequence; and/or sufficient helper functions to permit packaging of the recombinant AAV construct into the AAV capsid proteins.

In some embodiments, components to be cultured in a host cell to package an AAV construct in an AAV capsid may be provided to the host cell in trans. Alternatively, any one or more components (e.g., recombinant AAV construct, rep sequences, cap sequences, and/or helper functions) may be provided by a stable host cell that has been engineered to contain one or more such components using methods known to those of skill in the art. In some embodiments, such a stable host cell contains such component(s) under the control of an inducible promoter. In some embodiments, such component(s) may be under the control of a constitutive promoter. In some embodiments, a selected stable host cell may contain selected component(s) under the control of a constitutive promoter and other selected component(s) under the control of one or more inducible promoters. For example, a stable host cell may be generated that is derived from HEK293 cells (which contain E1 helper functions under the control of a constitutive promoter), but that contain the rep and/or cap proteins under the control of inducible promoters. Other stable host cells may be generated by one of skill in the art using routine methods.

Recombinant AAV construct, rep sequences, cap sequences, and helper functions required for producing an AAV of the disclosure may be delivered to a packaging host cell using any appropriate genetic element (e.g., construct). A selected genetic element may be delivered by any suitable method known in the art, e.g., to those with skill in nucleic acid manipulation and include genetic engineering, recombinant engineering, and synthetic techniques (see, e.g., Sambrook et al, Molecular Cloning: A Laboratory Manual, Cold Spring Harbor Press, Cold Spring Harbor, N.Y., which is incorporated in its entirety herein by reference). Similarly, methods of generating AAV particles are well known and any suitable method can be used with the present disclosure (see, e.g., K. Fisher et al, J. Virol., 70:520-532 (1993) and U.S. Pat. No. 5,478,745, which are incorporated in their entirety herein by reference).

In some embodiments, recombinant AAVs may be produced using a triple transfection method (e.g., as described in U.S. Pat. No. 6,001,650, which is incorporated in its entirety herein by reference). In some embodiments, recombinant AAVs are produced by transfecting a host cell with a recombinant AAV construct (comprising a coding sequence) to be packaged into AAV particles, an AAV helper function construct, and an accessory function construct. An AAV helper function construct encodes “AAV helper function” sequences (i.e., rep and cap), which function in trans for productive AAV replication and encapsidation. In some embodiments, the AAV helper function construct supports efficient AAV construct production without generating any detectable wild type AAV particles (i.e., AAV particles containing functional rep and cap genes). Non-limiting examples of constructs suitable for use with the present disclosure include pHLP19 (see, e.g., U.S. Pat. No. 6,001,650, which is incorporated in its entirety herein by reference) and pRep6cap6 construct (see, e.g., U.S. Pat. No. 6,156,303, which is incorporated in its entirety herein by reference). An accessory function construct encodes nucleotide sequences for non-AAV derived viral and/or cellular functions upon which AAV is dependent for replication (i.e., “accessory functions”). Accessory functions may include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any known helper viruses such as adenovirus, herpesvirus (other than herpes simplex virus type-1), and vaccinia virus.

Additional methods for generating and isolating AAV viral constructs suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAVs are separated from contaminating virus. Other systems do not require infection with helper virus to recover the AAV—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In some embodiments, viral construct titers post-purification are determined. In some embodiments, titers are determined using quantitative PCR. In certain embodiments, a TaqMan probe specific to a construct is utilized to determine construct levels. In certain embodiments, the TaqMan probe is represented by SEQ ID NO: 65, while forward and reverse amplifying primers are exemplified by SEQ ID NO: 66 and 67 respectively.

Exemplary TaqMan probe for quantification of constructs
(SEQ ID NO: 65)
/56-FAM/TAATTCCAA/ZEN/CCAGCAGAGTCAGGGC/3IABKFQ/
Exemplary forward qPCR primer for quantification of constructs
(SEQ ID NO: 66)
GATACAGCTAGAGTCCTGATTGC
Exemplary reverse qPCR primer for quantification of constructs
(SEQ ID NO: 67)
GATCTGCCAAGTACCTCACTATG

As described herein, in some embodiments, a viral construct of the present disclosure is an adeno-associated virus (AAV) construct. Several AAV serotypes have been characterized, including AAV1, AAV2, AAV3 (e.g., AAV3B), AAV4, AAV5, AAV6, AAV7, AAV8, AAV9, AAV10, AAV11, and AAV Anc80, as well as variants thereof. In some embodiments, an AAV particle is an AAV2/6, AAV2/8, AAV2/9, or AAV2/Anc80 particle (e.g., with AAV6, AAV8, AAV9 or Anc80 capsid and construct with AAV2 ITR). Other AAV particles and constructs are described in, e.g., Sharma et al., Brain Res Bull. 2010 Feb. 15; 81(2-3): 273, which is incorporated in its entirety herein by reference. Generally, any AAV particle may be used to deliver a coding sequence described herein. However, the serotypes have different tropisms, e.g., they preferentially infect different tissues. In some embodiments, an AAV construct is a self-complementary AAV construct.

The present disclosure provides, among other things, methods of making AAV-based constructs. In some embodiments, such methods include use of host cells. In some embodiments, a host cell is a mammalian cell. A host cell may be used as a recipient of an AAV helper construct, an AAV minigene plasmid, an accessory function construct, and/or other transfer DNA associated with the production of recombinant AAVs. The term includes the progeny of an original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

Additional methods for generating and isolating AAV particles suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAV particles are separated from contaminating virus. Other systems do not require infection with helper virus to recover the AAV particles—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In yet another system, a coding sequence flanked by ITRs and rep/cap genes are introduced into insect host cells by infection with baculovirus-based constructs. Such production systems are known in the art (see generally, e.g., Zhang et al., 2009, Human Gene Therapy 20:922-929, which is incorporated in its entirety herein by reference). Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065, each of which is incorporated in its entirety herein by reference.

Vision Loss

Among other things, the present disclosure provides methods. In some embodiments, a method comprises introducing a composition as described herein into the eye of a subject. For example, provided herein are methods that in some embodiments include administering to an eye of a subject (e.g., an animal, e.g., a mammal, e.g., a primate, e.g., a human) a therapeutically effective amount of any composition described herein. In some embodiments of any of these methods, the subject has been previously identified as having a defective eye cell target gene (e.g., a supporting and/or vision cell target gene having a mutation that results in a decrease in the expression and/or activity of a supporting and/or vision cell target protein encoded by the gene). Some embodiments of any of these methods further include, prior to the introducing or administering step, determining that the subject has a defective eye cell target gene. Some embodiments of any of these methods can further include detecting a mutation in an eye cell target gene in a subject. Some embodiments of any of the methods can further include identifying or diagnosing a subject as having vision loss.

In some embodiments, provided herein are methods of correcting an eye cell target gene defect (e.g., a defect in CLRN1) in an eye of a subject, e.g., an animal, e.g., a mammal, e.g., a primate, e.g., a human. In some embodiments, methods include administering to the eye of a subject a therapeutically effective amount of any of the compositions described herein, where the administering repairs and or ameliorates the eye cell target gene defect in any cell subset of the eye of a subject. In some embodiments, the eye target cell may be a sensory cell, e.g., an eye cell, and/or a non-sensory cell, e.g., a supporting cell, and/or all or any subset of eye cells.

Also provided herein are methods of increasing the expression level of an eye cell target protein in any subset of eye cells of a subject (e.g., an animal, e.g., a mammal, e.g., a primate, e.g., a human,) that include: administering to the eye of the subject a therapeutically effective amount of any of the compositions described herein, where the administering results in an increase in the expression level of the eye cell target protein (e.g., clarin 1 protein) in any cell subset of the eye of a subject. In some embodiments, the eye target cell may be a sensory cell, e.g., an eye cell, and/or a non-sensory cell, e.g., a supporting cell, and/or all or any subset of eye cells.

Also provided herein are methods of treating vision loss, e.g., caused by retinitis pigmentosa, in a subject (e.g., an animal, e.g., a mammal, e.g., a primate, e.g., a human) identified as having a defective eye cell target gene that include: administering to the eye of the subject a therapeutically effective amount of any of the compositions described herein. In some embodiments of any of the methods provided herein, the subject has Usher syndrome type III. In some embodiments of any of the methods provided herein, the subject has retinitis pigmentosa. In some embodiments of any of the methods provided herein, the subject is a human. Some embodiments of any of the methods provided herein further include, prior to the administering step, determining that the subject has a defective CLRN1 gene.

Also provided herein are methods of restoring synapses and/or preserving spiral ganglion nerves in a subject identified or diagnosed as having an eye disorder that include: administering to the eye of the subject a therapeutically effective amount of any of the compositions described herein.

Also provided herein are methods that include administering to an eye of a subject a therapeutically effective amount of any of the compositions described herein.

Also provided herein are methods comprising administering a composition disclosed herein, e.g., rAAV-CLRN1, for the treatment of a subject, e.g., mammal, e.g., human, e.g., patient, with Usher syndrome type III. In some embodiments, a composition disclosed herein is delivered via surgical delivery, e.g., to the eye.

Also provided herein are surgical methods for treatment of Usher syndrome type III or retinitis pigmentosa characterized by vision loss. In some embodiments, the methods include the steps of: introducing into an eye of a subject a first incision at a first incision point; and administering intra-ocularly a therapeutically effective amount of any of the compositions provided herein. In some embodiments, the composition is administered to the subject at the first incision point. In some embodiments, the composition is administered to the subject into or through the first incision.

In some embodiments of any of the methods described herein, any composition described herein is administered to the subject into or through the retina. In some embodiments of any of the methods described herein, the composition is administered using a medical device capable of creating a plurality of incisions in the retina. In some embodiments, the medical device includes a plurality of micro-needles. In some embodiments, the medical device includes a plurality of micro-needles including a generally circular first aspect, where each micro-needle has a diameter of at least about 10 microns. In some embodiments, the medical device includes a base and/or a reservoir capable of holding the composition. In some embodiments, the medical device includes a plurality of hollow micro-needles individually including a lumen capable of transferring the composition. In some embodiments, the medical device includes a means for generating at least a partial vacuum.

In some embodiments of any of the methods described herein, any composition described herein is administered to the subject via an intravitreal injection or subretinal injection, for example, as described by Ochakovski et al., “Retinal Gene Therapy: Surgical Vector Delivery in the Translation to Clinical Trials”, Front. Neurosci. Apr. 3, 2017, the contents of which are hereby incorporated by reference herein in its entirety. In some embodiments of any of the methods described herein, any composition described herein is administered to the subject as described by “OCT—Assisted Delivery of Luxturna” by Ninel Z Gregori and Janet Louise David, https://www.aao.org/clinical-video/oct-assisted-delivery-of-luxturna (Jul. 19, 2018), the contents of which are hereby incorporated by reference in its entirety. In some embodiments of any of the methods described herein, any composition described herein is administered to the subject via suprachoroidal delivery (e.g., using Orbit™ Subretinal Delivery System (Orbit SDS) (Gyroscope Therapeutics)).

In some embodiments, technologies of the present disclosure are used to treat subjects with or at risk of vision loss. For example, in some embodiments, a subject has an autosomal recessive vision loss attributed to at least one pathogenic variant of CLRN1. It will be understood by those in the art that many different mutations in CLRN1 can result in a pathogenic variant. In some such embodiments, a pathogenic variant causes or is at risk of causing vision loss.

In some embodiments, a subject experiencing vision loss will be evaluated to determine if and where one or more mutations may exist that may cause vision loss. In some such embodiments, the status of CLRN1 gene products or function (e.g., via protein or sequencing analyses) will be evaluated. In some embodiments of any of the methods described herein, the subject or animal is a mammal, in some embodiments the mammal is a domestic animal, a farm animal, a zoo animal, a non-human primate, or a human. In some embodiments of any of the methods described herein, the animal, subject, or mammal is an adult, a teenager, a juvenile, a child, a toddler, an infant, or a newborn. In some embodiments of any of the methods described herein, the animal, subject, or mammal is 1-5, 1-10, 1-20, 1-30, 1-40, 1-50, 1-60, 1-70, 1-80, 1-90, 1-100, 1-110, 2-5, 2-10, 10-20, 20-30, 30-40, 40-50, 50-60, 60-70, 70-80, 80-90, 90-100, 100-110, 10-30, 10-40, 10-50, 10-60, 10-70, 10-80, 10-90, 10-100, 10-110, 20-40, 20-50, 20-60, 20-70, 20-80, 20-90, 20-100, 20-110, 30-50, 30-60, 30-70, 30-80, 30-90, 30-100, 40-60, 40-70, 40-80, 40-90, 40-100, 50-70, 50-80, 50-90, 50-100, 60-80, 60-90, 60-100, 70-90, 70-100, 70-110, 80-100, 80-110, or 90-110 years of age. In some embodiments of any of the methods described herein, the subject or mammal is 1, 2, 3, 4, 5, 6, 7, 8, 9, 10, or 11 months of age.

In some embodiments of any of the methods described herein, the methods result in improvement in vision (e.g., any of the metrics for determining improvement in vision described herein) in a subject in need thereof for at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 100 days, at least 105 days, at least 110 days, at least 115 days, at least 120 days, at least 5 months, at least 6 months, at least 7 months, at least 8 months, at least 9 months, at least 10 months, at least 11 months, or at least 12 months.

In some embodiments a subject (e.g., an animal, e.g., a mammal, e.g., a human) has or is at risk of developing vision loss. In some embodiments a subject (e.g., an animal, e.g., a mammal, e.g., a human) has been previously identified as having a mutation in a CLRN1 gene. In some embodiments a subject (e.g., an animal, e.g., a mammal, e.g., a human) has any of the mutations in a CLRN1 gene that are described herein or are known in the art to be associated with vision loss.

In some embodiments, a subject (e.g., an animal, e.g., a mammal, e.g., a human) has been identified as being at risk of vision loss (e.g., at risk of being a carrier of a gene mutation, e.g., a CLRN1 mutation). In some such embodiments, a subject (e.g., an animal, e.g., a mammal, e.g., a human) may have certain risk factors of vision loss or risk of vision loss (e.g., known parental carrier, afflicted sibling, or symptoms of vision loss). In some such embodiments, a subject (e.g., an animal, e.g., a mammal, e.g., a human) has been identified as being a carrier of a mutation in a CLRN1 gene (e.g., via genetic testing) that has not previously been identified (i.e., is not a published or otherwise known variant of CLRN1). In some such embodiments, identified mutations may be novel (i.e., not previously described in the literature), and methods of treatment for a subject suffering from or susceptible to vision loss will be personalized to the mutation(s) of the particular patient. In some embodiments, a subject (e.g., an animal, e.g., a mammal, e.g., a human) has been identified as having a mutation in a CLRN1 gene and has been diagnosed with vision loss. In some embodiments, a subject (e.g., an animal, e.g., a mammal, e.g., a human) has been identified as having vision loss.

In some embodiments, successful treatment of vision loss can be determined in a subject using any of the conventional functional vision tests known in the art. Non-limiting examples of functional vision tests are various types of vision assays (e.g., eye muscle tests, visual acuity tests, refraction assessments, visual field tests, color vision tests, and retinal examination).

In some embodiments of any method provided herein, two or more doses of any composition described herein are introduced or administered into an eye of a subject. Some embodiments of any of these methods can include introducing or administering a first dose of a composition into an eye of a subject, assessing vision function of the subject following introduction or administration of a first dose, and administering an additional dose of a composition into the eye of the subject found not to have a vision function within a normal range (e.g., as determined using any test for vision known in the art).

In some embodiments of any method provided herein, the composition can be formulated for intra-ocular administration. In some embodiments of any of the methods described herein, the compositions described herein can be administered via intra-ocular administration, intravitreal administration, subretinal administration, suprachoroidal delivery, or local administration. In some embodiments of any of the methods described herein, the compositions are administered through the use of a medical device (e.g., any of the exemplary medical devices described herein).

In some embodiments, intra-ocular administration can be performed using any of the methods described herein or known in the art. For example, in some embodiments, a composition can be administered or introduced into the eye using the following surgical techniques.

A microscope-integrated intraoperative optical coherence tomography (OCT) is used to create a small pre-bleb before viral injection (e.g., to avoid a sub-retinal pigment epithelium (RPE) injection or suprachorodial injection as need). First, the hyaloid is stained and removed using a finesse loop and elevated with a soft silicone tip. A balanced salt solution is then injected into the subretinal space and an OCT scan is done to check for foveal thinning. Once the bleb is made, voretigene is introduced using a 25-gauge no-duel bore cannula. The fovea needs to be carefully monitored thereafter to avoid excessive stretching and macular hole formation. Scleral indentation can be used to rule out peripheral breaks. Finally, an air-fluid exchange is used to clear the virus (see, e.g., “OCT—Assisted Delivery of Luxturna” by Ninel Z Gregori and Janet Louise David, https://www.aao.org/clinical-video/oct-assisted-delivery-of-luxturna (Jul. 19, 2018), the contents of which are hereby incorporated by reference in its entirety).

As another example, as described herein, the Orbit™ Subretinal Delivery System (Orbit SDS) (Gyroscope Therapeutics) can be used as administering or introducing the described compositions into the eye via a suprachoroidal injection. See, e.g., “Orbit Subretinal Delivery System Instructions for Use June 2020”, https://www.orbitsds.com/wp-content/uploads/2020/08/AW1009028-Rev.-D-US-IFU-No-Cut-Lines.pdf, the contents of which are hereby incorporated by reference herein in its entirety).

In some embodiments of any method provided herein, a subject has or is at risk of developing vision loss. In some embodiments of any method provided herein, a subject has been previously identified as having a mutation in an eye cell target gene, a gene which may be expressed in supporting cells and/or eye cells.

In some embodiments of any method provided herein, a subject has been identified as being a carrier of a mutation in an eye cell target gene (e.g., via genetic testing). In some embodiments of any method provided herein, a subject has been identified as having a mutation in an eye cell target gene and has been diagnosed with vision loss (e.g., Usher Type III syndrome). In some embodiments of any of the methods described herein, the subject has been identified as having vision loss (e.g., Usher Type III syndrome). In some embodiments, successful treatment of vision loss (e.g., Usher Type III syndrome) can be determined in a subject using any of the conventional functional vision tests known in the art. Non-limiting examples of functional vision tests include various types of vision assays (e.g., eye muscle tests, visual acuity tests, refraction assessments, visual field tests, color vision tests, and retinal examination).

In some embodiments, a subject cell is in vitro. In some embodiments, a subject cell is originally obtained from a subject and is cultured ex vivo. In some embodiments, a subject cell has previously been determined to have a defective eye target gene. In some embodiments, a subject cell has previously been determined to have a defective eye cell target gene. In some embodiments, a subject cell has previously been determined to have a defective supporting cell target gene.

In some embodiments of these methods, following treatment e.g., one or two or more administrations of compositions described herein, there is an increase in expression of an active eye cell target protein (e.g., clarin 1 protein). In some embodiments, an increase in expression of an active eye target protein as described herein (e.g., clarin 1 protein) is relative to a control level, e.g., as compared to the level of expression of an eye cell target protein prior to introduction of the compositions comprising any construct(s) as described herein.

Methods of detecting expression and/or activity of a target protein (e.g., clarin 1 protein) are known in the art. In some embodiments, a level of expression of an eye cell target protein can be detected directly (e.g., detecting eye cell target protein or target mRNA. Non-limiting examples of techniques that can be used to detect expression and/or activity of a target RNA or protein (e.g., a CLRN1 gene product and/or clarin 1 protein or functional characteristic portion thereof) directly include: real-time PCR, Western blotting, immunoprecipitation, immunohistochemistry, mass spectrometry, or immunofluorescence. In some embodiments, expression of an eye cell target protein can be detected indirectly (e.g., through functional vision tests).

In some embodiments, safety and tolerability of a composition disclosed herein can be assessed in vision loss. In some embodiments, safety and tolerability of a composition disclosed herein, e.g., rAAV-CLRN1 can be assessed in a subject disclosed herein.

Administration

Provided herein are therapeutic delivery systems for treating Usher syndrome type III characterized by vision loss. In one aspect, a therapeutic delivery system includes: i) a medical device capable of creating one or a plurality of incisions in an eye of a subject in need thereof, and ii) an effective dose of a composition (e.g., any of the compositions described herein). In some embodiments, a medical device includes a plurality of micro-needles.

Also provided herein are therapeutic delivery systems for treating retinitis pigmentosa characterized by vision loss. In one aspect, a therapeutic delivery system includes: i) a medical device capable of creating one or a plurality of incisions in an eye of a subject in need thereof, and ii) an effective dose of a composition (e.g., any of the compositions described herein). In some embodiments, a medical device includes a plurality of micro-needles.

Also provided herein are surgical methods for treatment of Usher syndrome type III or retinitis pigmentosa characterized by vision loss. In some embodiments, a method the steps of: introducing into an eye of a subject a first incision at a first incision point; and administering intra-ocularly a therapeutically effective amount of any of the compositions provided herein. In some embodiments, a composition is administered to a subject at the first incision point. In some embodiments, a composition is administered to a subject into or through the first incision.

In some embodiments of any method provided herein, any of the compositions described herein is administered to the subject into or through the eye. In some embodiments of any method provided herein, the composition is administered using a medical device capable of creating a plurality of incisions in the eye. In some embodiments, a medical device includes a plurality of micro-needles. In some embodiments, a medical device includes a plurality of micro-needles including a generally circular first aspect, where each micro-needle has a diameter of at least about 10 microns. In some embodiments, a medical device includes a base and/or a reservoir capable of holding a composition. In some embodiments, a medical device includes a plurality of hollow micro-needles individually including a lumen capable of transferring a composition. In some embodiments, a medical device includes a means for generating at least a partial vacuum.

In some embodiments, any of the methods disclosed herein comprise a dose-escalation study to assess safety and tolerability in subjects, e.g., mammals, e.g., humans, e.g., patients, with vision loss. In some embodiments, a composition disclosed herein, e.g., rAAV-CLRN1, is administered at a dosing regimen disclosed herein. In some embodiments, the dosing regimen comprises either intraocular administrations of a dose, e.g., as described herein, of a composition disclosed herein, e.g., rAAV-CLRN1. In some embodiments, the dosing regimen comprises delivery in a volume of at least 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per eye. In some embodiments, the dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, or at most 0.05 mL per eye. In some embodiments, the dosing regimen comprises delivery in a volume of about 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per eye, depending on the population. In some embodiments, the dosing regimen comprises delivery in a volume of at least 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per eye. In some embodiments, the dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, or at most 0.05 mL per eye. In some embodiments, the dosing regimen comprises delivery in a volume of about 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per eye, depending on the population.

In some embodiments, a method disclosed herein evaluates the safety and tolerability of escalating doses of a composition disclosed herein, e.g., rAAV-CLRN1, administered via intraocular injection to a subject, e.g., 18 to 80 years of age, with vision loss.

In some embodiments, any of the methods disclosed herein comprise an evaluation of the safety and tolerability of a composition disclosed herein, e.g., rAAV-CLRN1. In some embodiments, evaluation of the efficacy of a composition disclosed herein, e.g., rAAV-CLRN1 to treat vision loss, is performed in a randomized, controlled setting (using a concurrent, non-intervention observation arm).

Methods of Treating a Subject

The present disclosure provides, among other things, that technologies described herein may be used to treat an underlying disease and/or symptoms in a subject suffering from or at risk of Usher syndrome type III or retinitis pigmentosa characterized by vision loss.

In some embodiments, a method comprises administering a construct (e.g., an rAAV construct) described herein, a particle (e.g., an rAAV particle), or a composition described herein to a subject. In some embodiments, a method is a method of treatment. In some embodiments, a subject is a subject suffering from or at risk of Usher syndrome type III or retinitis pigmentosa characterized by vision loss.

In some embodiments, administering a construct (e.g., an rAAV construct) described herein, a particle (e.g., an rAAV particle), or a composition described herein to a subject may alleviate and/or ameliorate one or more symptoms associated with Usher syndrome type III or retinitis pigmentosa characterized by vision loss. Symptoms can include, for example, vision loss, loss of peripheral vision, loss of central vision, or degeneration of eye cells.

In some embodiments, vision loss is associated with a gene mutation (e.g., a deletion mutation, a frameshift mutation, a nonsense mutation, a hypomorphic mutation, a hypermorphic mutation, a neomorphic mutation, aberrant over expression, aberrant under expression, etc.). In some embodiments, a subject suffering from or at risk of Usher syndrome type III or retinitis pigmentosa may have a mutation in a CLRN1 gene, which may be characterized as described herein.

In some embodiments, a subject is genetically and/or symptomatically characterized prior to, during, and/or after treatment with technologies described herein (e.g. real-time PCR, quantitative real-time PCR, Western blotting, immunoprecipitation, immunohistochemistry, mass spectrometry, or immunofluorescence, indirect phenotypic determination of expression of a gene and/or protein (e.g., through functional vision tests, eye muscle tests, visual acuity tests, refraction assessments, visual field tests, color vision tests, and retinal examination, etc.). In some embodiments, a subject suffering from or at risk of vision loss may have their associated disease state characterized through tissue sampling (e.g., comprising one or more eye cells, e.g., comprising one or more eye cells and/or one or more supporting cells). In some embodiments, tissues are evaluated via morphological analysis to determine morphology of eye cells and/or support cells before, during, and/or after administration of any technologies (e.g., methodologies, e.g., compositions, e.g., compositions comprising constructs, and/or particles, etc.) as described herein. In some such embodiments, standard immunohistochemical or histological analyses may be performed. In some embodiments, if cells are used in-vitro or ex-vivo, additional immunocytochemical or immunohistochemical analyses may be performed. In some embodiments, one or more assays of one or more proteins or transcripts (e.g., western blot, ELISA, polymerase chain reactions) may be performed on one or more samples from a subject or in-vitro cell populations.

In some embodiments, administering a construct (e.g., an rAAV construct) described herein, a particle (e.g., an rAAV particle), or a composition described herein to a subject improves a patients immunohistochemical evaluation (e.g., tests as described above) when compared to immunohistochemical tests performed prior to treatment with technologies described herein or when compared to a control population.

Treating a Subject to Improve Symptoms of Usher Type III Syndrome or Retinitis Pigmentosa

In some embodiments, a subject suffering from or at risk of Usher Type III Syndrome characterized by vision loss may receive a treatment regime that is characterized by vision function. In some embodiments, functionality of a treatment regime is characterized through vision function, wherein such vision function is determined in an individual using eye vision tests (e.g., functional vision tests, eye muscle tests, visual acuity tests, refraction assessments, visual field tests, color vision tests, and retinal examination, etc.) before, after, and/or during treatment with compositions and methods described herein. In some embodiments, functionality of a treatment regime is characterized through vision function, wherein such vision function is determined in an individual by measuring visual acuity as described herein before, after, and/or during treatment with compositions and methods described herein. In some such embodiments, vision measurements are taken from one or both eyes of a subject. In some such embodiments, measurements are compared to prior recordings for the same subject and/or known measurements on such response measurements used to define, e.g., vision loss versus acceptable vision ranges to be defined as normal vision. In some embodiments, a subject has the described measurements taken prior to receiving any treatment. In some embodiments, a subject treated with one or more technologies described herein will have improvements on vision measurements after treatment as compared to before treatment. In some embodiments, vision measurements are taken after treatment is administered and at regular follow-up intervals post-treatment. In some embodiments, treatment with technologies described herein improve a patient's test evaluation (e.g., tests as described above) when compared to tests performed prior to treatment with technologies described herein or when compared to a control population.

In some embodiments, a subject suffering from or at risk of retinitis pigmentosa characterized by vision loss may receive a treatment regime that is characterized by vision function. In some embodiments, functionality of a treatment regime is characterized through vision function, wherein such vision function is determined in an individual using eye vision tests (e.g., through functional vision tests, eye muscle tests, visual acuity tests, refraction assessments, visual field tests, color vision tests, and retinal examination, etc.) before, after, and/or during treatment with compositions and methods described herein. In some embodiments, functionality of a treatment regime is characterized through vision function, wherein such vision function is determined in an individual by measuring visual acuity as described herein before, after, and/or during treatment with compositions and methods described herein. In some such embodiments, vision measurements are taken from one or both eyes of a subject. In some such embodiments, measurements are compared to prior recordings for the same subject and/or known measurements on such response measurements used to define, e.g., vision loss versus acceptable vision ranges to be defined as normal vision. In some embodiments, a subject has the described measurements taken prior to receiving any treatment. In some embodiments, a subject treated with one or more technologies described herein will have improvements on vision measurements after treatment as compared to before treatment. In some embodiments, vision measurements are taken after treatment is administered and at regular follow-up intervals post-treatment. In some embodiments, treatment with technologies described herein improve a patient's test evaluation (e.g., tests as described above) when compared to tests performed prior to treatment with technologies described herein or when compared to a control population.

Evaluating Vision Loss and Recovery

In some embodiments, vision function is determined using measurements including electroretinogram, visual field testing, or genetic testing. In some embodiments, the retina of the eye of a subject is examined using an ophthalmoscope, a tool that allows for a wider, clear view of the retina. In some embodiments, measurements are taken from one or both eyes of a subject. In some such embodiments, measurements are compared to prior tests for the same subject and/or known reference measurements used to define, e.g., vision loss versus what is defined in the art as normal vision. In some embodiments, a subject has measurements performed prior to receiving any treatment. In some embodiments, a subject treated with one or more technologies described herein will have improvements on measurements performed after treatment as compared to before treatment. In some embodiments, measurements are taken after treatment is administered and at regular follow-up intervals post-treatment.

Methods of Characterizing a Disease State

The term “mutation in a CLRN1 gene” refers to a modification in a known consensus functional CLRN1 gene that results in the production of a clarin 1 protein having one or more of: a deletion in one or more amino acids, one or more amino acid substitutions, and one or more amino acid insertions as compared to the consensus functional clarin 1 protein, and/or results in a decrease in the expressed level of the encoded clarin 1 protein in a mammalian cell as compared to the expressed level of the encoded clarin 1 protein in a mammalian cell not having a mutation. In some embodiments, a mutation can result in the production of a clarin 1 protein having a deletion in one or more amino acids (e.g., 2, 3, 4, 5, 6, 7, 8, 9, 10, 11, 12, 13, 14, 15 16, 17, 18, 19, 20, or more amino acids). In some embodiments, the mutation can result in a frameshift in the CLRN1 gene. The term “frameshift” is known in the art to encompass any mutation in a coding sequence that results in a shift in the reading frame of the coding sequence. In some embodiments, a frameshift can result in a nonfunctional protein. In some embodiments, a point mutation can be a nonsense mutation (i.e., result in a premature stop codon in an exon of the gene). A nonsense mutation can result in the production of a truncated protein (as compared to a corresponding consensus functional protein) that may or may not be functional. In some embodiments, the mutation can result in the loss (or a decrease in the level) of expression of CLRN1 mRNA or clarin 1 protein or both the mRNA and protein. In some embodiments, the mutation can result in the production of an altered clarin 1 protein having a loss or decrease in one or more biological activities (functions) as compared to a consensus functional clarin 1 protein.

In some embodiments, the mutation is an insertion of one or more nucleotides into a CLRN1 gene. In some embodiments, the mutation is in a regulatory and/or control sequence of the clarin 1 gene, i.e., a portion of the gene that is not coding sequence. In some embodiments, a mutation in a regulatory and/or control sequence may be in a promoter or enhancer region and prevent or reduce the proper transcription of the CLRN1 gene. In some embodiments, a mutation is in a known heterologous gene known to interact with a clarin 1 protein, or the CLRN1 gene.

Methods of genotyping and/or detecting expression or activity of CLRN1 mRNA and/or clarin 1 protein are known in the art (see e.g., Ito et al., World J Otorhinolaryngol. 2013 May 28; 3(2): 26-34, and Roesch et al., Int J Mol Sci. 2018 January; 19(1): 209, each of which is incorporated in its entirety herein by reference). In some embodiments, level of expression of CLRN1 mRNA or clarin 1 protein may be detected directly (e.g., detecting clarin 1 protein, detecting CLRN1 mRNA etc.). Non-limiting examples of techniques that can be used to detect expression and/or activity of CLRN1 directly include, e.g., real-time PCR, quantitative real-time PCR, Western blotting, immunoprecipitation, immunohistochemistry, mass spectrometry, or immunofluorescence. In some embodiments, expression of CLRN1 and/or clarin 1 protein can be detected indirectly (e.g., through functional vision tests, eye muscle tests, visual acuity tests, refraction assessments, visual field tests, color vision tests, and retinal examination, etc.).

Evaluating CLRN1 Protein Concentration in Biological Samples

In some embodiments, methods described herein include evaluating CLRN1 protein concentrations in one or more biological samples form an individual before, during, and/or after treatment with compositions described herein.

In some embodiments of these methods, following treatment e.g., one or two or more administrations of compositions described herein, there is an increase in expression of a CLRN1 protein. In some embodiments, an increase in expression of an active CLRN1 protein as described herein when compared relative to a control level, e.g., as compared to the level of expression of a CLRN1 protein prior to introduction of the compositions comprising any construct(s) as described herein.

Methods of detecting expression and/or activity of a target RNA and/or protein are known in the art. In some embodiments, a level of expression of an eye target protein can be detected directly (e.g., detecting eye cell target protein or target mRNA). Non-limiting examples of techniques that can be used to detect expression and/or activity of a target RNA or protein directly include: real-time PCR, Western blotting, immunoprecipitation, immunohistochemistry, mass spectrometry, or immunofluorescence. In some embodiments, expression of an eye cell target protein can be detected indirectly (e.g., through functional vision tests).

In some embodiments, biodistribution and/or shedding analysis of rAAV particles is performed. In some embodiments, disclosed herein is a composition comprising an rAAV-CLRN1 replication-defective, rAAV particle designed to deliver a cDNA, e.g., to express a CLRN1 protein. In some embodiments, an rAAV particle as described herein is used to treat a subject, e.g., a human, e.g., a patient, with Usher syndrome type III. In some embodiments, a composition disclosed herein is administered via an intraocular route. In some embodiments, a composition disclosed herein is administered at a low dose of rAAV particles (e.g., as measured by vector genome qPCR analysis). In some embodiments, a composition disclosed herein is administered to a localized area of the body. In some embodiments, a composition disclosed herein, is expected to result in limited vascular spread and systemic exposure. In some embodiments, a composition disclosed herein results in higher levels of construct sequences in the eye. In some embodiments, a composition disclosed herein results in lower but detectable levels in most other tissues and fluids collected. In some embodiments, levels of construct sequences were generally decreased overall by six months. In some embodiments, levels of construct sequences decrease over time in blood samples, e.g., one month following intraocular administration of a composition disclosed herein.

In some embodiments of any of the methods disclosed herein, relevant fluids for biodistribution and shedding (e.g., blood, serum, urine, saliva, nasal and ear swabs, and CSF fluid) are collected and/or evaluated. In some embodiments of any of the methods disclosed herein, non-target tissues are collected and/or evaluated. In some embodiments, capsid variant is expected to determine tropism. In some embodiments, a composition disclosed herein comprises a capsid variant, e.g., AAV Anc80 capsid variant. In some embodiments, delivery of a composition disclosed herein comprising a capsid variant, e.g., via the same route of administration, in the same particle formulation, and/or at equivalent or lower particle doses, is not expected to result in differences in biodistribution and shedding patterns.

In some embodiments, a composition disclosed herein, e.g., rAAV-CLRN1, is dosed at a level less than about 2E12 total vg/cochlea. In some embodiments, a dose of a composition disclosed herein is a function of, e.g., limitations of volume and rAAV particle concentration.

In contrast, clinical trials using rAAV particles have delivered more than 1E14 vg/kg via systemic routes of administration, in some cases to participants younger than 6 months of age (AveXis 2019, incorporated herein in its entirety by reference). Other localized deliveries with relatively low doses of rAAV particles have not reported extensive biodistribution beyond the target area, e.g., throughout the body, or extensive rAAV particle shedding (excretion; generally measuring underlying construct DNA concentration through methods such as qPCR). For example, low levels of distribution beyond the ocular target area (specifically in the optic nerve of the particle-injected eye, optic chiasm, spleen and liver, and sporadically in the lymph nodes of study animals) have been reported for Luxturna® delivered bilaterally at a dose of 7.5E11 vg/eye. Similarly, in the Phase 3 clinical trial, rAAV particle was shed transiently and at low levels in tears of 45% of the participants; it was detected, also at low levels, in serum (but not whole blood) samples of 10% of the participants in the days immediately following subretinal administration of Luxturna® delivered bilaterally at a dose of 1.5E11 vg/eye (see, e.g., Russell 2017; Spark Therapeutics 2017, each of which is incorporated herein in its entirety by reference).

In some embodiments, any of the methods disclosed herein comprise evaluating a distribution of rAAV particle sequences in blood (e.g., serum and whole blood) over a time course following unilateral administration to the cochlea using, e.g., a validated qPCR method. In some embodiments, additional specimens (e.g., external auditory canal swabs, nasal swabs, saliva, and urine) will be collected for evaluation of shedding from the subject. In some embodiments, a specimen is collected from a subject until at least three consecutive negative samples are obtained.

In some embodiments, proteins correlating with vision loss are measured before, during, and/or after treatment with compositions and/or methods described herein.

In some embodiments, immunogenicity to AAV capsids and/or particles are measured. Immunogenicity to AAV capsids and/or particles delivered to localized areas, and in relatively low doses compared to systemic applications, have generally not yielded specific patterns of immune responses; importantly, responses observed through both humoral and cell-mediated immunological monitoring (e.g., through enzyme-linked immunosorbent assay [ELISA]/neutralizing antibody [NAb] and enzyme-linked immunosorbent spot [ELISPOT] assays, respectively) have predominantly been without clinical correlate for route(s) of administration (ROA) that afford some immunological protection (e.g., direct administration to the brain).

In some embodiments of any of the methods disclosed herein, an intraocular ROA is expected to provide a similar level of protection. In some embodiments of any of the methods disclosed herein, a subject will receive a short, peri-operative course of an immunomodulatory regimen, e.g., systemic oral corticosteroids, for approximately 17 days, beginning 3 days before administration of a compositions disclosed herein, e.g., rAAV-CLRN1. In some embodiments, the immunomodulatory regimen reduces inflammation related to the surgical administration procedure. In some embodiments, the immunomodulatory regimen can also further reduce the potential for an immune reaction to either a capsid (e.g., AAV Anc80) or the underlying construct (e.g., a transgene product, e.g., a CLRN1 protein).

In some embodiments, any method disclosed herein further comprise evaluating humoral immunity (e.g., antibody responses) in response to administration of a composition disclosed herein. In some embodiments, effect of pre-existing immunity, measured e.g., by serum NAb levels, on the transduction of a compositions disclosed herein when delivered via the intraocular ROA is evaluated. In some embodiments, pre-existing NAb levels do not inhibit transduction of AAV particles delivered by an intraocular route of administration. In some embodiments, any method disclosed herein further comprise evaluating serum for potential systemic humoral responses to both the AAV capsid and/or the transgene product (e.g., a protein). In some embodiments, responsive to the evaluation of systemic humoral responses, a treatment interval for bilateral intraocular administration of a composition disclosed herein, e.g., rAAV-CLRN1 can be developed.

In some embodiments, any method disclosed herein does not result in cytotoxic T cell responses, e.g., to either an AAV particle, capsid, and/or construct (e.g. underlying transgene) product from rAAV particles delivered via a localized route of administration (ROA), such as intraocular administration. For example, the labeling for Luxturna® notes that no subject had a clinically significant, cytotoxic T cell response (Spark Therapeutics 2017, incorporated herein in its entirety by reference); isolated positive interferon-gamma (IFN-gamma) ELISPOT assay results were obtained during the clinical development program (see, e.g., Bennett 2012, incorporated herein in its entirety by reference), but the significance of these isolated results is unknown, as no clinical inflammatory response was observed and no dose limiting toxicity was seen in the clinical program.

In certain embodiments, pharmacokinetics of any of the compositions or products of compositions described herein are measured and collected. In some embodiments of any of the methods disclosed herein, a composition disclosed herein is administered locally. In some embodiments, local delivery of a composition disclosed herein results in a decrease in likelihood of any one or more detrimental off-target effects. In some embodiments, local delivery of a composition disclosed herein does not results in any detrimental off-target effects. In some embodiments of any of the methods disclosed herein, a subject is followed-up by monitoring CLRN1 protein in serum (e.g., using an electrochemiluminescence assay), vital signs, urinalysis, and/or clinical chemistry. In some embodiments, monitoring of a subject administered a composition disclosed herein allows for early intervention and/or minimization of any off-target effects.

In some embodiments, following administration of a composition as described herein, serum can be collected and analyzed for CLRN1 protein measurement. In some embodiments, such measurements can take place prior to composition administration (Baseline), at week 2 following administration, and monthly for an appropriate duration (e.g., 3 months, 6 months, 1 year, 2 years, 3 years, 4 years, or greater than 5 years). In some embodiments, CLRN1 protein will not be detected in an individual's serum at baseline, or at any timepoint post-administration in individuals that received intraocular delivery of either vehicle or a low dose of rAAV-CLRN1 particles described herein. In some embodiments, an individual receiving a composition as described herein through a method as described herein at a higher dose of rAAV-CLRN1, CLRN1 protein may be detected in serum at levels either below the limit of detection or quantification or at levels below the reported biologically active range (11 ng/mL to 27 ng/mL [see, e.g., Genentech 2017 which is incorporated in its entirety herein by reference]).

In some embodiments of any method disclosed herein, a method comprises collection and/or evaluation of serum for presence of a CLRN1 protein using, (e.g., an electrochemiluminescence assay as described herein).

Production Methods

AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012), each of which is incorporated in its entirety herein by reference). Methods for generating and using AAV constructs are described, for example, in U.S. Pat. Nos. 5,139,941, 4,797,368 and PCT filing application US2019/060328, each of which is incorporated in its entirety herein by reference.

Methods for obtaining viral constructs are known in the art. For example, to produce AAV constructs, the methods typically involve culturing a host cell which contains a nucleic acid sequence encoding an AAV capsid protein or fragment thereof; a functional rep gene; a recombinant AAV construct composed of AAV inverted terminal repeats (ITRs) and a coding sequence; and/or sufficient helper functions to permit packaging of the recombinant AAV construct into the AAV capsid proteins.

In some embodiments, components to be cultured in a host cell to package an AAV construct in an AAV capsid may be provided to the host cell in trans. Alternatively, any one or more components (e.g., recombinant AAV construct, rep sequences, cap sequences, and/or helper functions) may be provided by a stable host cell that has been engineered to contain one or more such components using methods known to those of skill in the art. In some embodiments, such a stable host cell contains such component(s) under the control of an inducible promoter. In some embodiments, such component(s) may be under the control of a constitutive promoter. In some embodiments, a selected stable host cell may contain selected component(s) under the control of a constitutive promoter and other selected component(s) under the control of one or more inducible promoters. For example, a stable host cell may be generated that is derived from HEK293 cells (which contain E1 helper functions under the control of a constitutive promoter), but that contain the rep and/or cap proteins under the control of inducible promoters. Other stable host cells may be generated by one of skill in the art using routine methods.

Recombinant AAV construct, rep sequences, cap sequences, and helper functions required for producing an AAV of the disclosure may be delivered to a packaging host cell using any appropriate genetic element (e.g., construct). A selected genetic element may be delivered by any suitable method known in the art, e.g., to those with skill in nucleic acid manipulation and include genetic engineering, recombinant engineering, and synthetic techniques (see, e.g., Sambrook et al, Molecular Cloning: A Laboratory Manual, Cold Spring Harbor Press, Cold Spring Harbor, N.Y., which is incorporated in its entirety herein by reference). Similarly, methods of generating AAV particles are well known and any suitable method can be used with the present disclosure (see, e.g., K. Fisher et al, J. Virol., 70:520-532 (1993) and U.S. Pat. No. 5,478,745, which are incorporated in their entirety herein by reference).

In some embodiments, recombinant AAVs may be produced using a triple transfection method (e.g., as described in U.S. Pat. No. 6,001,650, which is incorporated in its entirety herein by reference). In some embodiments, recombinant AAVs are produced by transfecting a host cell with a recombinant AAV construct (comprising a coding sequence) to be packaged into AAV particles, an AAV helper function construct, and an accessory function construct. An AAV helper function construct encodes “AAV helper function” sequences (i.e., rep and cap), which function in trans for productive AAV replication and encapsidation. In some embodiments, the AAV helper function construct supports efficient AAV construct production without generating any detectable wild type AAV particles (i.e., AAV particles containing functional rep and cap genes). Non-limiting examples of constructs suitable for use with the present disclosure include pHLP19 (see, e.g., U.S. Pat. No. 6,001,650, which is incorporated in its entirety herein by reference) and pRep6cap6 construct (see, e.g., U.S. Pat. No. 6,156,303, which is incorporated in its entirety herein by reference). An accessory function construct encodes nucleotide sequences for non-AAV derived viral and/or cellular functions upon which AAV is dependent for replication (i.e., “accessory functions”). Accessory functions may include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any known helper viruses such as adenovirus, herpesvirus (other than herpes simplex virus type-1), and vaccinia virus.

Additional methods for generating and isolating AAV viral constructs suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAVs are separated from contaminating virus. Other systems do not require infection with helper virus to recover the AAV—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In some embodiments, viral construct titers post-purification are determined. In some embodiments, titers are determined using quantitative PCR. In certain embodiments, a TaqMan probe specific to a construct is utilized to determine construct levels. In certain embodiments, the TaqMan probe is represented by SEQ ID NO: 65, while forward and reverse amplifying primers are exemplified by SEQ ID NO: 66 and 67 respectively.

Exemplary TaqMan probe for quantification of constructs
(SEQ ID NO: 65)
/56-FAM/TAATTCCAA/ZEN/CCAGCAGAGTCAGGGC/3IABKFQ/
Exemplary forward qPCR primer for quantification of constructs
(SEQ ID NO: 66)
GATACAGCTAGAGTCCTGATTGC
Exemplary reverse qPCR primer for quantification of constructs
(SEQ ID NO: 67)
GATCTGCCAAGTACCTCACTATG

As described herein, in some embodiments, a viral construct of the present disclosure is an adeno-associated virus (AAV) construct. Several AAV serotypes have been characterized, including AAV1, AAV2, AAV3 (e.g., AAV3B), AAV4, AAV5, AAV6, AAV7, AAV8, AAV9, AAV10, AAV11, and AAV Anc80, as well as variants thereof. In some embodiments, an AAV particle is an AAV2/6, AAV2/8, AAV2/9, or AAV2/Anc80 particle (e.g., with AAV6, AAV8, AAV9 or Anc80 capsid and construct with AAV2 ITR). Other AAV particles and constructs are described in, e.g., Sharma et al., Brain Res Bull. 2010 Feb. 15; 81(2-3): 273, which is incorporated in its entirety herein by reference. Generally, any AAV particle may be used to deliver a coding sequence described herein. However, the serotypes have different tropisms, e.g., they preferentially infect different tissues. In some embodiments, an AAV construct is a self-complementary AAV construct.

The present disclosure provides, among other things, methods of making AAV-based constructs. In some embodiments, such methods include use of host cells. In some embodiments, a host cell is a mammalian cell. A host cell may be used as a recipient of an AAV helper construct, an AAV minigene plasmid, an accessory function construct, and/or other transfer DNA associated with the production of recombinant AAVs. The term includes the progeny of an original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

Additional methods for generating and isolating AAV particles suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAV particles are separated from contaminating virus. Other systems do not require infection with helper virus to recover the AAV particles—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In yet another system, a coding sequence flanked by ITRs and rep/cap genes are introduced into insect host cells by infection with baculovirus-based constructs. Such production systems are known in the art (see generally, e.g., Zhang et al., 2009, Human Gene Therapy 20:922-929, which is incorporated in its entirety herein by reference). Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065, each of which is incorporated in its entirety herein by reference.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather should be construed to encompass any and all variations that become evident as a result of the teaching provided herein.

It is believed that one or ordinary skill in the art can, using the preceding description and following Examples, as well as what is known in the art, to make and utilize technologies of the present disclosure.

### Example 1: Construction of Viral Constructs

This example provides a description of generating a viral construct as described herein. A recombinant AAV (rAAV) particle was generated by transfection with an adenovirus-free method as used by Xiao et al. J Virol. 73(5):3994-4003, 1999, which is incorporated in its entirety herein by reference. The cis plasmids with AAV ITRs, the trans plasmid with AAV Rep and Cap genes, and a helper plasmid with an essential region from an adenovirus genome were co-transfected in HEK293 cells. The rAAV construct expressed human clarin 1 under a single construct strategy using the constructs described. AAV Anc80 capsid was prepared to encapsulate a unique rAAV clarin 1 protein encoding construct.

Those of ordinary skill in the art will readily understand that similar constructs can be made in accordance with this example. For instance, rAAV constructs that express mammalian, primate, or human clarin 1 under single, dual, or multi construct strategies can be generated. AAV serotypes 1, 2, 3, 4, 5, 6, 7, 8, 9, rh8, rh10, rh39, rh43, and Anc80 can each be prepared to encapsulate four sets of clarin 1 constructs to test (i) a concatemerization-transplicing strategy, (ii) a hybrid intronic-homologous recombination-transplicing strategy, (iii) an exonic homologous recombination strategy, as summarized by Pryadkina et al., Meth. Clin. Devel. 2:15009, 2015, which is incorporated in its entirety herein by reference, and (iv) a single construct strategy.

### Example 2: Virus Production and Purification

Dual AAV vectors were produced by transient transfection of HEK293 cells grown in multi-level cell factories. The cells were co-transduced with helper plasmids for virus production encoding serotype 6 capsid proteins. Purification of cell lysates was performed by iodixanol density-gradient ultracentrifugation, followed by a second purification and concentration step by FPLC affinity-chromatography (see, e.g., Asai et al. 2015) Nat. Neurosci. 18 1584-1593; and Tereshchenko et al. 2014 Neurobiol. Dis. 65 35-42; each of which is hereby incorporated by reference herein in its entirety). For the trans-splicing approach, the 5′ vector achieved a concentration of ˜2.8×10⁸ transducing units/μL. The 3′ vector reached ˜1.4×10⁸ transducing units/μL. For the hybrid approach, both viruses were purified simultaneously in the same solutions, reaching slightly higher virus titers.

### Example 3: Generating and Purifying Viral Particles

This example provides a description of purification of a viral construct. A recombinant AAV (rAAV) was produced using a standard triple transfection protocol and purified (e.g., by two sequential cesium chloride (CsCl) density gradients, as described by Pryadkina et al., Mol. Ther. 2:15009, 2015, which is incorporated in its entirety herein by reference. At the end of second centrifugation, 11 fractions of 500 μL were recovered from the CsCl density gradient tube and purified through dialysis in 1×PBS. The fractions were analyzed by dot blot to determine those containing rAAV genomes. The viral genome number (vg) of each preparation was determined by a quantitative real-time PCR-based titration method using primers and probe corresponding to the ITR region of the AAV construct genome (see, e.g., Bartoli et al. Gene. Ther. 13:20-28, 2006, which is incorporated in its entirety herein by reference). Those of ordinary skill in the art will readily understand that alternative production and/or purifying processes can be conducted in accordance with this example. For instance, rAAV particles may be purified using various column chromatography methods known in the art, and/or viral genomes may be quantified using alternative primer sets.

### Example 4: Formulation of Viral Particles

This example relates to the preparation of compositions comprising rAAV particles, and a physiologically acceptable solution. An rAAV was produced and purified to a titer of 4.45¹² vg/mL and was then prepared at dilutions of 6×10⁴, 1.3×10⁵, 1.8×10⁵, 4.5×10⁹, and 1.3×10¹⁰ vg/mL in physiologically acceptable solution (e.g., commercially available 1×PBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% pluronic Acid F68).

Those of ordinary skill in the art will readily understand that alternative formulations can be prepared in accordance with this example. For instance, rAAV particles may be purified to an alternative titer, prepared at alternative dilutions, and suspended in alternative suitable solutions. For example, an rAAV can be produced and purified to a quantified titer and prepared at appropriate dilutions in a physiologically acceptable solution (e.g., artificial perilymph comprising NaCl, 120 mM; KCl, 3.5 mM; CaCl2), 1.5 mM; glucose, 5.5 mM; HEPES, 20 mM which is titrated with NaOH to adjust its pH to 7.5 (total Na⁺ concentration of 130 mM) as described in Chen et al., J Controlled Rel. 110:1-19, 2005, which is incorporated in its entirety herein by reference).

### Example 5: Device Description for Suitable Delivery of Compositions to the Inner Ear

This example relates to a device suitable for the delivery of rAAV particles to the inner ear. A composition comprising rAAV particles is delivered to the cochlea of a subject using a specialized microcatheter designed for consistent and safe penetration of the round window membrane (RWM). The microcatheter is shaped such that the surgeon performing the delivery procedure can enter the middle ear cavity via the external auditory canal and contact the end of the microcatheter with the RWM. The distal end of the microcatheter may include at least one microneedle with a diameter from about 10 microns to about 1,000 microns, which produces perforations in the RWM that are sufficient to allow rAAV particles construct as described (e.g., comprising an rAAV construct of the present disclosure) to enter the cochlear perilymph of the scala tympani at a rate which does not damage the inner ear (e.g., a physiologically acceptable rate, e.g., a rate of approximately 30 μL/min to approximately 90 μL/min), but small enough to heal without surgical repair. The remaining portion of the microcatheter, proximal to the microneedle(s), is loaded with the rAAV/artificial perilymph formulation at a defined titer (e.g., approximately 1×10¹² to 5×10¹³ vg/mL). The proximal end of the microcatheter is connected to a micromanipulator that allows for precise, low volume infusions of approximately 30 μL to approximately 100 μL.

The present Example also describes that a composition comprising rAAV particles is delivered to the cochlea of a subject using a specialized microcatheter designed for consistent and safe penetration of the round window membrane (RWM) is described in “Devices, systems, and methods for delivering fluid to the inner ear,” see, e.g., WO 2021/242926, the contents of which are hereby incorporated by reference in its entirety herein.

### Example 6: Device Description for Suitable Delivery of Compositions to the Eye

This example also relates to a device suitable for the delivery of rAAV particles to the eye. A composition comprising rAAV particles is delivered to the eye of a subject using a specialized microcatheter designed for consistent and safe penetration of the retina. The microcatheter is shaped such that the surgeon performing the delivery procedure can enter the eye and contact the end of the microcatheter with the retina. The distal end of the microcatheter may include at least one microneedle with a diameter from about 10 microns to about 1,000 microns, which produces perforations in the retina that are sufficient to allow rAAV particles construct as described (e.g., comprising an rAAV construct of the present disclosure) to enter the eye at a rate which does not damage the inner ear (e.g., a physiologically acceptable rate, e.g., a rate of approximately 30 μL/min to approximately 90 μL/min), but small enough to heal without surgical repair. The remaining portion of the microcatheter, proximal to the microneedle(s), is loaded with the rAAV/artificial perilymph formulation at a defined titer (e.g., approximately 1×10¹² to 5×10¹³ vg/mL). The proximal end of the microcatheter is connected to a micromanipulator that allows for precise, low volume infusions of approximately 30 μL to approximately 100 μL.

Among other things, the present Example describes that a microscope-integrated intraoperative optical coherence tomography (OCT) is used to create a small pre-bleb before viral injection to avoid sub-RPE or suprachorodial injection. First, the hyaloid is stained and removed using a finesse loop and elevated with a soft silicone tip. A balanced salt solution is then injected into the subretinal space and an OCT scan is done to check for foveal thinning. Once the bleb is made, voretigene is introduced using a 25-gauge no-duel bore cannula. The fovea needs to be carefully monitored thereafter to avoid excessive stretching and macular hole formation. Scleral indentation can be used to rule out peripheral breaks. Finally, an air-fluid exchange is used to clear the virus. See, e.g., “OCT—Assisted Delivery of Luxturna” by Ninel Z Gregori and Janet Louise David, https://www.aao.org/clinical-video/oct-assisted-delivery-of-luxturna (Jul. 19, 2018), the contents of which are hereby incorporated by reference in its entirety.

As another example, the Orbit™ Subretinal Delivery System (Orbit SDS) (Gyroscope Therapeutics) can be used as administering or introducing the described compositions into the eye (see “Orbit Subretinal Delivery System Instructions for Use June 2020”, https://www.orbitsds.com/wp-content/uploads/2020/08/AW1009028-Rev.-D-US—IFU-No-Cut-Lines.pdf, the contents of which are hereby incorporated by reference herein in its entirety). To summarize, first, the eye site is prepared for device placement. Next, using a blade, a scleral incision is created to expose the choroid. The incision can be about 3 mm in length adjacent to the choroid. The device is then placed for advancement of the needle that flows the composition into the eye. Flexible cannula is inserted into the eye using forceps. Prior to insertion, slide the Subretinal Delivery Device body toward the eye to provide additional slack and maintain a tangential path to the eye's curvature. While grasping the center of the posterior lip of the sclerotomy with toothed forceps and pulling away from the eye, insert the flexible cannula into the sclerotomy, advancing to the first hash mark, located 5 mm from the distal tip, then stopping.

### Example 7: Expression of CLRN1 Proteins Produced from Cell Lines

This example relates to the transduction and/or transfection of exemplary constructs, and expression of exemplary proteins described herein.

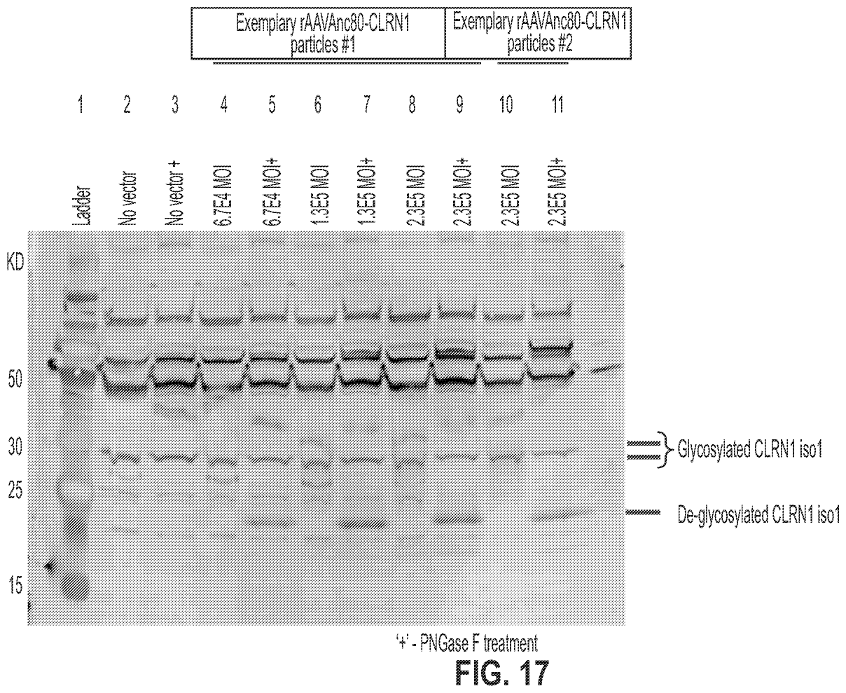

Cell lines (e.g., HEK239FT) were transduced with two types of exemplary rAAV Anc80-CLRN1 particles (an rAAV Anc80-CLRN1wt particle and an rAAV Anc80-CLRN1 codon-optimized particle), as described herein. For transfection events, HEK293FT cells were seeded overnight at 1.5E5 cells/well in a 24-well plate format with a culture volume of 500 μL. Approximately 800 ng of CLRN1 constructs (as described in Example 1) were transfected into cells using jetprime transfection reagent (Polyplus-transfection® SA). For transduction events, HEK293FT cells were seeded for 6 hours at 4×10⁴ cells/well in a 96-well plate format with a culture volume of 50 μL in the presence of 2 μM etoposide (Fisher Scientific 34120525MG), exemplary rAAV Anc80-CLRN1 particles (as described in Example 1) were added into the media at an MOI of 6.7×10⁴, 1.3×10⁵, or 2.3×10⁵ respectively (with PNGase F treatment as indicated). For transduced cells, supernatant was harvested at 72 hours post treatment for each sample. For protein expression analysis, 30 μL of samples were loaded into individual wells in a 4-12% Bis-Tris protein gel and standard western blotting procedures as known in the art were conducted. Banding patterns were determined using a fluorescent reader, with test anti-CLRN1 antibody (CLRN1 polyclonal Ab Thermofisher AB2042) as the primary detection probe, and anti-human IgG as the secondary detection probe (see FIG. 17). Glycoslated CLRN1 isoform A and de-glycosylated CLRN1 isoform A bands are shown (see FIG. 17)

### Example 8: Ex-Vivo Demonstration of CLRN1 Protein Production

This example relates to the introduction and expression analysis of rAAV constructs expressing a CLRN1 protein in mammalian cochlear explants grown in-vitro or ex-vivo. Cochlear explant culture models can provide a reliable experimental system to mimic the morphology and molecular characteristics of sensory hair cells and non-sensory supporting cells of the cochlea, in order, to study transduction and expression of rAAV particles within the intrinsic cellular environment found in-vivo.

Described herein are ex-vivo evaluations of CLRN1 protein expression from WT newborn mice cochlear explants transduced by rAAV (e.g., rAAV Anc80) particles comprising constructs rAAV-CLRN1 (as described in Example 1). In these experiments, an organ of Corti was dissected and mounted on coverslips, followed by incubation for three to four days with either vehicle or a range of doses of rAAV particles—e.g., rAAV Anc80-CLRN1 particles were transduced at 1.0×10¹⁰ vg/explant or 3.0×10¹⁰ vg/explant.

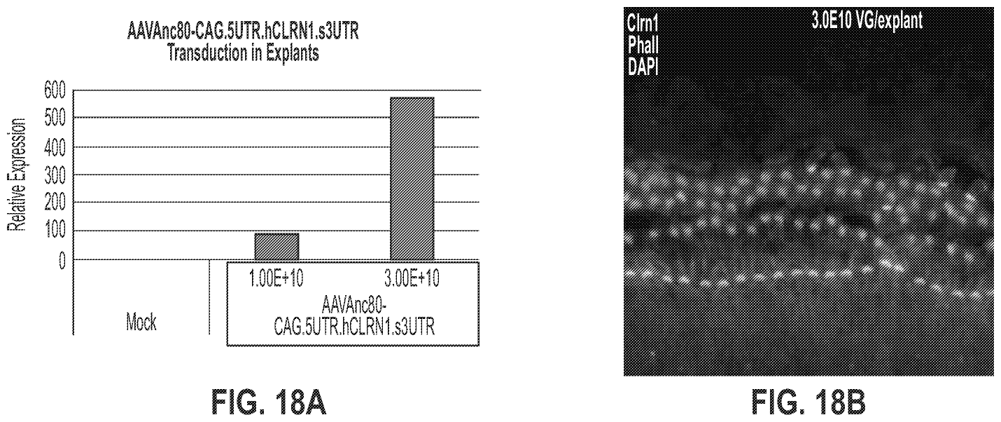

The explants were harvested and lysed for ribonucleic acid (RNA) expression analysis using quantitative real-time polymerase chain reaction (qRT-PCR) with Taqman primer-probes for GAPDH (housekeeping control) and for CLRN1 protein encoding nucleotides (mRNA products encoding CLRN1) (FIG. 18A).

CLRN1 RNA was detected in the explants receiving rAAV Anc80-CLRN1 particles, but not those in mock (FIG. 18A). These data suggest that administration of rAAV Anc80-CLRN1 particles comprising constructs encoding CLRN1 proteins to WT mouse cochlea ex-vivo yields expression of the human CLRN1 RNA from transduced cells. FIG. 18B shows a fluorescence image depicting expression of CLRN1 protein in the explants.

### EXEMPLARY EMBODIMENTS

Embodiment 1. A construct comprising a coding sequence operably linked to a promoter, wherein the coding sequence encodes a clarin 1 protein.

Embodiment 2. The construct of embodiment 1, wherein the coding sequence is a CLRN1 gene.

Embodiment 3. The construct of embodiment 2, wherein the CLRN1 gene is a primate CLRN1 gene.

Embodiment 4. The construct of embodiment 2 or 3, wherein the CLRN1 gene is a human CLRN1 gene.

Embodiment 5. The construct of embodiment 4, wherein the human CLRN1 gene comprises a nucleic acid sequence according to SEQ ID NO: 1, SEQ ID NO: 2, SEQ ID NO: 3, SEQ ID NO: 4, SEQ ID NO: 5, or SEQ ID NO: 19.

Embodiment 6. The construct of embodiment 4 or 5, wherein the human CLRN1 gene comprises a nucleic acid sequence according to SEQ ID NO: 1.

Embodiment 7. The construct of embodiment 4 or 5, wherein the human CLRN1 gene comprises a nucleic acid sequence according to SEQ ID NO: 19.

Embodiment 8 The construct of embodiment 1, wherein the clarin 1 protein is a primate clarin 1 protein.

Embodiment 9. The construct of embodiment 1 or 8, wherein the clarin 1 protein is a human clarin 1 protein.

Embodiment 10. The construct of embodiment 9, wherein the clarin 1 protein comprises an amino acid sequence according to SEQ ID NO: 10.

Embodiment 11. The construct of any one of claims 1-10, wherein the promoter is an inducible promoter, a constitutive promoter, or a tissue-specific promoter.

Embodiment 12. The construct of any one of embodiments 1-11, wherein the promotor is an inner ear cell-specific promoter.

Embodiment 13. The construct of embodiment 12, wherein the inner ear cell-specific promoter is a GJB2 promoter, a GJB6 promoter, a CLRN1 promoter, a TECTA promoter, a DFNA5 promoter, a COCH promoter, a NDP promoter, a SYN1 promoter, a GFAP promoter, a PLP promoter, a TAK1 promoter, a SOX21 promoter, a SOX2 promoter, a FGFR3 promoter, a PROX1 promoter, a GLAST1 promoter, a LGR5 promoter, a HES1 promoter, a HES5 promoter, a NOTCH1 promoter, a JAG1 promoter, a CDKN1A promoter, a CDKN1B promoter, a SOX10 promoter, a P75 promoter, a CD44 promoter, a HEY2 promoter, a LFNG promoter, or a S100b promoter.

Embodiment 14. The construct of any one of embodiments 1-11, wherein the promotor is an eye cell-specific promoter.

Embodiment 15. The construct of embodiment 14, wherein the eye cell-specific promoter is a CLRN1 promoter, a RPE65 promoter, a RLBP1 promoter, a VMD2 promoter, a IRBP promoter, a GNAT2 promoter, a PR1.7 promoter, a PR2.1 promoter, a HB569 promoter, a CAR promoter, a GRK1 promoter, a RK promoter, a B-PDE promoter, a GRM6 promoter, a Nefh promoter, a Tyh1 promoter, a SYN promoter, a GFAP promoter, or other opsin or rhodopsin promoter.

Embodiment 16. The construct of any one of embodiments 1-15, wherein the promoter is a CAG promoter, a CBA promoter, a CMV promoter, or a CB7 promoter.

Embodiment 17. The construct of embodiment 12 or 14, wherein the promoter comprises a nucleic acid sequence according to SEQ ID NO: 23.

Embodiment 18. The construct of any one of embodiments 1-17, further comprising a polyadenylation sequence.

Embodiment 19. The construct of any one of embodiments 18, wherein the polyadenylation sequence is ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG.

Embodiment 20. The construct of embodiment 18, wherein the polyadenylation sequence comprises a sequence according to SEQ ID NO: 44 or SEQ ID NO: 45.

Embodiment 21. The construct of embodiment 18, wherein the polyadenylation sequence comprises a sequence according to SEQ ID NO: 44.

Embodiment 22. The construct of any one of embodiments 1-21, further comprising two AAV inverted terminal repeats (ITRs), wherein the two AAV ITRs flank the coding sequence and promoter.

Embodiment 23. The construct of embodiment 22, wherein the two AAV ITRs are or are derived from AAV2 ITRs.

Embodiment 24. The construct of embodiment 22, wherein the two AAV ITRs comprise:
a 5′ ITR comprising a nucleic acid sequence according to SEQ ID NO: 21 and a 3′ ITR comprising a nucleic acid sequence according to SEQ ID NO: 22.

Embodiment 25. The construct of embodiment 1, wherein the construct comprises a nucleic acid sequence according to SEQ ID NO: 64.

Embodiment 26. The construct of embodiment 1, wherein the construct comprises a nucleic acid sequence according to SEQ ID NO: 68.

Embodiment 27. An AAV particle comprising the construct of any one of embodiments 1-26.

Embodiment 28. The AAV particle of embodiment 27, further comprising an AAV capsid, wherein the AAV capsid is or is derived from an AAV2, AAV3, AAV4, AAV5, AAV6, AAV7, AAV8, AAV9, AAV10, AAV-rh8, AAV-rh10, AAV-rh39, AAV-rh43 or AAV Anc80 capsid.

Embodiment 29. The AAV particle of embodiment 28, wherein the AAV capsid is an AAV Anc80 capsid.

Embodiment 30. A composition comprising the construct of any one of embodiments 1-26.

Embodiment 31. A composition comprising the AAV particle of any one of embodiments 27-29.

Embodiment 32. The composition of embodiment 30 or 31, wherein the composition is a pharmaceutical composition.

Embodiment 33. The composition of embodiment 32, further comprising a pharmaceutically acceptable carrier.

Embodiment 34. An ex vivo cell comprising a composition of any one of embodiments 30 or 31.

Embodiment 35. A method comprising, transfecting an ex vivo cell with:
(i) a construct of any one of embodiments 22-26; and
(ii) one or more helper plasmids collectively comprising an AAV Rep gene, AAV Cap gene, AAV VA gene, AAV E2a gene, and AAV E4 gene.

Embodiment 36. A method comprising:
introducing a construct of any one of embodiments 1-26, a particle of any one of embodiments 27-29, or a composition of embodiment 32 or 33 into the inner ear of a subject.

Embodiment 37. A method of treatment comprising:
introducing a construct of any one of embodiments 1-26, a particle of any one of embodiments 27-29, or a composition of embodiment 32 or 33 into the inner ear of a subject.

Embodiment 38. A method of treating Usher syndrome type III comprising:
introducing a construct of any one of embodiments 1-26, a particle of any one of embodiments 27-29, or a composition of embodiment 32 or 33 into the inner ear of a subject.

Embodiment 39. A method of treating hearing loss comprising:
introducing a construct of any one of embodiments 1-26, a particle of any one of embodiments 27-29, or a composition of embodiment 32 or 33 into the inner ear of a subject.

Embodiment 40. A method of treating deafness comprising:
introducing a construct of any one of embodiments 1-26, a particle of any one of embodiments 27-29, or a composition of embodiment 32 or 33 into the inner ear of a subject.

Embodiment 41. The method of any one of embodiments 36-40, wherein the composition of embodiment 32 or 33 is introduced into the cochlea of the subject.

Embodiment 42. The method of any one of embodiments 36-41, wherein the composition of embodiment 32 or 33 is introduced via a round window membrane injection.

Embodiment 43. The method of any one of embodiments 36-42, further comprising measuring a hearing level of the subject.

Embodiment 44. The method of embodiment 43, wherein a hearing level is measured by performing an auditory brainstem response (ABR) test.

Embodiment 45. The method of embodiment 43 or 44, further comprising comparing the hearing level of the subject to a reference hearing level.

Embodiment 46. The method of embodiment 45, wherein the reference hearing level is a published or historical reference hearing level.

Embodiment 47. The method of embodiment 45, wherein the hearing level of the subject is measured after the composition of embodiment 32 or 33 is introduced, and the reference hearing level is a hearing level of the subject that was measured before the composition of embodiment 32 or 33 was introduced.

Embodiment 48. The method of any one of embodiments 36-47, further comprising measuring a level of clarin 1 protein in the subject.

Embodiment 49. The method of embodiment 48, wherein the level of clarin 1 protein is measured in the inner ear of the subject.

Embodiment 50. The method of embodiment 48 or 49, wherein the level of clarin 1 protein is measured in the cochlea of the subject.

51. The method of any one of embodiments 48-50, further comprising comparing the level of clarin 1 protein in the subject to a reference clarin 1 protein level.

Embodiment 52. The method of embodiment 49, wherein the reference hearing level is a published or historical reference clarin 1 protein level.

Embodiment 53. The method of embodiment 45, wherein the level of clarin 1 protein in the subject is measured after the composition of embodiment 32 or 33 is introduced, and the reference clarin 1 protein level is a clarin 1 protein level of the subject that was measured before the composition of embodiment 32 or 33 was introduced.

Embodiment 54. A method comprising:
introducing a construct of any one of embodiments 1-26, a particle of any one of embodiments 27-29, or a composition of embodiment 32 or 33 into the eye of a subject.

Embodiment 55. A method of treatment comprising: introducing a construct of any one of embodiments 1-26, a particle of any one of embodiments 27-29, or a composition of embodiment 32 or 33 into the eye of a subject.

Embodiment 56. A method of treating Usher syndrome type III comprising: introducing a construct of any one of embodiments 1-26, a particle of any one of embodiments 27-29, or a composition of embodiment 32 or 33 into the eye of a subject.

Embodiment 57. A method of treating vision loss comprising:
introducing a construct of any one of embodiments 1-26, a particle of any one of embodiments 27-29, or a composition of embodiment 32 or 33 into the eye of a subject.

Embodiment 58. A method of treating retinitis pigmentosa comprising:
introducing a construct of any one of embodiments 1-26, a particle of any one of embodiments 27-29, or a composition of embodiment 32 or 33 into the eye of a subject.

Embodiment 59. The method of any one of embodiments 54-59, wherein the composition of embodiment 32 or 33 is introduced via an eye injection.

Embodiment 60. The method of any one of embodiments 54-59, further comprising measuring a vision level of the subject.

Embodiment 61. The method of embodiment 60, wherein a hearing level is measured by performing a visual acuity test.

Embodiment 62. The method of embodiment 60 or 61, further comprising comparing the vision level of the subject to a reference vision level.

Embodiment 63. The method of embodiment 62, wherein the reference vision level is a published or historical reference vision level.

Embodiment 64. The method of embodiment 62, wherein the vision level of the subject is measured after the composition of embodiment 32 or 33 is introduced, and the reference vision level is a vision level of the subject that was measured before the composition of embodiment 32 or 33 was introduced.

Embodiment 65. The method of any one of embodiments 54-64, further comprising measuring a level of clarin 1 protein in the subject.

Embodiment 66. The method of embodiment 65, wherein the level of clarin 1 protein is measured in the eye of the subject.

Embodiment 67. The method of any one of embodiments 65 or 66, further comprising comparing the level of clarin 1 protein in the subject to a reference clarin 1 protein level.

Embodiment 68. The method of embodiment 67, wherein the reference vision level is a published or historical reference clarin 1 protein level.

Embodiment 69. The method of embodiment 68, wherein the level of clarin 1 protein in the subject is measured after the composition of embodiment 32 or 33 is introduced, and the reference clarin 1 protein level is a clarin 1 protein level of the subject that was measured before the composition of embodiment 32 or 33 was introduced.

Embodiment 70. Use of a construct of any one of embodiments 1-26, an AAV particle of any one of embodiments 27-29, or a composition of any one of embodiments 30-33 for the treatment of hearing loss in a subject suffering from or at risk of hearing loss.

Embodiment 71. Use of a construct of any one of embodiments 1-26, an AAV particle of any one of embodiments 27-29, or a composition of any one of embodiments 30-33 in the manufacture of a medicament for the treatment of hearing loss.

Embodiment 72. Use of a construct of any one of embodiments 1-26, an AAV particle of any one of embodiments 27-29, or a composition of any one of embodiments 30-33 for the treatment of vision loss in a subject suffering from or at risk of vision loss.

Embodiment 73. Use of a construct of any one of embodiments 1-26, an AAV particle of any one of embodiments 27-29, or a composition of any one of embodiments 30-33 in the manufacture of a medicament for the treatment of vision loss.

Embodiment 74. Use of a construct of any one of embodiments 1-26, an AAV particle of any one of embodiments 27-29, or a composition of any one of embodiments 30-33 for the treatment of Usher syndrome type III in a subject suffering from or at risk of Usher syndrome type III.

Embodiment 75. Use of a construct of any one of embodiments 1-26, an AAV particle of any one of embodiments 27-29, or a composition of any one of embodiments 30-33 for the treatment of retinitis pigmentosa in a subject suffering from or at risk of retinitis pigmentosa.

Embodiment 76. Use of a construct of any one of embodiments 1-26, an AAV particle of any one of embodiments 27-29, or a composition of any one of embodiments 30-33 in the manufacture of a medicament for the treatment of Usher syndrome type III.

Embodiment 77. Use of a construct of any one of embodiments 1-26, an AAV particle of any one of embodiments 27-29, or a composition of any one of embodiments 30-33 in the manufacture of a medicament for the treatment of retinitis pigmentosa.

Embodiment 78. A construct of any one of embodiments 1-26, an AAV particle of any one of embodiments 27-29, or a composition of any one of embodiments 30-33 for use as a medicament.

Embodiment 79. A construct of any one of embodiments 1-26, an AAV particle of any one of embodiments 27-29, or a composition of any one of embodiments 30-33 for use in the treatment of hearing loss.

Embodiment 80. A construct of any one of embodiments 1-26, an AAV particle of any one of embodiments 27-29, or a composition of any one of embodiments 30-33 for use in the treatment of vision loss.

Embodiment 81. A construct of any one of embodiments 1-26, an AAV particle of any one of embodiments 27-29, or a composition of any one of embodiments 30-33 for use in the treatment of Usher syndrome type III.

Embodiment 82. A construct of any one of embodiments 1-26, an AAV particle of any one of embodiments 27-29, or a composition of any one of embodiments 30-33 for use in the treatment of retinitis pigmentosa.

Embodiment 83. A kit comprising a composition of any one of embodiments 30-33.

Embodiment 84. The kit of embodiment 83, wherein the composition is pre-loaded in a device.

Embodiment 85. The kit of embodiment 84, wherein the device is a microcatheter.

Embodiment 86. The kit of embodiment 85, wherein the microcatheter is shaped such that it can enter the middle ear cavity via the external auditory canal and contact the end of the microcatheter with the RWM.

Embodiment 87. The kit of embodiment 85 or 86, wherein a distal end of the microcatheter is comprised of at least one microneedle with diameter of between 10 and 1,000 microns.

Embodiment 88. The kit of any one of embodiments 83-87, further comprising a device.

Embodiment 89. The kit of embodiment 88, wherein the device is a device described in FIGS. 21-24 or a device as described in WO 2021/242926.

Embodiment 90. The kit of embodiment 89, wherein the device comprises a needle comprising a bent portion and an angled tip.

1. An adeno-associated virus (AAV) particle comprising a construct, wherein the construct comprises:
(i) a 5′ inverted terminal repeat (ITR);
(ii) a cytomegalovirus (CMV) enhancer;
(iii) a chicken β-actin (CBA) promoter;
(iv) a chimeric intron;
(v) a 5′ untranslated region (UTR);
(vi) a coding sequence comprising a sequence according to SEQ ID NO: 1, wherein the coding sequence is operably linked to the CBA promoter, and wherein the coding sequence encodes a clarin 1 protein;
(vii) a 3′ UTR;
(viii) a polyadenylation sequence; and
(ix) a 3′ ITR.

2.-9. (canceled)

10. The AAV particle of claim 1, wherein the 5′ ITR and the 3′ ITR flank the CMV enhancer and 3′ UTR, respectively.

11. The AAV particle of claim 1, wherein the construct comprises a nucleic acid sequence according to SEQ ID NO: 64 or 68.

12. (canceled)

13. The AAV particle of claim 1, further comprising an AAV capsid, wherein the AAV capsid is or is derived from an AAV Anc80 capsid.

14. A composition comprising the AAV particle of claim 1.

15. The composition of claim 14, wherein the composition is a pharmaceutical composition, wherein the pharmaceutical composition comprises a pharmaceutically acceptable carrier.

16. An ex vivo cell comprising a composition of claim 1.

17. A method comprising, transfecting an ex vivo cell with:
(i) a construct of claim 49; and
(ii) one or more helper plasmids collectively comprising an AAV Rep gene, AAV Cap gene, AAV VA gene, AAV E2a gene, and AAV E4 gene.

18. (canceled)

19. A method of treatment comprising:
introducing an AAV particle of claim 1 into the inner ear or the eye of a subject.

20. A method of treating Usher syndrome type III comprising:
introducing an AAV particle of claim 1, into the inner ear or the eye of a subject.

21. A method of treating hearing loss comprising:
introducing an AAV particle of claim 1 into the inner ear of a subject.

22. A method of treating deafness comprising:
introducing an AAV particle of claim 1 into the inner ear of a subject.

23. A method of treating vision loss comprising:
introducing an AAV particle of claim 1 into the eye of a subject.

24. A method of treating retinitis pigmentosa comprising:
introducing an AAV particle of claim 1 into the eye of a subject.

25.-37. (canceled)

38. A kit comprising a composition of claim 14.

39. The AAV particle of claim 1, wherein the 5′ ITR comprises a sequence according to SEQ ID NO: 21.

40. The AAV particle of claim 1, wherein the 3′ ITR comprises a sequence according to SEQ ID NO: 22.

41. The AAV particle of claim 1, wherein the CMV enhancer comprises a sequence according to SEQ ID NO: 38.

42. The AAV particle of claim 1, wherein the CBA promoter comprises a sequence according to SEQ ID NO: 23.

43. The AAV particle of claim 1, wherein the chimeric intron comprises a sequence according to SEQ ID NO: 39.

44. The AAV particle of claim 1, wherein the 5′ UTR comprises a sequence according to SEQ ID NO: 40.

45. The AAV particle of claim 1, wherein the clarin 1 protein comprises an amino acid sequence according to SEQ ID NO: 10.

46. The AAV particle of claim 1, wherein the 3′ UTR comprises a sequence according to SEQ ID NO: 41.

47. The AAV particle of claim 1, wherein the polyadenylation sequence comprises a sequence according to SEQ ID NO: 44.

48. An adeno-associated virus (AAV) particle comprising a construct, wherein the construct comprises:
(i) a 5′ inverted terminal repeat (ITR);
(ii) a cytomegalovirus (CMV) enhancer;
(iii) a chicken β-actin (CBA) promoter;
(iv) a chimeric intron;
(v) a 5′ untranslated region (UTR);
(vi) a coding sequence comprising a sequence according to SEQ ID NO: 19, wherein the coding sequence is operably linked to the CBA promoter, and wherein the coding sequence encodes a clarin 1 protein;
(vii) a 3′ UTR;
(viii) a polyadenylation sequence; and
(ix) a 3′ ITR.

49. A construct comprising:
(i) a 5′ inverted terminal repeat (ITR);
(ii) a cytomegalovirus (CMV) enhancer;
(iii) a chicken β-actin (CBA) promoter;
(iv) a chimeric intron;
(v) a 5′ untranslated region (UTR);
(vi) a coding sequence comprising a sequence according to SEQ ID NO: 1, wherein the coding sequence is operably linked to the CBA promoter, and wherein the coding sequence encodes a clarin 1 protein;
(vii) a 3′ UTR;
(viii) a polyadenylation sequence; and
(ix) a 3′ ITR.

50. A construct comprising:
(i) a 5′ inverted terminal repeat (ITR);
(ii) a cytomegalovirus (CMV) enhancer;
(iii) a chicken β-actin (CBA) promoter;
(iv) a chimeric intron;
(v) a 5′ untranslated region (UTR);
(vi) a coding sequence comprising a sequence according to SEQ ID NO: 19, wherein the coding sequence is operably linked to the CBA promoter, and wherein the coding sequence encodes a clarin 1 protein;
(vii) a 3′ UTR;
(viii) a polyadenylation sequence; and
(ix) a 3′ ITR.
